(19) United States
(12) Patent Application Publication
(10) Pub. No.: US 2018/0112203 A1
(43) Pub. Date: Apr. 26, 2018

(54) AMYLASE WITH MALTOGENIC PROPERTIES

(71) Applicant: DANISCO US INC., Palo Alto,, CA (US)

(72) Inventors: RICHARD R. BOTT, BURLINGAME, CA (US); LING HUA, HOCKESSIN, DE (US); ZHEN QIAN, SHANGHAI (CN); CHRISTOPHER L. RIFE, REDWOOD CITY, CA (US); JAYARAMA K. SHETTY, PLEASANTON, CA (US); ZHONGMEI TANG, SHANGHAI (CN); ZHEYONG YU, SHANGHAI (CN); BO ZHANG, SHANGHAI (CN)

(73) Assignee: DANISCO US INC., PALO ALTO, CA (US)

(21) Appl. No.: 14/441,617

(22) PCT Filed: Nov. 15, 2013
(86) PCT No.: PCT/US2013/070237

§ 371 Date: May 8, 2015

(30) Foreign Application Priority Data
Nov. 20, 2012 (CN) PCT/CN2012/084883

(51) Int. Cl. C12N 9/26

(52) U.S. Cl. CPC C12N 9/2411; C12Y 302/01133

(57) ABSTRACT

The present teachings provide an amylase with maltogenic properties. Nucleic acids encoding the maltogenic amylase and variants thereof, expression vectors, formulations, and host cells are also provided. Additional embodiments of the present teachings provide various methods of use and methods of manufacturing.

### CROSS-REFERENCE TO RELATED APPLICATIONS

This application claims benefit to international patent application no. PCT/CN2012/084883, filed on Nov. 20, 2012, the contents of which are hereby incorporated by reference in its entirety.

### FIELD OF THE INVENTION

The present teachings provide composition and methods relating to novel maltogenic amylases.

### BACKGROUND

Starch is a mixture of amylose (15-30% w/w) and amylopectin (70-85% w/w). Amylose is composed of linear chains of α-1,4-linked glucose units having a molecular weight (MW) from about 60,000 to about 800,000. Amylopectin is a branched polymer containing α-1,6 branch points every 24-30 glucose units; its MW may be as high as 100 million.

Sugars from starch, in the form of concentrated dextrose syrups, are currently produced by an enzyme catalyzed process involving: (1) liquefaction (or viscosity reduction) of solid starch with an α-amylase into dextrins having an average degree of polymerization of about 7-10, and (2) saccharification of the resulting liquefied starch (i.e. starch hydrolysate) with amyloglucosidase (also called glucoamylase or GA). The resulting syrup has a high glucose content. Much of the glucose syrup that is commercially produced is subsequently enzymatically isomerized to a dextrose/fructose mixture known as isosyrup. The resulting syrup also may be fermented with microorganisms, such as yeast, to produce commercial products including ethanol, citric acid, lactic acid, succinic acid, itaconic acid, monosodium glutamate, gluconates, lysine, other organic acids, other amino acids, and other biochemicals, for example. Fermentation and saccharification can be conducted simultaneously (i.e., an SSF process) to achieve greater economy and efficiency.

Alpha-amylases hydrolyze starch, glycogen, and related polysaccharides by cleaving internal α-1,4-glucosidic bonds at random. Alpha amylases, particularly from Bacilli, have been used for a variety of different purposes, including starch liquefaction and saccharification, textile desizing, starch modification in the paper and pulp industry, brewing, baking, production of syrups for the food industry, production of feedstocks for fermentation processes, and in animal feed to increase digestibility. These enzymes can also be used to remove starchy soils and stains during dishwashing and laundry washing.

Maltose, a di-saccharide composed of two D-glucopyranoses joined by a β-1,4′-glycosidic bond, has high commercial value in applications for the food/frozen foods, baking, brewing and beverage industries. Maltose is also a substrate for production of the non-caloric sugar sweetener, maltitol. High purity maltose or pure maltose is an active component of intravenous injection liquids for diabetic patients. Commercial processes for the production of syrup containing different levels of maltose content, i.e. <50% maltose (high conversion or low maltose syrup), 50-55% maltose (high maltose syrup), 70-75% maltose (very high maltose) and >80% maltose (ultra high maltose) have been established depending on the applications. A common factor for these processes is that they involve a dual enzyme process with two different steps, i.e. liquefaction and saccharification.

Historically, two enzyme steps are involved in the hydrolysis of starch to produce glucose or maltose syrups. The first step is a liquefaction step at high temperature, >95° C. and the second step is a saccharification step. In maltose production the second step is called malto-saccharification and usually takes place at a temperature at or below 60° C. In the liquefaction step, the insoluble starch granules are slurried in water, gelatinized with heat and hydrolyzed by a thermostable alpha-amylase (EC.3.2.1.1, α-1,4′-D-glucan glucanohydrolase) from Bacillus species, often in the presence of added calcium. Bacterial derived thermostable alpha-amylases from Bacillus licheniformis (for example, SPEZYME® FRED from DuPont-Genencor or Termamyl® L-120 from Novozymes), Bacillus stearothermophilus (for example SPEZYME® XTRA from DuPont-Genencor, Termamyl® SC, and Termamyl® SUPRA from Novozymes) or blends of Bacillus licheniformis and Bacillus stearothermophilus (for example Clearflow™ AA from DuPont-Genencor or Liquozyme® Supra from Novozymes) are used to first liquefy the starch at high temperature, >95° C. at pH 5.2-6.5 to a low DE (dextrose equivalent) soluble starch hydrolysate. In the malto-saccharification step, generally maltogenic enzymes such as a fungal alpha-amylase (for example, CLARASE® L from DuPont-Genencor or Fungamyl® 800L from Novozymes), a plant beta-amylase (for example, OPTIMALT® BBA from DuPont-Genencor or Betalase 1500L from Senson) are used at a much lower temperature to further hydrolyse the soluble starch hydrolysate. For maltose syrup containing greater than 60% maltose, a debranching enzyme like pullulanase (for example OPTIMAX® L-1000 from DuPont-Genencor, Promozyme® D2 from Novozymes or Promozyme® D6 from Novozymes) is added during malto-saccharification of liquefied starch.

### SUMMARY

In some embodiments, the present teachings provide an isolated nucleic acid comprising the nucleotide sequence of SEQ ID NO: 1 or of a degenerate variant of SEQ ID NO: 1.

In some embodiments, the present teachings provide an isolated nucleic acid comprising a sequence that encodes a polypeptide consisting of the amino acid sequence of SEQ ID NO: 3.

In some embodiments, the present teachings provide an isolated nucleic acid comprising a sequence that hybridizes under stringent conditions to a hybridization probe the nucleotide sequence of which consists of SEQ ID NO: 1, or the complement of SEQ ID NO: 1.

In some embodiments, the present teachings provide an isolated nucleic acid comprising a sequence at least 66%, 67%, 68%, 69%, 70%, 75%, 80%, 85%, 90%, 95%, 98%, 99%, or 99.5% identical to SEQ ID NO: 1. In some embodiments, the present teachings provide such an isolated nucleic acid wherein the nucleic acid encodes a polypeptide that has starch hydrolysis activity.

In some embodiments, the present teachings provide an isolated nucleic acid comprising a sequence that encodes a polypeptide at least 66%, 67%, 68%, 69%, 70%, 75%, 80%, 85%, 90%, 95%, 98%, 99%, or 99.5% identical to SEQ ID NO: 3, wherein the polypeptide has starch hydrolysis activity.

In some embodiments, the present teachings provide an isolated nucleic acid comprising a sequence that encodes a polypeptide comprising the sequence of SEQ ID NO: 3, or SEQ ID NO: 3 with up to 50 conservative amino acid substitutions, wherein the polypeptide has starch hydrolysis activity.

In some embodiments, the present teachings provide a purified polypeptide, the amino acid sequence of which comprises a sequence at least 66%, 67%, 68%, 69%, 70%, 75%, 80%, 85%, 90%, 95%, 98%, 99%, or 99.5% identical to SEQ ID NO: 3.

In some embodiments, the present teachings provide a purified polypeptide comprising the amino acid sequence of SEQ ID NO: 3, but with 0 to 20 conservative amino acid substitutions.

In some embodiments, the present teachings provide an expression vector comprising the nucleic acid sequence of any of claims 1-6 operably linked to an expression control sequence. In some embodiments, the present teachings provide a cultured cell comprising such a vector. In some embodiments, the present teachings provide cultured cell comprising any of the nucleic acids of the present teachings, operably linked to an expression control sequence. In some embodiments, the present teachings provide a cultured cell transfected with any of the vectors provided by the present teachings, or a progeny of said cell, wherein the cell expresses the nucleic acid to form a polypeptide.

In some embodiments, the present teachings provide a method of producing a protein, the method comprising culturing the cells provided by the present teachings under conditions permitting expression of the polypeptide.

In some embodiments, the present teachings provide a method of using the polypeptide of the present teachings, the method comprising including the polypeptide in any of: starch liquefaction, starch saccharification, textile desizing, starch modification in the paper and pulp industry, brewing, baking, production of syrups for the food industry, production of feedstocks for fermentation processes, animal feed, and, removal of starchy soils and/or stains during dishwashing and/or laundry washing.

In some embodiments, the present teachings provide a composition comprising the polypeptide of the present teachings, and at least one accessory enzyme selected from the group consisting of phytase, protease, pullulanase, β-amylase, isoamylase, a different amylase, alpha-glucosidase, cellulase, xylanase, hemicellulase, beta-glucosidase, transferase, pectinase, lipase, cutinase, esterase, choline oxidases, peroxidases/oxidases, pectate lyases, mannanases, cutinases, laccases, phospholipases, lysophospholipases, acyltransferases, perhydrolases, arylesterases, and redox enzymes.

These and other aspects and embodiments of the compositions and methods of the present teachings will be apparent from the present description and drawings.

### BRIEF DESCRIPTION OF THE DRAWINGS

FIG. 1, panels A and B depict some illustrative data according to some embodiments of the present teachings.

FIG. 2, panels A and B depict some illustrative data according to some embodiments of the present teachings.

FIG. 3 depicts some illustrative data according to some embodiments of the present teachings.

FIG. 4 depicts some illustrative data according to some embodiments of the present teachings.

FIG. 5 depicts some illustrative data according to some embodiments of the present teachings.

FIG. 6 depicts some illustrative data according to some embodiments of the present teachings.

FIG. 7 depicts some illustrative data according to some embodiments of the present teachings.

FIG. 8 depicts some illustrative data according to some embodiments of the present teachings.

FIG. 9 depicts an illustrative cloning map according to some embodiments of the present teachings.

### BRIEF DESCRIPTION OF THE SEQUENCES

SEQ ID NO: 1 sets forth the full-length nucleotide sequence for AmyMG.

SEQ ID NO: 2 sets forth the nucleotide sequence for the native signal sequence for AmyMG.

SEQ ID NO: 3 sets forth the full-length amino acid sequence for AmyMG.

SEQ ID NO: 4 sets forth the amino acid sequence for the native signal peptide.

SEQ ID NO: 5 sets forth the aprE signal nucleic acid sequence (underlined)+AGK nucleic acid sequence (italics).

SEQ ID NO: 6 sets forth the aprE signal amino acid sequence (underlined)+AGK amino acid sequence (italics).

### DETAILED DESCRIPTION

Described are compositions and methods relating to maltogenic amylase enzymes. This enzyme was discovered and analyzed by a combination of experimental approaches, as detailed in the Examples. Exemplary applications for the variant amylase enzymes are for starch liquefaction and saccharification, for cleaning starchy stains in laundry, dishwashing, and other applications, for textile processing (e.g., desizing), in animal feed for improving digestibility, and for baking and brewing. These and other aspects of the compositions and methods are described in detail, below.

Prior to describing the various aspects and embodiments of the present compositions and methods, the following definitions and abbreviations are described.

### Definitions and Abbreviations

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art. The following terms are provided below.

### Abbreviations and Acronyms

The following abbreviations/acronyms, when and if present, have the following meanings unless otherwise specified:
ABTS 2,2-azino-bis-3-ethylbenzothiazoline-6-sulfonic acid
AE or AEO alcohol ethoxylate
AES or AEOS alcohol ethoxysulfate
AkAA Aspergillus kawachii α-amylase
AnGA Aspergillus nigerglucoamylase
AOS α-olefinsulfonate
AS alkyl sulfate
cDNA complementary DNA
CMC carboxymethylcellulose
DE dextrose equivalent
DNA deoxyribonucleic acid
DPn degree of saccharide polymerization having n subunits
ds or DS dry solids
DTMPA diethylenetriaminepentaacetic acid
EC Enzyme Commission
EDTA ethylenediaminetetraacetic acid
EO ethylene oxide (polymer fragment)
EOF End of Fermentation
GA glucoamylase
GAU/g ds glucoamylase activity unit/gram dry solids
HFCS high fructose corn syrup
HgGA Humicola grisea glucoamylase
IPTG isopropyl β-D-thiogalactoside
IRS insoluble residual starch
kDa kiloDalton
LAS linear alkylbenzenesulfonate
LAT, BLA B. licheniformis amylase
MW molecular weight
MWU modified Wohlgemuth unit; 1.6×10⁻⁵ mg/MWU=unit of activity
NCBI National Center for Biotechnology Information
NOBS nonanoyloxybenzenesulfonate
NTA nitriloacetic acid
OxAm Purastar HPAM 5000L (Danisco US Inc.)
PAHBAH p-hydroxybenzoic acid hydrazide
PEG polyethyleneglycol
pl isoelectric point
PI performance index
ppm parts per million, e.g., μg protein per gram dry solid
PVA poly(vinyl alcohol)
PVP poly(vinylpyrrolidone)
RCF relative centrifugal/centripetal force (La, ×gravity)
RNA ribonucleic acid
SAS alkanesulfonate
SDS-PAGE sodium dodecyl sulfate polyacrylamide gel electrophoresis
SSF simultaneous saccharification and fermentation
SSU/g solid soluble starch unit/gram dry solids
sp. species
TAED tetraacetylethylenediamine
Tm melting temperature
TrGA Trichoderma reesei glucoamylase
w/v weight/volume
w/w weight/weight
v/v volume/volume
wt % weight percent
° C. degrees Centigrade
H₂O water
dH₂O or DI deionized water
dIH₂O deionized water, Milli-Q filtration
g or gm grams
μg micrograms
mg milligrams
kg kilograms
μL and μl microliters
mL and ml milliliters
mm millimeters
μm micrometer
M molar
mM millimolar
μM micromolar
U units
sec seconds
min(s) minute/minutes
hr(s) hour/hours
DO dissolved oxygen
Ncm Newton centimeter
ETOH ethanol
eq. equivalents
N normal
uPWA variant α-amylase derived from Pyrococcus woesei
PWA α-amylase from Pyrococcus woesei
MWCO molecular weight cut-off
SSRL Stanford Synchrotron Radiation Lightsource
PDB Protein Database
CAZy Carbohydrate-Active Enzymes database
Tris-HCl tris(hydroxymethyl)aminomethane hydrochloride
HEPES 4-(2-hydroxyethyl)-1-piperazineethanesulfonic acid

### Definitions

The term “maltogenic amylase” refers to enzymes that are capable of producing significant amounts of maltose from starch or hydrolyzed starch. There are several enzymes falling under this definition. Some examples are:
1) Fungal alpha-amylases include those obtained from filamentous fungal strains including but not limited to strains of Aspergillus (e.g., A. Niger, A. kawachi, and A. oryzae); Trichoderma sp. (e.g. Trichoderma reesie alpha-amylase, disclosed in EP 2132307), Rhisopus sp., Mucor sp., and Penicillium sp. Commercial fungal alpha-amylase from Aspergillus oryzae are CLARASE® L from DuPont-Genencor and Fungamyl® 800L from Novozymes.
2) Acid stable fungal amylase from Aspergillus niger (For example, from Shin Nihon Chemicals).
3) Beta-amylases are found in plant materials like wheat, barley, rye, shorgum, soy, sweet potato, rice and microorganisms like Bacillus cereus, Bacillus polymixa, Bacillus megaterium, Arabidopsis thaliana. The most common commercial beta-amylases are derived from barley and are sold under trade the names OPTIMALT® BBA by DuPont-Genencor and Betalase 1500L by Senson. A commercial soy beta-amylase is β-amylase#1500S from Nagase ChemteX Corporation.
4) Maltogenic amylases (E.C. 3.2.1.133) are produced by microorganisms Bacillus subtilis, Geobacillus stearothermophilus, Bacillus thermoalkalophilus, Lactobacillus gasseri, Thermus sp. Commercial maltogenic amylases include but not limited to Maltogenase® L from Novozymes, Veron® XTENDER from AB Enzymes and MAX-LIFE™ P100 from DuPont-Danisco.
5) Maltogenic amylases provided by the present teachings, as illustrated for example by SEQ ID NO: 1 and SEQ ID NO: 3, and variants thereof taught herein.

As used herein, the term “Enzyme units” refers to the amount of product formed per time under the specified conditions of the assay. For example, a “glucoamylase activity unit” (GAU) is defined as the amount of enzyme that produces 1 g of glucose per hour from soluble starch substrate (4% DS) at 60° C., pH 4.2. A “soluble starch unit” (SSU) is the amount of enzyme that produces 1 mg of glucose per minute from soluble starch substrate (4% DS) at pH 4.5, 50° C. As another example, maltogenic amylase activity can be measured in degrees Diastatic Power (DP°) Units. This assay is based on a 30-min hydrolysis of a starch substrate at pH 4.6 and 20° C. The reducing sugar groups produced on hydrolysis are measured in a titrimetric procedure using alkaline ferricyanide. One unit of diastase activity, expressed as degrees DP (DP°), is defined as the amount of enzyme, contained in 0.1 ml of a 5% solution of the sample enzyme preparation, that will produce sufficient reducing sugars to reduce 5 mL of Fehling's solution when the sample is incubated with 100 mL of the substrate for 1 hour at 20° C.

The term “starch” refers to any material comprised of the complex polysaccharide carbohydrates of plants, comprised of amylose and amylopectin with the formula (C₆H₁₀O₅)ₓ, wherein X can be any number. The term includes plant-based materials such as grains, cereal, grasses, tubers and roots, and more specifically materials obtained from wheat, barley, corn, rye, rice, sorghum, brans, cassava, millet, milo, potato, sweet potato, and tapioca. The term “starch” includes granular starch. The term “granular starch” refers to raw, i.e., uncooked starch, e.g., starch that has not been subject to gelatinization.

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

Reference to the wild-type polypeptide is understood to include the mature form of the polypeptide. A “mature” polypeptide or variant, thereof, is one in which a signal sequence is absent, for example, cleaved from an immature form of the polypeptide during or following expression of the polypeptide.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

In the case of the present maltogenic amylases, “activity” refers to maltogenic amylase activity, which can be measured as described herein.

The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding an amylase is a recombinant vector.

The terms “recovered,” “isolated,” and “separated,” refer to a compound, protein (polypeptides), cell, nucleic acid, amino acid, or other specified material or component that is removed from at least one other material or component with which it is naturally associated as found in nature. An “isolated” polypeptides, thereof, includes, but is not limited to, a culture broth containing secreted polypeptide expressed in a heterologous host cell.

The term “purified” refers to material (e.g., an isolated polypeptide or polynucleotide) that is in a relatively pure state, e.g., at least about 90% pure, at least about 95% pure, at least about 98% pure, or even at least about 99% pure.

The term “enriched” refers to material (e.g., an isolated polypeptide or polynucleotide) that is in about 50% pure, at least about 60% pure, at least about 70% pure, or even at least about 70% pure.

The terms “thermostable” and “thermostability,” with reference to an enzyme, refer to the ability of the enzyme to retain activity after exposure to an elevated temperature. The thermostability of an enzyme, such as a maltogenic amylase enzyme, is measured by its half-life (t_{1/2}) given in minutes, hours, or days, during which half the enzyme activity is lost under defined conditions. The half-life may be calculated by measuring residual amylase activity following exposure to (i.e., challenge by) an elevated temperature.

A “pH range,” with reference to an enzyme, refers to the range of pH values under which the enzyme exhibits catalytic activity.

The terms “pH stable” and “pH stability,” with reference to an enzyme, relate to the ability of the enzyme to retain activity over a wide range of pH values for a predetermined period of time (e.g., 15 min., 30 min., 1 hour).

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

“Hybridization” refers to the process by which one strand of nucleic acid forms a duplex with, i.e., base pairs with, a complementary strand, as occurs during blot hybridization techniques and PCR techniques. Stringent hybridization conditions are exemplified by hybridization under the following conditions: 65° C. and 0.1×SSC (where 1×SSC=0.15 M NaCl, 0.015 M Na₃ citrate, pH 7.0). Hybridized, duplex nucleic acids are characterized by a melting temperature (Tₘ), where one half of the hybridized nucleic acids are unpaired with the complementary strand. Mismatched nucleotides within the duplex lower the Tₘ. Very stringent hybridization conditions involve 68° C. and 0.1×SSC

A “synthetic” molecule is produced by in vitro chemical or enzymatic synthesis rather than by an organism.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection”, “transformation” or “transduction,” as known in the art.

A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

The term “expression” refers to the process by which a polypeptide is produced based on a nucleic acid sequence. The process includes both transcription and translation.

A “selective marker” or “selectable marker” refers to a gene capable of being expressed in a host to facilitate selection of host cells carrying the gene. Examples of selectable markers include but are not limited to antimicrobials (e.g., hygromycin, bleomycin, or chloramphenicol) and/or genes that confer a metabolic advantage, such as a nutritional advantage on the host cell.

A “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

“Biologically active” refers to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein.

As used herein, “water hardness” is a measure of the minerals (e.g., calcium and magnesium) present in water.

A “swatch” is a piece of material such as a fabric that has a stain applied thereto. The material can be, for example, fabrics made of cotton, polyester or mixtures of natural and synthetic fibers. The swatch can further be paper, such as filter paper or nitrocellulose, or a piece of a hard material such as ceramic, metal, or glass. For amylases, the stain is starch based, but can include blood, milk, ink, grass, tea, wine, spinach, gravy, chocolate, egg, cheese, clay, pigment, oil, or mixtures of these compounds.

A “smaller swatch” is a section of the swatch that has been cut with a single hole punch device, or has been cut with a custom manufactured 96-hole punch device, where the pattern of the multi-hole punch is matched to standard 96-well microtiter plates, or the section has been otherwise removed from the swatch. The swatch can be of textile, paper, metal, or other suitable material. The smaller swatch can have the stain affixed either before or after it is placed into the well of a 24-, 48- or 96-well microtiter plate. The smaller swatch can also be made by applying a stain to a small piece of material. For example, the smaller swatch can be a stained piece of fabric ⅝″ or 0.25″ in diameter. The custom manufactured punch is designed in such a manner that it delivers 96 swatches simultaneously to all wells of a 96-well plate. The device allows delivery of more than one swatch per well by simply loading the same 96-well plate multiple times. Multi-hole punch devices can be conceived of to deliver simultaneously swatches to any format plate, including but not limited to 24-well, 48-well, and 96-well plates. In another conceivable method, the soiled test platform can be a bead made of metal, plastic, glass, ceramic, or another suitable material that is coated with the soil substrate. The one or more coated beads are then placed into wells of 96-, 48-, or 24-well plates or larger formats, containing suitable buffer and enzyme.

As used herein, “a cultured cell material comprising an amylase” or similar language, refers to a cell lysate or supernatant (including media) that includes an amylase as a component. The cell material may be from a heterologous host that is grown in culture for the purpose of producing the amylase.

As used herein, “percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF.

Deletions are counted as non-identical residues, compared to a reference sequence. Deletions occurring at either termini are included. For example, a variant with five amino acid deletions of the C-terminus of the mature 617 residue polypeptide would have a percent sequence identity of 99% (612/617 identical residues x 100, rounded to the nearest whole number) relative to the mature polypeptide. Such a variant would be encompassed by a variant having “at least 99% sequence identity” to a mature polypeptide.

“Fused” polypeptide sequences are connected, i.e., operably linked, via a peptide bond between two subject polypeptide sequences.

The term “filamentous fungi” refers to all filamentous forms of the subdivision Eumycotina, particularly Pezizomycotina species.

The term “degree of polymerization” (DP) refers to the number (n) of anhydro-glucopyranose units in a given saccharide. Examples of DP1 are the monosaccharides glucose and fructose. Examples of DP2 are the disaccharides maltose and sucrose. The term “DE,” or “dextrose equivalent,” is defined as the percentage of reducing sugar, i.e., D-glucose, as a fraction of total carbohydrate in a syrup.

The term “dry solids content” (ds) refers to the total solids of a slurry in a dry weight percent basis. The term “slurry” refers to an aqueous mixture containing insoluble solids.

The phrase “simultaneous saccharification and fermentation (SSF)” refers to a process in the production of biochemicals in which a microbial organism, such as an ethanologenic microorganism, and at least one enzyme, such as an amylase, are present during the same process step. SSF includes the contemporaneous hydrolysis of starch substrates (granular, liquefied, or solubilized) to saccharides, including glucose, and the fermentation of the saccharides into alcohol or other biochemical or biomaterial in the same reactor vessel.

An “ethanologenic microorganism” refers to a microorganism with the ability to convert a sugar or oligosaccharide to ethanol.

The term “fermented beverage” refers to any beverage produced by a method comprising a fermentation process, such as a microbial fermentation, e.g., a bacterial and/or fungal fermentation. “Beer” is an example of such a fermented beverage, and the term “beer” is meant to comprise any fermented wort produced by fermentation/brewing of a starch-containing plant material. Often, beer is produced exclusively from malt or adjunct, or any combination of malt and adjunct. Examples of beers include: full malted beer, beer brewed under the “Reinheitsgebot,” ale, India pale ale, lager, pilsner, bitter, Happoshu (second beer), third beer, dry beer, near beer, light beer, low alcohol beer, low calorie beer, porter, bock, dopplebock, stout, porter, malt liquor, non-alcoholic beer, non-alcoholic malt liquor and the like, but also alternative cereal and malt beverages such as fruit flavored malt beverages, e.g., citrus flavored, such as lemon-, orange-, lime-, or berry-flavored malt beverages, liquor flavored malt beverages, e.g., vodka-, rum-, or tequila-flavored malt liquor, or coffee flavored malt beverages, such as caffeine-flavored malt liquor, and the like.

The term “malt” refers to any malted cereal grain, such as malted barley or wheat.

The term “adjunct” refers to any starch and/or sugar containing plant material that is not malt, such as barley or wheat malt. Examples of adjuncts include common corn grits, refined corn grits, brewer's milled yeast, rice, sorghum, refined corn starch, barley, barley starch, dehusked barley, wheat, wheat starch, torrified cereal, cereal flakes, rye, oats, potato, tapioca, cassava and syrups, such as corn syrup, sugar cane syrup, inverted sugar syrup, barley and/or wheat syrups, and the like.

The term “mash” refers to an aqueous slurry of any starch and/or sugar containing plant material, such as grist, e.g., comprising crushed barley malt, crushed barley, and/or other adjunct or a combination thereof, mixed with water later to be separated into wort and spent grains.

The term “wort” refers to the unfermented liquor run-off following extracting the grist during mashing.

“Iodine-positive starch” or “IPS” refers to (1) amylose that is not hydrolyzed after liquefaction and saccharification, or (2) a retrograded starch polymer. When saccharified starch or saccharide liquor is tested with iodine, the high DPn amylose or the retrograded starch polymer binds iodine and produces a characteristic blue color. The saccharide liquor is thus termed “iodine-positive saccharide,” “blue saccharide,” or “blue sac.”

The terms “retrograded starch” or “starch retrogradation” refer to changes that occur spontaneously in a starch paste or gel on ageing.

The term “about” refers to ±5% to the referenced value.

Additional Mutations

In some embodiments, the present maltogenic amylases further include one or more mutations that provide a further performance or stability benefit. Exemplary performance benefits include but are not limited to increased hydrolysis of a starch substrate, increased grain, cereal or other starch substrate liquifaction performance, increased cleaning performance, increased thermal stability, increased storage stability, increased solubility, an altered pH profile, decreased calcium dependence, increased specific activity, modified substrate specificity, modified substrate binding, modified pH-dependent activity, modified pH-dependent stability, increased oxidative stability, and increased expression. In some cases, the performance benefit is realized at a relatively low temperature. In some cases, the performance benefit is realized at relatively high temperature.

Furthermore, the present amylases may include any number of conservative amino acid substitutions. Exemplary conservative amino acid substitutions are listed in the following Table 1.

TABLE 1
Conservative amino acid substitutions

| For | | |
|---|---|---|
| Amino Acid | Code | Replace with any of |
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-I-thioazolidine-4-carboxylic acid, D-or |
| | | L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

The reader will appreciate that some of the above mentioned conservative mutations can be produced by genetic manipulation, while others are produced by introducing synthetic amino acids into a polypeptide by genetic or other means.

The present maltogenic amylase may be “precursor,” “immature,” or “full-length,” in which case they include a signal sequence, or “mature,” in which case they lack a signal sequence. Mature forms of the polypeptides are generally the most useful. Unless otherwise noted, the amino acid residue numbering used herein refers to the mature forms of the respective maltogenic amylase polypeptides. The present maltogenic amylase polypeptides may also be truncated to remove the N or C-termini, so long as the resulting polypeptides retain amylase activity.

The present maltogenic amylase may be a “chimeric” or “hybrid” polypeptide, in that it includes at least a portion of a first amylase polypeptide, and at least a portion of a second amylase polypeptide (such chimeric amylases have recently been “rediscovered” as domain-swap amylases). The present amylases may further include heterologous signal sequence, an epitope to allow tracking or purification, or the like. Exemplary heterologous signal sequences are from B. licheniformis amylase (LAT), B. subtilis (AmyE or AprE), and Streptomyces CeIA.

### Nucleotides Encoding Maltogenic Amylase Polypeptides

In another aspect, nucleic acids encoding a maltogenic amylase polypeptide are provided. The nucleic acid may encode a particular maltogenic amylase polypeptide, or a maltogenic amylase having a specified degree of amino acid sequence identity to the particular amylase.

In one example, the nucleic acid encodes a maltogenic amylase having at least 65%, at least 66%, at least 67%, at least 68%, at least 69%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or even at least 99% identity to SEQ ID NO: 1. It will be appreciated that due to the degeneracy of the genetic code, a plurality of nucleic acids may encode the same polypeptide.

In another example, the nucleic acid hybridizes under stringent or very stringent conditions to a nucleic acid encoding (or complementary to a nucleic acid encoding) a maltogenic amylase having at least 65%, at least 66%, at least 67%, at least 68%, at least 69%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or even at least 99% identity to SEQ ID NO: 1. Such stringent and very stringent hybridization conditions are described herein.

Nucleic acids may encode a “full-length” (“fl” or “FL”) maltogenic amylase, which includes a signal sequence, only the mature form of a maltogenic amylase, which lacks the signal sequence, or a truncated form of a maltogenic amylase, which lacks the N or C-terminus of the mature form.

A nucleic acid that encodes a maltogenic amylase can be operably linked to various promoters and regulators in a vector suitable for expressing the maltogenic amylase in host cells. Exemplary promoters are from B. licheniformis amylase (LAT), B. subtilis (AmyE or AprE), and Streptomyces CeIA. Such a nucleic acid can also be linked to other coding sequences, e.g., to encode a chimeric polypeptide.

Production of Variant Amylases

The present maltogenic amylases can be produced in host cells, for example, by secretion or intracellular expression. A cultured cell material (e.g., a whole-cell broth) comprising a maltogenic amylase can be obtained following secretion of the maltogenic amylase into the cell medium. Optionally, the maltogenic amylase can be isolated from the host cells, or even isolated from the cell broth, depending on the desired purity of the final maltogenic amylase. A gene encoding a maltogenic amylase can be cloned and expressed according to methods well known in the art. Suitable host cells include bacterial, fungal (including yeast and filamentous fungi), and plant cells (including algae). Particularly useful host cells include Aspergillus niger, Aspergillus oryzae or Trichoderma reesei. Other host cells include bacterial cells, e.g., Bacillus subtilis or B. licheniformis, as well as Streptomyces.

The host cell further may express a nucleic acid encoding a homologous or heterologous glucoamylase, i.e., a glucoamylase that is not the same species as the host cell, or one or more other enzymes. The glucoamylase may be a variant glucoamylase, such as one of the glucoamylase variants disclosed in U.S. Pat. No. 8,058,033 (Danisco US Inc.), for example. Additionally, the host may express one or more accessory enzymes, proteins, peptides. These may benefit liquefaction, saccharification, fermentation, SSF, etc processes. Furthermore, the host cell may produce biochemicals in addition to enzymes used to digest the various feedstock(s). Such host cells may be useful for fermentation or simultaneous saccharification and fermentation processes to reduce or eliminate the need to add enzymes.

Vectors

A DNA construct comprising a nucleic acid encoding maltogenic amylases can be constructed to be expressed in a host cell. Representative nucleic acids that encode maltogenic amylases include SEQ ID NO: 1. Because of the well-known degeneracy in the genetic code, variant polynucleotides that encode an identical amino acid sequence can be designed and made with routine skill. It is also well-known in the art to optimize codon use for a particular host cell. Nucleic acids encoding maltogenic amylases can be incorporated into a vector. Vectors can be transferred to a host cell using well-known transformation techniques, such as those disclosed below.

The vector may be any vector that can be transformed into and replicated within a host cell. For example, a vector comprising a nucleic acid encoding a maltogenic amylase can be transformed and replicated in a bacterial host cell as a means of propagating and amplifying the vector. The vector also may be transformed into an expression host, so that the encoding nucleic acids can be expressed as a functional maltogenic amylase. Host cells that serve as expression hosts can include filamentous fungi, for example. The Fungal Genetics Stock Center (FGSC) Catalogue of Strains lists suitable vectors for expression in fungal host cells. See FGSC, Catalogue of Strains, University of Missouri, at www.fgsc.net (last modified Jan. 17, 2007). A representative vector is pJG153, a promoterless Cre expression vector that can be replicated in a bacterial host. See Harrison et al. (June 2011) Applied Environ. Microbiol. 77: 3916-22. pJG153 can be modified with routine skill to comprise and express a nucleic acid encoding a maltogenic amylase.

A nucleic acid encoding a maltogenic amylase can be operably linked to a suitable promoter, which allows transcription in the host cell. The promoter may be any DNA sequence that shows transcriptional activity in the host cell of choice and may be derived from genes encoding proteins either homologous or heterologous to the host cell. Exemplary promoters for directing the transcription of the DNA sequence encoding a maltogenic amylase, especially in a bacterial host, are the promoter of the lac operon of E. coli, the Streptomyces coelicolor agarase gene dagA or celA promoters, the promoters of the Bacillus licheniformis α-amylase gene (amyL), the promoters of the Bacillus stearothermophilus maltogenic amylase gene (amyM), the promoters of the Bacillus amyloliquefaciens α-amylase (amyQ), the promoters of the Bacillus subtilis xylA and xylB genes etc. For transcription in a fungal host, examples of useful promoters are those derived from the gene encoding Aspergillus oryzae TAKA amylase, Rhizomucor miehei aspartic proteinase, Aspergillus niger neutral α-amylase, A. niger acid stable α-amylase, A. nigerglucoamylase, Rhizomucor miehei lipase, A. oryzae alkaline protease, A. oryzae triose phosphate isomerase, or A. nidulans acetamidase. When a gene encoding a maltogenic amylase is expressed in a bacterial species such as E. coli, a suitable promoter can be selected, for example, from a bacteriophage promoter including a T7 promoter and a phage lambda promoter. Examples of suitable promoters for the expression in a yeast species include but are not limited to the Gal 1 and Gal 10 promoters of Saccharomyces cerevisiae and the Pichia pastoris AOX1 or AOX2 promoters. cbh1 is an endogenous, inducible promoter from T. reesei. See Liu et al. (2008) “Improved heterologous gene expression in Trichoderma reesei by cellobiohydrolase I gene (cbh1) promoter optimization,” Acta Biochim. Biophys. Sin (Shanghai) 40(2): 158-65.

The coding sequence can be operably linked to a signal sequence. The DNA encoding the signal sequence may be the DNA sequence naturally associated with the maltogenic amylase gene to be expressed or from a different Genus or species. A signal sequence and a promoter sequence comprising a DNA construct or vector can be introduced into a fungal host cell and can be derived from the same source. For example, the signal sequence is the cbh1 signal sequence that is operably linked to a cbh1 promoter.

An expression vector may also comprise a suitable transcription terminator and, in eukaryotes, polyadenylation sequences operably linked to the DNA sequence encoding a variant amylase. Termination and polyadenylation sequences may suitably be derived from the same sources as the promoter.

The vector may further comprise a DNA sequence enabling the vector to replicate in the host cell. Examples of such sequences are the origins of replication of plasmids pUC19, pACYC177, pUB110, pE194, pAMB1, and pIJ702.

The vector may also comprise a selectable marker, e.g., a gene the product of which complements a defect in the isolated host cell, such as the daI genes from B. subtilis or B. licheniformis, or a gene that confers antibiotic resistance such as, e.g., ampicillin, kanamycin, chloramphenicol or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., International PCT Application WO 91/17243.

Intracellular expression may be advantageous in some respects, e.g., when using certain bacteria or fungi as host cells to produce large amounts of maltogenic amylase for subsequent enrichment or purification. Extracellular secretion of amylase into the culture medium can also be used to make a cultured cell material comprising the isolated maltogenic amylase.

The expression vector typically includes the components of a cloning vector, such as, for example, an element that permits autonomous replication of the vector in the selected host organism and one or more phenotypically detectable markers for selection purposes. The expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the maltogenic amylase to a host cell organelle such as a peroxisome, or to a particular host cell compartment. Such a targeting sequence includes but is not limited to the sequence, SKL. For expression under the direction of control sequences, the nucleic acid sequence of the amylase is operably linked to the control sequences in proper manner with respect to expression.

The procedures used to ligate the DNA construct encoding a maltogenic amylase, the promoter, terminator and other elements, respectively, and to insert them into suitable vectors containing the information necessary for replication, are well known to persons skilled in the art (see, e.g., Sambrook et al., MOLECULAR CLONING: A LABORATORY MANUAL, 2^{nd} ed., Cold Spring Harbor, 1989, and 3^{rd} ed., 2001).

Transformation and Culture of Host Cells

An isolated cell, either comprising a DNA construct or an expression vector, is advantageously used as a host cell in the recombinant production of a maltogenic amylase. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

Examples of suitable bacterial host organisms are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species. Suitable host organisms among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. A suitable procedure for transformation of Aspergillus host cells includes, for example, that described in EP 238023. A maltogenic amylase expressed by a fungal host cell can be glycosylated, i.e., will comprise a glycosyl moiety. The glycosylation pattern can be the same or different as present in the wild-type maltogenic amylase. The type and/or degree of glycosylation may impart changes in enzymatic and/or biochemical properties.

It is advantageous to delete genes from expression hosts, where the gene deficiency can be cured by the transformed expression vector. Known methods may be used to obtain a fungal host cell having one or more inactivated genes. Gene inactivation may be accomplished by complete or partial deletion, by insertional inactivation or by any other means that renders a gene nonfunctional for its intended purpose, such that the gene is prevented from expression of a functional protein. Any gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; and protoplast fusion. General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding a maltogenic amylase is stably integrated into a host cell chromosome. Transformants are then selected and purified by known techniques.

The preparation of Trichoderma sp. for transformation, for example, may involve the preparation of protoplasts from fungal mycelia. See Campbell et al. (1989) Curr. Genet. 16: 53-56. The mycelia can be obtained from germinated vegetative spores. The mycelia are treated with an enzyme that digests the cell wall, resulting in protoplasts. The protoplasts are protected by the presence of an osmotic stabilizer in the suspending medium. These stabilizers include sorbitol, mannitol, potassium chloride, magnesium sulfate, and the like. Usually the concentration of these stabilizers varies between 0.8 M and 1.2 M, e.g., a 1.2 M solution of sorbitol can be used in the suspension medium.

Uptake of DNA into the host Trichoderma sp. strain depends upon the calcium ion concentration. Generally, between about 10-50 mM CaCl₂ is used in an uptake solution. Additional suitable compounds include a buffering system, such as TE buffer (10 mM Tris, pH 7.4; 1 mM EDTA) or 10 mM MOPS, pH 6.0 and polyethylene glycol. The polyethylene glycol is believed to fuse the cell membranes, thus permitting the contents of the medium to be delivered into the cytoplasm of the Trichoderma sp. strain. This fusion frequently leaves multiple copies of the plasmid DNA integrated into the host chromosome.

Usually transformation of Trichoderma sp. uses protoplasts or cells that have been subjected to a permeability treatment, typically at a density of 10⁵ to 10⁷/mL, particularly 2×10⁶/mL. A volume of 100 μL of these protoplasts or cells in an appropriate solution (e.g., 1.2 M sorbitol and 50 mM CaCl₂) may be mixed with the desired DNA. Generally, a high concentration of PEG is added to the uptake solution. From 0.1 to 1 volume of 25% PEG 4000 can be added to the protoplast suspension; however, it is useful to add about 0.25 volumes to the protoplast suspension. Additives, such as dimethyl sulfoxide, heparin, spermidine, potassium chloride and the like, may also be added to the uptake solution to facilitate transformation. Similar procedures are available for other fungal host cells. See, e.g., U.S. Pat. No. 6,022,725.

### Expression

A method of producing a maltogenic amylase may comprise cultivating a host cell as described above under conditions conducive to the production of the enzyme and recovering the enzyme from the cells and/or culture medium.

The medium used to cultivate the cells may be any conventional medium suitable for growing the host cell in question and obtaining expression of a maltogenic amylase. Suitable media and media components are available from commercial suppliers or may be prepared according to published recipes (e.g., as described in catalogues of the American Type Culture Collection).

An enzyme secreted from the host cells can be used in a whole broth preparation. In the present methods, the preparation of a spent whole fermentation broth of a recombinant microorganism can be achieved using any cultivation method known in the art resulting in the expression of a maltogenic amylase. Fermentation may, therefore, be understood as comprising shake flask cultivation, small- or large-scale fermentation (including continuous, batch, fed-batch, or solid state fermentations) in laboratory or industrial fermenters performed in a suitable medium and under conditions allowing the amylase to be expressed or isolated. The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

An enzyme secreted from the host cells may conveniently be recovered from the culture medium by well-known procedures, including separating the cells from the medium by centrifugation or filtration, and precipitating proteinaceous components of the medium by means of a salt such as ammonium sulfate, followed by the use of chromatographic procedures such as ion exchange chromatography, affinity chromatography, or the like.

The polynucleotide encoding a maltogenic amylase in a vector can be operably linked to a control sequence that is capable of providing for the expression of the coding sequence by the host cell, i.e. the vector is an expression vector. The control sequences may be modified, for example by the addition of further transcriptional regulatory elements to make the level of transcription directed by the control sequences more responsive to transcriptional modulators. The control sequences may in particular comprise promoters.

Host cells may be cultured under suitable conditions that allow expression of a maltogenic amylase. Expression of the enzymes may be constitutive such that they are continually produced, or inducible, requiring a stimulus to initiate expression. In the case of inducible expression, protein production can be initiated when required by, for example, addition of an inducer substance to the culture medium, for example dexamethasone or IPTG or Sophorose. Polypeptides can also be produced recombinantly in an in vitro cell-free system, such as the TNT™ (Promega) rabbit reticulocyte system.

An expression host also can be cultured in the appropriate medium for the host, under aerobic conditions. Shaking or a combination of agitation and aeration can be provided, with production occurring at the appropriate temperature for that host, e.g., from about 25° C. to about 75° C. (e.g., 30° C. to 45° C.), depending on the needs of the host and production of the desired maltogenic amylase. Culturing can occur from about 12 to about 100 hours or greater (and any hour value there between, e.g., from 24 to 72 hours). Typically, the culture broth is at a pH of about 4.0 to about 8.0, again depending on the culture conditions needed for the host relative to production of a maltogenic amylase.

Methods for Enriching and Purifying Maltogenic Amylases

Fermentation, separation, and concentration techniques are well known in the art and conventional methods can be used in order to prepare a concentrated maltogenic amylase polypeptide-containing solution.

After fermentation, a fermentation broth is obtained, the microbial cells and various suspended solids, including residual raw fermentation materials, are removed by conventional separation techniques in order to obtain a maltogenic amylase solution. Filtration, centrifugation, microfiltration, rotary vacuum drum filtration, ultrafiltration, centrifugation followed by ultra-filtration, extraction, or chromatography, or the like, are generally used.

It is desirable to concentrate a maltogenic amylase polypeptide-containing solution in order to optimize recovery. Use of unconcentrated solutions requires increased incubation time in order to collect the enriched or purified enzyme precipitate.

The enzyme containing solution is concentrated using conventional concentration techniques until the desired enzyme level is obtained. Concentration of the enzyme containing solution may be achieved by any of the techniques discussed herein. Exemplary methods of enrichment and purification include but are not limited to rotary vacuum filtration and/or ultrafiltration.

The enzyme solution is concentrated into a concentrated enzyme solution until the enzyme activity of the concentrated maltogenic amylase polypeptide-containing solution is at a desired level.

Concentration may be performed using, e.g., a precipitation agent, such as a metal halide precipitation agent. Metal halide precipitation agents include but are not limited to alkali metal chlorides, alkali metal bromides and blends of two or more of these metal halides. Exemplary metal halides include sodium chloride, potassium chloride, sodium bromide, potassium bromide and blends of two or more of these metal halides. The metal halide precipitation agent, sodium chloride, can also be used as a preservative.

The metal halide precipitation agent is used in an amount effective to precipitate a maltogenic amylase. The selection of at least an effective amount and an optimum amount of metal halide effective to cause precipitation of the enzyme, as well as the conditions of the precipitation for maximum recovery including incubation time, pH, temperature and concentration of enzyme, will be readily apparent to one of ordinary skill in the art, after routine testing.

Generally, at least about 5% w/v (weight/volume) to about 25% w/v of metal halide is added to the concentrated enzyme solution, and usually at least 8% w/v. Generally, no more than about 25% w/v of metal halide is added to the concentrated enzyme solution and usually no more than about 20% w/v. The optimal concentration of the metal halide precipitation agent will depend, among others, on the nature of the specific maltogenic amylase polypeptide and on its concentration in the concentrated enzyme solution.

Another alternative way to precipitate the enzyme is to use organic compounds. Exemplary organic compound precipitating agents include: 4-hydroxybenzoic acid, alkali metal salts of 4-hydroxybenzoic acid, alkyl esters of 4-hydroxybenzoic acid, and blends of two or more of these organic compounds. The addition of the organic compound precipitation agents can take place prior to, simultaneously with or subsequent to the addition of the metal halide precipitation agent, and the addition of both precipitation agents, organic compound and metal halide, may be carried out sequentially or simultaneously.

Generally, the organic precipitation agents are selected from the group consisting of alkali metal salts of 4-hydroxybenzoic acid, such as sodium or potassium salts, and linear or branched alkyl esters of 4-hydroxybenzoic acid, wherein the alkyl group contains from 1 to 12 carbon atoms, and blends of two or more of these organic compounds. The organic compound precipitation agents can be, for example, linear or branched alkyl esters of 4-hydroxybenzoic acid, wherein the alkyl group contains from 1 to 10 carbon atoms, and blends of two or more of these organic compounds. Exemplary organic compounds are linear alkyl esters of 4-hydroxybenzoic acid, wherein the alkyl group contains from 1 to 6 carbon atoms, and blends of two or more of these organic compounds. Methyl esters of 4-hydroxybenzoic acid, propyl esters of 4-hydroxybenzoic acid, butyl ester of 4-hydroxybenzoic acid, ethyl ester of 4-hydroxybenzoic acid and blends of two or more of these organic compounds can also be used. Additional organic compounds also include but are not limited to 4-hydroxybenzoic acid methyl ester (named methyl PARABEN), 4-hydroxybenzoic acid propyl ester (named propyl PARABEN), which also are both amylase preservative agents. For further descriptions, see, e.g., U.S. Pat. No. 5,281,526.

Addition of the organic compound precipitation agent provides the advantage of high flexibility of the precipitation conditions with respect to pH, temperature, maltogenic amylase concentration, precipitation agent concentration, and time of incubation.

The organic compound precipitation agent is used in an amount effective to is improve precipitation of the enzyme by means of the metal halide precipitation agent. The selection of at least an effective amount and an optimum amount of organic compound precipitation agent, as well as the conditions of the precipitation for maximum recovery including incubation time, pH, temperature and concentration of enzyme, will be readily apparent to one of ordinary skill in the art, in light of the present disclosure, after routine testing.

Generally, at least about 0.01% w/v of organic compound precipitation agent is added to the concentrated enzyme solution and usually at least about 0.02% w/v. Generally, no more than about 0.3% w/v of organic compound precipitation agent is added to the concentrated enzyme solution and usually no more than about 0.2% w/v.

The concentrated polypeptide solution, containing the metal halide precipitation agent, and the organic compound precipitation agent, can be adjusted to a pH, which will, of necessity, depend on the enzyme to be enriched or purified. Generally, the pH is adjusted at a level near the isoelectric point of the amylase. The pH can be adjusted at a pH in a range from about 2.5 pH units below the isoelectric point (pl) up to about 2.5 pH units above the isoelectric point.

The incubation time necessary to obtain an enriched or purified enzyme precipitate depends on the nature of the specific enzyme, the concentration of enzyme, and the specific precipitation agent(s) and its (their) concentration. Generally, the time effective to precipitate the enzyme is between about 1 to about 30 hours; usually it does not exceed about 25 hours. In the presence of the organic compound precipitation agent, the time of incubation can still be reduced to less about 10 hours and in most cases even about 6 hours.

Generally, the temperature during incubation is between about 4° C. and about 50° C. Usually, the method is carried out at a temperature between about 10° C. and about 45° C. (e.g., between about 20° C. and about 40° C.). The optimal temperature for inducing precipitation varies according to the solution conditions and the enzyme or precipitation agent(s) used.

The overall recovery of enriched or purified enzyme precipitate, and the efficiency with which the process is conducted, is improved by agitating the solution comprising the enzyme, the added metal halide and the added organic compound. The agitation step is done both during addition of the metal halide and the organic compound, and during the subsequent incubation period. Suitable agitation methods include mechanical stirring or shaking, vigorous aeration, or any similar technique.

After the incubation period, the enriched or purified enzyme is then separated from the dissociated pigment and other impurities and collected by conventional separation techniques, such as filtration, centrifugation, microfiltration, rotary vacuum filtration, ultrafiltration, press filtration, cross membrane microfiltration, cross flow membrane microfiltration, or the like. Further enrichment or purification of the enzyme precipitate can be obtained by washing the precipitate with water. For example, the enriched or purified enzyme precipitate is washed with water containing the metal halide precipitation agent, or with water containing the metal halide and the organic compound precipitation agents.

During fermentation, a maltogenic amylase polypeptide accumulates in the culture broth. For the isolation, enrichment, or purification of the desired maltogenic amylase, the culture broth is centrifuged or filtered to eliminate cells, and the resulting cell-free liquid is used for enzyme enrichment or purification. In one embodiment, the cell-free broth is subjected to salting out using ammonium sulfate at about 70% saturation; the 70% saturation-precipitation fraction is then dissolved in a buffer and applied to a column such as a Sephadex G-100 column, and eluted to recover the enzyme-active fraction. For further enrichment or purification, a conventional procedure such as ion exchange chromatography may be used.

Enriched or purified enzymes are useful for laundry and cleaning applications. For example, they can be used in laundry detergents and spot removers. They can be made into a final product that is either liquid (solution, slurry) or solid (granular, powder).

A more specific example of enrichment or purification, is described in Sumitani et al. (2000) “New type of starch-binding domain: the direct repeat motif in the C-terminal region of Bacillus sp. 195 α-amylase contributes to starch binding and raw starch degrading,” Biochem. J. 350: 477-484, and is briefly summarized here. The enzyme obtained from 4 liters of a Streptomyces lividans TK24 culture supernatant is treated with (NH₄)₂SO₄ at 80% saturation. The precipitate is recovered by centrifugation at 10,000×g (20 min. and 4° C.) and re-dissolved in 20 mM Tris/HCl buffer (pH 7.0) containing 5 mM CaCl₂. The solubilized precipitate is then dialyzed against the same buffer. The dialyzed sample is then applied to a Sephacryl S-200 column, which had previously been equilibrated with 20 mM Tris/HCl buffer, (pH 7.0), 5 mM CaCl₂, and eluted at a linear flow rate of 7 mL/hr with the same buffer. Fractions from the column are collected and assessed for activity as judged by enzyme assay and SDS-PAGE. The protein is further purified as follows. A Toyopearl HW55 column (Tosoh Bioscience, Montgomeryville, Pa.; Cat. No. 19812) is equilibrated with 20 mM Tris/HCl buffer (pH 7.0) containing 5 mM CaCl₂ and 1.5 M (NH₄)₂SO₄. The enzyme is eluted with a linear gradient of 1.5 to 0 M (NH₄)₂SO₄ in 20 mM Tris/HCL buffer, pH 7.0 containing 5 mM CaCl₂. The active fractions are collected, and the enzyme precipitated with (NH₄)₂SO₄ at 80% saturation. The precipitate is recovered, re-dissolved, and dialyzed as described above. The dialyzed sample is then applied to a Mono Q HR5/5 column (Amersham Pharmacia; Cat. No. 17-5167-01) previously equilibrated with 20 mM Tris/HCl buffer (pH 7.0) containing 5 mM CaCl₂, at a flow rate of 60 mL/hour. The active fractions are collected and added to a 1.5 M (NH₄)₂SO₄ solution. The active enzyme fractions are re-chromatographed on a Toyopearl HW55 column, as before, to yield a homogeneous enzyme as determined by SDS-PAGE. See Sumitani et al. (2000) Biochem. J. 350: 477-484, for general discussion of the method and variations thereon.

For production scale recovery, maltogenic amylase polypeptides can be enriched or partially purified as generally described above by removing cells via flocculation with polymers. Alternatively, the enzyme can be enriched or purified by microfiltration followed by concentration by ultrafiltration using available membranes and equipment. However, for some applications, the enzyme does not need to be enriched or purified, and whole broth culture can be lysed and used without further treatment. The enzyme can then be processed, for example, into granules.

Compositions and Uses of Maltogenic Amylases

The maltogenic amylases provided by the present teachings are useful for a variety of industrial applications. For example, maltogenic amylases are useful in a starch conversion process, particularly in a saccharification process of a starch that has undergone liquefaction. The desired end-product may be any product that may be produced by the enzymatic conversion of the starch substrate. For example, the desired product may be a syrup rich in glucose and maltose, which can be used in other processes, such as the preparation of HFCS, or which can be converted into a number of other useful products, such as ascorbic acid intermediates (e.g., gluconate; 2-keto-L-gulonic acid; 5-keto-gluconate; and 2,5-diketogluconate); 1,3-propanediol; aromatic amino acids (e.g., tyrosine, phenylalanine and tryptophan); organic acids (e.g., lactate, pyruvate, succinate, isocitrate, and oxaloacetate); amino acids (e.g., serine and glycine); antibiotics; antimicrobials; enzymes; vitamins; and hormones.

The starch conversion process may be a precursor to, or simultaneous with, a fermentation process designed to produce alcohol for fuel or drinking (i.e., potable alcohol). One skilled in the art is aware of various fermentation conditions that may be used in the production of these end-products. Variant amylases are also useful in compositions and methods of food preparation. These various uses of variant amylases are described in more detail below.

It will be appreciated by one of ordinary skill in the art that various accessory enzymes can be used with the maltogenic enzymes of the present teachings, as will be the case in various applications and contexts.

In the field of grain processing to produce maltose syrups, the maltogenic amylases can be employed in any of a variety of applications, including those described in U.S. Provisional Application 61/616,990, filed Mar. 28, 2012.

Preparation of Starch Substrates from Plants

Those of general skill in the art are well aware of available methods that may be used to prepare starch substrates for use in the processes disclosed herein. For example, a useful starch substrate may be obtained from tubers, roots, stems, legumes, cereals or whole grain. More specifically, the granular starch may be obtained from corn, cobs, wheat, barley, rye, triticale, milo, sago, millet, cassava, tapioca, sorghum, rice, peas, bean, banana, or potatoes. Corn contains about 60-68% starch; barley contains about 55-65% starch; millet contains about 75-80% starch; wheat contains about 60-65% starch; and polished rice contains 70-72% starch. Specifically contemplated starch substrates are corn starch and wheat starch. The starch from a grain may be ground or whole and includes corn solids, such as kernels, bran and/or cobs. The starch may also be highly refined raw starch or feedstock from starch refinery processes. Various starches also are commercially available. For example, corn starch is available from Cerestar, Sigma, and Katayama Chemical Industry Co. (Japan); wheat starch is available from Sigma; sweet potato starch is available from Wako Pure Chemical Industry Co. (Japan); and potato starch is available from Nakaari Chemical Pharmaceutical Co. (Japan).

The starch substrate can be a crude starch from milled whole grain, which contains non-starch fractions, e.g., germ residues and fibers. Milling may comprise either wet milling or dry milling or grinding. In wet milling, whole grain is soaked in water or dilute acid to separate the grain into its component parts, e.g., starch, protein, germ, oil, kernel fibers. Wet milling efficiently separates the germ and meal (i.e., starch granules and protein) and is especially suitable for production of syrups. In dry milling or grinding, whole kernels are ground into a fine powder and often processed without fractionating the grain into its component parts. In some cases, oils from the kernels are recovered. Dry ground grain thus will comprise significant amounts of non-starch carbohydrate compounds, in addition to starch. Dry grinding of the starch substrate can be used for production of ethanol and other biochemicals. The starch to be processed may be a highly refined starch quality, for example, at least 90%, at least 95%, at least 97%, or at least 99.5% pure.

All references cited herein are herein incorporated by reference in their entirety for all purposes. In order to further illustrate the compositions and methods, and advantages thereof, the following specific examples are given with the understanding that they are illustrative rather than limiting.

Fermentation

The soluble starch hydrolysate, particularly a glucose rich syrup, can be fermented by contacting the starch hydrolysate with a fermenting organism typically at a temperature around 32° C., such as from 30° C. to 35° C. for alcohol-producing yeast. The temperature and pH of the fermentation will depend upon the fermenting organism. EOF products include metabolites, such as citric acid, lactic acid, succinic acid, monosodium glutamate, gluconic acid, sodium gluconate, calcium gluconate, potassium gluconate, itaconic acid and other carboxylic acids, glucono delta-lactone, sodium erythorbate, lysine and other amino acids, omega 3 fatty acid, butanol, isoprene, 1,3-propanediol and other biomaterials.

Ethanologenic microorganisms include yeast, such as Saccharomyces cerevisiae and bacteria, e.g., Zymomonas mobilis, expressing alcohol dehydrogenase and pyruvate decarboxylase. The ethanologenic microorganism can express xylose reductase and xylitol dehydrogenase, which convert xylose to xylulose. Improved strains of ethanologenic microorganisms, which can withstand higher temperatures, for example, are known in the art and can be used. See Liu et al. (2011) Sheng Wu Gong Cheng Xue Bao 27(7): 1049-56. Commercial sources of yeast include ETHANOL RED® (LeSaffre); Thermosacc® (Lallemand); RED STAR® (Red Star); FERMIOL® (DSM Specialties); and SUPERSTART® (Alltech). Microorganisms that produce other metabolites, such as citric acid and lactic acid, by fermentation are also known in the art. See, e.g., Papagianni (2007) “Advances in citric acid fermentation by Aspergillus niger: biochemical aspects, membrane transport and modeling,” Biotechnol. Adv. 25(3): 244-63; John et al. (2009) “Direct lactic acid fermentation: focus on simultaneous saccharification and lactic acid production,” Biotechnol. Adv. 27(2): 145-52.

The saccharification and fermentation processes may be carried out as an SSF process. Fermentation may comprise subsequent enrichment, purification, and recovery of ethanol, for example. During the fermentation, the ethanol content of the broth or “beer” may reach about 8-18% v/v, e.g., 14-15% v/v. The broth may be distilled to produce enriched, e.g., 96% pure, solutions of ethanol. Further, CO₂ generated by fermentation may be collected with a CO₂ scrubber, compressed, and marketed for other uses, e.g., carbonating beverage or dry ice production. Solid waste from the fermentation process may be used as protein-rich products, e.g., livestock feed.

As mentioned above, an SSF process can be conducted with fungal cells that express and secrete amylase continuously throughout SSF. The fungal cells expressing amylase also can be the fermenting microorganism, e.g., an ethanologenic microorganism. Ethanol production thus can be carried out using a fungal cell that expresses sufficient amylase so that less or no enzyme has to be added exogenously. The fungal host cell can be from an appropriately engineered fungal strain. Fungal host cells that express and secrete other enzymes, in addition to amylase, also can be used. Such cells may express glucoamylase and/or a pullulanase, phytase, alpha-glucosidase, isoamylase, beta-amylase cellulase, xylanase, other hemicellulases, protease, beta-glucosidase, pectinase, esterase, redox enzymes, transferase, or other enzyme.

A variation on this process is a “fed-batch fermentation” system, where the substrate is added in increments as the fermentation progresses. Fed-batch systems are useful when catabolite repression may inhibit the metabolism of the cells and where it is desirable to have limited amounts of substrate in the medium. The actual substrate concentration in fed-batch systems is estimated by the changes of measurable factors such as pH, dissolved oxygen and the partial pressure of waste gases, such as CO₂. Batch and fed-batch fermentations are common and well known in the art.

Continuous fermentation is an open system where a defined fermentation medium is added continuously to a bioreactor, and an equal amount of conditioned medium is removed simultaneously for processing. Continuous fermentation generally maintains the cultures at a constant high density where cells are primarily in log phase growth. Continuous fermentation permits modulation of cell growth and/or product concentration. For example, a limiting nutrient such as the carbon source or nitrogen source is maintained at a fixed rate and all other parameters are allowed to moderate. Because growth is maintained at a steady state, cell loss due to medium being drawn off should be balanced against the cell growth rate in the fermentation. Methods of optimizing continuous fermentation processes and maximizing the rate of product formation are well known in the art of industrial microbiology.

Compositions Comprising Maltogenic Amylases

The maltogenic amylases of the present teachings may be combined with a glucoamylase (EC 3.2.1.3), e.g., a Trichoderma glucoamylase or variant thereof. An exemplary glucoamylase is Trichoderma reesei glucoamylase (TrGA) and variants thereof that possess superior specific activity and thermal stability. See U.S. Published Applications Nos. 2006/0094080, 2007/0004018, and 2007/0015266 (Danisco US Inc.). Suitable variants of TrGA include those with glucoamylase activity and at least 80%, at least 90%, or at least 95% sequence identity to wild-type TrGA. Maltogenic amylases may advantageously increase the yield of glucose produced in a saccharification process catalyzed by TrGA.

Alternatively, the glucoamylase may be another glucoamylase derived from plants (including algae), fungi, or bacteria. For example, the glucoamylases may be Aspergillus niger G1 or G2 glucoamylase or its variants (e.g., Boel et al. (1984) EMBO J. 3: 1097-1102; WO 92/00381; WO 00/04136 (Novo Nordisk A/S)); and A. awamori glucoamylase (e.g., WO 84/02921 (Cetus Corp.)). Other contemplated Aspergillus glucoamylase include variants with enhanced thermal stability, e.g., G137A and G139A (Chen et al. (1996) Prot. Eng. 9:499-505); D257E and D293E/Q (Chen et al. (1995) Prot. Eng. 8: 575-582); N182 (Chen et al. (1994) Biochem. J. 301: 275-281); A246C (Fierobe et al. (1996) Biochemistry, 35: 8698-8704); and variants with Pro residues in positions A435 and S436 (Li et al. (1997) Protein Eng. 10: 1199-1204). Other contemplated glucoamylases include Talaromyces glucoamylases, in particular derived from T. emersonii (e.g., WO 99/28448 (Novo Nordisk A/S), T. leycettanus (e.g., U.S. Pat. No. RE 32,153 (CPC International, Inc.)), T. duponti, or T. thermophilus (e.g., U.S. Pat. No. 4,587,215). Contemplated bacterial glucoamylases include glucoamylases from the genus Clostridium, in particular C. thermoamylolyticum (e.g., EP 135,138 (CPC International, Inc.) and C. thermohydrosulfuricum (e.g., WO 86/01831 (Michigan Biotechnology Institute)). Suitable glucoamylases include the glucoamylases derived from Aspergillus oryzae, such as a glucoamylase shown in SEQ ID NO:2 in WO 00/04136 (Novo Nordisk A/S). Also suitable are commercial glucoamylases, such as AMG 200L; AMG 300 L; SAN™ SUPER and AMG™ E (Novozymes); OPTIDEX® 300 and OPTIDEX L-400 (Danisco US Inc.); AMIGASE™ and AMIGASE™ PLUS (DSM); G-ZYME® G900 (Enzyme Bio-Systems); and G-ZYMEθ G990 ZR (A. niger glucoamylase with a low protease content). Still other suitable glucoamylases include Aspergillus fumigatus glucoamylase, Talaromyces glucoamylase, Thielavia glucoamylase, Trametes glucoamylase, Thermomyces glucoamylase, Athelia glucoamylase, or Humicola glucoamylase (e.g., HgGA). Glucoamylases typically are added in an amount of about 0.1-2 glucoamylase units (GAU)/g ds, e.g., about 0.16 GAU/g ds, 0.23 GAU/g ds, or 0.33 GAU/g ds.

Other suitable enzymes that can be used with the maltogenic amylase of the present teachings include a phytase, protease, pullulanase, β-amylase, isoamylase, a different α-amylase, alpha-glucosidase, cellulase, xylanase, other hemicellulases, beta-glucosidase, transferase, pectinase, lipase, cutinase, esterase, redox enzymes, or a combination thereof. For example, a debranching enzyme, such as an isoamylase (EC 3.2.1.68), may be added in effective amounts well known to the person skilled in the art. A pullulanase (EC 3.2.1.41), e.g., Promozyme®, is also suitable. Pullulanase typically is added at 100 U/kg ds. Further suitable enzymes include proteases, such as fungal and bacterial proteases. Fungal proteases include those obtained from Aspergillus, such as A. niger, A. awamori, A. oryzae; Mucor (e.g., M. miehei); Rhizopus; and Trichoderma.

β-Amylases (EC 3.2.1.2) are exo-acting maltogenic amylases, which catalyze the hydrolysis of 1,4-α-glucosidic linkages into amylopectin and related glucose polymers, thereby releasing maltose. β-Amylases have been isolated from various plants and microorganisms. See Fogarty et al. (1979) in PROGRESS IN INDUSTRIAL MICROBIOLOGY, Vol. 15, pp. 112-115. These β-Amylases have optimum temperatures in the range from 40° C. to 65° C. and optimum pH in the range from about 4.5 to about 7.0. Contemplated β-amylases include, but are not limited to, β-amylases from barley Spezyme® BBA 1500, Spezyme® DBA, OptimaIt™ ME, OptimaIt™ BBA (Danisco US Inc.); and Novozym™ WBA (Novozymes A/S).

Compositions comprising the present maltogenic amylases may be aqueous or non-aqueous formulations, granules, powders, gels, slurries, pastes, etc., which may further comprise any one or more of the additional enzymes listed, herein, along with buffers, salts, preservatives, water, co-solvents, surfactants, and the like. Such compositions may work in combination with endogenous enzymes or other ingredients already present in a slurry, water bath, washing machine, food or drink product, etc, for example, endogenous plant (including algal) enzymes, residual enzymes from a prior processing step, and the like.

Compositions and Methods for Baking and Food Preparation

The present teachings also relate to a “food composition,” including but not limited to a food product, animal feed and/or food/feed additives, comprising a maltogenic amylase, and methods for preparing such a food composition comprising mixing the maltogenic amylase with one or more food ingredients, or uses thereof.

Furthermore, the present teachings relate to the use of a maltogenic amylase in the preparation of a food composition, wherein the food composition is baked subsequent to the addition of the polypeptide of the present invention. As used herein the term “baking composition” means any composition and/or additive prepared in the process of providing a baked food product, including but not limited to bakers flour, a dough, a baking additive and/or a baked product. The food composition or additive may be liquid or solid.

As used herein, the term “flour” means milled or ground cereal grain. The term “flour” also may mean Sago or tuber products that have been ground or mashed. In some embodiments, flour may also contain components in addition to the milled or mashed cereal or plant matter. An example of an additional component, although not intended to be limiting, is a leavening agent. Cereal grains include wheat, oat, rye, and barley. Tuber products include tapioca flour, cassava flour, and custard powder. The term “flour” also includes ground corn flour, maize-meal, rice flour, whole-meal flour, self-rising flour, tapioca flour, cassava flour, ground rice, enriched flower, and custard powder.

For the commercial and home use of flour for baking and food production, it is important to maintain an appropriate level of α-amylase activity in the flour. A level of activity that is too high may result in a product that is sticky and/or doughy and therefore unmarketable. Flour with insufficient α-amylase activity may not contain enough sugar for proper yeast function, resulting in dry, crumbly bread, or baked products. Accordingly, a maltogenic amylase, by itself or in combination with an α-amylase(s), may be added to the flour to augment the level of endogenous α-amylase activity in flour.

A maltogenic amylase can further be added alone or in a combination with other amylases to prevent or retard staling, i.e., crumb firming of baked products. The amount of anti-staling amylase will typically be in the range of 0.01-10 mg of enzyme protein per kg of flour, e.g., 0.5 mg/kg ds. Additional anti-staling amylases that can be used in combination with an amylase include an endo-amylase, e.g., a bacterial endo-amylase from Bacillus. The additional amylase can be another maltogenic α-amylase (EC 3.2.1.133), e.g., from Bacillus. Novamyl® is an exemplary maltogenic α-amylase from B. stearothermophilus strain NCIB 11837 and is described in Christophersen et al. (1997) Starch 50: 39-45. Other examples of anti-staling endo-amylases include bacterial α-amylases derived from Bacillus, such as B. licheniformis or B. amyloliquefaciens. The anti-staling amylase may be an exo-amylase, such as β-amylase, e.g., from plant sources, such as soy bean, or from microbial sources, such as Bacillus.

The baking composition comprising a maltogenic amylase further can comprise a phospholipase or enzyme with phospholipase activity. An enzyme with phospholipase activity has an activity that can be measured in Lipase Units (LU). The phospholipase may have A₁ or A₂ activity to remove fatty acid from the phospholipids, forming a lysophospholipid. It may or may not have lipase activity, i.e., activity on triglyceride substrates. The phospholipase typically has a temperature optimum in the range of 30-90° C., e.g., 30-70° C. The added phospholipases can be of animal origin, for example, from pancreas, e.g., bovine or porcine pancreas, snake venom or bee venom. Alternatively, the phospholipase may be of microbial origin, e.g., from filamentous fungi, yeast or bacteria, for example.

The phospholipase is added in an amount that improves the softness of the bread during the initial period after baking, particularly the first 24 hours. The amount of phospholipase will typically be in the range of 0.01-10 mg of enzyme protein per kg of flour, e.g., 0.1-5 mg/kg. That is, phospholipase activity generally will be in the range of 20-1000 LU/kg of flour, where a Lipase Unit is defined as the amount of enzyme required to release 1 μmol butyric acid per minute at 30° C., pH 7.0, with gum arabic as emulsifier and tributyrin as substrate.

Compositions of dough generally comprise wheat meal or wheat flour and/or other types of meal, flour or starch such as corn flour, cornstarch, rye meal, rye flour, oat flour, oatmeal, soy flour, sorghum meal, sorghum flour, potato meal, potato flour or potato starch. The dough may be fresh, frozen or par-baked. The dough can be a leavened dough or a dough to be subjected to leavening. The dough may be leavened in various ways, such as by adding chemical leavening agents, e.g., sodium bicarbonate or by adding a leaven, i.e., fermenting dough. Dough also may be leavened by adding a suitable yeast culture, such as a culture of Saccharomyces cerevisiae (baker's yeast), e.g., a commercially available strain of S. cerevisiae.

The dough may also comprise other conventional dough ingredients, e.g., proteins, such as milk powder, gluten, and soy; eggs (e.g., whole eggs, egg yolks or egg whites); an oxidant, such as ascorbic acid, potassium bromate, potassium iodate, azodicarbonamide (ADA) or ammonium persulfate; an amino acid such as L-cysteine; a sugar; or a salt, such as sodium chloride, calcium acetate, sodium sulfate or calcium sulfate. The dough further may comprise fat, e.g., triglyceride, such as granulated fat or shortening. The dough further may comprise an emulsifier such as mono- or diglycerides, diacetyl tartaric acid esters of mono- or diglycerides, sugar esters of fatty acids, polyglycerol esters of fatty acids, lactic acid esters of monoglycerides, acetic acid esters of monoglycerides, polyoxyethylene stearates, or lysolecithin. In particular, the dough can be made without addition of emulsifiers.

The dough product may be any processed dough product, including fried, deep fried, roasted, baked, steamed and boiled doughs, such as steamed bread and rice cakes. In one embodiment, the food product is a bakery product. Typical bakery (baked) products include bread—such as loaves, rolls, buns, bagels, pizza bases etc. pastry, pretzels, tortillas, cakes, cookies, biscuits, crackers etc.

Optionally, an additional enzyme may be used together with the anti-staling amylase and the phospholipase. The additional enzyme may be a second amylase, such as an amyloglucosidase, a β-amylase, a cyclodextrin glucanotransferase, or the additional enzyme may be a peptidase, in particular an exopeptidase, a transglutaminase, a lipase, a cellulase, a xylanase, a protease, a protein disulfide isomerase, e.g., a protein disulfide isomerase as disclosed in WO 95/00636, for example, a glycosyltransferase, a branching enzyme (1,4-α-glucan branching enzyme), a 4-α-glucanotransferase (dextrin glycosyltransferase) or an oxidoreductase, e.g., a peroxidase, a laccase, a glucose oxidase, a pyranose oxidase, a lipooxygenase, an L-amino acid oxidase or a carbohydrate oxidase. The additional enzyme(s) may be of any origin, including mammalian and plant, and particularly of microbial (bacterial, yeast or fungal) origin and may be obtained by techniques conventionally used in the art.

The xylanase is typically of microbial origin, e.g., derived from a bacterium or fungus, such as a strain of Aspergillus. Xylanases include Pentopan® and Novozym 384®, for example, which are commercially available xylanase preparations produced from Trichoderma reesei. The amyloglucosidase may be an A. niger amyloglucosidase (such as AMG®). Other useful amylase products include Grindamyl® A 1000 or A 5000 (Grindsted Products, Denmark) and Amylase® H or Amylase® P (DSM). The glucose oxidase may be a fungal glucose oxidase, in particular an Aspergillus niger glucose oxidase (such as Gluzyme®). An exemplary protease is Neutrase®.

The process may be used for any kind of baked product prepared from dough, either of a soft or a crisp character, either of a white, light or dark type. Examples are bread, particularly white, whole-meal or rye bread, typically in the form of loaves or rolls, such as, but not limited to, French baguette-type bread, pita bread, tortillas, cakes, pancakes, biscuits, cookies, pie crusts, crisp bread, steamed bread, pizza and the like.

A maltogenic amylase may be used in a pre-mix, comprising flour together with an anti-staling amylase, a phospholipase, and/or a phospholipid. The pre-mix may contain other dough-improving and/or bread-improving additives, e.g., any of the additives, including enzymes, mentioned above. A maltogenic amylase can be a component of an enzyme preparation comprising an anti-staling amylase and a phospholipase, for use as a baking additive.

The enzyme preparation is optionally in the form of a granulate or agglomerated powder. The preparation can have a narrow particle size distribution with more than 95% (by weight) of the particles in the range from 25 to 500 μm. Granulates and agglomerated powders may be prepared by conventional methods, e.g., by spraying an amylase onto a carrier in a fluid-bed granulator. The carrier may consist of particulate cores having a suitable particle size. The carrier may be soluble or insoluble, e.g., a salt (such as NaCl or sodium sulfate), a sugar (such as sucrose or lactose), a sugar alcohol (such as sorbitol), starch, rice, corn grits, or soy.

Enveloped particles, i.e., maltogenic amylase particles, can comprise a maltogenic amylase. To prepare enveloped maltogenic amylase particles, the enzyme is contacted with a food grade lipid in sufficient quantity to suspend all of the maltogenic amylase particles. Food grade lipids, as used herein, may be any naturally organic compound that is insoluble in water but is soluble in non-polar organic solvents such as hydrocarbon or diethyl ether. Suitable food grade lipids include, but are not limited to, triglycerides either in the form of fats or oils that are either saturated or unsaturated. Examples of fatty acids and combinations thereof which make up the saturated triglycerides include, but are not limited to, butyric (derived from milk fat), palmitic (derived from animal and plant fat), and/or stearic (derived from animal and plant fat). Examples of fatty acids and combinations thereof which make up the unsaturated triglycerides include, but are not limited to, palmitoleic (derived from animal and plant fat), oleic (derived from animal and plant fat), linoleic (derived from plant oils), and/or linolenic (derived from linseed oil). Other suitable food grade lipids include, but are not limited to, monoglycerides and diglycerides derived from the triglycerides discussed above, phospholipids and glycolipids.

The food grade lipid, particularly in the liquid form, is contacted with a powdered form of the maltogenic amylase particles in such a fashion that the lipid material covers at least a portion of the surface of at least a majority, e.g., 100% of the maltogenic amylase particles. Thus, each maltogenic amylase particle is individually enveloped in a lipid. For example, all or substantially all of the maltogenic amylase particles are provided with a thin, continuous, enveloping film of lipid. This can be accomplished by first pouring a quantity of lipid into a container, and then slurrying the maltogenic amylase particles so that the lipid thoroughly wets the surface of each maltogenic amylase particle. After a short period of stirring, the enveloped maltogenic amylase particles, carrying a substantial amount of the lipids on their surfaces, are recovered. The thickness of the coating so applied to the particles of maltogenic amylase can be controlled by selection of the type of lipid used and by repeating the operation in order to build up a thicker film, when desired.

The storing, handling and incorporation of the loaded delivery vehicle can be accomplished by means of a packaged mix. The packaged mix can comprise the enveloped maltogenic amylase. However, the packaged mix may further contain additional ingredients as required by the manufacturer or baker. After the enveloped maltogenic amylase has been incorporated into the dough, the baker continues through the normal production process for that product.

The advantages of enveloping the maltogenic amylase particles are two-fold. First, the food grade lipid protects the enzyme from thermal denaturation during the baking process for those enzymes that are heat labile. Consequently, while the maltogenic amylase is stabilized and protected during the proving and baking stages, it is released from the protective coating in the final baked good product, where it hydrolyzes the glucosidic linkages in polyglucans. The loaded delivery vehicle also provides a sustained release of the active enzyme into the baked good. That is, following the baking process, active maltogenic amylase is continually released from the protective coating at a rate that counteracts, and therefore reduces the rate of, staling mechanisms.

In general, the amount of lipid applied to the maltogenic amylase particles can vary from a few percent of the total weight of the maltogenic amylase to many times that weight, depending upon the nature of the lipid, the manner in which it is applied to the maltogenic amylase particles, the composition of the dough mixture to be treated, and the severity of the dough-mixing operation involved.

The loaded delivery vehicle, i.e., the lipid-enveloped enzyme, is added to the ingredients used to prepare a baked good in an effective amount to extend the shelf-life of the baked good. The baker computes the amount of enveloped maltogenic amylase, prepared as discussed above, that will be required to achieve the desired anti-staling effect. The amount of the enveloped maltogenic amylase required is calculated based on the concentration of enzyme enveloped and on the proportion of maltogenic amylase to flour specified. A wide range of concentrations has been found to be effective, although, as has been discussed, observable improvements in anti-staling do not correspond linearly with the maltogenic amylase concentration, but above certain minimal levels, large increases in maltogenic amylase concentration produce little additional improvement. The maltogenic amylase concentration actually used in a particular bakery production could be much higher than the minimum necessary to provide the baker with some insurance against inadvertent under-measurement errors by the baker. The lower limit of enzyme concentration is determined by the minimum anti-staling effect the baker wishes to achieve.

A method of preparing a baked good may comprise: a) preparing lipid-coated maltogenic amylase particles, where substantially all of the maltogenic amylase particles are coated; b) mixing a dough containing flour; c) adding the lipid-coated maltogenic amylase to the dough before the mixing is complete and terminating the mixing before the lipid coating is removed from the maltogenic amylase; d) proofing the dough; and e) baking the dough to provide the baked good, where the maltogenic amylase is inactive during the mixing, proofing and baking stages and is active in the baked good.

The enveloped maltogenic amylase can be added to the dough during the mix cycle, e.g., near the end of the mix cycle. The enveloped maltogenic amylase is added at a point in the mixing stage that allows sufficient distribution of the enveloped maltogenic amylase throughout the dough; however, the mixing stage is terminated before the protective coating becomes stripped from the maltogenic amylase particle(s). Depending on the type and volume of dough, and mixer action and speed, anywhere from one to six minutes or more might be required to mix the enveloped maltogenic amylase into the dough, but two to four minutes is average. Thus, several variables may determine the precise procedure. First, the quantity of enveloped maltogenic amylase should have a total volume sufficient to allow the enveloped maltogenic amylase to be spread throughout the dough mix. If the preparation of enveloped maltogenic amylase is highly concentrated, additional oil may need to be added to the pre-mix before the enveloped maltogenic amylase is added to the dough. Recipes and production processes may require specific modifications; however, good results generally can be achieved when 25% of the oil specified in a bread dough formula is held out of the dough and is used as a carrier for a concentrated enveloped α-amylase when added near the end of the mix cycle. In bread or other baked goods, particularly those having a low fat content, e.g., French-style breads, an enveloped maltogenic amylase mixture of approximately 1% of the dry flour weight is sufficient to admix the enveloped α-amylase properly with the dough. The range of suitable percentages is wide and depends on the formula, finished product, and production methodology requirements of the individual baker. Second, the enveloped maltogenic amylase suspension should be added to the mix with sufficient time for complete mixture into the dough, but not for such a time that excessive mechanical action strips the protective lipid coating from the enveloped maltogenic amylase particles.

In a further aspect of the invention, the food composition is an oil, meat, lard, composition comprising a maltogenic amylase. In this context the term “oil/meat/lard” composition” means any composition, based on, made from and/or containing oil, meat or lard, respectively. Another aspect the invention relates to a method of preparing an oil or meat or lard composition and/or additive comprising a maltogenic amylase, comprising mixing the polypeptide of the invention with a oil/meat/lard composition and/or additive ingredients.

In a further aspect of the invention, the food composition is an animal feed composition, animal feed additive and/or pet food comprising a maltogenic amylase and variants thereof. The present invention further relates to a method for preparing such an animal feed composition, animal feed additive composition and/or pet food comprising mixing a maltogenic amylase and variants thereof with one or more animal feed ingredients and/or animal feed additive ingredients and/or pet food ingredients. Furthermore, the present invention relates to the use of a maltogenic amylase in the preparation of an animal feed composition and/or animal feed additive composition and/or pet food.

The term “animal” includes all non-ruminant and ruminant animals. In a particular embodiment, the animal is a non-ruminant animal, such as a horse and a mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment the animal is a ruminant animal including, but not limited to, cattle, young calves, goats, sheep, giraffes, bison, moose, elk, yaks, water buffalo, deer, camels, alpacas, llamas, antelope, pronghorn and nilgai.

In the present context, it is intended that the term “pet food” is understood to mean a food for a household animal such as, but not limited to dogs, cats, gerbils, hamsters, chinchillas, fancy rats, guinea pigs; avian pets, such as canaries, parakeets, and parrots; reptile pets, such as turtles, lizards and snakes; and aquatic pets, such as tropical fish and frogs.

The terms “animal feed composition,” “feedstuff” and “fodder” are used interchangeably and may comprise one or more feed materials selected from the group comprising a) cereals, such as small grains (e.g., wheat, barley, rye, oats and combinations thereof) and/or large grains such as maize or sorghum; b) by products from cereals, such as corn gluten meal, Distillers Dried Grain Solubles (DDGS) (particularly corn based Distillers Dried Grain Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; e) minerals and vitamins.

Textile Desizing Compositions and Use

Also contemplated are compositions and methods of treating fabrics (e.g., to desize a textile) using a maltogenic amylase. Fabric-treating methods are well known in the art (see, e.g., U.S. Pat. No. 6,077,316). For example, the feel and appearance of a fabric can be improved by a method comprising contacting the fabric with a maltogenic amylase in a solution. The fabric can be treated with the solution under pressure.

A maltogenic amylase can be applied during or after the weaving of a textile, or during the desizing stage, or one or more additional fabric processing steps. During the weaving of textiles, the threads are exposed to considerable mechanical strain. Prior to weaving on mechanical looms, warp yarns are often coated with sizing starch or starch derivatives to increase their tensile strength and to prevent breaking. A maltogenic amylase can be applied during or after the weaving to remove these sizing starch or starch derivatives. After weaving, a maltogenic amylase can be used to remove the size coating before further processing the fabric to ensure a homogeneous and wash-proof result.

A maltogenic amylase can be used alone or with other desizing chemical reagents and/or desizing enzymes to desize fabrics, including cotton-containing fabrics, as detergent additives, e.g., in aqueous compositions. A maltogenic amylase also can be used in compositions and methods for producing a stonewashed look on indigo-dyed denim fabric and garments. For the manufacture of clothes, the fabric can be cut and sewn into clothes or garments, which are afterwards finished. In particular, for the manufacture of denim jeans, different enzymatic finishing methods have been developed. The finishing of denim garment normally is initiated with an enzymatic desizing step, during which garments are subjected to the action of amylolytic enzymes to provide softness to the fabric and make the cotton more accessible to the subsequent enzymatic finishing steps. A maltogenic amylase can be used in methods of finishing denim garments (e.g., a “bio-stoning process”), enzymatic desizing and providing softness to fabrics, and/or finishing process.

Cleaning Compositions

An aspect of the present compositions and methods is a cleaning composition that includes a maltogenic amylase as a component. A maltogenic amylase polypeptide can be used as a component in detergent compositions for hand washing, laundry washing, dishwashing, and other hard-surface cleaning.

Overview of Cleaning Compositions

Preferably, a maltogenic amylase is incorporated into detergents at or near a concentration conventionally used for amylase in detergents. For example, a maltogenic amylase polypeptide may be added in amount corresponding to 0.00001-1 mg (calculated as pure enzyme protein) of maltogenic amylase per liter of wash/dishwash liquor. Exemplary formulations are provided herein, as exemplified by the following:

A maltogenic amylase polypeptide may be a component of a detergent composition, as the only enzyme or with other enzymes including other amylolytic enzymes. As such, it may be included in the detergent composition in the form of a non-dusting granulate, a stabilized liquid, or a protected enzyme. Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1,000 to 20,000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in, for example, GB 1483591. Liquid enzyme preparations may, for instance, be stabilized by adding a polyol such as propylene glycol, a sugar or sugar alcohol, lactic acid or boric acid according to established methods. Other enzyme stabilizers are known in the art. Protected enzymes may be prepared according to the method disclosed in for example EP 238 216. Polyols have long been recognized as stabilizers of proteins, as well as improving protein solubility.

The detergent composition may be in any useful form, e.g., as powders, granules, pastes, or liquid. A liquid detergent may be aqueous, typically containing up to about 70% of water and 0% to about 30% of organic solvent. It may also be in the form of a compact gel type containing only about 30% water.

The detergent composition comprises one or more surfactants, each of which may be anionic, nonionic, cationic, or zwitterionic. The detergent will usually contain 0% to about 50% of anionic surfactant, such as linear alkylbenzenesulfonate (LAS); α-olefinsulfonate (AOS); alkyl sulfate (fatty alcohol sulfate) (AS); alcohol ethoxysulfate (AEOS or AES); secondary alkanesulfonates (SAS); α-sulfo fatty acid methyl esters; alkyl- or alkenylsuccinic acid; or soap. The composition may also contain 0% to about 40% of nonionic surfactant such as alcohol ethoxylate (AEO or AE), carboxylated alcohol ethoxylates, nonylphenol ethoxylate, alkylpolyglycoside, alkyldimethylamineoxide, ethoxylated fatty acid monoethanolamide, fatty acid monoethanolamide, or polyhydroxy alkyl fatty acid amide (as described for example in WO 92/06154).

The detergent composition may additionally comprise one or more other enzymes, such as proteases, another amylolytic enzyme, cutinase, lipase, cellulase, pectate lyase, perhydrolase, xylanase, peroxidase, and/or laccase in any combination.

The detergent may contain about 1% to about 65% of a detergent builder or complexing agent such as zeolite, diphosphate, triphosphate, phosphonate, citrate, nitrilotriacetic acid (NTA), ethylenediaminetetraacetic acid (EDTA), diethylenetriaminepentaacetic acid (DTMPA), alkyl- or alkenylsuccinic acid, soluble silicates or layered silicates (e.g., SKS-6 from Hoechst). The detergent may also be unbuilt, i.e. essentially free of detergent builder. The enzymes can be used in any composition compatible with the stability of the enzyme. Enzymes generally can be protected against deleterious components by known forms of encapsulation, for example, by granulation or sequestration in hydro gels. Enzymes, and specifically maltoogenic amylases, either with or without starch binding domains, can be used in a variety of compositions including laundry and dishwashing applications, surface cleaners, as well as in compositions for ethanol production from starch or biomass.

The detergent may comprise one or more polymers. Examples include carboxymethylcellulose (CMC), poly(vinylpyrrolidone) (PVP), polyethyleneglycol (PEG), poly(vinyl alcohol) (PVA), polycarboxylates such as polyacrylates, maleic/acrylic acid copolymers and lauryl methacrylate/acrylic acid copolymers.

The detergent may contain a bleaching system, which may comprise a H₂O₂ source such as perborate or percarbonate, which may be combined with a peracid-forming bleach activator such as tetraacetylethylenediamine (TAED) or nonanoyloxybenzenesulfonate (NOBS). Alternatively, the bleaching system may comprise peroxyacids (e.g., the amide, imide, or sulfone type peroxyacids). The bleaching system can also be an enzymatic bleaching system, for example, perhydrolase, such as that described in International PCT Application WO 2005/056783.

The enzymes of the detergent composition may be stabilized using conventional stabilizing agents, e.g., a polyol such as propylene glycol or glycerol; a sugar or sugar alcohol; lactic acid; boric acid or a boric acid derivative such as, e.g., an aromatic borate ester; and the composition may be formulated as described in, e.g., WO 92/19709 and WO 92/19708.

The detergent may also contain other conventional detergent ingredients such as e.g., fabric conditioners including clays, foam boosters, suds suppressors, anti-corrosion agents, soil-suspending agents, anti-soil redeposition agents, dyes, bactericides, tarnish inhibiters, optical brighteners, or perfumes.

The pH (measured in aqueous solution at use concentration) is usually neutral or alkaline, e.g., pH about 7.0 to about 11.0.

Particular forms of detergent compositions for inclusion of the present α-amylase are described, below.

Heavy Duty Liquid (HDL) Laundry Detergent Composition

Exemplary HDL laundry detergent compositions includes a detersive surfactant (10%-40% wt/wt), including an anionic detersive surfactant (selected from a group of linear or branched or random chain, substituted or unsubstituted alkyl sulphates, alkyl sulphonates, alkyl alkoxylated sulphate, alkyl phosphates, alkyl phosphonates, alkyl carboxylates, and/or mixtures thereof), and optionally non-ionic surfactant (selected from a group of linear or branched or random chain, substituted or unsubstituted alkyl alkoxylated alcohol, for example a C₈-C₁₈ alkyl ethoxylated alcohol and/or C₆-C₁₂ alkyl phenol alkoxylates), wherein the weight ratio of anionic detersive surfactant (with a hydrophilic index (HIc) of from 6.0 to 9) to non-ionic detersive surfactant is greater than 1:1. Suitable detersive surfactants also include cationic detersive surfactants (selected from a group of alkyl pyridinium compounds, alkyl quarternary ammonium compounds, alkyl quarternary phosphonium compounds, alkyl ternary sulphonium compounds, and/or mixtures thereof); zwitterionic and/or amphoteric detersive surfactants (selected from a group of alkanolamine sulpho-betaines); ampholytic surfactants; semi-polar non-ionic surfactants and mixtures thereof.

The composition may optionally include, a surfactancy boosting polymer consisting of amphiphilic alkoxylated grease cleaning polymers (selected from a group of alkoxylated polymers having branched hydrophilic and hydrophobic properties, such as alkoxylated polyalkylenimines in the range of 0.05 wt %-10 wt %) and/or random graft polymers (typically comprising of hydrophilic backbone comprising monomers selected from the group consisting of: unsaturated C₁-C₆ carboxylic acids, ethers, alcohols, aldehydes, ketones, esters, sugar units, alkoxy units, maleic anhydride, saturated polyalcohols such as glycerol, and mixtures thereof; and hydrophobic side chain(s) selected from the group consisting of: C₄-C₂₅ alkyl group, polypropylene, polybutylene, vinyl ester of a saturated C₁-C₆ mono-carboxylic acid, C₁-C₆ alkyl ester of acrylic or methacrylic acid, and mixtures thereof.

The composition may include additional polymers such as soil release polymers (include anionically end-capped polyesters, for example SRP1, polymers comprising at least one monomer unit selected from saccharide, dicarboxylic acid, polyol and combinations thereof, in random or block configuration, ethylene terephthalate-based polymers and co-polymers thereof in random or block configuration, for example Repel-o-tex SF, SF-2 and SRP6, Texcare SRA100, SRA300, SRN100, SRN170, SRN240, SRN300 and SRN325, Marloquest SL), anti-redeposition polymers (0.1 wt % to 10 wt %, include carboxylate polymers, such as polymers comprising at least one monomer selected from acrylic acid, maleic acid (or maleic anhydride), fumaric acid, itaconic acid, aconitic acid, mesaconic acid, citraconic acid, methylenemalonic acid, and any mixture thereof, vinylpyrrolidone homopolymer, and/or polyethylene glycol, molecular weight in the range of from 500 to 100,000 Da); cellulosic polymer (including those selected from alkyl cellulose, alkyl alkoxyalkyl cellulose, carboxyalkyl cellulose, alkyl carboxyalkyl cellulose examples of which include carboxymethyl cellulose, methyl cellulose, methyl hydroxyethyl cellulose, methyl carboxymethyl cellulose, and mixtures thereof) and polymeric carboxylate (such as maleate/acrylate random copolymer or polyacrylate homopolymer).

The composition may further include saturated or unsaturated fatty acid, preferably saturated or unsaturated C₁₂-C₂₄ fatty acid (0 wt % to 10 wt %); deposition aids (examples for which include polysaccharides, preferably cellulosic polymers, poly diallyl dimethyl ammonium halides (DADMAC), and co-polymers of DAD MAC with vinyl pyrrolidone, acrylamides, imidazoles, imidazolinium halides, and mixtures thereof, in random or block configuration, cationic guar gum, cationic cellulose such as cationic hydoxyethyl cellulose, cationic starch, cationic polyacylamides, and mixtures thereof.

The composition may further include dye transfer inhibiting agents, examples of which include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents, examples of which include ethylene-diamine-tetraacetic acid (EDTA), diethylene triamine penta methylene phosphonic acid (DTPMP), hydroxy-ethane diphosphonic acid (HEDP), ethylenediamine N,N′-disuccinic acid (EDDS), methyl glycine diacetic acid (MGDA), diethylene triamine penta acetic acid (DTPA), propylene diamine tetracetic acid (PDT A), 2-hydroxypyridine-N-oxide (HPNO), or methyl glycine diacetic acid (MGDA), glutamic acid N,N-diacetic acid (N,N-dicarboxymethyl glutamic acid tetrasodium salt (GLDA), nitrilotriacetic acid (NTA), 4,5-dihydroxy-m-benzenedisulfonic acid, citric acid and any salts thereof, N-hydroxyethylethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP), and derivatives thereof.

The composition preferably includes enzymes (generally about 0.01 wt % active enzyme to 0.03 wt % active enzyme) selected from proteases, amylases, lipases, cellulases, choline oxidases, peroxidases/oxidases, pectate lyases, mannanases, cutinases, laccases, phospholipases, lysophospholipases, acyltransferases, perhydrolases, arylesterases, and any mixture thereof. The composition may include an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

The composition optionally includes silicone or fatty-acid based suds suppressors; hueing dyes, calcium and magnesium cations, visual signaling ingredients, anti-foam (0.001 wt % to about 4.0 wt %), and/or structurant/thickener (0.01 wt % to 5 wt %, selected from the group consisting of diglycerides and triglycerides, ethylene glycol distearate, microcrystalline cellulose, cellulose based materials, microfiber cellulose, biopolymers, xanthan gum, gellan gum, and mixtures thereof).

The composition can be any liquid form, for example a liquid or gel form, or any combination thereof. The composition may be in any unit dose form, for example a pouch.

Heavy Duty Dry/Solid (HDD) Laundry Detergent Composition

Exemplary HDD laundry detergent compositions includes a detersive surfactant, including anionic detersive surfactants (e.g., linear or branched or random chain, substituted or unsubstituted alkyl sulphates, alkyl sulphonates, alkyl alkoxylated sulphate, alkyl phosphates, alkyl phosphonates, alkyl carboxylates and/or mixtures thereof), non-ionic detersive surfactant (e.g., linear or branched or random chain, substituted or unsubstituted C₈-C₁₈ alkyl ethoxylates, and/or C₆-C₁₂ alkyl phenol alkoxylates), cationic detersive surfactants (e.g., alkyl pyridinium compounds, alkyl quaternary ammonium compounds, alkyl quaternary phosphonium compounds, alkyl ternary sulphonium compounds, and mixtures thereof), zwitterionic and/or amphoteric detersive surfactants (e.g., alkanolamine sulpho-betaines), ampholytic surfactants, semi-polar non-ionic surfactants, and mixtures thereof; builders including phosphate free builders (for example zeolite builders examples which include zeolite A, zeolite X, zeolite P and zeolite MAP in the range of 0 wt % to less than 10 wt %), phosphate builders (for example sodium tri-polyphosphate in the range of 0 wt % to less than 10 wt %), citric acid, citrate salts and nitrilotriacetic acid, silicate salt (e.g., sodium or potassium silicate or sodium meta-silicate in the range of 0 wt % to less than 10 wt %, or layered silicate (SKS-6)); carbonate salt (e.g., sodium carbonate and/or sodium bicarbonate in the range of 0 wt % to less than 80 wt %); and bleaching agents including photobleaches (e.g., sulfonated zinc phthalocyanines, sulfonated aluminum phthalocyanines, xanthenes dyes, and mixtures thereof) hydrophobic or hydrophilic bleach activators (e.g., dodecanoyl oxybenzene sulfonate, decanoyl oxybenzene sulfonate, decanoyl oxybenzoic acid or salts thereof, 3,5,5-trimethy hexanoyl oxybenzene sulfonate, tetraacetyl ethylene diamine-TAED, nonanoyloxybenzene sulfonate-NOBS, nitrile quats, and mixtures thereof), sources of hydrogen peroxide (e.g., inorganic perhydrate salts examples of which include mono or tetra hydrate sodium salt of perborate, percarbonate, persulfate, perphosphate, or persilicate), preformed hydrophilic and/or hydrophobic peracids (e.g., percarboxylic acids and salts, percarbonic acids and salts, perimidic acids and salts, peroxymonosulfuric acids and salts, and mixtures thereof), and/or bleach catalysts (e.g., imine bleach boosters (examples of which include iminium cations and polyions), iminium zwitterions, modified amines, modified amine oxides, N-sulphonyl imines, N-phosphonyl imines, N-acyl imines, thiadiazole dioxides, perfluoroimines, cyclic sugar ketones, and mixtures thereof, and metal-containing bleach catalysts (e.g., copper, iron, titanium, ruthenium, tungsten, molybdenum, or manganese cations along with an auxiliary metal cations such as zinc or aluminum and a sequestrate such as ethylenediaminetetraacetic acid, ethylenediaminetetra(methylenephosphonic acid), and water-soluble salts thereof).

The composition preferably includes enzymes, e.g., proteases, amylases, lipases, cellulases, choline oxidases, peroxidases/oxidases, pectate lyases, mannanases, cutinases, laccases, phospholipases, lysophospholipases, acyltransferase, perhydrolase, arylesterase, and any mixture thereof.

The composition may optionally include additional detergent ingredients including perfume microcapsules, starch encapsulated perfume accord, hueing agents, additional polymers, including fabric integrity and cationic polymers, dye-lock ingredients, fabric-softening agents, brighteners (for example C.I. Fluorescent brighteners), flocculating agents, chelating agents, alkoxylated polyamines, fabric deposition aids, and/or cyclodextrin.

Automatic Dishwashing (ADW) Detergent Composition

Exemplary ADW detergent composition includes non-ionic surfactants, including ethoxylated non-ionic surfactants, alcohol alkoxylated surfactants, epoxy-capped poly(oxyalkylated) alcohols, or amine oxide surfactants present in amounts from 0 to 10% by weight; builders in the range of 5-60% including phosphate builders (e.g., mono-phosphates, di-phosphates, tri-polyphosphates, other oligomeric-poylphosphates, sodium tripolyphosphate-STPP) and phosphate-free builders (e.g., amino acid-based compounds including methyl-glycine-diacetic acid (MGDA) and salts and derivatives thereof, glutamic-N,N-diacetic acid (GLDA) and salts and derivatives thereof, iminodisuccinic acid (IDS) and salts and derivatives thereof, carboxy methyl inulin and salts and derivatives thereof, nitrilotriacetic acid (NTA), diethylene triamine penta acetic acid (DTPA), B-alaninediacetic acid (B-ADA) and their salts, homopolymers and copolymers of poly-carboxylic acids and their partially or completely neutralized salts, monomeric polycarboxylic acids and hydroxycarboxylic acids and their salts in the range of 0.5% to 50% by weight; sulfonated/carboxylated polymers in the range of about 0.1% to about 50% by weight to provide dimensional stability; drying aids in the range of about 0.1% to about 10% by weight (e.g., polyesters, especially anionic polyesters, optionally together with further monomers with 3 to 6 functionalities—typically acid, alcohol or ester functionalities which are conducive to polycondensation, polycarbonate-, polyurethane- and/or polyurea-polyorganosiloxane compounds or precursor compounds, thereof, particularly of the reactive cyclic carbonate and urea type); silicates in the range from about 1% to about 20% by weight (including sodium or potassium silicates for example sodium disilicate, sodium meta-silicate and crystalline phyllosilicates); inorganic bleach (e.g., perhydrate salts such as perborate, percarbonate, perphosphate, persulfate and persilicate salts) and organic bleach (e.g., organic peroxyacids, including diacyl and tetraacylperoxides, especially diperoxydodecanedioc acid, di peroxytetradecanedioc acid, and diperoxyhexadecanedioc acid); bleach activators (i.e., organic peracid precursors in the range from about 0.1% to about 10% by weight); bleach catalysts (e.g., manganese triazacyclononane and related complexes, Co, Cu, Mn, and Fe bispyridylamine and related complexes, and pentamine acetate cobalt(III) and related complexes); metal care agents in the range from about 0.1% to 5% by weight (e.g., benzatriazoles, metal salts and complexes, and/or silicates); enzymes in the range from about 0.01 to 5.0 mg of active enzyme per gram of automatic dishwashing detergent composition (e.g., proteases, amylases, lipases, cellulases, choline oxidases, peroxidases/oxidases, pectate lyases, mannanases, cutinases, laccases, phospholipases, lysophospholipases, acyltransferase, perhydrolase, arylesterase, and mixtures thereof); and enzyme stabilizer components (e.g., oligosaccharides, polysaccharides, and inorganic divalent metal salts).

The present maltogenic amylase polypeptide may be incorporated at a concentration conventionally employed in detergents. It is at present contemplated that, in the detergent composition, the enzyme may be added in an amount corresponding to 0.00001-1.0 mg (calculated as pure enzyme protein) of amylase polypeptide per liter of wash liquor.

The detergent composition may also contain other conventional detergent ingredients, e.g., deflocculant material, filler material, foam depressors, anti-corrosion agents, soil-suspending agents, sequestering agents, anti-soil redeposition agents, dehydrating agents, dyes, bactericides, fluorescers, thickeners, and perfumes.

The detergent composition may be formulated as a hand (manual) or machine (automatic) laundry detergent composition, including a laundry additive composition suitable for pre-treatment of stained fabrics and a rinse added fabric softener composition, or be formulated as a detergent composition for use in general household hard surface cleaning operations, or be formulated for manual or automatic dishwashing operations.

Any of the cleaning compositions described, herein, may include any number of additional enzymes. In general the enzyme(s) should be compatible with the selected detergent, (e.g., with respect to pH-optimum, compatibility with other enzymatic and non-enzymatic ingredients, and the like), and the enzyme(s) should be present in effective amounts. The following enzymes are provided as examples.

Proteases:

Suitable proteases include those of animal, vegetable or microbial origin. Chemically modified or protein engineered mutants are included, as well as naturally processed proteins. The protease may be a serine protease or a metalloprotease, an alkaline microbial protease, a trypsin-like protease, or a chymotrypsin-like protease. Examples of alkaline proteases are subtilisins, especially those derived from Bacillus, e.g., subtilisin Novo, subtilisin Carlsberg, subtilisin 309, subtilisin 147, and subtilisin 168 (see, e.g., WO 89/06279). Examples of trypsin-like proteases are trypsin (e.g., of porcine or bovine origin), and Fusarium proteases (see, e.g., WO 89/06270 and WO 94/25583). Examples of useful proteases also include but are not limited to the variants described in WO 92/19729, WO 98/20115, WO 98/20116, and WO 98/34946. Commercially available protease enzymes include but are not limited to: ALCALASE®, SAVINASE®, PRIMASE™, DURALASE™, ESPERASE®, KANNASE™, and BLAZE™ (Novo Nordisk A/S and Novozymes A/S); MAXATASE®, MAXACAL™, MAXAPEM™, PROP ERASE®, PURAFECT®, PURAFECT OXP™, FN2™, and FN3™ (Danisco US Inc.). Other exemplary proteases include NprE from Bacillus amyloliquifaciens and ASP from Cellulomonas sp. strain 69B4.

Lipases:

Suitable lipases include those of bacterial or fungal origin. Chemically modified, proteolytically modified, or protein engineered mutants are included. Examples of useful lipases include but are not limited to lipases from Humicola (synonym Thermomyces), e.g., from H. lanuginosa (T lanuginosus) (see e.g., EP 258068 and EP 305216), from H. insolens (see e.g., WO 96/13580); a Pseudomonas lipase (e.g., from P. alcaligenes or P. pseudoalcaligenes; see, e.g., EP 218 272), P. cepacia (see e.g., EP 331 376), P. stutzeri (see e.g., GB 1,372,034), P. fluorescens, Pseudomonas sp. strain SD 705 (see e.g., WO 95/06720 and WO 96/27002), P. wisconsinensis (see e.g., WO 96/12012); a Bacillus lipase (e.g., from B. subtilis; see e.g., Dartois et al. Biochemica et Biophysica Acta, 1131: 253-360 (1993)), B. stearothermophilus (see e.g., JP 64/744992), or B. pumilus (see e.g., WO 91/16422). Additional lipase variants contemplated for use in the formulations include those described for example in: WO 92/05249, WO 94/01541, WO 95/35381, WO 96/00292, WO 95/30744, WO 94/25578, WO 95/14783, WO 95/22615, WO 97/04079, WO 97/07202, EP 407225, and EP 260105. Some commercially available lipase enzymes include LIPOLASE® and LIPOLASE ULTRA™ (Novo Nordisk A/S and Novozymes A/S).

Polyesterases:

Suitable polyesterases can be included in the composition, such as those described in, for example, WO 01/34899, WO 01/14629, and U.S. Pat. No. 6,933,140.

Amylases:

The compositions can be combined with other amylases, such as non-production enhanced amylase. These can include commercially available amylases, such as but not limited to STAINZYME®, NATALASE®, DURAMYL®, TERMAMYL®, FUNGAMYL® and BAN™ (Novo Nordisk A/S and Novozymes A/S); RAPIDASE®, POWERASE®, and PURASTAR® (from Danisco US Inc.).

Cellulases:

Cellulases can be added to the compositions. Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed for example in U.S. Pat. Nos. 4,435,307; 5,648,263; 5,691,178; 5,776,757; and WO 89/09259. Exemplary cellulases contemplated for use are those having color care benefit for the textile. Examples of such cellulases are cellulases described in for example EP 0495257, EP 0531372, WO 96/11262, WO 96/29397, and WO 98/08940. Other examples are cellulase variants, such as those described in WO 94/07998; WO 98/12307; WO 95/24471; PCT/DK98/00299; EP 531315; U.S. Pat. Nos. 5,457,046; 5,686,593; and 5,763,254. Commercially available cellulases include CELLUZYME® and CAREZYME® (Novo Nordisk A/S and Novozymes A/S); CLAZINASE® and PURADAX HA® (Danisco US Inc.); and KAC-500(B)™ (Kao Corporation).

Peroxidases/Oxidases:

Suitable peroxidases/oxidases contemplated for use in the compositions include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include for example GUARDZYME™ (Novo Nordisk A/S and Novozymes A/S).

The detergent composition can also comprise 2,6-β-D-fructan hydrolase, which is effective for removal/cleaning of biofilm present on household and/or industrial textile/laundry.

The detergent enzyme(s) may be included in a detergent composition by adding separate additives containing one or more enzymes, or by adding a combined additive comprising all of these enzymes. A detergent additive, i.e. a separate additive or a combined additive, can be formulated e.g., as a granulate, a liquid, a slurry, and the like. Exemplary detergent additive formulations include but are not limited to granulates, in particular non-dusting granulates, liquids, in particular stabilized liquids or slurries.

Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (e.g., polyethyleneglycol, PEG) with mean molar weights of 1,000 to 20,000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in, for example, GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

The detergent composition may be in any convenient form, e.g., a bar, a tablet, a powder, a granule, a paste, or a liquid. A liquid detergent may be aqueous, typically containing up to about 70% water, and 0% to about 30% organic solvent. Compact detergent gels containing about 30% or less water are also contemplated. The detergent composition can optionally comprise one or more surfactants, which may be non-ionic, including semi-polar and/or anionic and/or cationic and/or zwitterionic. The surfactants can be present in a wide range, from about 0.1% to about 60% by weight.

When included therein the detergent will typically contain from about 1% to about 40% of an anionic surfactant, such as linear alkylbenzenesulfonate, α-olefinsulfonate, alkyl sulfate (fatty alcohol sulfate), alcohol ethoxysulfate, secondary alkanesulfonate, α-sulfo fatty acid methyl ester, alkyl- or alkenylsuccinic acid, or soap.

When included therein, the detergent will usually contain from about 0.2% to about 40% of a non-ionic surfactant such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl-N-alkyl derivatives of glucosamine (“glucamides”).

The detergent may contain 0% to about 65% of a detergent builder or complexing agent such as zeolite, diphosphate, triphosphate, phosphonate, carbonate, citrate, nitrilotriacetic acid, ethylenediaminetetraacetic acid (EDTA), diethylenetriaminepentaacetic acid, alkyl- or alkenylsuccinic acid, soluble silicates or layered silicates (e.g., SKS-6 from Hoechst).

The detergent may comprise one or more polymers. Exemplary polymers include carboxymethylcellulose (CMC), poly(vinylpyrrolidone) (PVP), poly(ethylene glycol) (PEG), poly(vinyl alcohol) (PVA), poly(vinylpyridine-N-oxide), poly(vinylimidazole), polycarboxylates e.g., polyacrylates, maleic/acrylic acid copolymers), and lauryl methacrylate/acrylic acid copolymers.

The enzyme(s) of the detergent composition may be stabilized using conventional stabilizing agents, e.g., as polyol (e.g., propylene glycol or glycerol), a sugar or sugar alcohol, lactic acid, boric acid, or a boric acid derivative (e.g., an aromatic borate ester), or a phenyl boronic acid derivative (e.g., 4-formylphenyl boronic acid). The composition may be formulated as described in WO 92/19709 and WO 92/19708.

It is contemplated that in the detergent compositions, in particular the maltogenic amylase of the present teachings, may be added in an amount corresponding to about 0.01 to about 100 mg of enzyme protein per liter of wash liquor (e.g., about 0.05 to about 5.0 mg of enzyme protein per liter of wash liquor or 0.1 to about 1.0 mg of enzyme protein per liter of wash liquor).

Methods of Assessing Amylase Activity in Detergent Compositions

Numerous α-amylase cleaning assays are known in the art, including swatch and micro-swatch assays.

Brewing Compositions

The present maltogenic amylase may be a component of a brewing composition used in a process of brewing, i.e., making a fermented malt beverage. Non-fermentable carbohydrates form the majority of the dissolved solids in the final beer. This residue remains because of the inability of malt amylases to hydrolyze the alpha-1,6-linkages of the starch. The non-fermentable carbohydrates contribute about 50 calories per 12 ounces of beer. An amylase, in combination with a glucoamylase and optionally a pullulanase and/or isoamylase, assist in converting the starch into dextrins and fermentable sugars, lowering the residual non-fermentable carbohydrates in the final beer.

The principal raw materials used in making these beverages are water, hops and malt. In addition, adjuncts such as common corn grits, refined corn grits, brewer's milled yeast, rice, sorghum, refined corn starch, barley, barley starch, dehusked barley, wheat, wheat starch, torrified cereal, cereal flakes, rye, oats, potato, tapioca, and syrups, such as corn syrup, sugar cane syrup, inverted sugar syrup, barley and/or wheat syrups, and the like may be used as a source of starch.

For a number of reasons, the malt, which is produced principally from selected varieties of barley, has the greatest effect on the overall character and quality of the beer. First, the malt is the primary flavoring agent in beer. Second, the malt provides the major portion of the fermentable sugar. Third, the malt provides the proteins, which will contribute to the body and foam character of the beer. Fourth, the malt provides the necessary enzymatic activity during mashing. Hops also contribute significantly to beer quality, including flavoring. In particular, hops (or hops constituents) add desirable bittering substances to the beer. In addition, the hops act as protein precipitants, establish preservative agents and aid in foam formation and stabilization.

Grains, such as barley, oats, wheat, as well as plant components, such as corn, hops, and rice, also are used for brewing, both in industry and for home brewing. The components used in brewing may be unmalted or may be malted, i.e., partially germinated, resulting in an increase in the levels of enzymes, including α-amylase. For successful brewing, adequate levels of α-amylase enzyme activity are necessary to ensure the appropriate levels of sugars for fermentation. An amylase, by itself or in combination with another α-amylase(s), accordingly may be added to the components used for brewing.

As used herein, the term “stock” means grains and plant components that are crushed or broken. For example, barley used in beer production is a grain that has been coarsely ground or crushed to yield a consistency appropriate for producing a mash for fermentation. As used herein, the term “stock” includes any of the aforementioned types of plants and grains in crushed or coarsely ground forms. The methods described herein may be used to determine maltogenic amylase activity levels in both flours and stock.

Processes for making beer are well known in the art. See, e.g., Wolfgang Kunze (2004) “Technology Brewing and Malting,” Research and Teaching Institute of Brewing, Berlin (VLB), 3rd edition. Briefly, the process involves: (a) preparing a mash, (b) filtering the mash to prepare a wort, and (c) fermenting the wort to obtain a fermented beverage, such as beer. Typically, milled or crushed malt is mixed with water and held for a period of time under controlled temperatures to permit the enzymes present in the malt to convert the starch present in the malt into fermentable sugars. The mash is then transferred to a mash filter where the liquid is separated from the grain residue. This sweet liquid is called “wort,” and the left over grain residue is called “spent grain.” The mash is typically subjected to an extraction, which involves adding water to the mash in order to recover the residual soluble extract from the spent grain. The wort is then boiled vigorously to sterilize the wort and help develop the color, flavor and odor. Hops are added at some point during the boiling. The wort is cooled and transferred to a fermentor.

The wort is then contacted in a fermentor with yeast. The fermentor may be chilled to stop fermentation. The yeast flocculates and is removed. Finally, the beer is cooled and stored for a period of time, during which the beer clarifies and its flavor develops, and any material that might impair the appearance, flavor and shelf life of the beer settles out. The beer usually contains from about 2% to about 10% v/v alcohol, although beer with a higher alcohol content, e.g., 18% v/v, may be obtained. Prior to packaging, the beer is carbonated and, optionally, filtered and pasteurized.

The brewing composition comprising a maltogenic amylase, in combination with a glucoamylase and optionally a pullulanase and/or isoamylase, may be added to the mash of step (a) above, i.e., during the preparation of the mash. Alternatively, or in addition, the brewing composition may be added to the mash of step (b) above, i.e., during the filtration of the mash. Alternatively, or in addition, the brewing composition may be added to the wort of step (c) above, i.e., during the fermenting of the wort.

A fermented beverage, such as a beer, can be produced by one of the methods above. The fermented beverage can be a beer, such as full malted beer, beer brewed under the “Reinheitsgebot,” ale, IPA, lager, bitter, Happoshu (second beer), third beer, dry beer, near beer, light beer, low alcohol beer, low calorie beer, porter, bock beer, stout, malt liquor, non-alcoholic beer, non-alcoholic malt liquor and the like, but also alternative cereal and malt beverages such as fruit flavored malt beverages, e.g., citrus flavored, such as lemon-, orange-, lime-, or berry-flavored malt beverages, liquor flavored malt beverages, e.g., vodka-, rum-, or tequila-flavored malt liquor, or coffee flavored malt beverages, such as caffeine-flavored malt liquor, and the like.

Reduction of Iodine-Positive Starch

The maltogenic amylases of the present teachings may reduce the iodine-positive starch (IPS), when used in a method of liquefaction and/or saccharification. One source of IPS is from amylose that escapes hydrolysis and/or from retrograded starch polymer. Starch retrogradation occurs spontaneously in a starch paste, or gel on aging, because of the tendency of starch molecules to bind to one another followed by an increase in crystallinity. Solutions of low concentration become increasingly cloudy due to the progressive association of starch molecules into larger articles. Spontaneous precipitation takes place and the precipitated starch appears to be reverting to its original condition of cold-water insolubility. Pastes of higher concentration on cooling set to a gel, which on aging becomes steadily firmer due to the increasing association of the starch molecules. This arises because of the strong tendency for hydrogen bond formation between hydroxy groups on adjacent starch molecules. See J. A. Radley, ed., STARCH AND ITS DERIVATIVES 194-201 (Chapman and Hall, London (1968)).

The presence of IPS in saccharide liquor negatively affects final product quality and represents a major issue with downstream processing. IPS plugs or slows filtration system, and fouls the carbon columns used for purification. When IPS reaches sufficiently high levels, it may leak through the carbon columns and decrease production efficiency. Additionally, it may results in hazy final product upon storage, which is unacceptable for final product quality. The amount of IPS can be reduced by isolating the saccharification tank and blending the contents back. IPS nevertheless will accumulate in carbon columns and filter systems, among other things. The use of the maltogenic amylases of the present teachings is expected to improve overall process performance by reducing the amount of IPS.

### Examples

A putative novel amylase was identified from a metagenomic library constructed by conventional cloning techniques. Protein sequence analysis indicates that the amylase belongs to the glycosyl hydrolase family 13 (GH13), and shows less than 66% sequence identity to known proteins in the public NCBI database.

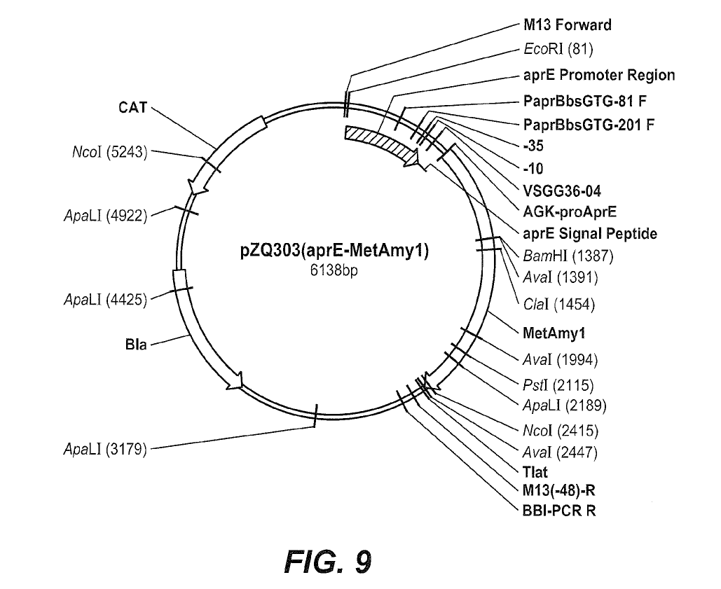

Following identification of the putative amylase in silico, the gene was cloned using conventional molecular biology PCR techniques and expressed in Bacillus subtilis. The plasmid cloning map is shown in FIG. 9.

### Cloning and Expression of AmyMG

The full gene sequence is SEQ ID NO:1.

CACACCCCGACGACCCGGCAGGCCGATTACTACGGCACGCTGGAGCCGTT
TGCGCGTGAAGCGGTGTACTTCGTGATGACCGATCGCTTCGTCAACGGCG
ACCCCGGCAACGACCACCGCGACCAAGGCGGCGCCCTGGGCACGTTCGAC
ATCCCGCTGCCGCCATGCAATGGCGTGTCCGGCAACATCGGCTACCTGGG
TGGCGACTTCAAGGGCCTGGCCGATCATCTGGATTACATCCGCGAAATGG
GCTTCACCGCGGTGTGGATCACGCCGATCGTGGACAATCCGGACCAGCGC
TTCACTGGCGGCAGCGCACCAACCTGCGGCGGCATTCTGGCTGACCAGGG
CAAAGCCGGCTATCACGGCTACTGGGGCGTGAATTTCTACCAAGTGGACG
AGCACCTGCCCAGCCCAGGCATGGACTTCCGCGACCTGGCGGCGGCGATG
CATCGCAAGGGCATGAAGCTGGTGCTGGACATCGTGGGGAACCACGGCTC
GCCGGCCTGGGGCATGGCCTTCGACCAGCCCAAGTTCGGCAAGATCTACG
ACAAGGACGGCACGCTGATTGCCGATCACCAGAACCTGCCGCCGCAGCAG
CTGGATCCCGAGCACAACCCGCTGCACCGCTTCTACAACACGGTCGGCCC
GGTGGACGGGGCCAAGGGATCGATCTTCGACGGCAATCTGGCCCAGCTGT
CGGATCTCAATGAACGCAACCCGGACGTGCTGGACTATCTGGTCGGGGCC
TATCTGCAATGGATCGACCAGGGTGCCGATGCGTTTCGCATCGACACCAT
CGCCTGGATGCCGGACAGCTTCTGGCAGGCCTTCACCACCCGCATCCGGG
CAAAGCACCCCGGCTTTTTCATGTTCGGCGAGGCCTTCGACTACGACGCC
GCCAGGATTGCCACCCACACCCTGCCCGGCCACGGCGAAACCAGCGTGCT
GGACTTCCCGATGAAACAGGCGATGGAAGAGGTCTTCGGGCGCAAGCAGG
CCGGTTTTGAACGGATGATACCGGCGCTGCATCTGACTGGCGGCCCGTAT
GCCAACCCCTACGAGCTGGCCACCTTCTACGACAATCACGACATGCCGCG
GCTGGATGCCAGCGATGAAGGCTTCATTGATGCACACAACTGGCTGTTCA
CCGCGCGTGGCATTCCGGTGGTCTATTACGGCTCGGAAATGGGCTTCATG
CGCGGCCGACCCGAGCACGGCGGCAACCGCAACTACTTCGGCACCGAAGG
CATTGCCGCCGCCAAGGCAAGCCCGATCCGGGCAGCGCTGACCCGCATTG
CGCAGGTGCGTGCCGCTTCACCAGCGCTGCAGCgCGGGCTGCAACTCAAT
CTGGAGCTGCAAGGCAACCGCGCCGCGTTCTATCGGGTGTACCAGCACGA
CGGTGTGCACCAGATCGCGCTGGTCCTGCTCAACAAGGGCGACGCCCCGG
AACACTTTGCCGTCCAGACGATGCTGCAACCCGGCcGCTGGCATGACGCG
ATTGGCGGTGAGACGCTGACCATACAGGCTGGCGAGGCGCTGCACGCCGA
GGTTCCGGCGCATGGCGTGCGGGTCTTTCTGCTCGACGCCCAGGTGACTG
AGCCGACGCTGGCGGCTGCGCTGGATGCCGCCATGGCCGATGCACGCCGG
TCACGGTAA

The nucleic acid fragment encoding the native signal peptide is SEQ ID NO: 2.

ATGATCACCATGCCCCTGCGTTCTGCCCGCCTCGGCCTGAGTTTGCTTTG
CGCGCTTGCCTCGACGGCCTGTGCA

The full protein sequence is SEQ ID NO:3.

HTPTTRQADYYGTLEPFAREAVYFVMTDRFVNGDPGNDHRDQGGALGTFD
IPLPPCNGVSGNIGYLGGDFKGLADHLDYIREMGFTAVWITPIVDNPDQR
FTGGSAPTCGGILADQGKAGYHGYWGVNFYQVDEHLPSPGMDFRDLAAAM
HRKGMKLVLDIVGNHGSPAWGMAFDQPKFGKIYDKDGTLIADHQNLPPQQ
LDPEHNPLHRFYNTVGPVDGAKGSIFDGNLAQLSDLNERNPDVLDYLVGA
YLQWIDQGADAFRIDTIAWMPDSFWQAFTTRIRAKHPGFFMFGEAFDYDA
ARIATHTLPGHGETSVLDFPMKQAMEEVFGRKQAGFERMIPALHLTGGPY
ANPYELATFYDNHDMPRLDASDEGFIDAHNWLFTARGIPVVYYGSEMGFM
RGRPEHGGNRNYFGTEGIAAAKASPIRAALTRIAQVRAASPALQRGLQLN
LELQGNRAAFYRVYQHDGVHQIALVLLNKGDAPEHFAVQTMLQPGRWHDA
IGGETLTIQAGEALHAEVPAHGVRVFLLDAQVTEPTLAAALDAAMADARR
SR

The native signal peptide is SEQ ID NO: 4.

MITMPLRSARLGLSLLCALASTACA

The mature gene sequence was identified by conventional procedures, PCR amplified, digested by BssHii/XhoI, and ligated into the p2JM plasmid digested with same restriction enzymes. As shown in the plasmid map of FIG. 9, the AmyMG gene was put under the control of the aprE promoter. The aprE signal sequence was used to direct protein secretion. In addition, nucleotides coding for three additional amino acids (AGK) were placed between the aprE signal sequence and the mature AmyMG gene to facilitate secretion of the target protein. The resulting plasmid was used to transform competent Bacillus subtilis cells. Expression evaluation of AmyMG showed that the enzyme expresses very well in both strains.

The aprE signal nucleic acid sequence (underlined)+AGK nucleic acid sequence (italics) is SEQ ID NO: 5.

gtgagaagcaaaaaattgtggatcagcttgttgtttgcgttaacgttaat
ctttacgatggcgttcagcaacatgagcgcgcaggcagctggtaaa

The aprE signal amino acid sequence (underlined)+AGK amino acid sequence (italics) is SEQ ID NO: 6.

MRSKKLWISLLFALALIFTMAFGSTSSAQAAGK

### Product Profile Analysis and Biochemical Characterization of AmyMG

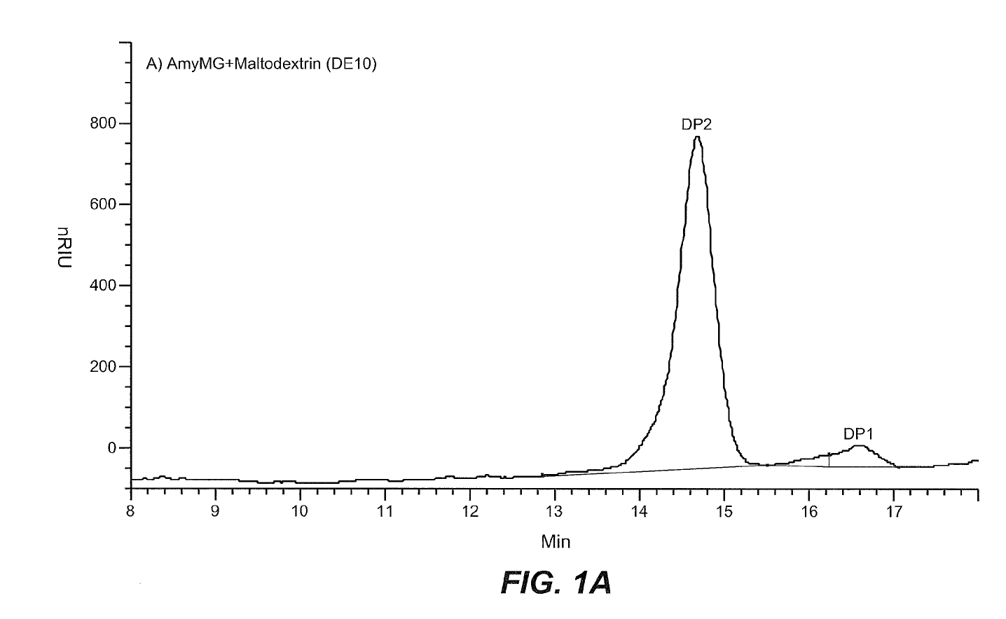

Preliminary product profile analysis showed that AmyMG exhibited DP2 as its major product, with the composition (%) higher than 90% (if one only considered the product profile from DP1 to DP7) (FIG. 1 and Table 1).

FIG. 1 shows the typical chromatograms of oligosaccharide product profile analysis of A) AmyMG (10 ppm) incubated with Maltodextrin (DE10) (0.5%, w/v) under pH 5.3 at 50° C. for 2 h and B) the mixture of standard compounds of DP1 to DP 7 (0.0125%, w/v). HPLC separation was done using an Agilent 1200 series HPLC system with an Aminex HPX-42A column (300 mm×7.8 mm) at 85° C. The sample (10 μL) was subjected to the HPLC column and separated with an isocratic gradient of Milli-Q water as the mobile phase at a flow rate of 0.6 mL/min. The oligosaccharide products were detected using a refractive index detector.

TABLE 2
The oligosaccharide product compositions (%) of AmyMG

| | Na-Citrate (pH 5.3) | | | | | | | HEPES (pH 8.2) | | | | | | |
|---|---|---|---|---|---|---|---|---|---|---|---|---|---|---|
| | Product composition (%) | | | | | | | Product composition (%) | | | | | | |
| Substrate | DP1 | DP2 | DP3 | DP4 | DP5 | DP6 | DP7 | DP1 | DP2 | DP3 | DP4 | DP5 | DP6 | DP7 |
| DP7 | 17 | 83 | 0 | 0 | 0 | 0 | — | 18 | 82 | 0 | 0 | 0 | 0 | — |
| Amylopectin | 3 | 96 | 0 | 1 | 0 | 0 | 0 | 3 | 97 | 0 | 0 | 0 | 0 | 0 |
| Maltodextrin | 6 | 94 | 0 | 0 | 0 | 0 | 0 | 11 | 89 | 0 | 0 | 0 | 0 | 0 |
| (DE10) | | | | | | | | | | | | | | |

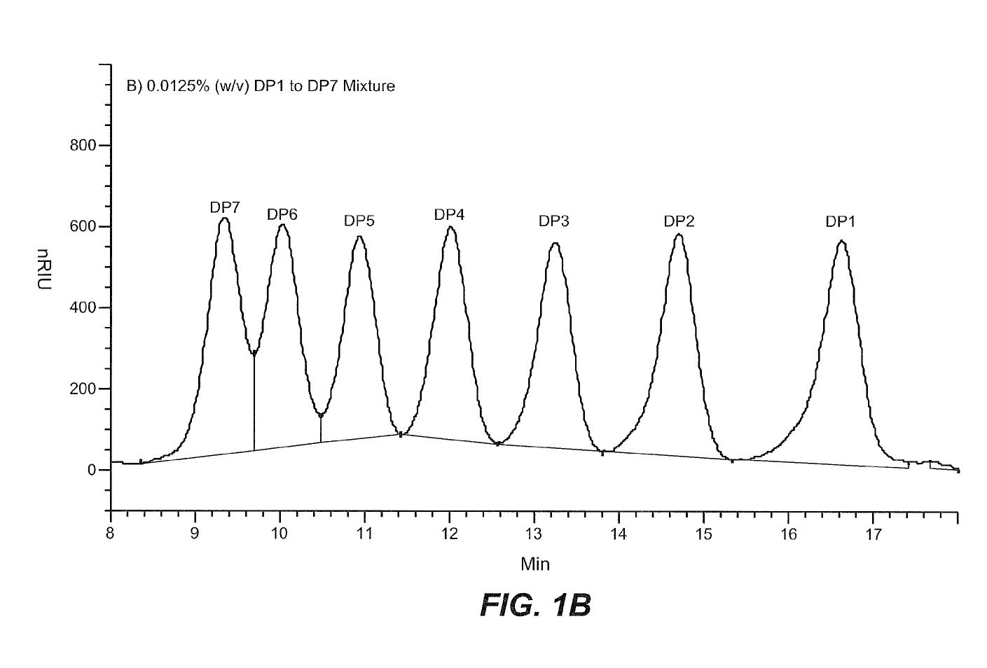

Ion exchange chromatography results confirmed that AmyMG showed maltogenic activity with maltose as its major product (FIG. 2). The typical chromatograms of oligosaccharide product profile analysis of AmyMG (10 ppm) incubated with Maltodextrin (DE10) (15%, w/v) under pH 5.3 at 50° C. for 24 h. The separation was done using a Dionex ICS-5000 ion exchange chromatography with a CarboPac PA 200 column at 30° C. The sample (25 μL) was subjected to the column and separated with the gradient: 0-10 min, 50 mM NaOH; 10-15 min, 50-100 mM NaOH; 15-35 min, 100 mM NaOH, 0-200 mM NaAc; 35-45 min, 50 mM NaOH, at a flow rate of 0.5 mL/min. The oligosaccharide products were detected using a pulsed amperometric detector.

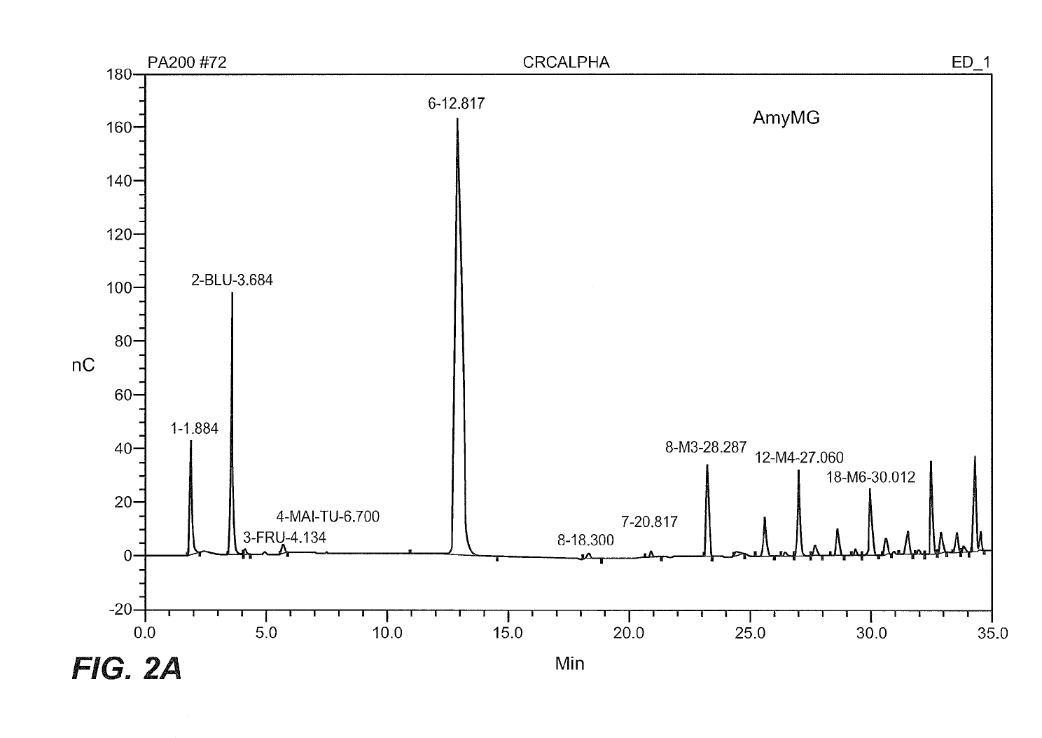

More detailed product profile analysis (FIG. 3) with different incubation time points showed the peak area of DP2 increased while the peak area of DP10+ decreased with the extension of incubation time from 0 h up to 48 h, indicating that maltose is the major product of AmyMG by hydrolyzing Maltrin040. As shown, the typical chromatograms of oligosaccharide product profile analysis of AmyMG (25 ppm) incubated with Maltrin040 (30%, w/v) under pH 5.3 at 50° C. HPLC separation was done using an Agilent 1200 series HPLC system with an Aminex HPX-42A column (300 mm×7.8 mm) at 85° C. The sample (10 μL) was subjected to the HPLC column and separated with an isocratic gradient of Milli-Q water as the mobile phase at a flow rate of 0.6 mL/min. The oligosaccharide products were detected using a refractive index detector.

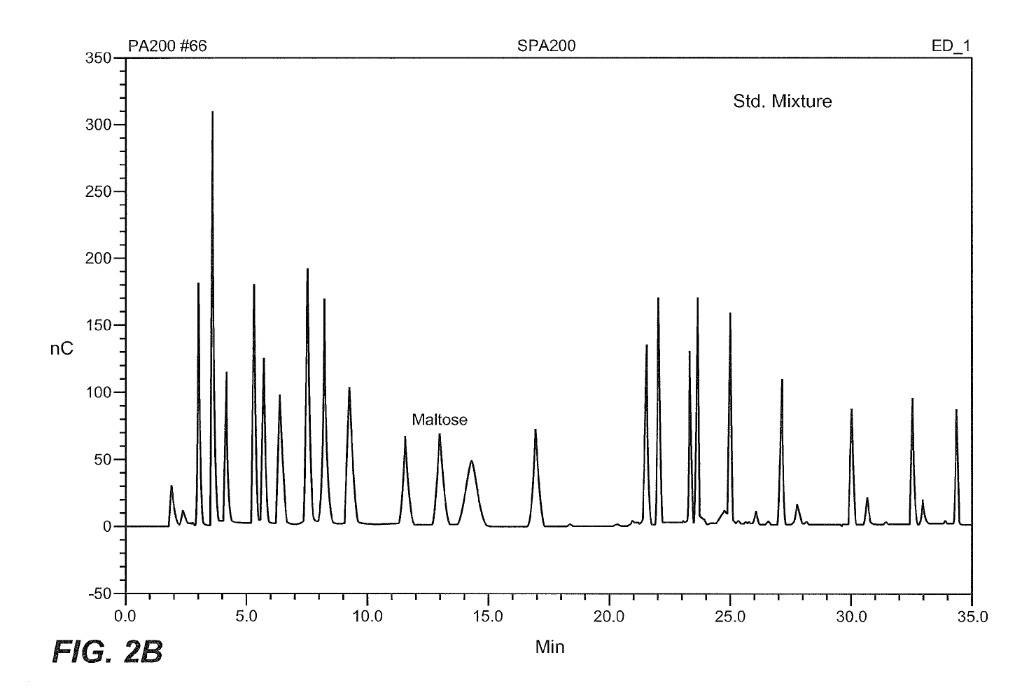

In order to figure out the exact anomeric form of maltose produced from maltodextrin catalyzed by AmyMG, the real time NMR assay was done by analyzing the enzyme product at different reaction times (FIG. 4). The results suggested that the product is alpha-maltose and AmyMG is a maltogenic alpha-amylase. As shown, ¹H-NMR of the product from Maltrin040 incubated with AmyMG. The reaction was done by incubating the mixture of 10 mg Maltrin040 that was dissolved into 1 mL of D20 and 33 ppm of AmyMG at 25° C. The NMR assay was done by a Bruker NMR spectrometer that operated at 500 MHz in D20.

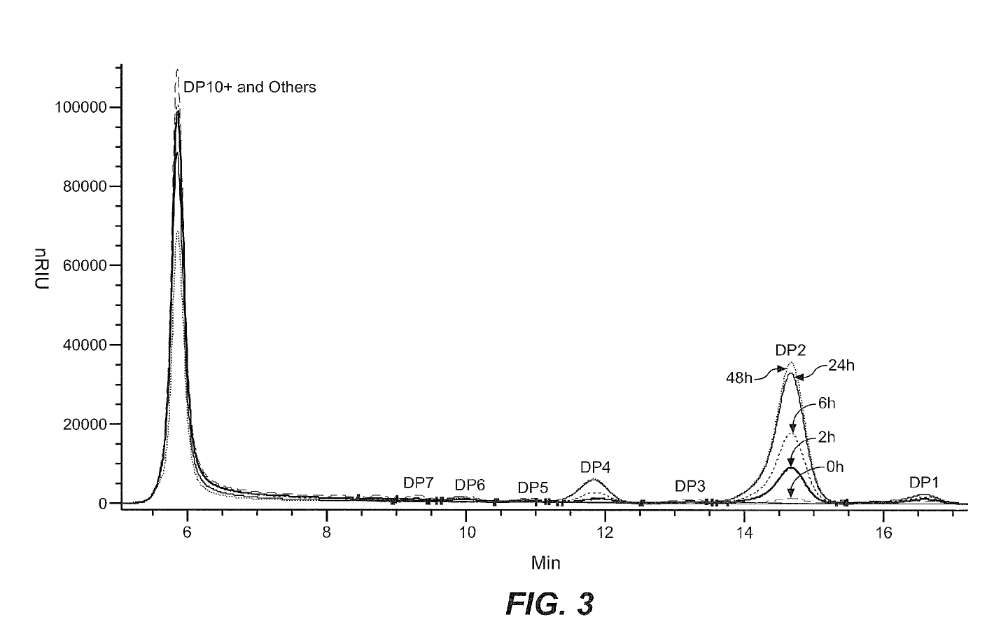

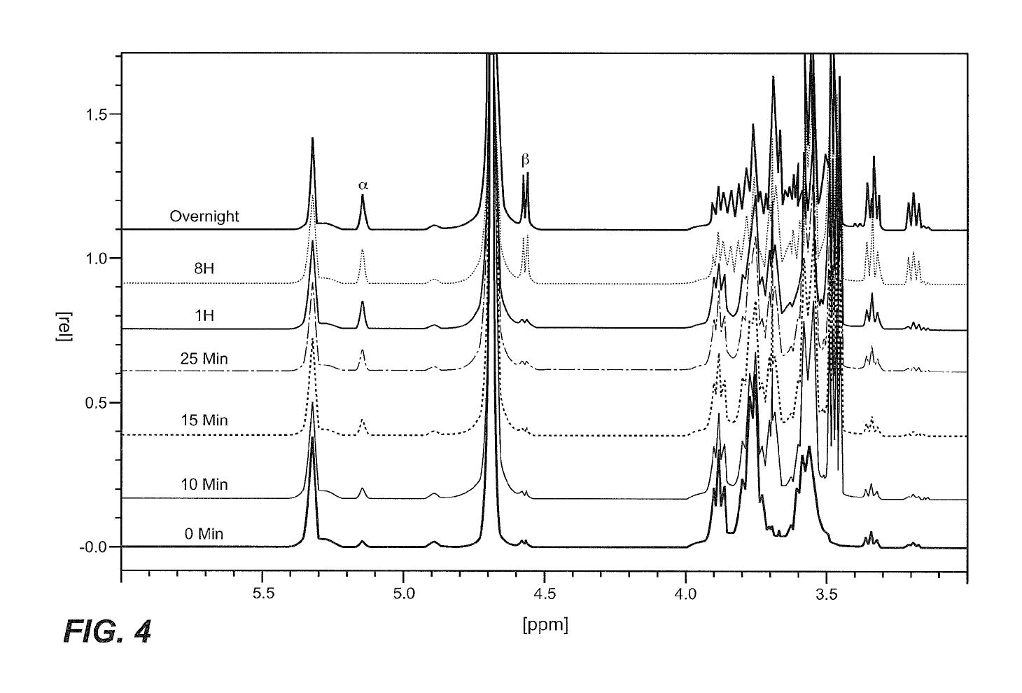

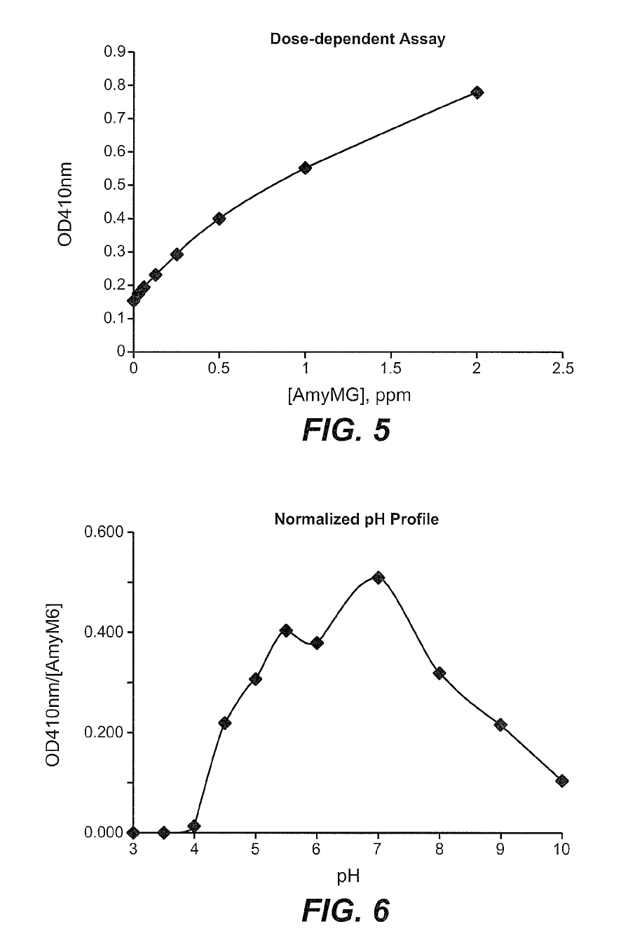

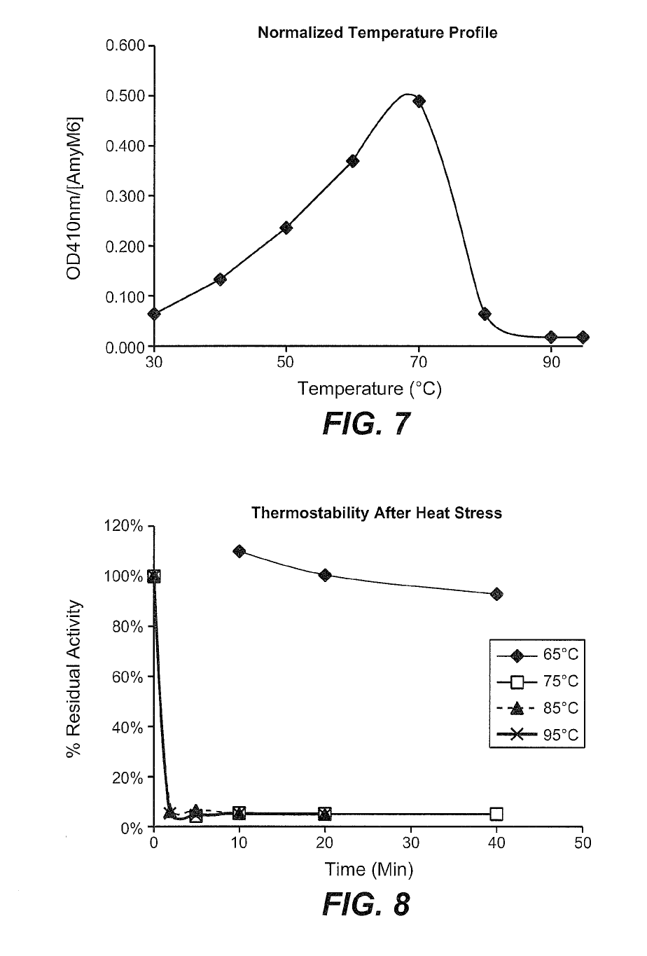

AmyMG was tested for alpha-amylase characterization (Amylopectin/PAHBAH method), including dose dependent assay (FIG. 5, a dose-dependant assay for AmyMG using amylopectin as the substrate), pH (FIG. 6, showing a normalized pH profile), temperature profile (FIG. 7, showing a normalized temperature profile), and, a thermostability assay (FIG. 8, showing the thermostability of AmyMG). The specific activity of this enzyme towards amylopectin is 218.4 U/mg, with optimum pH at 7 and optimum temperature at 63° C.

Thus, in some embodiments, the present teachings provide a polypeptide with maltogenic activity wherein at least 70%, at least 75%, at least 80%, at least 85%, at least 90%, or at least 95% of the maximum enzyme activity is present in the pH range of 6-8, as assessed by DP° Units.

Additionally, in some embodiments, the present teachings provide a polypeptide with maximum activity, as assessed by DP° Units, at 60° C.-70° C., or 62° C.-64° C.

Additionally, in some embodiments, the present teachings provide a polypeptide that produces at least 80%, at least 85%, at least 90%, or at least 95% DP2 of the DP1 to DP7 products resulting from a hydrolysis reaction performed according to the procedures depicted in FIG. 3.

Finally, in some embodiments, the polypeptides of the present teachings can be engineered to provide various improved properties. Such engineering efforts can be guided by the atomic coordinate structural information provided in Table 3.

TABLE 3

| | | | | | | | | | | | |
|---|---|---|---|---|---|---|---|---|---|---|---|
| ATOM | 1 | N | GLN | A | 8 | 114.737 | 81.675 | 18.562 | 1.00 | 37.71 | N |
| ATOM | 2 | CA | GLN | A | 8 | 114.425 | 83.092 | 18.184 | 1.00 | 34.91 | C |
| ATOM | 3 | CB | GLN | A | 8 | 115.667 | 83.800 | 17.610 | 1.00 | 42.85 | C |
| ATOM | 4 | CG | GLN | A | 8 | 115.634 | 85.314 | 17.761 | 1.00 | 49.20 | C |
| ATOM | 5 | CD | GLN | A | 8 | 115.695 | 85.734 | 19.218 | 1.00 | 59.92 | C |
| ATOM | 6 | OE1 | GLN | A | 8 | 114.734 | 85.547 | 19.968 | 1.00 | 58.08 | O |
| ATOM | 7 | NE2 | GLN | A | 8 | 116.832 | 86.289 | 19.633 | 1.00 | 55.51 | N |
| ATOM | 8 | C | GLN | A | 8 | 113.216 | 83.263 | 17.240 | 1.00 | 26.15 | C |
| ATOM | 9 | O | GLN | A | 8 | 112.182 | 83.767 | 17.675 | 1.00 | 24.80 | O |
| ATOM | 10 | N | ALA | A | 9 | 113.343 | 82.897 | 15.960 | 1.00 | 21.01 | N |
| ATOM | 11 | CA | ALA | A | 9 | 112.213 | 82.903 | 15.027 | 1.00 | 18.94 | C |
| ATOM | 12 | CB | ALA | A | 9 | 112.613 | 82.251 | 13.712 | 1.00 | 19.85 | C |
| ATOM | 13 | C | ALA | A | 9 | 111.041 | 82.146 | 15.671 | 1.00 | 15.96 | C |
| ATOM | 14 | O | ALA | A | 9 | 111.263 | 81.146 | 16.368 | 1.00 | 16.65 | O |
| ATOM | 15 | N | ASP | A | 10 | 109.823 | 82.651 | 15.494 | 1.00 | 13.87 | N |
| ATOM | 16 | CA | ASP | A | 10 | 108.612 | 81.920 | 15.913 | 1.00 | 15.61 | C |
| ATOM | 17 | CB | ASP | A | 10 | 107.434 | 82.870 | 16.119 | 1.00 | 19.46 | C |
| ATOM | 18 | CG | ASP | A | 10 | 107.469 | 83.592 | 17.448 | 1.00 | 23.75 | C |
| ATOM | 19 | OD1 | ASP | A | 10 | 107.906 | 83.023 | 18.466 | 1.00 | 20.82 | O |
| ATOM | 20 | OD2 | ASP | A | 10 | 107.009 | 84.743 | 17.468 | 1.00 | 32.09 | O |
| ATOM | 21 | C | ASP | A | 10 | 108.159 | 80.918 | 14.856 | 1.00 | 12.27 | C |
| ATOM | 22 | O | ASP | A | 10 | 107.934 | 81.294 | 13.725 | 1.00 | 12.47 | O |
| ATOM | 23 | N | TYR | A | 11 | 107.976 | 79.664 | 15.244 | 1.00 | 11.78 | N |
| ATOM | 24 | CA | TYR | A | 11 | 107.418 | 78.635 | 14.367 | 1.00 | 10.00 | C |
| ATOM | 25 | CB | TYR | A | 11 | 108.508 | 77.669 | 13.941 | 1.00 | 10.62 | C |
| ATOM | 26 | CG | TYR | A | 11 | 109.637 | 78.276 | 13.132 | 1.00 | 10.60 | C |
| ATOM | 27 | CD1 | TYR | A | 11 | 109.376 | 79.006 | 11.986 | 1.00 | 12.14 | C |
| ATOM | 28 | CE1 | TYR | A | 11 | 110.393 | 79.548 | 11.225 | 1.00 | 11.89 | C |
| ATOM | 29 | CZ | TYR | A | 11 | 111.713 | 79.360 | 11.601 | 1.00 | 13.20 | C |
| ATOM | 30 | OH | TYR | A | 11 | 112.721 | 79.895 | 10.822 | 1.00 | 13.22 | O |
| ATOM | 31 | CE2 | TYR | A | 11 | 112.008 | 78.630 | 12.736 | 1.00 | 12.02 | C |
| ATOM | 32 | CD2 | TYR | A | 11 | 110.962 | 78.079 | 13.485 | 1.00 | 12.17 | C |
| ATOM | 33 | C | TYR | A | 11 | 106.294 | 77.907 | 15.116 | 1.00 | 10.63 | C |
| ATOM | 34 | O | TYR | A | 11 | 106.515 | 77.293 | 16.167 | 1.00 | 10.75 | O |
| ATOM | 35 | N | TYR | A | 12 | 105.067 | 78.040 | 14.612 | 1.00 | 10.78 | N |
| ATOM | 36 | CA | TYR | A | 12 | 103.913 | 77.379 | 15.195 | 1.00 | 10.87 | C |
| ATOM | 37 | CB | TYR | A | 12 | 103.411 | 78.087 | 16.449 | 1.00 | 11.39 | C |
| ATOM | 38 | CG | TYR | A | 12 | 103.189 | 79.570 | 16.322 | 1.00 | 12.09 | C |
| ATOM | 39 | CD1 | TYR | A | 12 | 102.050 | 80.066 | 15.729 | 1.00 | 13.04 | C |
| ATOM | 40 | CE1 | TYR | A | 12 | 101.834 | 81.424 | 15.603 | 1.00 | 13.98 | C |
| ATOM | 41 | CZ | TYR | A | 12 | 102.771 | 82.298 | 16.120 | 1.00 | 14.07 | C |
| ATOM | 42 | OH | TYR | A | 12 | 102.570 | 83.635 | 16.006 | 1.00 | 19.59 | O |
| ATOM | 43 | CE2 | TYR | A | 12 | 103.906 | 81.846 | 16.726 | 1.00 | 13.46 | C |
| ATOM | 44 | CD2 | TYR | A | 12 | 104.110 | 80.476 | 16.841 | 1.00 | 14.72 | C |
| ATOM | 45 | C | TYR | A | 12 | 102.783 | 77.285 | 14.178 | 1.00 | 10.60 | C |
| ATOM | 46 | O | TYR | A | 12 | 102.615 | 78.167 | 13.319 | 1.00 | 8.70 | O |
| ATOM | 47 | N | GLY | A | 13 | 102.040 | 76.190 | 14.257 | 1.00 | 9.04 | N |
| ATOM | 48 | CA | GLY | A | 13 | 100.830 | 76.045 | 13.454 | 1.00 | 9.97 | C |
| ATOM | 49 | C | GLY | A | 13 | 99.734 | 76.942 | 13.991 | 1.00 | 9.94 | C |
| ATOM | 50 | O | GLY | A | 13 | 99.001 | 77.567 | 13.229 | 1.00 | 9.84 | O |
| ATOM | 51 | N | THR | A | 14 | 99.632 | 76.993 | 15.312 | 1.00 | 9.70 | N |
| ATOM | 52 | CA | THR | A | 14 | 98.663 | 77.856 | 15.980 | 1.00 | 10.20 | C |
| ATOM | 53 | CB | THR | A | 14 | 97.245 | 77.241 | 15.994 | 1.00 | 10.01 | C |
| ATOM | 54 | OG1 | THR | A | 14 | 96.360 | 78.172 | 16.611 | 1.00 | 10.36 | O |
| ATOM | 55 | CG2 | THR | A | 14 | 97.189 | 75.925 | 16.776 | 1.00 | 8.48 | C |
| ATOM | 56 | C | THR | A | 14 | 99.067 | 78.090 | 17.419 | 1.00 | 10.24 | C |
| ATOM | 57 | O | THR | A | 14 | 99.709 | 77.220 | 18.020 | 1.00 | 9.49 | O |
| ATOM | 58 | N | LEU | A | 15 | 98.661 | 79.244 | 17.964 | 1.00 | 10.52 | N |
| ATOM | 59 | CA | LEU | A | 15 | 98.798 | 79.553 | 19.390 | 1.00 | 11.07 | C |
| ATOM | 60 | CB | LEU | A | 15 | 99.342 | 80.990 | 19.605 | 1.00 | 12.17 | C |
| ATOM | 61 | CG | LEU | A | 15 | 100.793 | 81.221 | 19.135 | 1.00 | 14.65 | C |
| ATOM | 62 | CD1 | LEU | A | 15 | 101.260 | 82.655 | 19.445 | 1.00 | 13.18 | C |
| ATOM | 63 | CD2 | LEU | A | 15 | 101.724 | 80.178 | 19.758 | 1.00 | 13.48 | C |
| ATOM | 64 | C | LEU | A | 15 | 97.459 | 79.397 | 20.128 | 1.00 | 12.49 | C |
| ATOM | 65 | O | LEU | A | 15 | 97.373 | 79.717 | 21.299 | 1.00 | 12.03 | O |
| ATOM | 66 | N | GLU | A | 16 | 96.411 | 78.891 | 19.462 | 1.00 | 12.24 | N |
| ATOM | 67 | CA | GLU | A | 16 | 95.139 | 78.627 | 20.163 | 1.00 | 11.45 | C |
| ATOM | 68 | CB | GLU | A | 16 | 94.117 | 78.040 | 19.189 | 1.00 | 12.06 | C |
| ATOM | 69 | CG | GLU | A | 16 | 92.920 | 77.366 | 19.815 | 1.00 | 13.55 | C |
| ATOM | 70 | CD | GLU | A | 16 | 92.156 | 78.277 | 20.763 | 1.00 | 15.28 | C |
| ATOM | 71 | OE1 | GLU | A | 16 | 91.845 | 79.402 | 20.326 | 1.00 | 14.11 | O |
| ATOM | 72 | OE2 | GLU | A | 16 | 91.851 | 77.844 | 21.918 | 1.00 | 12.98 | O |
| ATOM | 73 | C | GLU | A | 16 | 95.392 | 77.679 | 21.343 | 1.00 | 10.66 | C |
| ATOM | 74 | O | GLU | A | 16 | 95.931 | 76.586 | 21.153 | 1.00 | 10.99 | O |
| ATOM | 75 | N | PRO | A | 17 | 95.031 | 78.099 | 22.573 | 1.00 | 12.38 | N |
| ATOM | 76 | CA | PRO | A | 17 | 95.229 | 77.237 | 23.730 | 1.00 | 11.70 | C |
| ATOM | 77 | CB | PRO | A | 17 | 94.522 | 78.016 | 24.857 | 1.00 | 15.92 | C |
| ATOM | 78 | CG | PRO | A | 17 | 94.750 | 79.456 | 24.485 | 1.00 | 15.36 | C |
| ATOM | 79 | CD | PRO | A | 17 | 94.605 | 79.467 | 22.979 | 1.00 | 13.18 | C |
| ATOM | 80 | C | PRO | A | 17 | 94.617 | 75.842 | 23.584 | 1.00 | 12.59 | C |
| ATOM | 81 | O | PRO | A | 17 | 95.176 | 74.874 | 24.084 | 1.00 | 10.90 | O |
| ATOM | 82 | N | PHE | A | 18 | 93.473 | 75.723 | 22.911 | 1.00 | 10.98 | N |
| ATOM | 83 | CA | PHE | A | 18 | 92.771 | 74.431 | 22.893 | 1.00 | 10.65 | C |
| ATOM | 84 | CB | PHE | A | 18 | 91.300 | 74.592 | 22.466 | 1.00 | 11.32 | C |
| ATOM | 85 | CG | PHE | A | 18 | 90.399 | 73.608 | 23.124 | 1.00 | 11.42 | C |
| ATOM | 86 | CD1 | PHE | A | 18 | 89.862 | 73.884 | 24.366 | 1.00 | 12.54 | C |
| ATOM | 87 | CE1 | PHE | A | 18 | 89.031 | 72.960 | 24.998 | 1.00 | 14.32 | C |
| ATOM | 88 | CZ | PHE | A | 18 | 88.755 | 71.758 | 24.381 | 1.00 | 12.10 | C |
| ATOM | 89 | CE2 | PHE | A | 18 | 89.291 | 71.476 | 23.144 | 1.00 | 12.87 | C |
| ATOM | 90 | CD2 | PHE | A | 18 | 90.118 | 72.399 | 22.524 | 1.00 | 11.38 | C |
| ATOM | 91 | C | PHE | A | 18 | 93.467 | 73.352 | 22.046 | 1.00 | 10.19 | C |
| ATOM | 92 | O | PHE | A | 18 | 93.154 | 72.163 | 22.153 | 1.00 | 9.58 | O |
| ATOM | 93 | N | ALA | A | 19 | 94.467 | 73.744 | 21.266 | 1.00 | 11.62 | N |
| ATOM | 94 | CA | ALA | A | 19 | 95.189 | 72.795 | 20.451 | 1.00 | 12.01 | C |
| ATOM | 95 | CB | ALA | A | 19 | 96.140 | 73.525 | 19.489 | 1.00 | 12.82 | C |
| ATOM | 96 | C | ALA | A | 19 | 95.939 | 71.753 | 21.275 | 1.00 | 11.44 | C |
| ATOM | 97 | O | ALA | A | 19 | 96.264 | 70.661 | 20.767 | 1.00 | 12.23 | O |
| ATOM | 98 | N | ARG | A | 20 | 96.239 | 72.063 | 22.524 | 1.00 | 10.20 | N |
| ATOM | 99 | CA | ARG | A | 20 | 96.929 | 71.113 | 23.389 | 1.00 | 12.13 | C |
| ATOM | 100 | CB | ARG | A | 20 | 97.459 | 71.771 | 24.681 | 1.00 | 14.90 | C |
| ATOM | 101 | CG | ARG | A | 20 | 96.379 | 72.227 | 25.683 | 1.00 | 22.99 | C |
| ATOM | 102 | CD | ARG | A | 20 | 96.968 | 72.828 | 26.976 | 1.00 | 26.81 | C |
| ATOM | 103 | NE | ARG | A | 20 | 97.421 | 71.776 | 27.886 | 1.00 | 36.93 | N |
| ATOM | 104 | CZ | ARG | A | 20 | 96.644 | 71.115 | 28.753 | 1.00 | 43.46 | C |
| ATOM | 105 | NH1 | ARG | A | 20 | 95.347 | 71.380 | 28.870 | 1.00 | 39.73 | N |
| ATOM | 106 | NH2 | ARG | A | 20 | 97.172 | 70.172 | 29.519 | 1.00 | 44.22 | N |
| ATOM | 107 | C | ARG | A | 20 | 96.045 | 69.954 | 23.780 | 1.00 | 11.42 | C |
| ATOM | 108 | O | ARG | A | 20 | 96.552 | 68.960 | 24.302 | 1.00 | 9.84 | O |
| ATOM | 109 | N | GLU | A | 21 | 94.730 | 70.098 | 23.574 | 1.00 | 10.02 | N |
| ATOM | 110 | CA | GLU | A | 21 | 93.772 | 69.145 | 24.120 | 1.00 | 10.20 | C |
| ATOM | 111 | CB | GLU | A | 21 | 92.403 | 69.807 | 24.361 | 1.00 | 10.61 | C |
| ATOM | 112 | CG | GLU | A | 21 | 92.428 | 71.104 | 25.167 | 1.00 | 11.29 | C |
| ATOM | 113 | CD | GLU | A | 21 | 92.854 | 70.952 | 26.607 | 1.00 | 13.95 | C |
| ATOM | 114 | OE1 | GLU | A | 21 | 92.766 | 69.823 | 27.154 | 1.00 | 16.27 | O |
| ATOM | 115 | OE2 | GLU | A | 21 | 93.226 | 71.998 | 27.215 | 1.00 | 14.14 | O |
| ATOM | 116 | C | GLU | A | 21 | 93.579 | 67.955 | 23.172 | 1.00 | 9.85 | C |
| ATOM | 117 | O | GLU | A | 21 | 93.870 | 68.044 | 21.988 | 1.00 | 9.19 | O |
| ATOM | 118 | N | ALA | A | 22 | 93.096 | 66.848 | 23.730 | 1.00 | 8.78 | N |
| ATOM | 119 | CA | ALA | A | 22 | 92.593 | 65.712 | 22.987 | 1.00 | 8.73 | C |
| ATOM | 120 | CB | ALA | A | 22 | 93.543 | 64.520 | 23.099 | 1.00 | 7.76 | C |
| ATOM | 121 | C | ALA | A | 22 | 91.244 | 65.378 | 23.595 | 1.00 | 9.11 | C |
| ATOM | 122 | O | ALA | A | 22 | 91.118 | 65.264 | 24.825 | 1.00 | 8.96 | O |
| ATOM | 123 | N | VAL | A | 23 | 90.250 | 65.196 | 22.726 | 1.00 | 9.91 | N |
| ATOM | 124 | CA | VAL | A | 23 | 88.816 | 65.108 | 23.110 | 1.00 | 9.02 | C |
| ATOM | 125 | CB | VAL | A | 23 | 87.955 | 66.035 | 22.243 | 1.00 | 7.58 | C |
| ATOM | 126 | CG1 | VAL | A | 23 | 86.471 | 65.954 | 22.657 | 1.00 | 8.23 | C |
| ATOM | 127 | CG2 | VAL | A | 23 | 88.484 | 67.474 | 22.301 | 1.00 | 7.05 | C |
| ATOM | 128 | C | VAL | A | 23 | 88.236 | 63.700 | 22.987 | 1.00 | 9.21 | C |
| ATOM | 129 | O | VAL | A | 23 | 88.382 | 63.056 | 21.958 | 1.00 | 9.50 | O |
| ATOM | 130 | N | TYR | A | 24 | 87.580 | 63.238 | 24.052 | 1.00 | 9.44 | N |
| ATOM | 131 | CA | TYR | A | 24 | 86.818 | 61.989 | 24.058 | 1.00 | 8.12 | C |
| ATOM | 132 | CB | TYR | A | 24 | 87.082 | 61.245 | 25.356 | 1.00 | 7.57 | C |
| ATOM | 133 | CG | TYR | A | 24 | 86.537 | 59.849 | 25.446 | 1.00 | 8.57 | C |
| ATOM | 134 | CD1 | TYR | A | 24 | 87.149 | 58.797 | 24.783 | 1.00 | 8.16 | C |
| ATOM | 135 | CE1 | TYR | A | 24 | 86.678 | 57.505 | 24.909 | 1.00 | 8.46 | C |
| ATOM | 136 | CZ | TYR | A | 24 | 85.583 | 57.244 | 25.703 | 1.00 | 9.22 | C |
| ATOM | 137 | OH | TYR | A | 24 | 85.107 | 55.949 | 25.829 | 1.00 | 9.23 | O |
| ATOM | 138 | CE2 | TYR | A | 24 | 84.939 | 58.275 | 26.352 | 1.00 | 8.75 | C |
| ATOM | 139 | CD2 | TYR | A | 24 | 85.420 | 59.569 | 26.231 | 1.00 | 7.98 | C |
| ATOM | 140 | C | TYR | A | 24 | 85.353 | 62.400 | 23.990 | 1.00 | 7.92 | C |
| ATOM | 141 | O | TYR | A | 24 | 84.898 | 63.193 | 24.803 | 1.00 | 8.03 | O |
| ATOM | 142 | N | PHE | A | 25 | 84.641 | 61.900 | 22.991 | 1.00 | 8.21 | N |
| ATOM | 143 | CA | PHE | A | 25 | 83.280 | 62.320 | 22.721 | 1.00 | 8.18 | C |
| ATOM | 144 | CB | PHE | A | 25 | 83.144 | 62.689 | 21.242 | 1.00 | 8.40 | C |
| ATOM | 145 | CG | PHE | A | 25 | 81.709 | 62.743 | 20.739 | 1.00 | 8.66 | C |
| ATOM | 146 | CD1 | PHE | A | 25 | 80.696 | 63.328 | 21.500 | 1.00 | 9.58 | C |
| ATOM | 147 | CE1 | PHE | A | 25 | 79.383 | 63.358 | 21.027 | 1.00 | 9.05 | C |
| ATOM | 148 | CZ | PHE | A | 25 | 79.088 | 62.816 | 19.801 | 1.00 | 8.26 | C |
| ATOM | 149 | CE2 | PHE | A | 25 | 80.084 | 62.252 | 19.045 | 1.00 | 8.37 | C |
| ATOM | 150 | CD2 | PHE | A | 25 | 81.381 | 62.208 | 19.518 | 1.00 | 8.39 | C |
| ATOM | 151 | C | PHE | A | 25 | 82.311 | 61.213 | 23.151 | 1.00 | 8.63 | C |
| ATOM | 152 | O | PHE | A | 25 | 82.355 | 60.090 | 22.636 | 1.00 | 8.21 | O |
| ATOM | 153 | N | VAL | A | 26 | 81.459 | 61.555 | 24.113 | 1.00 | 9.31 | N |
| ATOM | 154 | CA | VAL | A | 26 | 80.425 | 60.653 | 24.615 | 1.00 | 9.72 | C |
| ATOM | 155 | CB | VAL | A | 26 | 80.466 | 60.446 | 26.154 | 1.00 | 8.23 | C |
| ATOM | 156 | CG1 | VAL | A | 26 | 81.757 | 59.776 | 26.598 | 1.00 | 7.98 | C |
| ATOM | 157 | CG2 | VAL | A | 26 | 80.294 | 61.762 | 26.914 | 1.00 | 7.37 | C |
| ATOM | 158 | C | VAL | A | 26 | 79.036 | 61.152 | 24.255 | 1.00 | 11.35 | C |
| ATOM | 159 | O | VAL | A | 26 | 78.736 | 62.349 | 24.348 | 1.00 | 10.46 | O |
| ATOM | 160 | N | MET | A | 27 | 78.173 | 60.214 | 23.856 | 1.00 | 11.76 | N |
| ATOM | 161 | CA | MET | A | 27 | 76.753 | 60.442 | 23.995 | 1.00 | 10.64 | C |
| ATOM | 162 | CB | MET | A | 27 | 75.931 | 59.590 | 23.020 | 1.00 | 10.86 | C |
| ATOM | 163 | CG | MET | A | 27 | 76.252 | 59.893 | 21.569 | 1.00 | 11.29 | C |
| ATOM | 164 | SD | MET | A | 27 | 74.897 | 59.499 | 20.447 | 1.00 | 15.44 | S |
| ATOM | 165 | CE | MET | A | 27 | 73.641 | 60.680 | 20.974 | 1.00 | 12.17 | C |
| ATOM | 166 | C | MET | A | 27 | 76.471 | 60.071 | 25.434 | 1.00 | 9.42 | C |
| ATOM | 167 | O | MET | A | 27 | 76.507 | 58.884 | 25.808 | 1.00 | 9.40 | O |
| ATOM | 168 | N | THR | A | 28 | 76.180 | 61.084 | 26.247 | 1.00 | 8.45 | N |
| ATOM | 169 | CA | THR | A | 28 | 76.110 | 60.889 | 27.690 | 1.00 | 7.76 | C |
| ATOM | 170 | CB | THR | A | 28 | 75.717 | 62.188 | 28.370 | 1.00 | 6.89 | C |
| ATOM | 171 | OG1 | THR | A | 28 | 76.648 | 63.190 | 27.968 | 1.00 | 7.28 | O |
| ATOM | 172 | CG2 | THR | A | 28 | 75.745 | 62.030 | 29.895 | 1.00 | 6.66 | C |
| ATOM | 173 | C | THR | A | 28 | 75.150 | 59.759 | 28.107 | 1.00 | 7.56 | C |
| ATOM | 174 | O | THR | A | 28 | 75.475 | 58.929 | 28.976 | 1.00 | 7.08 | O |
| ATOM | 175 | N | ASP | A | 29 | 74.007 | 59.685 | 27.433 | 1.00 | 7.84 | N |
| ATOM | 176 | CA | ASP | A | 29 | 72.986 | 58.674 | 27.754 | 1.00 | 7.69 | C |
| ATOM | 177 | CB | ASP | A | 29 | 71.700 | 58.919 | 26.974 | 1.00 | 8.29 | C |
| ATOM | 178 | CG | ASP | A | 29 | 70.632 | 57.883 | 27.301 | 1.00 | 10.64 | C |
| ATOM | 179 | OD1 | ASP | A | 29 | 70.103 | 57.872 | 28.458 | 1.00 | 12.48 | O |
| ATOM | 180 | OD2 | ASP | A | 29 | 70.353 | 57.039 | 26.419 | 1.00 | 11.07 | O |
| ATOM | 181 | C | ASP | A | 29 | 73.468 | 57.249 | 27.485 | 1.00 | 7.72 | C |
| ATOM | 182 | O | ASP | A | 29 | 72.907 | 56.278 | 28.012 | 1.00 | 7.59 | O |
| ATOM | 183 | N | ARG | A | 30 | 74.480 | 57.125 | 26.641 | 1.00 | 7.20 | N |
| ATOM | 184 | CA | ARG | A | 30 | 74.952 | 55.837 | 26.162 | 1.00 | 7.50 | C |
| ATOM | 185 | CB | ARG | A | 30 | 74.920 | 55.807 | 24.630 | 1.00 | 7.56 | C |
| ATOM | 186 | CG | ARG | A | 30 | 73.550 | 56.253 | 24.070 | 1.00 | 9.17 | C |
| ATOM | 187 | CD | ARG | A | 30 | 72.466 | 55.295 | 24.557 | 1.00 | 8.86 | C |
| ATOM | 188 | NE | ARG | A | 30 | 71.119 | 55.514 | 24.018 | 1.00 | 8.92 | N |
| ATOM | 189 | CZ | ARG | A | 30 | 70.680 | 55.058 | 22.842 | 1.00 | 9.72 | C |
| ATOM | 190 | NH1 | ARG | A | 30 | 71.499 | 54.428 | 21.991 | 1.00 | 9.20 | N |
| ATOM | 191 | NH2 | ARG | A | 30 | 69.425 | 55.273 | 22.493 | 1.00 | 9.24 | N |
| ATOM | 192 | C | ARG | A | 30 | 76.346 | 55.501 | 26.675 | 1.00 | 7.67 | C |
| ATOM | 193 | O | ARG | A | 30 | 76.925 | 54.501 | 26.243 | 1.00 | 8.07 | O |
| ATOM | 194 | N | PHE | A | 31 | 76.872 | 56.288 | 27.611 | 1.00 | 7.18 | N |
| ATOM | 195 | CA | PHE | A | 31 | 78.207 | 55.982 | 28.186 | 1.00 | 7.41 | C |
| ATOM | 196 | CB | PHE | A | 31 | 79.034 | 57.257 | 28.374 | 1.00 | 7.88 | C |
| ATOM | 197 | CG | PHE | A | 31 | 80.440 | 57.012 | 28.842 | 1.00 | 6.80 | C |
| ATOM | 198 | CD1 | PHE | A | 31 | 81.313 | 56.267 | 28.073 | 1.00 | 6.81 | C |
| ATOM | 199 | CE1 | PHE | A | 31 | 82.598 | 56.001 | 28.500 | 1.00 | 7.26 | C |
| ATOM | 200 | CZ | PHE | A | 31 | 83.041 | 56.504 | 29.708 | 1.00 | 8.10 | C |
| ATOM | 201 | CE2 | PHE | A | 31 | 82.179 | 57.260 | 30.494 | 1.00 | 8.20 | C |
| ATOM | 202 | CD2 | PHE | A | 31 | 80.881 | 57.524 | 30.041 | 1.00 | 6.85 | C |
| ATOM | 203 | C | PHE | A | 31 | 78.088 | 55.141 | 29.467 | 1.00 | 7.98 | C |
| ATOM | 204 | O | PHE | A | 31 | 78.222 | 53.923 | 29.398 | 1.00 | 8.68 | O |
| ATOM | 205 | N | VAL | A | 32 | 77.808 | 55.739 | 30.626 | 1.00 | 8.35 | N |
| ATOM | 206 | CA | VAL | A | 32 | 77.642 | 54.953 | 31.861 | 1.00 | 7.55 | C |
| ATOM | 207 | CB | VAL | A | 32 | 78.937 | 54.908 | 32.728 | 1.00 | 7.27 | C |
| ATOM | 208 | CG1 | VAL | A | 32 | 78.729 | 54.076 | 34.014 | 1.00 | 6.36 | C |
| ATOM | 209 | CG2 | VAL | A | 32 | 80.103 | 54.351 | 31.905 | 1.00 | 7.01 | C |
| ATOM | 210 | C | VAL | A | 32 | 76.499 | 55.491 | 32.714 | 1.00 | 8.33 | C |
| ATOM | 211 | O | VAL | A | 32 | 76.424 | 56.699 | 33.016 | 1.00 | 7.21 | O |
| ATOM | 212 | N | ASN | A | 33 | 75.576 | 54.595 | 33.054 | 1.00 | 8.65 | N |
| ATOM | 213 | CA | ASN | A | 33 | 74.448 | 54.927 | 33.919 | 1.00 | 8.72 | C |
| ATOM | 214 | CB | ASN | A | 33 | 73.284 | 53.940 | 33.699 | 1.00 | 9.37 | C |
| ATOM | 215 | CG | ASN | A | 33 | 72.068 | 54.236 | 34.577 | 1.00 | 10.57 | C |
| ATOM | 216 | OD1 | ASN | A | 33 | 72.071 | 55.169 | 35.402 | 1.00 | 11.90 | O |
| ATOM | 217 | ND2 | ASN | A | 33 | 71.015 | 53.471 | 34.380 | 1.00 | 8.33 | N |
| ATOM | 218 | C | ASN | A | 33 | 74.994 | 54.804 | 35.329 | 1.00 | 9.69 | C |
| ATOM | 219 | O | ASN | A | 33 | 75.061 | 53.712 | 35.860 | 1.00 | 7.47 | O |
| ATOM | 220 | N | GLY | A | 34 | 75.395 | 55.918 | 35.931 | 1.00 | 9.95 | N |
| ATOM | 221 | CA | GLY | A | 34 | 75.913 | 55.884 | 37.292 | 1.00 | 12.12 | C |
| ATOM | 222 | C | GLY | A | 34 | 74.819 | 55.969 | 38.343 | 1.00 | 12.68 | C |
| ATOM | 223 | O | GLY | A | 34 | 75.054 | 55.654 | 39.478 | 1.00 | 10.41 | O |
| ATOM | 224 | N | ASP | A | 35 | 73.620 | 56.380 | 37.960 | 1.00 | 11.76 | N |
| ATOM | 225 | CA | ASP | A | 35 | 72.556 | 56.583 | 38.916 | 1.00 | 10.54 | C |
| ATOM | 226 | CB | ASP | A | 35 | 72.474 | 58.078 | 39.257 | 1.00 | 11.10 | C |
| ATOM | 227 | CG | ASP | A | 35 | 71.409 | 58.396 | 40.311 | 1.00 | 14.76 | C |
| ATOM | 228 | OD1 | ASP | A | 35 | 70.843 | 57.458 | 40.896 | 1.00 | 13.11 | O |
| ATOM | 229 | OD2 | ASP | A | 35 | 71.154 | 59.593 | 40.547 | 1.00 | 15.92 | O |
| ATOM | 230 | C | ASP | A | 35 | 71.232 | 56.072 | 38.323 | 1.00 | 10.65 | C |
| ATOM | 231 | O | ASP | A | 35 | 70.640 | 56.733 | 37.500 | 1.00 | 8.08 | O |
| ATOM | 232 | N | PRO | A | 36 | 70.756 | 54.890 | 38.748 | 1.00 | 12.71 | N |
| ATOM | 233 | CA | PRO | A | 36 | 69.449 | 54.454 | 38.202 | 1.00 | 12.84 | C |
| ATOM | 234 | CB | PRO | A | 36 | 69.282 | 53.022 | 38.723 | 1.00 | 16.77 | C |
| ATOM | 235 | CG | PRO | A | 36 | 70.570 | 52.639 | 39.374 | 1.00 | 17.55 | C |
| ATOM | 236 | CD | PRO | A | 36 | 71.444 | 53.846 | 39.530 | 1.00 | 14.77 | C |
| ATOM | 237 | C | PRO | A | 36 | 68.282 | 55.318 | 38.641 | 1.00 | 10.21 | C |
| ATOM | 238 | O | PRO | A | 36 | 67.231 | 55.293 | 38.015 | 1.00 | 11.01 | O |
| ATOM | 239 | N | GLY | A | 37 | 68.469 | 56.107 | 39.684 | 1.00 | 9.71 | N |
| ATOM | 240 | CA | GLY | A | 37 | 67.371 | 56.865 | 40.274 | 1.00 | 9.82 | C |
| ATOM | 241 | C | GLY | A | 37 | 66.935 | 58.041 | 39.421 | 1.00 | 10.34 | C |
| ATOM | 242 | O | GLY | A | 37 | 65.861 | 58.577 | 39.623 | 1.00 | 9.92 | O |
| ATOM | 243 | N | ASN | A | 38 | 67.738 | 58.444 | 38.436 | 1.00 | 10.06 | N |
| ATOM | 244 | CA | ASN | A | 38 | 67.296 | 59.500 | 37.503 | 1.00 | 9.39 | C |
| ATOM | 245 | CB | ASN | A | 38 | 68.383 | 60.581 | 37.382 | 1.00 | 8.58 | C |
| ATOM | 246 | CG | ASN | A | 38 | 69.672 | 60.042 | 36.769 | 1.00 | 8.41 | C |
| ATOM | 247 | OD1 | ASN | A | 38 | 69.678 | 58.989 | 36.141 | 1.00 | 8.21 | O |
| ATOM | 248 | ND2 | ASN | A | 38 | 70.773 | 60.744 | 36.986 | 1.00 | 8.14 | N |
| ATOM | 249 | C | ASN | A | 38 | 66.887 | 58.982 | 36.090 | 1.00 | 10.78 | C |
| ATOM | 250 | O | ASN | A | 38 | 66.684 | 59.784 | 35.143 | 1.00 | 9.29 | O |
| ATOM | 251 | N | ASP | A | 39 | 66.742 | 57.667 | 35.940 | 1.00 | 9.62 | N |
| ATOM | 252 | CA | ASP | A | 39 | 66.450 | 57.084 | 34.619 | 1.00 | 9.27 | C |
| ATOM | 253 | CB | ASP | A | 39 | 66.578 | 55.568 | 34.681 | 1.00 | 10.46 | C |
| ATOM | 254 | CG | ASP | A | 39 | 68.017 | 55.098 | 34.786 | 1.00 | 10.85 | C |
| ATOM | 255 | OD1 | ASP | A | 39 | 68.998 | 55.898 | 34.764 | 1.00 | 10.52 | O |
| ATOM | 256 | OD2 | ASP | A | 39 | 68.157 | 53.880 | 34.883 | 1.00 | 12.39 | O |
| ATOM | 257 | C | ASP | A | 39 | 65.060 | 57.445 | 34.072 | 1.00 | 9.97 | C |
| ATOM | 258 | O | ASP | A | 39 | 64.848 | 57.441 | 32.873 | 1.00 | 8.89 | O |
| ATOM | 259 | N | HIS | A | 40 | 64.102 | 57.714 | 34.963 | 1.00 | 11.07 | N |
| ATOM | 260 | CA | HIS | A | 40 | 62.726 | 58.040 | 34.569 | 1.00 | 9.48 | C |
| ATOM | 261 | CB | HIS | A | 40 | 62.674 | 59.424 | 33.915 | 1.00 | 9.93 | C |
| ATOM | 262 | CG | HIS | A | 40 | 62.912 | 60.562 | 34.887 | 1.00 | 10.71 | C |
| ATOM | 263 | ND1 | HIS | A | 40 | 64.149 | 60.898 | 35.329 | 1.00 | 10.36 | N |
| ATOM | 264 | CE1 | HIS | A | 40 | 64.046 | 61.924 | 36.211 | 1.00 | 9.83 | C |
| ATOM | 265 | NE2 | HIS | A | 40 | 62.757 | 62.248 | 36.321 | 1.00 | 11.22 | N |
| ATOM | 266 | CD2 | HIS | A | 40 | 62.023 | 61.440 | 35.508 | 1.00 | 10.32 | C |
| ATOM | 267 | C | HIS | A | 40 | 62.167 | 56.971 | 33.674 | 1.00 | 10.33 | C |
| ATOM | 268 | O | HIS | A | 40 | 61.626 | 57.254 | 32.609 | 1.00 | 8.28 | O |
| ATOM | 269 | N | ARG | A | 41 | 62.288 | 55.710 | 34.093 | 1.00 | 10.34 | N |
| ATOM | 270 | CA | ARG | A | 41 | 61.934 | 54.575 | 33.212 | 1.00 | 12.39 | C |
| ATOM | 271 | CB | ARG | A | 41 | 62.300 | 53.217 | 33.861 | 1.00 | 16.40 | C |
| ATOM | 272 | CG | ARG | A | 41 | 63.811 | 53.020 | 33.956 | 1.00 | 23.60 | C |
| ATOM | 273 | CD | ARG | A | 41 | 64.263 | 51.753 | 34.702 | 1.00 | 25.64 | C |
| ATOM | 274 | NE | ARG | A | 41 | 65.733 | 51.754 | 34.724 | 1.00 | 30.83 | N |
| ATOM | 275 | CZ | ARG | A | 41 | 66.530 | 50.916 | 34.052 | 1.00 | 39.84 | C |
| ATOM | 276 | NH1 | ARG | A | 41 | 66.027 | 49.918 | 33.323 | 1.00 | 35.55 | N |
| ATOM | 277 | NH2 | ARG | A | 41 | 67.858 | 51.068 | 34.119 | 1.00 | 31.93 | N |
| ATOM | 278 | C | ARG | A | 41 | 60.477 | 54.561 | 32.763 | 1.00 | 11.82 | C |
| ATOM | 279 | O | ARG | A | 41 | 60.201 | 54.121 | 31.680 | 1.00 | 13.51 | O |
| ATOM | 280 | N | ASP | A | 42 | 59.542 | 55.067 | 33.555 | 1.00 | 11.44 | N |
| ATOM | 281 | CA | ASP | A | 42 | 58.147 | 55.034 | 33.123 | 1.00 | 13.15 | C |
| ATOM | 282 | CB | ASP | A | 42 | 57.222 | 54.616 | 34.279 | 1.00 | 17.56 | C |
| ATOM | 283 | CG | ASP | A | 42 | 57.116 | 55.666 | 35.367 | 1.00 | 24.61 | C |
| ATOM | 284 | OD1 | ASP | A | 42 | 57.542 | 56.815 | 35.146 | 1.00 | 34.56 | O |
| ATOM | 285 | OD2 | ASP | A | 42 | 56.594 | 55.351 | 36.461 | 1.00 | 34.16 | O |
| ATOM | 286 | C | ASP | A | 42 | 57.673 | 56.351 | 32.473 | 1.00 | 11.61 | C |
| ATOM | 287 | O | ASP | A | 42 | 56.514 | 56.487 | 32.134 | 1.00 | 10.98 | O |
| ATOM | 288 | N | GLN | A | 43 | 58.574 | 57.301 | 32.280 | 1.00 | 11.71 | N |
| ATOM | 289 | CA | GLN | A | 43 | 58.196 | 58.624 | 31.763 | 1.00 | 11.62 | C |
| ATOM | 290 | CB | GLN | A | 43 | 59.440 | 59.508 | 31.675 | 1.00 | 12.35 | C |
| ATOM | 291 | CG | GLN | A | 43 | 59.251 | 60.838 | 30.952 | 1.00 | 11.65 | C |
| ATOM | 292 | CD | GLN | A | 43 | 60.564 | 61.586 | 30.802 | 1.00 | 8.67 | C |
| ATOM | 293 | OE1 | GLN | A | 43 | 61.348 | 61.308 | 29.880 | 1.00 | 8.21 | O |
| ATOM | 294 | NE2 | GLN | A | 43 | 60.802 | 62.545 | 31.689 | 1.00 | 6.92 | N |
| ATOM | 295 | C | GLN | A | 43 | 57.516 | 58.486 | 30.397 | 1.00 | 12.63 | C |
| ATOM | 296 | O | GLN | A | 43 | 58.061 | 57.837 | 29.496 | 1.00 | 10.97 | O |
| ATOM | 297 | N | GLY | A | 44 | 56.318 | 59.075 | 30.268 | 1.00 | 12.17 | N |
| ATOM | 298 | CA | GLY | A | 44 | 55.587 | 59.090 | 29.005 | 1.00 | 11.64 | C |
| ATOM | 299 | C | GLY | A | 44 | 54.684 | 57.890 | 28.717 | 1.00 | 12.56 | C |
| ATOM | 300 | O | GLY | A | 44 | 53.997 | 57.886 | 27.708 | 1.00 | 11.13 | O |
| ATOM | 301 | N | GLY | A | 45 | 54.715 | 56.854 | 29.551 | 1.00 | 12.20 | N |
| ATOM | 302 | CA | GLY | A | 45 | 53.802 | 55.703 | 29.382 | 1.00 | 13.62 | C |
| ATOM | 303 | C | GLY | A | 45 | 54.015 | 54.996 | 28.038 | 1.00 | 14.01 | C |
| ATOM | 304 | O | GLY | A | 45 | 55.151 | 54.639 | 27.681 | 1.00 | 12.57 | O |
| ATOM | 305 | N | ALA | A | 46 | 52.926 | 54.811 | 27.288 | 1.00 | 11.00 | N |
| ATOM | 306 | CA | ALA | A | 46 | 52.977 | 54.192 | 25.952 | 1.00 | 11.74 | C |
| ATOM | 307 | CB | ALA | A | 46 | 51.547 | 53.981 | 25.439 | 1.00 | 10.90 | C |
| ATOM | 308 | C | ALA | A | 46 | 53.770 | 55.097 | 24.988 | 1.00 | 11.26 | C |
| ATOM | 309 | O | ALA | A | 46 | 54.331 | 54.640 | 24.005 | 1.00 | 11.81 | O |
| ATOM | 310 | N | LEU | A | 47 | 53.824 | 56.392 | 25.272 | 1.00 | 10.12 | N |
| ATOM | 311 | CA | LEU | A | 47 | 54.630 | 57.313 | 24.452 | 1.00 | 12.21 | C |
| ATOM | 312 | CB | LEU | A | 47 | 53.886 | 58.641 | 24.261 | 1.00 | 12.67 | C |
| ATOM | 313 | CG | LEU | A | 47 | 52.528 | 58.507 | 23.558 | 1.00 | 13.74 | C |
| ATOM | 314 | CD1 | LEU | A | 47 | 51.905 | 59.883 | 23.411 | 1.00 | 13.95 | C |
| ATOM | 315 | CD2 | LEU | A | 47 | 52.682 | 57.807 | 22.212 | 1.00 | 12.69 | C |
| ATOM | 316 | C | LEU | A | 47 | 55.969 | 57.543 | 25.174 | 1.00 | 10.46 | C |
| ATOM | 317 | O | LEU | A | 47 | 56.521 | 58.632 | 25.164 | 1.00 | 11.30 | O |
| ATOM | 318 | N | GLY | A | 48 | 56.486 | 56.483 | 25.768 | 1.00 | 9.33 | N |
| ATOM | 319 | CA | GLY | A | 48 | 57.547 | 56.588 | 26.747 | 1.00 | 11.14 | C |
| ATOM | 320 | C | GLY | A | 48 | 58.881 | 56.922 | 26.102 | 1.00 | 10.67 | C |
| ATOM | 321 | O | GLY | A | 48 | 59.114 | 56.625 | 24.932 | 1.00 | 11.69 | O |
| ATOM | 322 | N | THR | A | 49 | 59.762 | 57.496 | 26.897 | 1.00 | 10.50 | N |
| ATOM | 323 | CA | THR | A | 49 | 61.084 | 57.895 | 26.435 | 1.00 | 9.90 | C |
| ATOM | 324 | CB | THR | A | 49 | 61.455 | 59.229 | 27.095 | 1.00 | 10.00 | C |
| ATOM | 325 | OG1 | THR | A | 49 | 61.221 | 59.107 | 28.511 | 1.00 | 8.33 | O |
| ATOM | 326 | CG2 | THR | A | 49 | 60.615 | 60.392 | 26.512 | 1.00 | 8.41 | C |
| ATOM | 327 | C | THR | A | 49 | 62.195 | 56.905 | 26.755 | 1.00 | 9.60 | C |
| ATOM | 328 | O | THR | A | 49 | 63.308 | 57.005 | 26.175 | 1.00 | 8.74 | O |
| ATOM | 329 | N | PHE | A | 50 | 61.928 | 55.943 | 27.648 | 1.00 | 10.00 | N |
| ATOM | 330 | CA | PHE | A | 50 | 62.978 | 55.023 | 28.067 | 1.00 | 9.04 | C |
| ATOM | 331 | CB | PHE | A | 50 | 62.885 | 54.707 | 29.539 | 1.00 | 9.49 | C |
| ATOM | 332 | CG | PHE | A | 50 | 64.168 | 54.168 | 30.125 | 1.00 | 9.26 | C |
| ATOM | 333 | CD1 | PHE | A | 50 | 65.170 | 55.033 | 30.544 | 1.00 | 9.95 | C |
| ATOM | 334 | CE1 | PHE | A | 50 | 66.358 | 54.539 | 31.079 | 1.00 | 11.12 | C |
| ATOM | 335 | CZ | PHE | A | 50 | 66.560 | 53.171 | 31.188 | 1.00 | 11.10 | C |
| ATOM | 336 | CE2 | PHE | A | 50 | 65.555 | 52.291 | 30.788 | 1.00 | 10.93 | C |
| ATOM | 337 | CD2 | PHE | A | 50 | 64.365 | 52.799 | 30.262 | 1.00 | 9.67 | C |
| ATOM | 338 | C | PHE | A | 50 | 62.988 | 53.733 | 27.267 | 1.00 | 10.78 | C |
| ATOM | 339 | O | PHE | A | 50 | 61.946 | 53.116 | 27.105 | 1.00 | 11.80 | O |
| ATOM | 340 | N | ASP | A | 51 | 64.188 | 53.345 | 26.794 | 1.00 | 11.49 | N |
| ATOM | 341 | CA | ASP | A | 51 | 64.482 | 52.029 | 26.166 | 1.00 | 9.54 | C |
| ATOM | 342 | CB | ASP | A | 51 | 64.505 | 50.962 | 27.220 | 1.00 | 10.45 | C |
| ATOM | 343 | CG | ASP | A | 51 | 65.172 | 49.689 | 26.753 | 1.00 | 11.12 | C |
| ATOM | 344 | OD1 | ASP | A | 51 | 65.426 | 49.481 | 25.545 | 1.00 | 9.80 | O |
| ATOM | 345 | OD2 | ASP | A | 51 | 65.442 | 48.873 | 27.633 | 1.00 | 15.31 | O |
| ATOM | 346 | C | ASP | A | 51 | 63.502 | 51.637 | 25.083 | 1.00 | 9.86 | C |
| ATOM | 347 | O | ASP | A | 51 | 62.473 | 50.997 | 25.354 | 1.00 | 8.71 | O |
| ATOM | 348 | N | ILE | A | 52 | 63.813 | 52.053 | 23.857 | 1.00 | 9.39 | N |
| ATOM | 349 | CA | ILE | A | 52 | 62.968 | 51.829 | 22.697 | 1.00 | 8.39 | C |
| ATOM | 350 | CB | ILE | A | 52 | 62.484 | 53.158 | 22.073 | 1.00 | 8.12 | C |
| ATOM | 351 | CG1 | ILE | A | 52 | 61.810 | 54.041 | 23.133 | 1.00 | 7.93 | C |
| ATOM | 352 | CD1 | ILE | A | 52 | 61.567 | 55.486 | 22.695 | 1.00 | 7.27 | C |
| ATOM | 353 | CG2 | ILE | A | 52 | 61.508 | 52.888 | 20.905 | 1.00 | 6.95 | C |
| ATOM | 354 | C | ILE | A | 52 | 63.794 | 51.030 | 21.687 | 1.00 | 9.20 | C |
| ATOM | 355 | O | ILE | A | 52 | 64.460 | 51.606 | 20.843 | 1.00 | 9.82 | O |
| ATOM | 356 | N | PRO | A | 53 | 63.784 | 49.695 | 21.798 | 1.00 | 10.75 | N |
| ATOM | 357 | CA | PRO | A | 53 | 64.610 | 48.880 | 20.916 | 1.00 | 10.80 | C |
| ATOM | 358 | CB | PRO | A | 53 | 64.512 | 47.459 | 21.507 | 1.00 | 12.29 | C |
| ATOM | 359 | CG | PRO | A | 53 | 63.856 | 47.608 | 22.821 | 1.00 | 13.76 | C |
| ATOM | 360 | CD | PRO | A | 53 | 63.093 | 48.894 | 22.826 | 1.00 | 12.07 | C |
| ATOM | 361 | C | PRO | A | 53 | 64.082 | 48.862 | 19.505 | 1.00 | 11.93 | C |
| ATOM | 362 | O | PRO | A | 53 | 62.878 | 48.866 | 19.303 | 1.00 | 11.09 | O |
| ATOM | 363 | N | LEU | A | 54 | 64.994 | 48.848 | 18.547 | 1.00 | 12.47 | N |
| ATOM | 364 | CA | LEU | A | 54 | 64.671 | 48.619 | 17.170 | 1.00 | 12.45 | C |
| ATOM | 365 | CB | LEU | A | 54 | 65.573 | 49.462 | 16.269 | 1.00 | 12.54 | C |
| ATOM | 366 | CG | LEU | A | 54 | 65.460 | 50.972 | 16.440 | 1.00 | 13.96 | C |
| ATOM | 367 | CD1 | LEU | A | 54 | 66.534 | 51.671 | 15.601 | 1.00 | 14.06 | C |
| ATOM | 368 | CD2 | LEU | A | 54 | 64.060 | 51.454 | 16.088 | 1.00 | 13.08 | C |
| ATOM | 369 | C | LEU | A | 54 | 64.845 | 47.136 | 16.800 | 1.00 | 12.65 | C |
| ATOM | 370 | O | LEU | A | 54 | 65.644 | 46.391 | 17.425 | 1.00 | 11.14 | O |
| ATOM | 371 | N | PRO | A | 55 | 64.127 | 46.705 | 15.759 | 1.00 | 12.63 | N |
| ATOM | 372 | CA | PRO | A | 55 | 64.404 | 45.388 | 15.169 | 1.00 | 14.57 | C |
| ATOM | 373 | CB | PRO | A | 55 | 63.486 | 45.354 | 13.942 | 1.00 | 15.45 | C |
| ATOM | 374 | CG | PRO | A | 55 | 62.385 | 46.338 | 14.259 | 1.00 | 14.72 | C |
| ATOM | 375 | CD | PRO | A | 55 | 63.024 | 47.410 | 15.082 | 1.00 | 12.19 | C |
| ATOM | 376 | C | PRO | A | 55 | 65.885 | 45.260 | 14.757 | 1.00 | 14.76 | C |
| ATOM | 377 | O | PRO | A | 55 | 66.444 | 46.208 | 14.243 | 1.00 | 12.76 | O |
| ATOM | 378 | N | PRO | A | 56 | 66.517 | 44.102 | 15.003 | 1.00 | 15.11 | N |
| ATOM | 379 | CA | PRO | A | 56 | 67.954 | 44.066 | 14.773 | 1.00 | 16.52 | C |
| ATOM | 380 | CB | PRO | A | 56 | 68.372 | 42.661 | 15.226 | 1.00 | 17.32 | C |
| ATOM | 381 | CG | PRO | A | 56 | 67.112 | 41.852 | 15.270 | 1.00 | 18.80 | C |
| ATOM | 382 | CD | PRO | A | 56 | 66.007 | 42.830 | 15.546 | 1.00 | 16.27 | C |
| ATOM | 383 | C | PRO | A | 56 | 68.319 | 44.262 | 13.325 | 1.00 | 17.80 | C |
| ATOM | 384 | O | PRO | A | 56 | 67.532 | 43.962 | 12.439 | 1.00 | 14.03 | O |
| ATOM | 385 | N | CYS | A | 57 | 69.523 | 44.766 | 13.115 | 1.00 | 19.16 | N |
| ATOM | 386 | CA | CYS | A | 57 | 70.044 | 45.023 | 11.791 | 1.00 | 22.01 | C |
| ATOM | 387 | CB | CYS | A | 57 | 70.167 | 46.535 | 11.580 | 1.00 | 34.43 | C |
| ATOM | 388 | SG | CYS | A | 57 | 71.206 | 46.936 | 10.171 | 1.00 | 49.84 | S |
| ATOM | 389 | C | CYS | A | 57 | 71.403 | 44.343 | 11.723 | 1.00 | 17.97 | C |
| ATOM | 390 | O | CYS | A | 57 | 72.285 | 44.641 | 12.518 | 1.00 | 13.58 | O |
| ATOM | 391 | N | ASN | A | 58 | 71.550 | 43.373 | 10.827 | 1.00 | 17.43 | N |
| ATOM | 392 | CA | ASN | A | 58 | 72.755 | 42.529 | 10.790 | 1.00 | 18.31 | C |
| ATOM | 393 | CB | ASN | A | 58 | 73.898 | 43.266 | 10.086 | 1.00 | 22.78 | C |
| ATOM | 394 | CG | ASN | A | 58 | 73.557 | 43.599 | 8.649 | 1.00 | 25.97 | C |
| ATOM | 395 | OD1 | ASN | A | 58 | 73.669 | 44.746 | 8.215 | 1.00 | 30.21 | O |
| ATOM | 396 | ND2 | ASN | A | 58 | 73.099 | 42.603 | 7.918 | 1.00 | 26.44 | N |
| ATOM | 397 | C | ASN | A | 58 | 73.215 | 42.063 | 12.158 | 1.00 | 16.62 | C |
| ATOM | 398 | O | ASN | A | 58 | 74.391 | 42.180 | 12.515 | 1.00 | 13.20 | O |
| ATOM | 399 | N | GLY | A | 59 | 72.272 | 41.559 | 12.939 | 1.00 | 16.73 | N |
| ATOM | 400 | CA | GLY | A | 59 | 72.592 | 40.921 | 14.213 | 1.00 | 16.76 | C |
| ATOM | 401 | C | GLY | A | 59 | 72.719 | 41.825 | 15.429 | 1.00 | 15.94 | C |
| ATOM | 402 | O | GLY | A | 59 | 72.911 | 41.331 | 16.529 | 1.00 | 15.63 | O |
| ATOM | 403 | N | VAL | A | 60 | 72.580 | 43.136 | 15.258 | 1.00 | 15.07 | N |
| ATOM | 404 | CA | VAL | A | 60 | 72.674 | 44.055 | 16.381 | 1.00 | 14.25 | C |
| ATOM | 405 | CB | VAL | A | 60 | 73.967 | 44.916 | 16.310 | 1.00 | 16.22 | C |
| ATOM | 406 | CG1 | VAL | A | 60 | 73.908 | 46.018 | 17.356 | 1.00 | 14.41 | C |
| ATOM | 407 | CG2 | VAL | A | 60 | 75.210 | 44.056 | 16.532 | 1.00 | 13.03 | C |
| ATOM | 408 | C | VAL | A | 60 | 71.445 | 44.963 | 16.403 | 1.00 | 14.55 | C |
| ATOM | 409 | O | VAL | A | 60 | 71.011 | 45.468 | 15.372 | 1.00 | 15.71 | O |
| ATOM | 410 | N | SER | A | 61 | 70.875 | 45.149 | 17.580 | 1.00 | 13.04 | N |
| ATOM | 411 | CA | SER | A | 61 | 69.716 | 46.022 | 17.760 | 1.00 | 13.92 | C |
| ATOM | 412 | CB | SER | A | 61 | 68.715 | 45.358 | 18.735 | 1.00 | 16.79 | C |
| ATOM | 413 | OG | SER | A | 61 | 67.721 | 46.284 | 19.166 | 1.00 | 22.16 | O |
| ATOM | 414 | C | SER | A | 61 | 70.151 | 47.382 | 18.292 | 1.00 | 12.07 | C |
| ATOM | 415 | O | SER | A | 61 | 70.854 | 47.471 | 19.298 | 1.00 | 13.14 | O |
| ATOM | 416 | N | GLY | A | 62 | 69.776 | 48.442 | 17.593 | 1.00 | 11.47 | N |
| ATOM | 417 | CA | GLY | A | 62 | 69.943 | 49.791 | 18.112 | 1.00 | 11.67 | C |
| ATOM | 418 | C | GLY | A | 62 | 68.742 | 50.194 | 18.946 | 1.00 | 10.60 | C |
| ATOM | 419 | O | GLY | A | 62 | 67.814 | 49.418 | 19.111 | 1.00 | 9.27 | O |
| ATOM | 420 | N | ASN | A | 63 | 68.758 | 51.426 | 19.444 | 1.00 | 9.89 | N |
| ATOM | 421 | CA | ASN | A | 63 | 67.763 | 51.889 | 20.416 | 1.00 | 10.25 | C |
| ATOM | 422 | CB | ASN | A | 63 | 68.261 | 51.617 | 21.847 | 1.00 | 9.76 | C |
| ATOM | 423 | CG | ASN | A | 63 | 67.137 | 51.549 | 22.862 | 1.00 | 9.92 | C |
| ATOM | 424 | OD1 | ASN | A | 63 | 66.575 | 52.579 | 23.252 | 1.00 | 8.35 | O |
| ATOM | 425 | ND2 | ASN | A | 63 | 66.820 | 50.336 | 23.321 | 1.00 | 9.25 | N |
| ATOM | 426 | C | ASN | A | 63 | 67.569 | 53.377 | 20.166 | 1.00 | 9.97 | C |
| ATOM | 427 | O | ASN | A | 63 | 68.551 | 54.092 | 19.875 | 1.00 | 10.00 | O |
| ATOM | 428 | N | ILE | A | 64 | 66.319 | 53.827 | 20.236 | 1.00 | 8.58 | N |
| ATOM | 429 | CA | ILE | A | 64 | 65.974 | 55.225 | 20.019 | 1.00 | 8.85 | C |
| ATOM | 430 | CB | ILE | A | 64 | 65.096 | 55.422 | 18.747 | 1.00 | 9.33 | C |
| ATOM | 431 | CG1 | ILE | A | 64 | 63.729 | 54.746 | 18.825 | 1.00 | 10.39 | C |
| ATOM | 432 | CD1 | ILE | A | 64 | 62.865 | 54.975 | 17.570 | 1.00 | 9.04 | C |
| ATOM | 433 | CG2 | ILE | A | 64 | 65.862 | 54.919 | 17.522 | 1.00 | 8.98 | C |
| ATOM | 434 | C | ILE | A | 64 | 65.391 | 55.904 | 21.267 | 1.00 | 9.04 | C |
| ATOM | 435 | O | ILE | A | 64 | 64.819 | 56.989 | 21.184 | 1.00 | 10.11 | O |
| ATOM | 436 | N | GLY | A | 65 | 65.573 | 55.292 | 22.432 | 1.00 | 8.54 | N |
| ATOM | 437 | CA | GLY | A | 65 | 65.170 | 55.915 | 23.699 | 1.00 | 7.46 | C |
| ATOM | 438 | C | GLY | A | 65 | 66.325 | 56.136 | 24.643 | 1.00 | 7.13 | C |
| ATOM | 439 | O | GLY | A | 65 | 67.495 | 55.830 | 24.319 | 1.00 | 7.21 | O |
| ATOM | 440 | N | TYR | A | 66 | 66.021 | 56.671 | 25.821 | 1.00 | 6.49 | N |
| ATOM | 441 | CA | TYR | A | 66 | 67.043 | 56.821 | 26.835 | 1.00 | 7.42 | C |
| ATOM | 442 | CB | TYR | A | 66 | 66.581 | 57.694 | 28.007 | 1.00 | 7.97 | C |
| ATOM | 443 | CG | TYR | A | 66 | 66.436 | 59.182 | 27.744 | 1.00 | 6.94 | C |
| ATOM | 444 | CD1 | TYR | A | 66 | 65.335 | 59.679 | 27.102 | 1.00 | 6.70 | C |
| ATOM | 445 | CE1 | TYR | A | 66 | 65.178 | 61.059 | 26.898 | 1.00 | 7.52 | C |
| ATOM | 446 | CZ | TYR | A | 66 | 66.122 | 61.950 | 27.381 | 1.00 | 6.30 | C |
| ATOM | 447 | OH | TYR | A | 66 | 65.950 | 63.298 | 27.236 | 1.00 | 6.38 | O |
| ATOM | 448 | CE2 | TYR | A | 66 | 67.223 | 61.456 | 28.041 | 1.00 | 7.41 | C |
| ATOM | 449 | CD2 | TYR | A | 66 | 67.368 | 60.086 | 28.234 | 1.00 | 6.55 | C |
| ATOM | 450 | C | TYR | A | 66 | 67.399 | 55.426 | 27.379 | 1.00 | 8.28 | C |
| ATOM | 451 | O | TYR | A | 66 | 66.519 | 54.538 | 27.553 | 1.00 | 7.15 | O |
| ATOM | 452 | N | LEU | A | 67 | 68.694 | 55.243 | 27.633 | 1.00 | 8.66 | N |
| ATOM | 453 | CA | LEU | A | 67 | 69.190 | 54.027 | 28.261 | 1.00 | 8.10 | C |
| ATOM | 454 | CB | LEU | A | 67 | 70.163 | 53.308 | 27.319 | 1.00 | 8.12 | C |
| ATOM | 455 | CG | LEU | A | 67 | 69.433 | 52.587 | 26.158 | 1.00 | 7.40 | C |
| ATOM | 456 | CD1 | LEU | A | 67 | 70.429 | 51.912 | 25.188 | 1.00 | 7.21 | C |
| ATOM | 457 | CD2 | LEU | A | 67 | 68.402 | 51.600 | 26.727 | 1.00 | 7.16 | C |
| ATOM | 458 | C | LEU | A | 67 | 69.798 | 54.262 | 29.648 | 1.00 | 8.35 | C |
| ATOM | 459 | O | LEU | A | 67 | 70.173 | 53.306 | 30.298 | 1.00 | 7.99 | O |
| ATOM | 460 | N | GLY | A | 68 | 69.866 | 55.523 | 30.100 | 1.00 | 8.46 | N |
| ATOM | 461 | CA | GLY | A | 68 | 70.217 | 55.844 | 31.489 | 1.00 | 7.95 | C |
| ATOM | 462 | C | GLY | A | 68 | 71.595 | 56.416 | 31.776 | 1.00 | 7.86 | C |
| ATOM | 463 | O | GLY | A | 68 | 71.880 | 56.765 | 32.907 | 1.00 | 8.36 | O |
| ATOM | 464 | N | GLY | A | 69 | 72.449 | 56.556 | 30.762 | 1.00 | 8.50 | N |
| ATOM | 465 | CA | GLY | A | 69 | 73.783 | 57.136 | 30.956 | 1.00 | 7.95 | C |
| ATOM | 466 | C | GLY | A | 69 | 73.696 | 58.565 | 31.448 | 1.00 | 7.55 | C |
| ATOM | 467 | O | GLY | A | 69 | 72.831 | 59.314 | 31.024 | 1.00 | 6.85 | O |
| ATOM | 468 | N | ASP | A | 70 | 74.560 | 58.951 | 32.381 | 1.00 | 8.04 | N |
| ATOM | 469 | CA | ASP | A | 70 | 74.328 | 60.212 | 33.078 | 1.00 | 8.48 | C |
| ATOM | 470 | CB | ASP | A | 70 | 73.298 | 59.999 | 34.195 | 1.00 | 8.88 | C |
| ATOM | 471 | CG | ASP | A | 70 | 73.687 | 58.869 | 35.150 | 1.00 | 9.84 | C |
| ATOM | 472 | OD1 | ASP | A | 70 | 74.914 | 58.609 | 35.274 | 1.00 | 10.67 | O |
| ATOM | 473 | OD2 | ASP | A | 70 | 72.767 | 58.255 | 35.746 | 1.00 | 10.62 | O |
| ATOM | 474 | C | ASP | A | 70 | 75.608 | 60.877 | 33.573 | 1.00 | 8.59 | C |
| ATOM | 475 | O | ASP | A | 70 | 76.697 | 60.358 | 33.358 | 1.00 | 9.98 | O |
| ATOM | 476 | N | PHE | A | 71 | 75.468 | 62.048 | 34.196 | 1.00 | 9.01 | N |
| ATOM | 477 | CA | PHE | A | 71 | 76.618 | 62.807 | 34.697 | 1.00 | 8.64 | C |
| ATOM | 478 | CB | PHE | A | 71 | 76.196 | 64.118 | 35.348 | 1.00 | 9.16 | C |
| ATOM | 479 | CG | PHE | A | 71 | 75.640 | 65.159 | 34.390 | 1.00 | 9.25 | C |
| ATOM | 480 | CD1 | PHE | A | 71 | 75.812 | 65.069 | 33.015 | 1.00 | 9.79 | C |
| ATOM | 481 | CE1 | PHE | A | 71 | 75.301 | 66.058 | 32.177 | 1.00 | 9.12 | C |
| ATOM | 482 | CZ | PHE | A | 71 | 74.639 | 67.157 | 32.703 | 1.00 | 8.06 | C |
| ATOM | 483 | CE2 | PHE | A | 71 | 74.461 | 67.253 | 34.052 | 1.00 | 9.63 | C |
| ATOM | 484 | CD2 | PHE | A | 71 | 74.976 | 66.272 | 34.892 | 1.00 | 9.71 | C |
| ATOM | 485 | C | PHE | A | 71 | 77.398 | 62.007 | 35.712 | 1.00 | 9.92 | C |
| ATOM | 486 | O | PHE | A | 71 | 78.632 | 61.988 | 35.653 | 1.00 | 10.26 | O |
| ATOM | 487 | N | LYS | A | 72 | 76.689 | 61.323 | 36.626 | 1.00 | 9.44 | N |
| ATOM | 488 | CA | LYS | A | 72 | 77.370 | 60.524 | 37.645 | 1.00 | 10.90 | C |
| ATOM | 489 | CB | LYS | A | 72 | 76.330 | 59.961 | 38.599 | 1.00 | 15.21 | C |
| ATOM | 490 | CG | LYS | A | 72 | 76.803 | 58.855 | 39.519 | 1.00 | 20.58 | C |
| ATOM | 491 | CD | LYS | A | 72 | 77.341 | 59.372 | 40.807 | 1.00 | 24.80 | C |
| ATOM | 492 | CE | LYS | A | 72 | 77.427 | 58.245 | 41.827 | 1.00 | 27.45 | C |
| ATOM | 493 | NZ | LYS | A | 72 | 78.820 | 58.284 | 42.294 | 1.00 | 25.67 | N |
| ATOM | 494 | C | LYS | A | 72 | 78.238 | 59.404 | 37.059 | 1.00 | 10.76 | C |
| ATOM | 495 | O | LYS | A | 72 | 79.369 | 59.207 | 37.494 | 1.00 | 9.54 | O |
| ATOM | 496 | N | GLY | A | 73 | 77.691 | 58.657 | 36.106 | 1.00 | 9.89 | N |
| ATOM | 497 | CA | GLY | A | 73 | 78.440 | 57.596 | 35.443 | 1.00 | 9.50 | C |
| ATOM | 498 | C | GLY | A | 73 | 79.681 | 58.114 | 34.737 | 1.00 | 9.49 | C |
| ATOM | 499 | O | GLY | A | 73 | 80.728 | 57.472 | 34.757 | 1.00 | 8.42 | O |
| ATOM | 500 | N | LEU | A | 74 | 79.564 | 59.289 | 34.124 | 1.00 | 10.36 | N |
| ATOM | 501 | CA | LEU | A | 74 | 80.691 | 59.910 | 33.426 | 1.00 | 9.14 | C |
| ATOM | 502 | CB | LEU | A | 74 | 80.226 | 61.090 | 32.554 | 1.00 | 8.52 | C |
| ATOM | 503 | CG | LEU | A | 74 | 81.304 | 61.914 | 31.834 | 1.00 | 8.84 | C |
| ATOM | 504 | CD1 | LEU | A | 74 | 82.273 | 61.059 | 30.977 | 1.00 | 10.65 | C |
| ATOM | 505 | CD2 | LEU | A | 74 | 80.677 | 63.028 | 31.000 | 1.00 | 7.62 | C |
| ATOM | 506 | C | LEU | A | 74 | 81.753 | 60.353 | 34.452 | 1.00 | 9.81 | C |
| ATOM | 507 | O | LEU | A | 74 | 82.950 | 60.059 | 34.283 | 1.00 | 8.81 | O |
| ATOM | 508 | N | ALA | A | 75 | 81.315 | 61.047 | 35.501 | 1.00 | 8.68 | N |
| ATOM | 509 | CA | ALA | A | 75 | 82.230 | 61.568 | 36.539 | 1.00 | 9.78 | C |
| ATOM | 510 | CB | ALA | A | 75 | 81.462 | 62.294 | 37.655 | 1.00 | 8.66 | C |
| ATOM | 511 | C | ALA | A | 75 | 83.005 | 60.398 | 37.138 | 1.00 | 10.79 | C |
| ATOM | 512 | O | ALA | A | 75 | 84.224 | 60.477 | 37.292 | 1.00 | 12.06 | O |
| ATOM | 513 | N | ASP | A | 76 | 82.295 | 59.319 | 37.460 | 1.00 | 9.31 | N |
| ATOM | 514 | CA | ASP | A | 76 | 82.917 | 58.134 | 38.053 | 1.00 | 11.46 | C |
| ATOM | 515 | CB | ASP | A | 76 | 81.867 | 57.057 | 38.344 | 1.00 | 12.69 | C |
| ATOM | 516 | CG | ASP | A | 76 | 80.948 | 57.397 | 39.502 | 1.00 | 15.11 | C |
| ATOM | 517 | OD1 | ASP | A | 76 | 81.246 | 58.296 | 40.316 | 1.00 | 15.40 | O |
| ATOM | 518 | OD2 | ASP | A | 76 | 79.902 | 56.730 | 39.565 | 1.00 | 19.57 | O |
| ATOM | 519 | C | ASP | A | 76 | 83.932 | 57.471 | 37.139 | 1.00 | 10.81 | C |
| ATOM | 520 | O | ASP | A | 76 | 84.696 | 56.641 | 37.581 | 1.00 | 10.99 | O |
| ATOM | 521 | N | HIS | A | 77 | 83.950 | 57.785 | 35.852 | 1.00 | 10.46 | N |
| ATOM | 522 | CA | HIS | A | 77 | 84.919 | 57.111 | 34.953 | 1.00 | 10.51 | C |
| ATOM | 523 | CB | HIS | A | 77 | 84.161 | 56.175 | 34.016 | 1.00 | 10.91 | C |
| ATOM | 524 | CG | HIS | A | 77 | 83.519 | 55.052 | 34.756 | 1.00 | 12.17 | C |
| ATOM | 525 | ND1 | HIS | A | 77 | 82.228 | 55.079 | 35.129 | 1.00 | 13.06 | N |
| ATOM | 526 | CE1 | HIS | A | 77 | 81.952 | 53.975 | 35.854 | 1.00 | 14.09 | C |
| ATOM | 527 | NE2 | HIS | A | 77 | 83.088 | 53.264 | 35.982 | 1.00 | 15.67 | N |
| ATOM | 528 | CD2 | HIS | A | 77 | 84.074 | 53.902 | 35.333 | 1.00 | 12.48 | C |
| ATOM | 529 | C | HIS | A | 77 | 85.838 | 58.059 | 34.234 | 1.00 | 11.61 | C |
| ATOM | 530 | O | HIS | A | 77 | 86.426 | 57.730 | 33.199 | 1.00 | 12.44 | O |
| ATOM | 531 | N | LEU | A | 78 | 86.016 | 59.246 | 34.799 | 1.00 | 10.69 | N |
| ATOM | 532 | CA | LEU | A | 78 | 86.935 | 60.197 | 34.188 | 1.00 | 9.80 | C |
| ATOM | 533 | CB | LEU | A | 78 | 86.851 | 61.569 | 34.859 | 1.00 | 9.78 | C |
| ATOM | 534 | CG | LEU | A | 78 | 85.520 | 62.289 | 34.728 | 1.00 | 10.17 | C |
| ATOM | 535 | CD1 | LEU | A | 78 | 85.455 | 63.491 | 35.658 | 1.00 | 9.98 | C |
| ATOM | 536 | CD2 | LEU | A | 78 | 85.365 | 62.704 | 33.278 | 1.00 | 8.35 | C |
| ATOM | 537 | C | LEU | A | 78 | 88.364 | 59.670 | 34.247 | 1.00 | 10.80 | C |
| ATOM | 538 | O | LEU | A | 78 | 89.158 | 59.972 | 33.350 | 1.00 | 10.68 | O |
| ATOM | 539 | N | ASP | A | 79 | 88.713 | 58.887 | 35.270 | 1.00 | 10.17 | N |
| ATOM | 540 | CA | ASP | A | 79 | 90.067 | 58.320 | 35.338 | 1.00 | 12.18 | C |
| ATOM | 541 | CB | ASP | A | 79 | 90.345 | 57.628 | 36.685 | 1.00 | 14.77 | C |
| ATOM | 542 | CG | ASP | A | 79 | 90.595 | 58.635 | 37.808 | 1.00 | 22.62 | C |
| ATOM | 543 | OD1 | ASP | A | 79 | 90.902 | 59.821 | 37.522 | 1.00 | 28.51 | O |
| ATOM | 544 | OD2 | ASP | A | 79 | 90.482 | 58.245 | 38.983 | 1.00 | 32.76 | O |
| ATOM | 545 | C | ASP | A | 79 | 90.340 | 57.361 | 34.192 | 1.00 | 10.54 | C |
| ATOM | 546 | O | ASP | A | 79 | 91.427 | 57.349 | 33.605 | 1.00 | 10.10 | O |
| ATOM | 547 | N | TYR | A | 80 | 89.347 | 56.575 | 33.855 | 1.00 | 10.50 | N |
| ATOM | 548 | CA | TYR | A | 80 | 89.452 | 55.668 | 32.692 | 1.00 | 11.17 | C |
| ATOM | 549 | CB | TYR | A | 80 | 88.166 | 54.852 | 32.567 | 1.00 | 10.47 | C |
| ATOM | 550 | CG | TYR | A | 80 | 87.945 | 54.222 | 31.208 | 1.00 | 11.09 | C |
| ATOM | 551 | CD1 | TYR | A | 80 | 88.549 | 53.031 | 30.883 | 1.00 | 11.31 | C |
| ATOM | 552 | CE1 | TYR | A | 80 | 88.364 | 52.447 | 29.656 | 1.00 | 12.06 | C |
| ATOM | 553 | CZ | TYR | A | 80 | 87.562 | 53.035 | 28.731 | 1.00 | 11.32 | C |
| ATOM | 554 | OH | TYR | A | 80 | 87.380 | 52.400 | 27.529 | 1.00 | 11.71 | O |
| ATOM | 555 | CE2 | TYR | A | 80 | 86.925 | 54.228 | 29.017 | 1.00 | 10.47 | C |
| ATOM | 556 | CD2 | TYR | A | 80 | 87.129 | 54.818 | 30.258 | 1.00 | 11.11 | C |
| ATOM | 557 | C | TYR | A | 80 | 89.710 | 56.437 | 31.378 | 1.00 | 11.71 | C |
| ATOM | 558 | O | TYR | A | 80 | 90.493 | 55.992 | 30.521 | 1.00 | 10.23 | O |
| ATOM | 559 | N | ILE | A | 81 | 89.050 | 57.591 | 31.239 | 1.00 | 10.10 | N |
| ATOM | 560 | CA | ILE | A | 81 | 89.204 | 58.453 | 30.060 | 1.00 | 9.52 | C |
| ATOM | 561 | CB | ILE | A | 81 | 88.086 | 59.520 | 30.024 | 1.00 | 9.50 | C |
| ATOM | 562 | CG1 | ILE | A | 81 | 86.723 | 58.866 | 29.806 | 1.00 | 9.90 | C |
| ATOM | 563 | CD1 | ILE | A | 81 | 85.527 | 59.795 | 29.987 | 1.00 | 9.62 | C |
| ATOM | 564 | CG2 | ILE | A | 81 | 88.340 | 60.546 | 28.933 | 1.00 | 8.47 | C |
| ATOM | 565 | C | ILE | A | 81 | 90.590 | 59.136 | 30.063 | 1.00 | 10.89 | C |
| ATOM | 566 | O | ILE | A | 81 | 91.333 | 59.084 | 29.088 | 1.00 | 10.33 | O |
| ATOM | 567 | N | ARG | A | 82 | 90.929 | 59.776 | 31.173 | 1.00 | 11.96 | N |
| ATOM | 568 | CA | ARG | A | 82 | 92.170 | 60.502 | 31.289 | 1.00 | 13.12 | C |
| ATOM | 569 | CB | ARG | A | 82 | 92.257 | 61.192 | 32.643 | 1.00 | 17.16 | C |
| ATOM | 570 | CG | ARG | A | 82 | 93.471 | 62.089 | 32.809 | 1.00 | 24.97 | C |
| ATOM | 571 | CD | ARG | A | 82 | 93.534 | 62.787 | 34.154 | 1.00 | 33.12 | C |
| ATOM | 572 | NE | ARG | A | 82 | 93.284 | 61.889 | 35.292 | 1.00 | 40.22 | N |
| ATOM | 573 | CZ | ARG | A | 82 | 93.544 | 62.195 | 36.564 | 1.00 | 40.67 | C |
| ATOM | 574 | NH1 | ARG | A | 82 | 94.097 | 63.360 | 36.887 | 1.00 | 36.36 | N |
| ATOM | 575 | NH2 | ARG | A | 82 | 93.271 | 61.314 | 37.516 | 1.00 | 41.45 | N |
| ATOM | 576 | C | ARG | A | 82 | 93.379 | 59.596 | 31.105 | 1.00 | 13.92 | C |
| ATOM | 577 | O | ARG | A | 82 | 94.382 | 60.037 | 30.557 | 1.00 | 11.59 | O |
| ATOM | 578 | N | GLU | A | 83 | 93.318 | 58.352 | 31.576 | 1.00 | 13.09 | N |
| ATOM | 579 | CA | GLU | A | 83 | 94.507 | 57.495 | 31.470 | 1.00 | 16.54 | C |
| ATOM | 580 | CB | GLU | A | 83 | 94.453 | 56.324 | 32.449 | 1.00 | 21.64 | C |
| ATOM | 581 | CG | GLU | A | 83 | 93.624 | 55.146 | 32.035 | 1.00 | 28.98 | C |
| ATOM | 582 | CD | GLU | A | 83 | 93.406 | 54.136 | 33.176 | 1.00 | 34.23 | C |
| ATOM | 583 | OE1 | GLU | A | 83 | 93.801 | 54.398 | 34.331 | 1.00 | 36.99 | O |
| ATOM | 584 | OE2 | GLU | A | 83 | 92.799 | 53.078 | 32.914 | 1.00 | 31.15 | O |
| ATOM | 585 | C | GLU | A | 83 | 94.801 | 57.059 | 30.028 | 1.00 | 14.04 | C |
| ATOM | 586 | O | GLU | A | 83 | 95.934 | 56.677 | 29.720 | 1.00 | 11.54 | O |
| ATOM | 587 | N | MET | A | 84 | 93.812 | 57.172 | 29.129 | 1.00 | 11.61 | N |
| ATOM | 588 | CA | MET | A | 84 | 94.061 | 56.954 | 27.693 | 1.00 | 12.16 | C |
| ATOM | 589 | CB | MET | A | 84 | 92.760 | 56.693 | 26.932 | 1.00 | 10.60 | C |
| ATOM | 590 | CG | MET | A | 84 | 92.128 | 55.398 | 27.359 | 1.00 | 10.96 | C |
| ATOM | 591 | SD | MET | A | 84 | 90.704 | 54.942 | 26.397 | 1.00 | 11.99 | S |
| ATOM | 592 | CE | MET | A | 84 | 89.468 | 55.997 | 27.179 | 1.00 | 10.41 | C |
| ATOM | 593 | C | MET | A | 84 | 94.789 | 58.118 | 27.030 | 1.00 | 11.00 | C |
| ATOM | 594 | O | MET | A | 84 | 95.168 | 58.008 | 25.879 | 1.00 | 13.79 | O |
| ATOM | 595 | N | GLY | A | 85 | 94.904 | 59.242 | 27.730 | 1.00 | 11.45 | N |
| ATOM | 596 | CA | GLY | A | 85 | 95.580 | 60.440 | 27.215 | 1.00 | 11.01 | C |
| ATOM | 597 | C | GLY | A | 85 | 94.653 | 61.562 | 26.751 | 1.00 | 10.97 | C |
| ATOM | 598 | O | GLY | A | 85 | 95.111 | 62.560 | 26.179 | 1.00 | 11.10 | O |
| ATOM | 599 | N | PHE | A | 86 | 93.355 | 61.435 | 27.030 | 1.00 | 10.66 | N |
| ATOM | 600 | CA | PHE | A | 86 | 92.405 | 62.508 | 26.710 | 1.00 | 9.23 | C |
| ATOM | 601 | CB | PHE | A | 86 | 90.986 | 61.931 | 26.577 | 1.00 | 8.82 | C |
| ATOM | 602 | CG | PHE | A | 86 | 90.857 | 60.984 | 25.427 | 1.00 | 9.20 | C |
| ATOM | 603 | CD1 | PHE | A | 86 | 90.776 | 61.471 | 24.121 | 1.00 | 9.07 | C |
| ATOM | 604 | CE1 | PHE | A | 86 | 90.694 | 60.598 | 23.042 | 1.00 | 8.92 | C |
| ATOM | 605 | CZ | PHE | A | 86 | 90.703 | 59.220 | 23.257 | 1.00 | 9.67 | C |
| ATOM | 606 | CE2 | PHE | A | 86 | 90.795 | 58.730 | 24.557 | 1.00 | 9.45 | C |
| ATOM | 607 | CD2 | PHE | A | 86 | 90.877 | 59.611 | 25.623 | 1.00 | 9.13 | C |
| ATOM | 608 | C | PHE | A | 86 | 92.467 | 63.572 | 27.788 | 1.00 | 9.38 | C |
| ATOM | 609 | O | PHE | A | 86 | 92.593 | 63.243 | 28.951 | 1.00 | 9.10 | O |
| ATOM | 610 | N | THR | A | 87 | 92.330 | 64.838 | 27.403 | 1.00 | 9.49 | N |
| ATOM | 611 | CA | THR | A | 87 | 92.333 | 65.935 | 28.353 | 1.00 | 10.37 | C |
| ATOM | 612 | CB | THR | A | 87 | 93.479 | 66.888 | 28.012 | 1.00 | 13.23 | C |
| ATOM | 613 | OG1 | THR | A | 87 | 93.245 | 67.376 | 26.696 | 1.00 | 13.80 | O |
| ATOM | 614 | CG2 | THR | A | 87 | 94.867 | 66.153 | 28.076 | 1.00 | 12.69 | C |
| ATOM | 615 | C | THR | A | 87 | 91.037 | 66.734 | 28.362 | 1.00 | 10.11 | C |
| ATOM | 616 | O | THR | A | 87 | 90.935 | 67.713 | 29.096 | 1.00 | 11.86 | O |
| ATOM | 617 | N | ALA | A | 88 | 90.056 | 66.326 | 27.547 | 1.00 | 9.26 | N |
| ATOM | 618 | CA | ALA | A | 88 | 88.782 | 67.001 | 27.412 | 1.00 | 8.97 | C |
| ATOM | 619 | CB | ALA | A | 88 | 88.832 | 68.081 | 26.331 | 1.00 | 7.85 | C |
| ATOM | 620 | C | ALA | A | 88 | 87.713 | 65.981 | 27.064 | 1.00 | 8.38 | C |
| ATOM | 621 | O | ALA | A | 88 | 87.999 | 65.001 | 26.402 | 1.00 | 7.89 | O |
| ATOM | 622 | N | VAL | A | 89 | 86.484 | 66.250 | 27.488 | 1.00 | 7.77 | N |
| ATOM | 623 | CA | VAL | A | 89 | 85.333 | 65.430 | 27.150 | 1.00 | 7.88 | C |
| ATOM | 624 | CB | VAL | A | 89 | 84.742 | 64.750 | 28.369 | 1.00 | 7.81 | C |
| ATOM | 625 | CG1 | VAL | A | 89 | 83.621 | 63.809 | 27.937 | 1.00 | 7.23 | C |
| ATOM | 626 | CG2 | VAL | A | 89 | 85.835 | 63.992 | 29.131 | 1.00 | 8.30 | C |
| ATOM | 627 | C | VAL | A | 89 | 84.237 | 66.271 | 26.505 | 1.00 | 8.68 | C |
| ATOM | 628 | O | VAL | A | 89 | 83.821 | 67.297 | 27.047 | 1.00 | 10.69 | O |
| ATOM | 629 | N | TRP | A | 90 | 83.795 | 65.827 | 25.345 | 1.00 | 8.80 | N |
| ATOM | 630 | CA | TRP | A | 90 | 82.647 | 66.432 | 24.645 | 1.00 | 9.05 | C |
| ATOM | 631 | CB | TRP | A | 90 | 82.965 | 66.553 | 23.141 | 1.00 | 8.66 | C |
| ATOM | 632 | CG | TRP | A | 90 | 81.820 | 66.563 | 22.143 | 1.00 | 8.98 | C |
| ATOM | 633 | CD1 | TRP | A | 90 | 80.475 | 66.749 | 22.391 | 1.00 | 8.53 | C |
| ATOM | 634 | NE1 | TRP | A | 90 | 79.755 | 66.666 | 21.233 | 1.00 | 10.03 | N |
| ATOM | 635 | CE2 | TRP | A | 90 | 80.572 | 66.463 | 20.168 | 1.00 | 9.10 | C |
| ATOM | 636 | CD2 | TRP | A | 90 | 81.926 | 66.417 | 20.676 | 1.00 | 8.23 | C |
| ATOM | 637 | CE3 | TRP | A | 90 | 82.984 | 66.178 | 19.776 | 1.00 | 8.44 | C |
| ATOM | 638 | CZ3 | TRP | A | 90 | 82.699 | 66.058 | 18.416 | 1.00 | 8.17 | C |
| ATOM | 639 | CH2 | TRP | A | 90 | 81.373 | 66.109 | 17.943 | 1.00 | 9.05 | C |
| ATOM | 640 | CZ2 | TRP | A | 90 | 80.286 | 66.285 | 18.819 | 1.00 | 9.11 | C |
| ATOM | 641 | C | TRP | A | 90 | 81.440 | 65.590 | 25.015 | 1.00 | 7.90 | C |
| ATOM | 642 | O | TRP | A | 90 | 81.422 | 64.348 | 24.841 | 1.00 | 8.27 | O |
| ATOM | 643 | N | ILE | A | 91 | 80.485 | 66.256 | 25.649 | 1.00 | 7.66 | N |
| ATOM | 644 | CA | ILE | A | 91 | 79.228 | 65.659 | 26.041 | 1.00 | 7.40 | C |
| ATOM | 645 | CB | ILE | A | 91 | 78.918 | 65.864 | 27.546 | 1.00 | 7.67 | C |
| ATOM | 646 | CG1 | ILE | A | 91 | 78.493 | 67.323 | 27.834 | 1.00 | 7.56 | C |
| ATOM | 647 | CD1 | ILE | A | 91 | 78.135 | 67.600 | 29.300 | 1.00 | 6.81 | C |
| ATOM | 648 | CG2 | ILE | A | 91 | 80.108 | 65.406 | 28.419 | 1.00 | 7.40 | C |
| ATOM | 649 | C | ILE | A | 91 | 78.099 | 66.224 | 25.165 | 1.00 | 7.63 | C |
| ATOM | 650 | O | ILE | A | 91 | 78.171 | 67.372 | 24.682 | 1.00 | 6.31 | O |
| ATOM | 651 | N | THR | A | 92 | 77.074 | 65.394 | 24.950 | 1.00 | 7.17 | N |
| ATOM | 652 | CA | THR | A | 92 | 75.894 | 65.784 | 24.184 | 1.00 | 7.86 | C |
| ATOM | 653 | CB | THR | A | 92 | 75.000 | 64.561 | 23.863 | 1.00 | 7.77 | C |
| ATOM | 654 | OG1 | THR | A | 92 | 74.892 | 63.711 | 25.021 | 1.00 | 6.92 | O |
| ATOM | 655 | CG2 | THR | A | 92 | 75.618 | 63.748 | 22.716 | 1.00 | 7.73 | C |
| ATOM | 656 | C | THR | A | 92 | 75.120 | 66.869 | 24.940 | 1.00 | 8.10 | C |
| ATOM | 657 | O | THR | A | 92 | 75.376 | 67.110 | 26.127 | 1.00 | 6.99 | O |
| ATOM | 658 | N | PRO | A | 93 | 74.248 | 67.603 | 24.231 | 1.00 | 8.13 | N |
| ATOM | 659 | CA | PRO | A | 93 | 73.575 | 68.734 | 24.878 | 1.00 | 7.93 | C |
| ATOM | 660 | CB | PRO | A | 93 | 72.618 | 69.240 | 23.800 | 1.00 | 7.98 | C |
| ATOM | 661 | CG | PRO | A | 93 | 73.285 | 68.843 | 22.527 | 1.00 | 8.35 | C |
| ATOM | 662 | CD | PRO | A | 93 | 73.847 | 67.480 | 22.822 | 1.00 | 8.00 | C |
| ATOM | 663 | C | PRO | A | 93 | 72.821 | 68.314 | 26.125 | 1.00 | 8.83 | C |
| ATOM | 664 | O | PRO | A | 93 | 72.265 | 67.198 | 26.188 | 1.00 | 9.93 | O |
| ATOM | 665 | N | ILE | A | 94 | 72.835 | 69.206 | 27.102 | 1.00 | 8.49 | N |
| ATOM | 666 | CA | ILE | A | 94 | 72.417 | 68.917 | 28.456 | 1.00 | 8.19 | C |
| ATOM | 667 | CB | ILE | A | 94 | 73.391 | 69.548 | 29.495 | 1.00 | 7.74 | C |
| ATOM | 668 | CG1 | ILE | A | 94 | 73.431 | 71.092 | 29.388 | 1.00 | 7.40 | C |
| ATOM | 669 | CD1 | ILE | A | 94 | 74.265 | 71.742 | 30.478 | 1.00 | 7.41 | C |
| ATOM | 670 | CG2 | ILE | A | 94 | 74.802 | 68.970 | 29.327 | 1.00 | 6.92 | C |
| ATOM | 671 | C | ILE | A | 94 | 71.037 | 69.454 | 28.758 | 1.00 | 8.68 | C |
| ATOM | 672 | O | ILE | A | 94 | 70.513 | 69.175 | 29.849 | 1.00 | 7.38 | O |
| ATOM | 673 | N | VAL | A | 95 | 70.452 | 70.222 | 27.825 | 1.00 | 8.70 | N |
| ATOM | 674 | CA | VAL | A | 95 | 69.220 | 70.974 | 28.111 | 1.00 | 8.89 | C |
| ATOM | 675 | CB | VAL | A | 95 | 69.027 | 72.148 | 27.136 | 1.00 | 9.88 | C |
| ATOM | 676 | CG1 | VAL | A | 95 | 70.243 | 73.077 | 27.203 | 1.00 | 9.93 | C |
| ATOM | 677 | CG2 | VAL | A | 95 | 68.785 | 71.625 | 25.724 | 1.00 | 8.69 | C |
| ATOM | 678 | C | VAL | A | 95 | 67.984 | 70.088 | 28.087 | 1.00 | 8.23 | C |
| ATOM | 679 | O | VAL | A | 95 | 67.997 | 69.019 | 27.487 | 1.00 | 9.61 | O |
| ATOM | 680 | N | ASP | A | 96 | 66.913 | 70.508 | 28.749 | 1.00 | 8.61 | N |
| ATOM | 681 | CA | ASP | A | 96 | 65.703 | 69.664 | 28.823 | 1.00 | 7.56 | C |
| ATOM | 682 | CB | ASP | A | 96 | 64.590 | 70.332 | 29.648 | 1.00 | 7.93 | C |
| ATOM | 683 | CG | ASP | A | 96 | 63.355 | 69.443 | 29.808 | 1.00 | 8.23 | C |
| ATOM | 684 | OD1 | ASP | A | 96 | 63.489 | 68.190 | 29.980 | 1.00 | 8.77 | O |
| ATOM | 685 | OD2 | ASP | A | 96 | 62.228 | 69.985 | 29.796 | 1.00 | 7.69 | O |
| ATOM | 686 | C | ASP | A | 96 | 65.194 | 69.336 | 27.428 | 1.00 | 7.56 | C |
| ATOM | 687 | O | ASP | A | 96 | 64.994 | 70.233 | 26.624 | 1.00 | 7.31 | O |
| ATOM | 688 | N | ASN | A | 97 | 65.018 | 68.041 | 27.144 | 1.00 | 8.12 | N |
| ATOM | 689 | CA | ASN | A | 97 | 64.407 | 67.568 | 25.884 | 1.00 | 7.65 | C |
| ATOM | 690 | CB | ASN | A | 97 | 65.234 | 66.442 | 25.249 | 1.00 | 7.08 | C |
| ATOM | 691 | CG | ASN | A | 97 | 66.444 | 66.945 | 24.432 | 1.00 | 7.51 | C |
| ATOM | 692 | OD1 | ASN | A | 97 | 66.673 | 66.451 | 23.339 | 1.00 | 6.84 | O |
| ATOM | 693 | ND2 | ASN | A | 97 | 67.212 | 67.899 | 24.963 | 1.00 | 6.08 | N |
| ATOM | 694 | C | ASN | A | 97 | 63.015 | 67.059 | 26.258 | 1.00 | 8.12 | C |
| ATOM | 695 | O | ASN | A | 97 | 62.697 | 66.915 | 27.454 | 1.00 | 8.47 | O |
| ATOM | 696 | N | PRO | A | 98 | 62.157 | 66.810 | 25.265 | 1.00 | 8.50 | N |
| ATOM | 697 | CA | PRO | A | 98 | 60.791 | 66.405 | 25.614 | 1.00 | 8.16 | C |
| ATOM | 698 | CB | PRO | A | 98 | 60.109 | 66.292 | 24.252 | 1.00 | 8.27 | C |
| ATOM | 699 | CG | PRO | A | 98 | 60.910 | 67.217 | 23.355 | 1.00 | 8.40 | C |
| ATOM | 700 | CD | PRO | A | 98 | 62.317 | 66.944 | 23.802 | 1.00 | 8.79 | C |
| ATOM | 701 | C | PRO | A | 98 | 60.665 | 65.088 | 26.404 | 1.00 | 8.40 | C |
| ATOM | 702 | O | PRO | A | 98 | 61.570 | 64.262 | 26.398 | 1.00 | 8.90 | O |
| ATOM | 703 | N | ASP | A | 99 | 59.536 | 64.937 | 27.085 | 1.00 | 8.91 | N |
| ATOM | 704 | CA | ASP | A | 99 | 59.307 | 63.858 | 28.053 | 1.00 | 10.21 | C |
| ATOM | 705 | CB | ASP | A | 99 | 58.765 | 64.455 | 29.352 | 1.00 | 11.40 | C |
| ATOM | 706 | CG | ASP | A | 99 | 59.751 | 65.439 | 30.001 | 1.00 | 11.08 | C |
| ATOM | 707 | OD1 | ASP | A | 99 | 60.934 | 65.470 | 29.627 | 1.00 | 9.70 | O |
| ATOM | 708 | OD2 | ASP | A | 99 | 59.343 | 66.204 | 30.876 | 1.00 | 11.83 | O |
| ATOM | 709 | C | ASP | A | 99 | 58.357 | 62.800 | 27.489 | 1.00 | 9.78 | C |
| ATOM | 710 | O | ASP | A | 99 | 57.805 | 62.000 | 28.230 | 1.00 | 9.41 | O |
| ATOM | 711 | N | GLN | A | 100 | 58.196 | 62.814 | 26.168 | 1.00 | 9.73 | N |
| ATOM | 712 | CA | GLN | A | 100 | 57.585 | 61.738 | 25.386 | 1.00 | 10.34 | C |
| ATOM | 713 | CB | GLN | A | 100 | 56.166 | 62.111 | 24.915 | 1.00 | 9.23 | C |
| ATOM | 714 | CG | GLN | A | 100 | 55.161 | 62.353 | 26.049 | 1.00 | 9.49 | C |
| ATOM | 715 | CD | GLN | A | 100 | 53.773 | 62.763 | 25.568 | 1.00 | 10.49 | C |
| ATOM | 716 | OE1 | GLN | A | 100 | 53.595 | 63.210 | 24.417 | 1.00 | 10.91 | O |
| ATOM | 717 | NE2 | GLN | A | 100 | 52.763 | 62.627 | 26.467 | 1.00 | 8.16 | N |
| ATOM | 718 | C | GLN | A | 100 | 58.443 | 61.468 | 24.161 | 1.00 | 10.06 | C |
| ATOM | 719 | O | GLN | A | 100 | 59.230 | 62.319 | 23.759 | 1.00 | 11.00 | O |
| ATOM | 720 | N | ARG | A | 101 | 58.265 | 60.300 | 23.562 | 1.00 | 9.65 | N |
| ATOM | 721 | CA | ARG | A | 101 | 58.781 | 60.044 | 22.213 | 1.00 | 9.72 | C |
| ATOM | 722 | CB | ARG | A | 101 | 58.788 | 58.557 | 21.859 | 1.00 | 9.97 | C |
| ATOM | 723 | CG | ARG | A | 101 | 57.405 | 57.950 | 21.685 | 1.00 | 10.82 | C |
| ATOM | 724 | CD | ARG | A | 101 | 57.491 | 56.459 | 21.389 | 1.00 | 11.39 | C |
| ATOM | 725 | NE | ARG | A | 101 | 57.797 | 55.699 | 22.601 | 1.00 | 12.66 | N |
| ATOM | 726 | CZ | ARG | A | 101 | 57.859 | 54.369 | 22.674 | 1.00 | 15.60 | C |
| ATOM | 727 | NH1 | ARG | A | 101 | 57.615 | 53.615 | 21.611 | 1.00 | 17.16 | N |
| ATOM | 728 | NH2 | ARG | A | 101 | 58.166 | 53.777 | 23.821 | 1.00 | 13.77 | N |
| ATOM | 729 | C | ARG | A | 101 | 57.948 | 60.787 | 21.169 | 1.00 | 9.87 | C |
| ATOM | 730 | O | ARG | A | 101 | 56.778 | 61.115 | 21.408 | 1.00 | 10.29 | O |
| ATOM | 731 | N | PHE | A | 102 | 58.576 | 61.041 | 20.014 | 1.00 | 8.92 | N |
| ATOM | 732 | CA | PHE | A | 102 | 57.954 | 61.798 | 18.916 | 1.00 | 9.48 | C |
| ATOM | 733 | CB | PHE | A | 102 | 59.055 | 62.345 | 18.010 | 1.00 | 9.36 | C |
| ATOM | 734 | CG | PHE | A | 102 | 58.565 | 63.258 | 16.899 | 1.00 | 8.41 | C |
| ATOM | 735 | CD1 | PHE | A | 102 | 57.745 | 64.339 | 17.165 | 1.00 | 8.84 | C |
| ATOM | 736 | CE1 | PHE | A | 102 | 57.339 | 65.193 | 16.148 | 1.00 | 8.66 | C |
| ATOM | 737 | CZ | PHE | A | 102 | 57.798 | 64.988 | 14.858 | 1.00 | 8.96 | C |
| ATOM | 738 | CE2 | PHE | A | 102 | 58.622 | 63.941 | 14.595 | 1.00 | 9.20 | C |
| ATOM | 739 | CD2 | PHE | A | 102 | 59.015 | 63.081 | 15.625 | 1.00 | 9.33 | C |
| ATOM | 740 | C | PHE | A | 102 | 56.959 | 60.956 | 18.094 | 1.00 | 10.34 | C |
| ATOM | 741 | O | PHE | A | 102 | 57.301 | 59.854 | 17.632 | 1.00 | 10.41 | O |
| ATOM | 742 | N | THR | A | 103 | 55.727 | 61.452 | 17.915 | 1.00 | 10.57 | N |
| ATOM | 743 | CA | THR | A | 103 | 54.721 | 60.690 | 17.136 | 1.00 | 9.76 | C |
| ATOM | 744 | CB | THR | A | 103 | 53.315 | 60.712 | 17.789 | 1.00 | 10.24 | C |
| ATOM | 745 | OG1 | THR | A | 103 | 52.801 | 62.051 | 17.784 | 1.00 | 10.00 | O |
| ATOM | 746 | CG2 | THR | A | 103 | 53.383 | 60.193 | 19.221 | 1.00 | 8.11 | C |
| ATOM | 747 | C | THR | A | 103 | 54.624 | 61.152 | 15.681 | 1.00 | 11.29 | C |
| ATOM | 748 | O | THR | A | 103 | 53.801 | 60.660 | 14.944 | 1.00 | 10.52 | O |
| ATOM | 749 | N | GLY | A | 104 | 55.499 | 62.054 | 15.241 | 1.00 | 10.63 | N |
| ATOM | 750 | CA | GLY | A | 104 | 55.557 | 62.418 | 13.818 | 1.00 | 11.41 | C |
| ATOM | 751 | C | GLY | A | 104 | 56.723 | 61.734 | 13.121 | 1.00 | 11.50 | C |
| ATOM | 752 | O | GLY | A | 104 | 57.230 | 60.713 | 13.597 | 1.00 | 12.94 | O |
| ATOM | 753 | N | GLY | A | 105 | 57.153 | 62.310 | 12.005 | 1.00 | 11.93 | N |
| ATOM | 754 | CA | GLY | A | 105 | 58.451 | 62.001 | 11.422 | 1.00 | 12.45 | C |
| ATOM | 755 | C | GLY | A | 105 | 58.443 | 60.770 | 10.535 | 1.00 | 11.99 | C |
| ATOM | 756 | O | GLY | A | 105 | 57.410 | 60.211 | 10.236 | 1.00 | 12.76 | O |
| ATOM | 757 | N | SER | A | 106 | 59.615 | 60.370 | 10.090 | 1.00 | 13.86 | N |
| ATOM | 758 | CA | SER | A | 106 | 59.743 | 59.201 | 9.243 | 1.00 | 15.64 | C |
| ATOM | 759 | CB | SER | A | 106 | 60.402 | 59.601 | 7.932 | 1.00 | 21.06 | C |
| ATOM | 760 | OG | SER | A | 106 | 61.592 | 60.283 | 8.227 | 1.00 | 33.58 | O |
| ATOM | 761 | C | SER | A | 106 | 60.514 | 58.092 | 9.959 | 1.00 | 13.96 | C |
| ATOM | 762 | O | SER | A | 106 | 61.283 | 58.335 | 10.894 | 1.00 | 12.62 | O |
| ATOM | 763 | N | ALA | A | 107 | 60.265 | 56.860 | 9.526 | 1.00 | 16.61 | N |
| ATOM | 764 | CA | ALA | A | 107 | 60.715 | 55.655 | 10.221 | 1.00 | 16.25 | C |
| ATOM | 765 | CB | ALA | A | 107 | 59.991 | 54.423 | 9.678 | 1.00 | 17.15 | C |
| ATOM | 766 | C | ALA | A | 107 | 62.225 | 55.466 | 10.090 | 1.00 | 17.43 | C |
| ATOM | 767 | O | ALA | A | 107 | 62.806 | 55.670 | 9.026 | 1.00 | 15.32 | O |
| ATOM | 768 | N | PRO | A | 108 | 62.874 | 55.096 | 11.192 | 1.00 | 18.09 | N |
| ATOM | 769 | CA | PRO | A | 108 | 64.308 | 54.883 | 11.165 | 1.00 | 15.95 | C |
| ATOM | 770 | CB | PRO | A | 108 | 64.656 | 54.723 | 12.651 | 1.00 | 17.38 | C |
| ATOM | 771 | CG | PRO | A | 108 | 63.410 | 54.141 | 13.254 | 1.00 | 18.61 | C |
| ATOM | 772 | CD | PRO | A | 108 | 62.294 | 54.849 | 12.527 | 1.00 | 18.07 | C |
| ATOM | 773 | C | PRO | A | 108 | 64.646 | 53.635 | 10.376 | 1.00 | 18.29 | C |
| ATOM | 774 | O | PRO | A | 108 | 63.933 | 52.655 | 10.436 | 1.00 | 16.49 | O |
| ATOM | 775 | N | THR | A | 109 | 65.697 | 53.686 | 9.574 | 1.00 | 21.18 | N |
| ATOM | 776 | CA | THR | A | 109 | 66.147 | 52.510 | 8.872 | 1.00 | 21.58 | C |
| ATOM | 777 | CB | THR | A | 109 | 65.984 | 52.635 | 7.340 | 1.00 | 26.72 | C |
| ATOM | 778 | OG1 | THR | A | 109 | 66.829 | 53.675 | 6.855 | 1.00 | 29.49 | O |
| ATOM | 779 | CG2 | THR | A | 109 | 64.545 | 52.945 | 6.942 | 1.00 | 33.30 | C |
| ATOM | 780 | C | THR | A | 109 | 67.628 | 52.364 | 9.210 | 1.00 | 24.94 | C |
| ATOM | 781 | O | THR | A | 109 | 68.252 | 53.288 | 9.757 | 1.00 | 25.33 | O |
| ATOM | 782 | N | CYS | A | 110 | 68.196 | 51.223 | 8.856 | 1.00 | 24.91 | N |
| ATOM | 783 | CA | CYS | A | 110 | 69.579 | 50.941 | 9.206 | 1.00 | 39.83 | C |
| ATOM | 784 | CB | CYS | A | 110 | 69.771 | 49.443 | 9.309 | 1.00 | 49.43 | C |
| ATOM | 785 | SG | CYS | A | 110 | 71.304 | 48.984 | 10.127 | 1.00 | 57.18 | S |
| ATOM | 786 | C | CYS | A | 110 | 70.480 | 51.540 | 8.140 | 1.00 | 36.26 | C |
| ATOM | 787 | O | CYS | A | 110 | 70.895 | 50.878 | 7.222 | 1.00 | 43.68 | O |
| ATOM | 788 | N | GLY | A | 111 | 70.780 | 52.815 | 8.268 | 1.00 | 45.28 | N |
| ATOM | 789 | CA | GLY | A | 111 | 71.282 | 53.574 | 7.132 | 1.00 | 45.13 | C |
| ATOM | 790 | C | GLY | A | 111 | 70.829 | 54.982 | 7.369 | 1.00 | 51.84 | C |
| ATOM | 791 | O | GLY | A | 111 | 69.719 | 55.207 | 7.847 | 1.00 | 68.71 | O |
| ATOM | 792 | N | GLY | A | 112 | 71.678 | 55.934 | 7.023 | 1.00 | 61.20 | N |
| ATOM | 793 | CA | GLY | A | 112 | 71.602 | 57.273 | 7.603 | 1.00 | 62.75 | C |
| ATOM | 794 | C | GLY | A | 112 | 70.648 | 58.259 | 6.955 | 1.00 | 60.42 | C |
| ATOM | 795 | O | GLY | A | 112 | 71.066 | 59.353 | 6.559 | 1.00 | 79.36 | O |
| ATOM | 796 | N | ILE | A | 113 | 69.372 | 57.900 | 6.834 | 1.00 | 49.88 | N |
| ATOM | 797 | CA | ILE | A | 113 | 68.366 | 58.943 | 6.669 | 1.00 | 47.96 | C |
| ATOM | 798 | CB | ILE | A | 113 | 66.986 | 58.484 | 6.122 | 1.00 | 59.41 | C |
| ATOM | 799 | CG1 | ILE | A | 113 | 67.064 | 57.179 | 5.306 | 1.00 | 64.07 | C |
| ATOM | 800 | CD1 | ILE | A | 113 | 65.701 | 56.600 | 4.957 | 1.00 | 62.95 | C |
| ATOM | 801 | CG2 | ILE | A | 113 | 66.372 | 59.608 | 5.288 | 1.00 | 41.47 | C |
| ATOM | 802 | C | ILE | A | 113 | 68.203 | 59.451 | 8.100 | 1.00 | 39.00 | C |
| ATOM | 803 | O | ILE | A | 113 | 68.182 | 58.647 | 9.053 | 1.00 | 39.36 | O |
| ATOM | 804 | N | LEU | A | 114 | 68.138 | 60.764 | 8.260 | 1.00 | 29.33 | N |
| ATOM | 805 | CA | LEU | A | 114 | 67.740 | 61.371 | 9.528 | 1.00 | 24.96 | C |
| ATOM | 806 | CB | LEU | A | 114 | 67.936 | 62.884 | 9.458 | 1.00 | 27.04 | C |
| ATOM | 807 | CG | LEU | A | 114 | 67.777 | 63.738 | 10.711 | 1.00 | 32.37 | C |
| ATOM | 808 | CD1 | LEU | A | 114 | 68.312 | 65.144 | 10.463 | 1.00 | 27.25 | C |
| ATOM | 809 | CD2 | LEU | A | 114 | 66.316 | 63.798 | 11.126 | 1.00 | 31.93 | C |
| ATOM | 810 | C | LEU | A | 114 | 66.266 | 60.986 | 9.738 | 1.00 | 30.91 | C |
| ATOM | 811 | O | LEU | A | 114 | 65.444 | 61.280 | 8.882 | 1.00 | 32.11 | O |
| ATOM | 812 | N | ALA | A | 115 | 65.938 | 60.317 | 10.854 | 1.00 | 22.31 | N |
| ATOM | 813 | CA | ALA | A | 115 | 64.601 | 59.687 | 11.022 | 1.00 | 17.88 | C |
| ATOM | 814 | CB | ALA | A | 115 | 64.625 | 58.229 | 10.534 | 1.00 | 16.41 | C |
| ATOM | 815 | C | ALA | A | 115 | 64.188 | 59.757 | 12.472 | 1.00 | 15.02 | C |
| ATOM | 816 | O | ALA | A | 115 | 64.820 | 59.135 | 13.334 | 1.00 | 15.07 | O |
| ATOM | 817 | N | ASP | A | 116 | 63.163 | 60.559 | 12.752 | 1.00 | 13.37 | N |
| ATOM | 818 | CA | ASP | A | 116 | 62.755 | 60.875 | 14.143 | 1.00 | 11.94 | C |
| ATOM | 819 | CB | ASP | A | 116 | 62.602 | 62.399 | 14.288 | 1.00 | 13.11 | C |
| ATOM | 820 | CG | ASP | A | 116 | 63.939 | 63.128 | 14.215 | 1.00 | 17.42 | C |
| ATOM | 821 | OD1 | ASP | A | 116 | 64.960 | 62.454 | 14.445 | 1.00 | 18.27 | O |
| ATOM | 822 | OD2 | ASP | A | 116 | 63.968 | 64.362 | 13.963 | 1.00 | 18.41 | O |
| ATOM | 823 | C | ASP | A | 116 | 61.482 | 60.178 | 14.645 | 1.00 | 9.91 | C |
| ATOM | 824 | O | ASP | A | 116 | 61.099 | 60.351 | 15.794 | 1.00 | 11.02 | O |
| ATOM | 825 | N | GLN | A | 117 | 60.825 | 59.394 | 13.806 | 1.00 | 10.99 | N |
| ATOM | 826 | CA | GLN | A | 117 | 59.582 | 58.729 | 14.191 | 1.00 | 11.26 | C |
| ATOM | 827 | CB | GLN | A | 117 | 59.030 | 57.961 | 12.988 | 1.00 | 12.70 | C |
| ATOM | 828 | CG | GLN | A | 117 | 57.681 | 57.320 | 13.209 | 1.00 | 12.29 | C |
| ATOM | 829 | CD | GLN | A | 117 | 57.202 | 56.602 | 11.973 | 1.00 | 13.35 | C |
| ATOM | 830 | OE1 | GLN | A | 117 | 56.837 | 57.232 | 10.974 | 1.00 | 19.23 | O |
| ATOM | 831 | NE2 | GLN | A | 117 | 57.181 | 55.308 | 12.029 | 1.00 | 10.56 | N |
| ATOM | 832 | C | GLN | A | 117 | 59.790 | 57.773 | 15.366 | 1.00 | 11.00 | C |
| ATOM | 833 | O | GLN | A | 117 | 60.649 | 56.884 | 15.314 | 1.00 | 9.15 | O |
| ATOM | 834 | N | GLY | A | 118 | 59.005 | 57.966 | 16.430 | 1.00 | 11.48 | N |
| ATOM | 835 | CA | GLY | A | 118 | 59.034 | 57.065 | 17.608 | 1.00 | 9.34 | C |
| ATOM | 836 | C | GLY | A | 118 | 60.262 | 57.255 | 18.488 | 1.00 | 8.98 | C |
| ATOM | 837 | O | GLY | A | 118 | 60.474 | 56.490 | 19.421 | 1.00 | 9.55 | O |
| ATOM | 838 | N | LYS | A | 119 | 61.025 | 58.319 | 18.248 | 1.00 | 8.15 | N |
| ATOM | 839 | CA | LYS | A | 119 | 62.330 | 58.538 | 18.888 | 1.00 | 7.91 | C |
| ATOM | 840 | CB | LYS | A | 119 | 63.339 | 59.042 | 17.843 | 1.00 | 8.40 | C |
| ATOM | 841 | CG | LYS | A | 119 | 64.707 | 59.476 | 18.364 | 1.00 | 9.03 | C |
| ATOM | 842 | CD | LYS | A | 119 | 65.685 | 59.851 | 17.232 | 1.00 | 8.89 | C |
| ATOM | 843 | CE | LYS | A | 119 | 66.210 | 58.623 | 16.473 | 1.00 | 9.64 | C |
| ATOM | 844 | NZ | LYS | A | 119 | 66.918 | 58.935 | 15.184 | 1.00 | 9.41 | N |
| ATOM | 845 | C | LYS | A | 119 | 62.194 | 59.513 | 20.045 | 1.00 | 8.61 | C |
| ATOM | 846 | O | LYS | A | 119 | 61.330 | 60.404 | 20.022 | 1.00 | 10.22 | O |
| ATOM | 847 | N | ALA | A | 120 | 63.012 | 59.308 | 21.077 | 1.00 | 7.51 | N |
| ATOM | 848 | CA | ALA | A | 120 | 63.098 | 60.222 | 22.209 | 1.00 | 7.13 | C |
| ATOM | 849 | CB | ALA | A | 120 | 63.038 | 59.429 | 23.513 | 1.00 | 6.28 | C |
| ATOM | 850 | C | ALA | A | 120 | 64.395 | 61.036 | 22.153 | 1.00 | 7.14 | C |
| ATOM | 851 | O | ALA | A | 120 | 65.351 | 60.647 | 21.470 | 1.00 | 6.72 | O |
| ATOM | 852 | N | GLY | A | 121 | 64.425 | 62.135 | 22.901 | 1.00 | 6.81 | N |
| ATOM | 853 | CA | GLY | A | 121 | 65.573 | 63.034 | 22.957 | 1.00 | 8.27 | C |
| ATOM | 854 | C | GLY | A | 121 | 66.733 | 62.599 | 23.829 | 1.00 | 8.37 | C |
| ATOM | 855 | O | GLY | A | 121 | 67.340 | 63.440 | 24.512 | 1.00 | 8.71 | O |
| ATOM | 856 | N | TYR | A | 122 | 67.052 | 61.293 | 23.813 | 1.00 | 7.94 | N |
| ATOM | 857 | CA | TYR | A | 122 | 68.169 | 60.752 | 24.598 | 1.00 | 7.85 | C |
| ATOM | 858 | CB | TYR | A | 122 | 68.257 | 59.243 | 24.429 | 1.00 | 7.36 | C |
| ATOM | 859 | CG | TYR | A | 122 | 68.793 | 58.827 | 23.065 | 1.00 | 8.14 | C |
| ATOM | 860 | CD1 | TYR | A | 122 | 70.154 | 58.724 | 22.841 | 1.00 | 9.19 | C |
| ATOM | 861 | CE1 | TYR | A | 122 | 70.661 | 58.394 | 21.585 | 1.00 | 9.17 | C |
| ATOM | 862 | CZ | TYR | A | 122 | 69.802 | 58.127 | 20.549 | 1.00 | 8.00 | C |
| ATOM | 863 | OH | TYR | A | 122 | 70.303 | 57.803 | 19.310 | 1.00 | 7.95 | O |
| ATOM | 864 | CE2 | TYR | A | 122 | 68.437 | 58.237 | 20.747 | 1.00 | 8.48 | C |
| ATOM | 865 | CD2 | TYR | A | 122 | 67.942 | 58.592 | 22.003 | 1.00 | 8.41 | C |
| ATOM | 866 | C | TYR | A | 122 | 69.509 | 61.407 | 24.209 | 1.00 | 8.49 | C |
| ATOM | 867 | O | TYR | A | 122 | 70.467 | 61.414 | 24.990 | 1.00 | 9.08 | O |
| ATOM | 868 | N | HIS | A | 123 | 69.541 | 61.905 | 22.976 | 1.00 | 8.09 | N |
| ATOM | 869 | CA | HIS | A | 123 | 70.703 | 62.505 | 22.340 | 1.00 | 8.06 | C |
| ATOM | 870 | CB | HIS | A | 123 | 70.578 | 62.357 | 20.829 | 1.00 | 7.45 | C |
| ATOM | 871 | CG | HIS | A | 123 | 69.227 | 62.750 | 20.311 | 1.00 | 8.31 | C |
| ATOM | 872 | ND1 | HIS | A | 123 | 68.819 | 64.030 | 20.303 | 1.00 | 7.16 | N |
| ATOM | 873 | CE1 | HIS | A | 123 | 67.558 | 64.081 | 19.841 | 1.00 | 8.00 | C |
| ATOM | 874 | NE2 | HIS | A | 123 | 67.144 | 62.824 | 19.576 | 1.00 | 8.19 | N |
| ATOM | 875 | CD2 | HIS | A | 123 | 68.147 | 61.979 | 19.859 | 1.00 | 8.31 | C |
| ATOM | 876 | C | HIS | A | 123 | 70.913 | 63.946 | 22.723 | 1.00 | 8.09 | C |
| ATOM | 877 | O | HIS | A | 123 | 72.015 | 64.469 | 22.542 | 1.00 | 8.47 | O |
| ATOM | 878 | N | GLY | A | 124 | 69.866 | 64.582 | 23.252 | 1.00 | 8.41 | N |
| ATOM | 879 | CA | GLY | A | 124 | 69.897 | 65.966 | 23.755 | 1.00 | 8.27 | C |
| ATOM | 880 | C | GLY | A | 124 | 69.664 | 67.107 | 22.760 | 1.00 | 8.38 | C |
| ATOM | 881 | O | GLY | A | 124 | 69.557 | 68.254 | 23.174 | 1.00 | 10.78 | O |
| ATOM | 882 | N | TYR | A | 125 | 69.553 | 66.802 | 21.472 | 1.00 | 8.01 | N |
| ATOM | 883 | CA | TYR | A | 125 | 69.474 | 67.838 | 20.411 | 1.00 | 8.90 | C |
| ATOM | 884 | CB | TYR | A | 125 | 69.973 | 67.287 | 19.078 | 1.00 | 9.02 | C |
| ATOM | 885 | CG | TYR | A | 125 | 71.420 | 66.886 | 19.093 | 1.00 | 9.71 | C |
| ATOM | 886 | CD1 | TYR | A | 125 | 72.439 | 67.857 | 19.184 | 1.00 | 11.89 | C |
| ATOM | 887 | CE1 | TYR | A | 125 | 73.765 | 67.500 | 19.209 | 1.00 | 11.14 | C |
| ATOM | 888 | CZ | TYR | A | 125 | 74.099 | 66.139 | 19.154 | 1.00 | 11.90 | C |
| ATOM | 889 | OH | TYR | A | 125 | 75.397 | 65.737 | 19.181 | 1.00 | 11.84 | O |
| ATOM | 890 | CE2 | TYR | A | 125 | 73.109 | 65.171 | 19.076 | 1.00 | 11.88 | C |
| ATOM | 891 | CD2 | TYR | A | 125 | 71.785 | 65.550 | 19.043 | 1.00 | 9.99 | C |
| ATOM | 892 | C | TYR | A | 125 | 68.098 | 68.475 | 20.199 | 1.00 | 9.13 | C |
| ATOM | 893 | O | TYR | A | 125 | 67.929 | 69.328 | 19.305 | 1.00 | 9.20 | O |
| ATOM | 894 | N | TRP | A | 126 | 67.134 | 68.092 | 21.033 | 1.00 | 8.70 | N |
| ATOM | 895 | CA | TRP | A | 126 | 65.784 | 68.631 | 20.948 | 1.00 | 8.45 | C |
| ATOM | 896 | CB | TRP | A | 126 | 64.786 | 67.497 | 20.828 | 1.00 | 8.19 | C |
| ATOM | 897 | CG | TRP | A | 126 | 64.948 | 66.595 | 19.647 | 1.00 | 7.80 | C |
| ATOM | 898 | CD1 | TRP | A | 126 | 65.703 | 66.793 | 18.501 | 1.00 | 7.99 | C |
| ATOM | 899 | NE1 | TRP | A | 126 | 65.559 | 65.735 | 17.643 | 1.00 | 8.77 | N |
| ATOM | 900 | CE2 | TRP | A | 126 | 64.714 | 64.818 | 18.163 | 1.00 | 8.26 | C |
| ATOM | 901 | CD2 | TRP | A | 126 | 64.280 | 65.316 | 19.450 | 1.00 | 7.54 | C |
| ATOM | 902 | CE3 | TRP | A | 126 | 63.409 | 64.562 | 20.198 | 1.00 | 8.14 | C |
| ATOM | 903 | CZ3 | TRP | A | 126 | 62.983 | 63.341 | 19.699 | 1.00 | 8.06 | C |
| ATOM | 904 | CH2 | TRP | A | 126 | 63.393 | 62.880 | 18.459 | 1.00 | 8.34 | C |
| ATOM | 905 | CZ2 | TRP | A | 126 | 64.285 | 63.598 | 17.672 | 1.00 | 8.33 | C |
| ATOM | 906 | C | TRP | A | 126 | 65.433 | 69.473 | 22.149 | 1.00 | 8.53 | C |
| ATOM | 907 | O | TRP | A | 126 | 64.646 | 69.066 | 23.004 | 1.00 | 8.34 | O |
| ATOM | 908 | N | GLY | A | 127 | 66.007 | 70.670 | 22.222 | 1.00 | 8.52 | N |
| ATOM | 909 | CA | GLY | A | 127 | 65.856 | 71.519 | 23.371 | 1.00 | 8.25 | C |
| ATOM | 910 | C | GLY | A | 127 | 64.478 | 72.126 | 23.556 | 1.00 | 8.41 | C |
| ATOM | 911 | O | GLY | A | 127 | 63.875 | 72.583 | 22.599 | 1.00 | 8.48 | O |
| ATOM | 912 | N | VAL | A | 128 | 64.006 | 72.132 | 24.810 | 1.00 | 8.05 | N |
| ATOM | 913 | CA | AVAL | A | 128 | 62.735 | 72.722 | 25.228 | 0.50 | 7.57 | C |
| ATOM | 914 | CA | BVAL | A | 128 | 62.752 | 72.796 | 25.150 | 0.50 | 8.37 | C |
| ATOM | 915 | CB | AVAL | A | 128 | 61.847 | 71.645 | 25.883 | 0.50 | 7.06 | C |
| ATOM | 916 | CB | BVAL | A | 128 | 61.597 | 71.801 | 25.476 | 0.50 | 9.15 | C |
| ATOM | 917 | CG1 | AVAL | A | 128 | 60.541 | 72.240 | 26.427 | 0.50 | 6.29 | C |
| ATOM | 918 | CG1 | BVAL | A | 128 | 61.962 | 70.400 | 25.015 | 0.50 | 9.16 | C |
| ATOM | 919 | CG2 | AVAL | A | 128 | 61.545 | 70.549 | 24.870 | 0.50 | 6.90 | C |
| ATOM | 920 | CG2 | BVAL | A | 128 | 61.169 | 71.823 | 26.941 | 0.50 | 9.05 | C |
| ATOM | 921 | C | VAL | A | 128 | 62.981 | 73.865 | 26.209 | 1.00 | 8.10 | C |
| ATOM | 922 | O | VAL | A | 128 | 62.403 | 74.939 | 26.113 | 1.00 | 9.16 | O |
| ATOM | 923 | N | ASN | A | 129 | 63.859 | 73.627 | 27.171 | 1.00 | 8.52 | N |
| ATOM | 924 | CA | ASN | A | 129 | 64.257 | 74.687 | 28.090 | 1.00 | 9.76 | C |
| ATOM | 925 | CB | ASN | A | 129 | 63.715 | 74.475 | 29.504 | 1.00 | 9.54 | C |
| ATOM | 926 | CG | ASN | A | 129 | 63.887 | 75.714 | 30.370 | 1.00 | 11.86 | C |
| ATOM | 927 | OD1 | ASN | A | 129 | 64.876 | 76.456 | 30.223 | 1.00 | 11.47 | O |
| ATOM | 928 | ND2 | ASN | A | 129 | 62.928 | 75.953 | 31.282 | 1.00 | 10.82 | N |
| ATOM | 929 | C | ASN | A | 129 | 65.764 | 74.750 | 28.122 | 1.00 | 9.14 | C |
| ATOM | 930 | O | ASN | A | 129 | 66.407 | 73.896 | 28.725 | 1.00 | 8.31 | O |
| ATOM | 931 | N | PHE | A | 130 | 66.324 | 75.752 | 27.459 | 1.00 | 8.93 | N |
| ATOM | 932 | CA | PHE | A | 130 | 67.783 | 75.830 | 27.286 | 1.00 | 9.13 | C |
| ATOM | 933 | CB | PHE | A | 130 | 68.126 | 76.736 | 26.089 | 1.00 | 9.44 | C |
| ATOM | 934 | CG | PHE | A | 130 | 67.823 | 76.108 | 24.739 | 1.00 | 9.95 | C |
| ATOM | 935 | CD1 | PHE | A | 130 | 66.509 | 75.854 | 24.328 | 1.00 | 8.88 | C |
| ATOM | 936 | CE1 | PHE | A | 130 | 66.244 | 75.287 | 23.100 | 1.00 | 10.21 | C |
| ATOM | 937 | CZ | PHE | A | 130 | 67.284 | 74.958 | 22.239 | 1.00 | 10.77 | C |
| ATOM | 938 | CE2 | PHE | A | 130 | 68.591 | 75.198 | 22.633 | 1.00 | 11.17 | C |
| ATOM | 939 | CD2 | PHE | A | 130 | 68.851 | 75.777 | 23.877 | 1.00 | 10.30 | C |
| ATOM | 940 | C | PHE | A | 130 | 68.473 | 76.312 | 28.572 | 1.00 | 8.31 | C |
| ATOM | 941 | O | PHE | A | 130 | 69.695 | 76.311 | 28.655 | 1.00 | 8.16 | O |
| ATOM | 942 | N | TYR | A | 131 | 67.682 | 76.706 | 29.579 | 1.00 | 8.67 | N |
| ATOM | 943 | CA | TYR | A | 131 | 68.204 | 77.137 | 30.871 | 1.00 | 9.14 | C |
| ATOM | 944 | CB | TYR | A | 131 | 67.446 | 78.364 | 31.385 | 1.00 | 9.45 | C |
| ATOM | 945 | CG | TYR | A | 131 | 67.771 | 79.608 | 30.586 | 1.00 | 10.55 | C |
| ATOM | 946 | CD1 | TYR | A | 131 | 67.201 | 79.817 | 29.350 | 1.00 | 10.18 | C |
| ATOM | 947 | CE1 | TYR | A | 131 | 67.483 | 80.947 | 28.610 | 1.00 | 11.25 | C |
| ATOM | 948 | CZ | TYR | A | 131 | 68.363 | 81.875 | 29.088 | 1.00 | 11.51 | C |
| ATOM | 949 | OH | TYR | A | 131 | 68.660 | 82.957 | 28.302 | 1.00 | 12.41 | O |
| ATOM | 950 | CE2 | TYR | A | 131 | 68.962 | 81.701 | 30.314 | 1.00 | 12.46 | C |
| ATOM | 951 | CD2 | TYR | A | 131 | 68.672 | 80.555 | 31.058 | 1.00 | 12.49 | C |
| ATOM | 952 | C | TYR | A | 131 | 68.165 | 76.046 | 31.942 | 1.00 | 10.15 | C |
| ATOM | 953 | O | TYR | A | 131 | 68.588 | 76.290 | 33.034 | 1.00 | 9.65 | O |
| ATOM | 954 | N | GLN | A | 132 | 67.650 | 74.863 | 31.633 | 1.00 | 10.60 | N |
| ATOM | 955 | CA | GLN | A | 132 | 67.530 | 73.796 | 32.635 | 1.00 | 10.64 | C |
| ATOM | 956 | CB | GLN | A | 132 | 66.059 | 73.529 | 32.986 | 1.00 | 12.22 | C |
| ATOM | 957 | CG | GLN | A | 132 | 65.381 | 74.646 | 33.750 | 1.00 | 15.14 | C |
| ATOM | 958 | CD | GLN | A | 132 | 63.929 | 74.334 | 34.139 | 1.00 | 15.81 | C |
| ATOM | 959 | OE1 | GLN | A | 132 | 63.416 | 73.222 | 33.943 | 1.00 | 19.68 | O |
| ATOM | 960 | NE2 | GLN | A | 132 | 63.275 | 75.319 | 34.685 | 1.00 | 16.95 | N |
| ATOM | 961 | C | GLN | A | 132 | 68.137 | 72.521 | 32.084 | 1.00 | 9.69 | C |
| ATOM | 962 | O | GLN | A | 132 | 67.989 | 72.206 | 30.904 | 1.00 | 10.13 | O |
| ATOM | 963 | N | VAL | A | 133 | 68.795 | 71.787 | 32.960 | 1.00 | 10.21 | N |
| ATOM | 964 | CA | VAL | A | 133 | 69.376 | 70.502 | 32.632 | 1.00 | 10.16 | C |
| ATOM | 965 | CB | VAL | A | 133 | 70.437 | 70.114 | 33.675 | 1.00 | 11.32 | C |
| ATOM | 966 | CG1 | VAL | A | 133 | 70.946 | 68.707 | 33.419 | 1.00 | 12.83 | C |
| ATOM | 967 | CG2 | VAL | A | 133 | 71.586 | 71.103 | 33.599 | 1.00 | 13.12 | C |
| ATOM | 968 | C | VAL | A | 133 | 68.282 | 69.460 | 32.527 | 1.00 | 8.89 | C |
| ATOM | 969 | O | VAL | A | 133 | 67.335 | 69.468 | 33.281 | 1.00 | 7.09 | O |
| ATOM | 970 | N | ASP | A | 134 | 68.370 | 68.593 | 31.536 | 1.00 | 8.88 | N |
| ATOM | 971 | CA | ASP | A | 134 | 67.397 | 67.527 | 31.412 | 1.00 | 9.39 | C |
| ATOM | 972 | CB | ASP | A | 134 | 67.660 | 66.665 | 30.179 | 1.00 | 9.63 | C |
| ATOM | 973 | CG | ASP | A | 134 | 66.513 | 65.686 | 29.932 | 1.00 | 9.77 | C |
| ATOM | 974 | OD1 | ASP | A | 134 | 66.530 | 64.554 | 30.510 | 1.00 | 9.09 | O |
| ATOM | 975 | OD2 | ASP | A | 134 | 65.579 | 66.102 | 29.227 | 1.00 | 8.12 | O |
| ATOM | 976 | C | ASP | A | 134 | 67.412 | 66.628 | 32.657 | 1.00 | 9.22 | C |
| ATOM | 977 | O | ASP | A | 134 | 68.477 | 66.309 | 33.179 | 1.00 | 10.21 | O |
| ATOM | 978 | N | GLU | A | 135 | 66.224 | 66.240 | 33.118 | 1.00 | 9.96 | N |
| ATOM | 979 | CA | GLU | A | 135 | 66.047 | 65.459 | 34.343 | 1.00 | 9.72 | C |
| ATOM | 980 | CB | GLU | A | 135 | 64.532 | 65.295 | 34.660 | 1.00 | 12.37 | C |
| ATOM | 981 | CG | GLU | A | 135 | 63.748 | 64.378 | 33.718 | 1.00 | 11.48 | C |
| ATOM | 982 | CD | GLU | A | 135 | 63.500 | 64.952 | 32.336 | 1.00 | 12.01 | C |
| ATOM | 983 | OE1 | GLU | A | 135 | 63.634 | 66.184 | 32.127 | 1.00 | 12.59 | O |
| ATOM | 984 | OE2 | GLU | A | 135 | 63.190 | 64.152 | 31.433 | 1.00 | 9.85 | O |
| ATOM | 985 | C | GLU | A | 135 | 66.732 | 64.091 | 34.379 | 1.00 | 8.97 | C |
| ATOM | 986 | O | GLU | A | 135 | 66.926 | 63.511 | 35.456 | 1.00 | 10.04 | O |
| ATOM | 987 | N | HIS | A | 136 | 67.071 | 63.542 | 33.221 | 1.00 | 8.92 | N |
| ATOM | 988 | CA | HIS | A | 136 | 67.820 | 62.289 | 33.178 | 1.00 | 8.25 | C |
| ATOM | 989 | CB | HIS | A | 136 | 67.767 | 61.695 | 31.801 | 1.00 | 8.45 | C |
| ATOM | 990 | CG | HIS | A | 136 | 66.390 | 61.264 | 31.372 | 1.00 | 8.53 | C |
| ATOM | 991 | ND1 | HIS | A | 136 | 65.501 | 62.107 | 30.821 | 1.00 | 8.93 | N |
| ATOM | 992 | CE1 | HIS | A | 136 | 64.387 | 61.418 | 30.486 | 1.00 | 9.04 | C |
| ATOM | 993 | NE2 | HIS | A | 136 | 64.574 | 60.126 | 30.812 | 1.00 | 8.94 | N |
| ATOM | 994 | CD2 | HIS | A | 136 | 65.796 | 59.995 | 31.353 | 1.00 | 8.84 | C |
| ATOM | 995 | C | HIS | A | 136 | 69.258 | 62.399 | 33.603 | 1.00 | 8.98 | C |
| ATOM | 996 | O | HIS | A | 136 | 69.872 | 61.397 | 33.982 | 1.00 | 11.05 | O |
| ATOM | 997 | N | LEU | A | 137 | 69.832 | 63.593 | 33.557 | 1.00 | 9.41 | N |
| ATOM | 998 | CA | LEU | A | 137 | 71.313 | 63.711 | 33.625 | 1.00 | 9.45 | C |
| ATOM | 999 | CB | LEU | A | 137 | 71.790 | 64.812 | 32.680 | 1.00 | 9.45 | C |
| ATOM | 1000 | CG | LEU | A | 137 | 71.488 | 64.536 | 31.197 | 1.00 | 10.52 | C |
| ATOM | 1001 | CD1 | LEU | A | 137 | 71.891 | 65.726 | 30.311 | 1.00 | 9.09 | C |
| ATOM | 1002 | CD2 | LEU | A | 137 | 72.152 | 63.258 | 30.713 | 1.00 | 9.69 | C |
| ATOM | 1003 | C | LEU | A | 137 | 71.965 | 63.895 | 35.022 | 1.00 | 9.36 | C |
| ATOM | 1004 | O | LEU | A | 137 | 73.008 | 63.272 | 35.303 | 1.00 | 7.32 | O |
| ATOM | 1005 | N | PRO | A | 138 | 71.376 | 64.744 | 35.891 | 1.00 | 9.50 | N |
| ATOM | 1006 | CA | PRO | A | 138 | 72.044 | 65.010 | 37.139 | 1.00 | 10.29 | C |
| ATOM | 1007 | CB | PRO | A | 138 | 71.597 | 66.438 | 37.451 | 1.00 | 10.28 | C |
| ATOM | 1008 | CG | PRO | A | 138 | 70.174 | 66.447 | 36.998 | 1.00 | 10.84 | C |
| ATOM | 1009 | CD | PRO | A | 138 | 70.152 | 65.551 | 35.774 | 1.00 | 10.44 | C |
| ATOM | 1010 | C | PRO | A | 138 | 71.627 | 64.055 | 38.271 | 1.00 | 11.03 | C |
| ATOM | 1011 | O | PRO | A | 138 | 70.660 | 63.298 | 38.143 | 1.00 | 10.18 | O |
| ATOM | 1012 | N | SER | A | 139 | 72.404 | 64.072 | 39.348 | 1.00 | 11.65 | N |
| ATOM | 1013 | CA | SER | A | 139 | 72.102 | 63.317 | 40.552 | 1.00 | 12.27 | C |
| ATOM | 1014 | CB | SER | A | 139 | 73.058 | 62.126 | 40.699 | 1.00 | 12.48 | C |
| ATOM | 1015 | OG | SER | A | 139 | 73.018 | 61.239 | 39.606 | 1.00 | 13.25 | O |
| ATOM | 1016 | C | SER | A | 139 | 72.369 | 64.277 | 41.717 | 1.00 | 13.85 | C |
| ATOM | 1017 | O | SER | A | 139 | 73.116 | 65.243 | 41.560 | 1.00 | 12.37 | O |
| ATOM | 1018 | N | PRO | A | 140 | 71.846 | 63.960 | 42.906 | 1.00 | 17.20 | N |
| ATOM | 1019 | CA | PRO | A | 140 | 72.307 | 64.706 | 44.083 | 1.00 | 18.70 | C |
| ATOM | 1020 | CB | PRO | A | 140 | 71.583 | 64.009 | 45.245 | 1.00 | 20.40 | C |
| ATOM | 1021 | CG | PRO | A | 140 | 70.373 | 63.364 | 44.600 | 1.00 | 17.87 | C |
| ATOM | 1022 | CD | PRO | A | 140 | 70.911 | 62.880 | 43.281 | 1.00 | 17.00 | C |
| ATOM | 1023 | C | PRO | A | 140 | 73.806 | 64.557 | 44.214 | 1.00 | 17.28 | C |
| ATOM | 1024 | O | PRO | A | 140 | 74.338 | 63.472 | 44.037 | 1.00 | 17.34 | O |
| ATOM | 1025 | N | GLY | A | 141 | 74.502 | 65.643 | 44.482 | 1.00 | 16.43 | N |
| ATOM | 1026 | CA | GLY | A | 141 | 75.962 | 65.587 | 44.489 | 1.00 | 18.74 | C |
| ATOM | 1027 | C | GLY | A | 141 | 76.631 | 65.520 | 43.123 | 1.00 | 18.04 | C |
| ATOM | 1028 | O | GLY | A | 141 | 77.845 | 65.438 | 43.067 | 1.00 | 22.48 | O |
| ATOM | 1029 | N | MET | A | 142 | 75.860 | 65.535 | 42.024 | 1.00 | 16.24 | N |
| ATOM | 1030 | CA | MET | A | 142 | 76.437 | 65.521 | 40.675 | 1.00 | 13.09 | C |
| ATOM | 1031 | CB | MET | A | 142 | 76.781 | 64.093 | 40.237 | 1.00 | 14.71 | C |
| ATOM | 1032 | CG | MET | A | 142 | 77.649 | 64.002 | 38.983 | 1.00 | 16.11 | C |
| ATOM | 1033 | SD | MET | A | 142 | 79.099 | 65.060 | 39.002 | 1.00 | 17.36 | S |
| ATOM | 1034 | CE | MET | A | 142 | 80.244 | 64.352 | 40.174 | 1.00 | 12.41 | C |
| ATOM | 1035 | C | MET | A | 142 | 75.525 | 66.196 | 39.673 | 1.00 | 12.42 | C |
| ATOM | 1036 | O | MET | A | 142 | 74.959 | 65.562 | 38.786 | 1.00 | 10.34 | O |
| ATOM | 1037 | N | ASP | A | 143 | 75.377 | 67.505 | 39.827 | 1.00 | 13.19 | N |
| ATOM | 1038 | CA | ASP | A | 143 | 74.710 | 68.325 | 38.829 | 1.00 | 11.91 | C |
| ATOM | 1039 | CB | ASP | A | 143 | 73.987 | 69.510 | 39.498 | 1.00 | 15.02 | C |
| ATOM | 1040 | CG | ASP | A | 143 | 74.926 | 70.434 | 40.277 | 1.00 | 16.73 | C |
| ATOM | 1041 | OD1 | ASP | A | 143 | 76.166 | 70.322 | 40.147 | 1.00 | 17.79 | O |
| ATOM | 1042 | OD2 | ASP | A | 143 | 74.413 | 71.287 | 41.030 | 1.00 | 16.51 | O |
| ATOM | 1043 | C | ASP | A | 143 | 75.750 | 68.762 | 37.797 | 1.00 | 11.44 | C |
| ATOM | 1044 | O | ASP | A | 143 | 76.911 | 68.347 | 37.862 | 1.00 | 9.33 | O |
| ATOM | 1045 | N | PHE | A | 144 | 75.341 | 69.565 | 36.809 | 1.00 | 11.16 | N |
| ATOM | 1046 | CA | PHE | A | 144 | 76.271 | 70.003 | 35.769 | 1.00 | 10.14 | C |
| ATOM | 1047 | CB | PHE | A | 144 | 75.571 | 70.904 | 34.742 | 1.00 | 11.49 | C |
| ATOM | 1048 | CG | PHE | A | 144 | 76.494 | 71.361 | 33.623 | 1.00 | 11.77 | C |
| ATOM | 1049 | CD1 | PHE | A | 144 | 77.016 | 70.449 | 32.730 | 1.00 | 11.66 | C |
| ATOM | 1050 | CE1 | PHE | A | 144 | 77.876 | 70.852 | 31.737 | 1.00 | 10.97 | C |
| ATOM | 1051 | CZ | PHE | A | 144 | 78.233 | 72.182 | 31.626 | 1.00 | 11.63 | C |
| ATOM | 1052 | CE2 | PHE | A | 144 | 77.713 | 73.111 | 32.507 | 1.00 | 11.40 | C |
| ATOM | 1053 | CD2 | PHE | A | 144 | 76.859 | 72.696 | 33.499 | 1.00 | 10.90 | C |
| ATOM | 1054 | C | PHE | A | 144 | 77.529 | 70.687 | 36.323 | 1.00 | 10.74 | C |
| ATOM | 1055 | O | PHE | A | 144 | 78.641 | 70.350 | 35.932 | 1.00 | 11.37 | O |
| ATOM | 1056 | N | ARG | A | 145 | 77.351 | 71.644 | 37.231 | 1.00 | 10.47 | N |
| ATOM | 1057 | CA | ARG | A | 145 | 78.458 | 72.297 | 37.912 | 1.00 | 12.14 | C |
| ATOM | 1058 | CB | ARG | A | 145 | 77.922 | 73.190 | 39.018 | 1.00 | 16.55 | C |
| ATOM | 1059 | CG | ARG | A | 145 | 77.857 | 74.661 | 38.704 | 1.00 | 18.46 | C |
| ATOM | 1060 | CD | ARG | A | 145 | 77.467 | 75.487 | 39.948 | 1.00 | 15.76 | C |
| ATOM | 1061 | NE | ARG | A | 145 | 76.872 | 76.736 | 39.487 | 1.00 | 21.12 | N |
| ATOM | 1062 | CZ | ARG | A | 145 | 77.564 | 77.813 | 39.125 | 1.00 | 24.33 | C |
| ATOM | 1063 | NH1 | ARG | A | 145 | 78.902 | 77.839 | 39.201 | 1.00 | 26.15 | N |
| ATOM | 1064 | NH2 | ARG | A | 145 | 76.917 | 78.889 | 38.701 | 1.00 | 24.96 | N |
| ATOM | 1065 | C | ARG | A | 145 | 79.431 | 71.304 | 38.562 | 1.00 | 12.33 | C |
| ATOM | 1066 | O | ARG | A | 145 | 80.653 | 71.465 | 38.464 | 1.00 | 11.76 | O |
| ATOM | 1067 | N | ASP | A | 146 | 78.881 | 70.307 | 39.258 | 1.00 | 10.65 | N |
| ATOM | 1068 | CA | ASP | A | 146 | 79.705 | 69.263 | 39.893 | 1.00 | 11.11 | C |
| ATOM | 1069 | CB | ASP | A | 146 | 78.855 | 68.280 | 40.714 | 1.00 | 12.33 | C |
| ATOM | 1070 | CG | ASP | A | 146 | 78.088 | 68.968 | 41.842 | 1.00 | 13.59 | C |
| ATOM | 1071 | OD1 | ASP | A | 146 | 78.683 | 69.833 | 42.501 | 1.00 | 15.25 | O |
| ATOM | 1072 | OD2 | ASP | A | 146 | 76.902 | 68.651 | 42.041 | 1.00 | 14.56 | O |
| ATOM | 1073 | C | ASP | A | 146 | 80.493 | 68.477 | 38.848 | 1.00 | 10.17 | C |
| ATOM | 1074 | O | ASP | A | 146 | 81.654 | 68.133 | 39.062 | 1.00 | 9.83 | O |
| ATOM | 1075 | N | LEU | A | 147 | 79.864 | 68.184 | 37.719 | 1.00 | 9.45 | N |
| ATOM | 1076 | CA | LEU | A | 147 | 80.555 | 67.437 | 36.687 | 1.00 | 8.76 | C |
| ATOM | 1077 | CB | LEU | A | 147 | 79.593 | 67.022 | 35.581 | 1.00 | 8.79 | C |
| ATOM | 1078 | CG | LEU | A | 147 | 80.239 | 66.363 | 34.341 | 1.00 | 7.88 | C |
| ATOM | 1079 | CD1 | LEU | A | 147 | 80.720 | 64.964 | 34.710 | 1.00 | 7.67 | C |
| ATOM | 1080 | CD2 | LEU | A | 147 | 79.222 | 66.316 | 33.185 | 1.00 | 7.01 | C |
| ATOM | 1081 | C | LEU | A | 147 | 81.702 | 68.265 | 36.112 | 1.00 | 9.10 | C |
| ATOM | 1082 | O | LEU | A | 147 | 82.817 | 67.767 | 35.928 | 1.00 | 8.89 | O |
| ATOM | 1083 | N | ALA | A | 148 | 81.451 | 69.528 | 35.802 | 1.00 | 10.28 | N |
| ATOM | 1084 | CA | ALA | A | 148 | 82.539 | 70.346 | 35.279 | 1.00 | 10.21 | C |
| ATOM | 1085 | CB | ALA | A | 148 | 82.043 | 71.711 | 34.787 | 1.00 | 10.97 | C |
| ATOM | 1086 | C | ALA | A | 148 | 83.659 | 70.478 | 36.334 | 1.00 | 9.79 | C |
| ATOM | 1087 | O | ALA | A | 148 | 84.838 | 70.371 | 35.998 | 1.00 | 9.35 | O |
| ATOM | 1088 | N | ALA | A | 149 | 83.305 | 70.636 | 37.604 | 1.00 | 9.63 | N |
| ATOM | 1089 | CA | ALA | A | 149 | 84.327 | 70.721 | 38.679 | 1.00 | 9.33 | C |
| ATOM | 1090 | CB | ALA | A | 149 | 83.714 | 71.104 | 40.040 | 1.00 | 8.38 | C |
| ATOM | 1091 | C | ALA | A | 149 | 85.097 | 69.389 | 38.803 | 1.00 | 11.27 | C |
| ATOM | 1092 | O | ALA | A | 149 | 86.296 | 69.388 | 39.110 | 1.00 | 12.08 | O |
| ATOM | 1093 | N | ALA | A | 150 | 84.432 | 68.255 | 38.580 | 1.00 | 11.83 | N |
| ATOM | 1094 | CA | ALA | A | 150 | 85.137 | 66.948 | 38.658 | 1.00 | 14.01 | C |
| ATOM | 1095 | CB | ALA | A | 150 | 84.173 | 65.766 | 38.578 | 1.00 | 12.75 | C |
| ATOM | 1096 | C | ALA | A | 150 | 86.166 | 66.870 | 37.546 | 1.00 | 13.00 | C |
| ATOM | 1097 | O | ALA | A | 150 | 87.300 | 66.469 | 37.761 | 1.00 | 13.57 | O |
| ATOM | 1098 | N | MET | A | 151 | 85.791 | 67.332 | 36.371 | 1.00 | 13.01 | N |
| ATOM | 1099 | CA | MET | A | 151 | 86.733 | 67.356 | 35.256 | 1.00 | 13.02 | C |
| ATOM | 1100 | CB | MET | A | 151 | 86.045 | 67.768 | 33.956 | 1.00 | 12.26 | C |
| ATOM | 1101 | CG | MET | A | 151 | 85.024 | 66.752 | 33.479 | 1.00 | 14.82 | C |
| ATOM | 1102 | SD | MET | A | 151 | 84.476 | 67.113 | 31.811 | 1.00 | 15.18 | S |
| ATOM | 1103 | CE | MET | A | 151 | 83.191 | 65.868 | 31.584 | 1.00 | 16.42 | C |
| ATOM | 1104 | C | MET | A | 151 | 87.886 | 68.286 | 35.545 | 1.00 | 11.45 | C |
| ATOM | 1105 | O | MET | A | 151 | 89.034 | 67.930 | 35.310 | 1.00 | 13.59 | O |
| ATOM | 1106 | N | HIS | A | 152 | 87.584 | 69.466 | 36.057 | 1.00 | 11.04 | N |
| ATOM | 1107 | CA | HIS | A | 152 | 88.624 | 70.454 | 36.356 | 1.00 | 11.67 | C |
| ATOM | 1108 | CB | HIS | A | 152 | 87.999 | 71.778 | 36.792 | 1.00 | 11.43 | C |
| ATOM | 1109 | CG | HIS | A | 152 | 87.153 | 72.432 | 35.697 | 1.00 | 12.31 | C |
| ATOM | 1110 | ND1 | HIS | A | 152 | 86.192 | 73.335 | 35.960 | 1.00 | 12.29 | N |
| ATOM | 1111 | CE1 | HIS | A | 152 | 85.607 | 73.730 | 34.810 | 1.00 | 10.76 | C |
| ATOM | 1112 | NE2 | HIS | A | 152 | 86.164 | 73.044 | 33.806 | 1.00 | 12.01 | N |
| ATOM | 1113 | CD2 | HIS | A | 152 | 87.133 | 72.233 | 34.321 | 1.00 | 11.22 | C |
| ATOM | 1114 | C | HIS | A | 152 | 89.627 | 69.974 | 37.364 | 1.00 | 13.31 | C |
| ATOM | 1115 | O | HIS | A | 152 | 90.830 | 70.152 | 37.159 | 1.00 | 11.45 | O |
| ATOM | 1116 | N | ARG | A | 153 | 89.169 | 69.345 | 38.447 | 1.00 | 13.29 | N |
| ATOM | 1117 | CA | ARG | A | 153 | 90.106 | 68.770 | 39.427 | 1.00 | 17.06 | C |
| ATOM | 1118 | CB | ARG | A | 153 | 89.358 | 68.107 | 40.606 | 1.00 | 21.01 | C |
| ATOM | 1119 | CG | ARG | A | 153 | 88.677 | 69.115 | 41.516 | 1.00 | 22.99 | C |
| ATOM | 1120 | CD | ARG | A | 153 | 88.226 | 68.456 | 42.808 | 1.00 | 31.60 | C |
| ATOM | 1121 | NE | ARG | A | 153 | 87.257 | 67.388 | 42.550 | 1.00 | 29.94 | N |
| ATOM | 1122 | CZ | ARG | A | 153 | 85.938 | 67.557 | 42.442 | 1.00 | 30.44 | C |
| ATOM | 1123 | NH1 | ARG | A | 153 | 85.382 | 68.762 | 42.568 | 1.00 | 31.35 | N |
| ATOM | 1124 | NH2 | ARG | A | 153 | 85.166 | 66.507 | 42.198 | 1.00 | 30.71 | N |
| ATOM | 1125 | C | ARG | A | 153 | 91.068 | 67.758 | 38.822 | 1.00 | 16.63 | C |
| ATOM | 1126 | O | ARG | A | 153 | 92.159 | 67.543 | 39.366 | 1.00 | 16.28 | O |
| ATOM | 1127 | N | LYS | A | 154 | 90.670 | 67.126 | 37.718 | 1.00 | 13.26 | N |
| ATOM | 1128 | CA | LYS | A | 154 | 91.524 | 66.177 | 37.030 | 1.00 | 12.93 | C |
| ATOM | 1129 | CB | LYS | A | 154 | 90.715 | 64.973 | 36.581 | 1.00 | 15.97 | C |
| ATOM | 1130 | CG | LYS | A | 154 | 90.275 | 64.098 | 37.756 | 1.00 | 19.37 | C |
| ATOM | 1131 | CD | LYS | A | 154 | 89.440 | 62.932 | 37.259 | 1.00 | 23.42 | C |
| ATOM | 1132 | CE | LYS | A | 154 | 88.702 | 62.211 | 38.383 | 1.00 | 30.99 | C |
| ATOM | 1133 | NZ | LYS | A | 154 | 89.622 | 61.640 | 39.392 | 1.00 | 27.38 | N |
| ATOM | 1134 | C | LYS | A | 154 | 92.263 | 66.794 | 35.855 | 1.00 | 15.47 | C |
| ATOM | 1135 | O | LYS | A | 154 | 92.841 | 66.069 | 35.038 | 1.00 | 14.55 | O |
| ATOM | 1136 | N | GLY | A | 155 | 92.276 | 68.123 | 35.781 | 1.00 | 13.30 | N |
| ATOM | 1137 | CA | GLY | A | 155 | 92.968 | 68.802 | 34.696 | 1.00 | 13.43 | C |
| ATOM | 1138 | C | GLY | A | 155 | 92.278 | 68.614 | 33.356 | 1.00 | 14.51 | C |
| ATOM | 1139 | O | GLY | A | 155 | 92.912 | 68.790 | 32.342 | 1.00 | 15.46 | O |
| ATOM | 1140 | N | MET | A | 156 | 90.977 | 68.293 | 33.347 | 1.00 | 11.95 | N |
| ATOM | 1141 | CA | MET | A | 156 | 90.241 | 68.077 | 32.090 | 1.00 | 11.76 | C |
| ATOM | 1142 | CB | MET | A | 156 | 89.457 | 66.771 | 32.162 | 1.00 | 12.29 | C |
| ATOM | 1143 | CG | MET | A | 156 | 90.355 | 65.578 | 32.470 | 1.00 | 13.09 | C |
| ATOM | 1144 | SD | MET | A | 156 | 89.457 | 64.061 | 32.800 | 1.00 | 15.41 | S |
| ATOM | 1145 | CE | MET | A | 156 | 89.132 | 63.520 | 31.122 | 1.00 | 13.69 | C |
| ATOM | 1146 | C | MET | A | 156 | 89.309 | 69.243 | 31.716 | 1.00 | 11.04 | C |
| ATOM | 1147 | O | MET | A | 156 | 88.832 | 69.997 | 32.595 | 1.00 | 13.61 | O |
| ATOM | 1148 | N | LYS | A | 157 | 89.054 | 69.408 | 30.421 | 1.00 | 9.98 | N |
| ATOM | 1149 | CA | LYS | A | 157 | 88.124 | 70.444 | 29.967 | 1.00 | 10.72 | C |
| ATOM | 1150 | CB | LYS | A | 157 | 88.680 | 71.228 | 28.777 | 1.00 | 13.10 | C |
| ATOM | 1151 | CG | LYS | A | 157 | 90.049 | 71.891 | 28.971 | 1.00 | 17.30 | C |
| ATOM | 1152 | CD | LYS | A | 157 | 89.950 | 73.166 | 29.769 | 1.00 | 17.60 | C |
| ATOM | 1153 | CE | LYS | A | 157 | 91.171 | 74.056 | 29.568 | 1.00 | 19.32 | C |
| ATOM | 1154 | NZ | LYS | A | 157 | 92.405 | 73.243 | 29.573 | 1.00 | 22.94 | N |
| ATOM | 1155 | C | LYS | A | 157 | 86.803 | 69.801 | 29.546 | 1.00 | 10.51 | C |
| ATOM | 1156 | O | LYS | A | 157 | 86.773 | 68.680 | 28.992 | 1.00 | 9.65 | O |
| ATOM | 1157 | N | LEU | A | 158 | 85.723 | 70.537 | 29.773 | 1.00 | 10.14 | N |
| ATOM | 1158 | CA | LEU | A | 158 | 84.393 | 70.116 | 29.372 | 1.00 | 9.72 | C |
| ATOM | 1159 | CB | LEU | A | 158 | 83.385 | 70.362 | 30.504 | 1.00 | 11.39 | C |
| ATOM | 1160 | CG | LEU | A | 158 | 82.001 | 69.714 | 30.336 | 1.00 | 11.70 | C |
| ATOM | 1161 | CD1 | LEU | A | 158 | 81.338 | 69.510 | 31.699 | 1.00 | 9.61 | C |
| ATOM | 1162 | CD2 | LEU | A | 158 | 81.110 | 70.545 | 29.399 | 1.00 | 9.56 | C |
| ATOM | 1163 | C | LEU | A | 158 | 83.987 | 70.852 | 28.118 | 1.00 | 9.43 | C |
| ATOM | 1164 | O | LEU | A | 158 | 83.848 | 72.073 | 28.132 | 1.00 | 9.84 | O |
| ATOM | 1165 | N | VAL | A | 159 | 83.811 | 70.095 | 27.038 | 1.00 | 8.74 | N |
| ATOM | 1166 | CA | VAL | A | 159 | 83.271 | 70.592 | 25.786 | 1.00 | 8.60 | C |
| ATOM | 1167 | CB | VAL | A | 159 | 83.963 | 69.928 | 24.552 | 1.00 | 8.80 | C |
| ATOM | 1168 | CG1 | VAL | A | 159 | 83.401 | 70.504 | 23.248 | 1.00 | 9.13 | C |
| ATOM | 1169 | CG2 | VAL | A | 159 | 85.473 | 70.131 | 24.591 | 1.00 | 7.66 | C |
| ATOM | 1170 | C | VAL | A | 159 | 81.758 | 70.345 | 25.754 | 1.00 | 9.14 | C |
| ATOM | 1171 | O | VAL | A | 159 | 81.314 | 69.202 | 25.730 | 1.00 | 9.70 | O |
| ATOM | 1172 | N | LEU | A | 160 | 80.970 | 71.419 | 25.764 | 1.00 | 9.39 | N |
| ATOM | 1173 | CA | LEU | A | 160 | 79.514 | 71.306 | 25.711 | 1.00 | 9.75 | C |
| ATOM | 1174 | CB | LEU | A | 160 | 78.849 | 72.395 | 26.546 | 1.00 | 8.89 | C |
| ATOM | 1175 | CG | LEU | A | 160 | 77.322 | 72.349 | 26.614 | 1.00 | 10.13 | C |
| ATOM | 1176 | CD1 | LEU | A | 160 | 76.814 | 71.088 | 27.327 | 1.00 | 8.66 | C |
| ATOM | 1177 | CD2 | LEU | A | 160 | 76.850 | 73.588 | 27.385 | 1.00 | 9.44 | C |
| ATOM | 1178 | C | LEU | A | 160 | 79.024 | 71.398 | 24.264 | 1.00 | 9.38 | C |
| ATOM | 1179 | O | LEU | A | 160 | 79.311 | 72.353 | 23.546 | 1.00 | 9.29 | O |
| ATOM | 1180 | N | ASP | A | 161 | 78.311 | 70.375 | 23.839 | 1.00 | 9.16 | N |
| ATOM | 1181 | CA | ASP | A | 161 | 77.601 | 70.376 | 22.565 | 1.00 | 8.48 | C |
| ATOM | 1182 | CB | ASP | A | 161 | 77.138 | 68.943 | 22.333 | 1.00 | 9.13 | C |
| ATOM | 1183 | CG | ASP | A | 161 | 76.791 | 68.627 | 20.905 | 1.00 | 11.27 | C |
| ATOM | 1184 | OD1 | ASP | A | 161 | 76.360 | 69.536 | 20.151 | 1.00 | 11.78 | O |
| ATOM | 1185 | OD2 | ASP | A | 161 | 76.950 | 67.415 | 20.544 | 1.00 | 11.60 | O |
| ATOM | 1186 | C | ASP | A | 161 | 76.399 | 71.311 | 22.716 | 1.00 | 8.96 | C |
| ATOM | 1187 | O | ASP | A | 161 | 75.605 | 71.137 | 23.664 | 1.00 | 7.22 | O |
| ATOM | 1188 | N | ILE | A | 162 | 76.256 | 72.266 | 21.796 | 1.00 | 7.98 | N |
| ATOM | 1189 | CA | ILE | A | 162 | 75.198 | 73.244 | 21.836 | 1.00 | 8.99 | C |
| ATOM | 1190 | CB | ILE | A | 162 | 75.706 | 74.656 | 22.235 | 1.00 | 10.59 | C |
| ATOM | 1191 | CG1 | ILE | A | 162 | 76.518 | 75.336 | 21.119 | 1.00 | 10.61 | C |
| ATOM | 1192 | CD1 | ILE | A | 162 | 76.726 | 76.821 | 21.359 | 1.00 | 12.11 | C |
| ATOM | 1193 | CG2 | ILE | A | 162 | 76.572 | 74.586 | 23.494 | 1.00 | 9.93 | C |
| ATOM | 1194 | C | ILE | A | 162 | 74.424 | 73.353 | 20.530 | 1.00 | 8.62 | C |
| ATOM | 1195 | O | ILE | A | 162 | 74.966 | 73.150 | 19.438 | 1.00 | 10.83 | O |
| ATOM | 1196 | N | VAL | A | 163 | 73.144 | 73.695 | 20.653 | 1.00 | 7.41 | N |
| ATOM | 1197 | CA | VAL | A | 163 | 72.280 | 73.932 | 19.499 | 1.00 | 7.61 | C |
| ATOM | 1198 | CB | VAL | A | 163 | 71.066 | 72.997 | 19.488 | 1.00 | 6.78 | C |
| ATOM | 1199 | CG1 | VAL | A | 163 | 70.202 | 73.251 | 18.245 | 1.00 | 6.43 | C |
| ATOM | 1200 | CG2 | VAL | A | 163 | 71.546 | 71.556 | 19.529 | 1.00 | 6.38 | C |
| ATOM | 1201 | C | VAL | A | 163 | 71.776 | 75.376 | 19.480 | 1.00 | 8.62 | C |
| ATOM | 1202 | O | VAL | A | 163 | 71.347 | 75.908 | 20.508 | 1.00 | 8.53 | O |
| ATOM | 1203 | N | GLY | A | 164 | 71.820 | 75.994 | 18.301 | 1.00 | 7.93 | N |
| ATOM | 1204 | CA | GLY | A | 164 | 71.130 | 77.274 | 18.081 | 1.00 | 7.48 | C |
| ATOM | 1205 | C | GLY | A | 164 | 70.020 | 77.198 | 17.051 | 1.00 | 7.00 | C |
| ATOM | 1206 | O | GLY | A | 164 | 69.088 | 77.984 | 17.083 | 1.00 | 7.88 | O |
| ATOM | 1207 | N | ASN | A | 165 | 70.110 | 76.252 | 16.130 | 1.00 | 8.26 | N |
| ATOM | 1208 | CA | ASN | A | 165 | 69.221 | 76.215 | 14.979 | 1.00 | 8.67 | C |
| ATOM | 1209 | CB | ASN | A | 165 | 69.703 | 75.160 | 13.969 | 1.00 | 8.87 | C |
| ATOM | 1210 | CG | ASN | A | 165 | 68.745 | 75.002 | 12.813 | 1.00 | 9.62 | C |
| ATOM | 1211 | OD1 | ASN | A | 165 | 68.580 | 75.937 | 12.041 | 1.00 | 9.53 | O |
| ATOM | 1212 | ND2 | ASN | A | 165 | 68.105 | 73.814 | 12.680 | 1.00 | 7.11 | N |
| ATOM | 1213 | C | ASN | A | 165 | 67.774 | 75.920 | 15.314 | 1.00 | 8.35 | C |
| ATOM | 1214 | O | ASN | A | 165 | 66.854 | 76.413 | 14.647 | 1.00 | 8.60 | O |
| ATOM | 1215 | N | HIS | A | 166 | 67.559 | 75.074 | 16.313 | 1.00 | 7.83 | N |
| ATOM | 1216 | CA | HIS | A | 166 | 66.229 | 74.506 | 16.508 | 1.00 | 7.65 | C |
| ATOM | 1217 | CB | HIS | A | 166 | 66.091 | 73.263 | 15.628 | 1.00 | 8.29 | C |
| ATOM | 1218 | CG | HIS | A | 166 | 67.172 | 72.236 | 15.862 | 1.00 | 8.07 | C |
| ATOM | 1219 | ND1 | HIS | A | 166 | 68.272 | 72.169 | 15.098 | 1.00 | 8.29 | N |
| ATOM | 1220 | CE1 | HIS | A | 166 | 69.057 | 71.155 | 15.518 | 1.00 | 7.93 | C |
| ATOM | 1221 | NE2 | HIS | A | 166 | 68.455 | 70.563 | 16.554 | 1.00 | 6.83 | N |
| ATOM | 1222 | CD2 | HIS | A | 166 | 67.272 | 71.207 | 16.785 | 1.00 | 7.64 | C |
| ATOM | 1223 | C | HIS | A | 166 | 65.915 | 74.135 | 17.900 | 1.00 | 8.08 | C |
| ATOM | 1224 | O | HIS | A | 166 | 66.823 | 74.019 | 18.754 | 1.00 | 7.94 | O |
| ATOM | 1225 | N | GLY | A | 167 | 64.606 | 74.009 | 18.154 | 1.00 | 8.96 | N |
| ATOM | 1226 | CA | GLY | A | 167 | 64.071 | 73.474 | 19.403 | 1.00 | 8.66 | C |
| ATOM | 1227 | C | GLY | A | 167 | 63.925 | 71.971 | 19.236 | 1.00 | 8.82 | C |
| ATOM | 1228 | O | GLY | A | 167 | 64.912 | 71.242 | 19.214 | 1.00 | 9.81 | O |
| ATOM | 1229 | N | SER | A | 168 | 62.696 | 71.518 | 19.072 | 1.00 | 8.21 | N |
| ATOM | 1230 | CA | SER | A | 168 | 62.386 | 70.101 | 19.014 | 1.00 | 8.23 | C |
| ATOM | 1231 | CB | SER | A | 168 | 61.710 | 69.691 | 20.325 | 1.00 | 8.48 | C |
| ATOM | 1232 | OG | SER | A | 168 | 60.578 | 70.497 | 20.627 | 1.00 | 8.24 | O |
| ATOM | 1233 | C | SER | A | 168 | 61.476 | 69.829 | 17.829 | 1.00 | 8.48 | C |
| ATOM | 1234 | O | SER | A | 168 | 60.941 | 70.772 | 17.218 | 1.00 | 9.27 | O |
| ATOM | 1235 | N | PRO | A | 169 | 61.282 | 68.546 | 17.478 | 1.00 | 8.51 | N |
| ATOM | 1236 | CA | PRO | A | 169 | 60.333 | 68.214 | 16.433 | 1.00 | 8.01 | C |
| ATOM | 1237 | CB | PRO | A | 169 | 60.291 | 66.691 | 16.485 | 1.00 | 8.56 | C |
| ATOM | 1238 | CG | PRO | A | 169 | 61.668 | 66.311 | 16.939 | 1.00 | 8.29 | C |
| ATOM | 1239 | CD | PRO | A | 169 | 62.009 | 67.360 | 17.955 | 1.00 | 8.76 | C |
| ATOM | 1240 | C | PRO | A | 169 | 58.973 | 68.856 | 16.740 | 1.00 | 8.71 | C |
| ATOM | 1241 | O | PRO | A | 169 | 58.507 | 68.782 | 17.875 | 1.00 | 9.30 | O |
| ATOM | 1242 | N | ALA | A | 170 | 58.350 | 69.477 | 15.742 | 1.00 | 8.29 | N |
| ATOM | 1243 | CA | ALA | A | 170 | 57.331 | 70.516 | 16.016 | 1.00 | 8.65 | C |
| ATOM | 1244 | CB | ALA | A | 170 | 57.906 | 71.887 | 15.643 | 1.00 | 7.77 | C |
| ATOM | 1245 | C | ALA | A | 170 | 55.975 | 70.320 | 15.342 | 1.00 | 8.72 | C |
| ATOM | 1246 | O | ALA | A | 170 | 55.021 | 71.017 | 15.691 | 1.00 | 11.53 | O |
| ATOM | 1247 | N | TRP | A | 171 | 55.877 | 69.411 | 14.377 | 1.00 | 9.44 | N |
| ATOM | 1248 | CA | TRP | A | 171 | 54.588 | 69.135 | 13.712 | 1.00 | 9.22 | C |
| ATOM | 1249 | CB | TRP | A | 171 | 54.250 | 70.198 | 12.631 | 1.00 | 9.17 | C |
| ATOM | 1250 | CG | TRP | A | 171 | 55.097 | 70.062 | 11.407 | 1.00 | 10.08 | C |
| ATOM | 1251 | CD1 | TRP | A | 171 | 54.828 | 69.330 | 10.270 | 1.00 | 12.31 | C |
| ATOM | 1252 | NE1 | TRP | A | 171 | 55.861 | 69.430 | 9.371 | 1.00 | 13.50 | N |
| ATOM | 1253 | CE2 | TRP | A | 171 | 56.849 | 70.206 | 9.869 | 1.00 | 11.29 | C |
| ATOM | 1254 | CD2 | TRP | A | 171 | 56.434 | 70.628 | 11.194 | 1.00 | 9.86 | C |
| ATOM | 1255 | CE3 | TRP | A | 171 | 57.256 | 71.492 | 11.914 | 1.00 | 11.00 | C |
| ATOM | 1256 | CZ3 | TRP | A | 171 | 58.503 | 71.842 | 11.364 | 1.00 | 10.59 | C |
| ATOM | 1257 | CH2 | TRP | A | 171 | 58.887 | 71.395 | 10.106 | 1.00 | 10.19 | C |
| ATOM | 1258 | CZ2 | TRP | A | 171 | 58.063 | 70.580 | 9.330 | 1.00 | 11.61 | C |
| ATOM | 1259 | C | TRP | A | 171 | 54.525 | 67.751 | 13.157 | 1.00 | 8.65 | C |
| ATOM | 1260 | O | TRP | A | 171 | 55.510 | 67.003 | 13.202 | 1.00 | 10.22 | O |
| ATOM | 1261 | N | GLY | A | 172 | 53.344 | 67.384 | 12.663 | 1.00 | 10.12 | N |
| ATOM | 1262 | CA | GLY | A | 172 | 53.087 | 66.056 | 12.107 | 1.00 | 10.47 | C |
| ATOM | 1263 | C | GLY | A | 172 | 52.718 | 65.028 | 13.153 | 1.00 | 12.29 | C |
| ATOM | 1264 | O | GLY | A | 172 | 52.660 | 63.826 | 12.858 | 1.00 | 12.87 | O |
| ATOM | 1265 | N | MET | A | 173 | 52.471 | 65.491 | 14.374 | 1.00 | 12.39 | N |
| ATOM | 1266 | CA | MET | A | 173 | 52.213 | 64.600 | 15.497 | 1.00 | 11.92 | C |
| ATOM | 1267 | CB | MET | A | 173 | 52.595 | 65.286 | 16.817 | 1.00 | 11.23 | C |
| ATOM | 1268 | CG | MET | A | 173 | 54.105 | 65.396 | 16.983 | 1.00 | 11.15 | C |
| ATOM | 1269 | SD | MET | A | 173 | 54.626 | 66.456 | 18.324 | 1.00 | 11.59 | S |
| ATOM | 1270 | CE | MET | A | 173 | 54.753 | 68.069 | 17.495 | 1.00 | 10.82 | C |
| ATOM | 1271 | C | MET | A | 173 | 50.739 | 64.172 | 15.521 | 1.00 | 12.16 | C |
| ATOM | 1272 | O | MET | A | 173 | 49.894 | 64.888 | 14.996 | 1.00 | 10.97 | O |
| ATOM | 1273 | N | ALA | A | 174 | 50.457 | 63.042 | 16.178 | 1.00 | 12.44 | N |
| ATOM | 1274 | CA | ALA | A | 174 | 49.095 | 62.508 | 16.344 | 1.00 | 11.08 | C |
| ATOM | 1275 | CB | ALA | A | 174 | 49.149 | 61.126 | 16.988 | 1.00 | 11.37 | C |
| ATOM | 1276 | C | ALA | A | 174 | 48.192 | 63.417 | 17.163 | 1.00 | 11.00 | C |
| ATOM | 1277 | O | ALA | A | 174 | 46.966 | 63.341 | 17.051 | 1.00 | 12.83 | O |
| ATOM | 1278 | N | PHE | A | 175 | 48.792 | 64.249 | 18.001 | 1.00 | 10.60 | N |
| ATOM | 1279 | CA | PHE | A | 175 | 48.102 | 65.258 | 18.798 | 1.00 | 11.40 | C |
| ATOM | 1280 | CB | PHE | A | 175 | 47.381 | 64.652 | 20.033 | 1.00 | 12.77 | C |
| ATOM | 1281 | CG | PHE | A | 175 | 48.310 | 64.086 | 21.087 | 1.00 | 12.88 | C |
| ATOM | 1282 | CD1 | PHE | A | 175 | 48.895 | 62.858 | 20.924 | 1.00 | 12.18 | C |
| ATOM | 1283 | CE1 | PHE | A | 175 | 49.759 | 62.344 | 21.884 | 1.00 | 14.01 | C |
| ATOM | 1284 | CZ | PHE | A | 175 | 50.025 | 63.063 | 23.029 | 1.00 | 11.54 | C |
| ATOM | 1285 | CE2 | PHE | A | 175 | 49.427 | 64.280 | 23.216 | 1.00 | 12.37 | C |
| ATOM | 1286 | CD2 | PHE | A | 175 | 48.574 | 64.797 | 22.257 | 1.00 | 12.29 | C |
| ATOM | 1287 | C | PHE | A | 175 | 49.148 | 66.282 | 19.200 | 1.00 | 11.29 | C |
| ATOM | 1288 | O | PHE | A | 175 | 50.334 | 66.092 | 18.933 | 1.00 | 11.82 | O |
| ATOM | 1289 | N | ASP | A | 176 | 48.739 | 67.371 | 19.840 | 1.00 | 10.78 | N |
| ATOM | 1290 | CA | ASP | A | 176 | 49.675 | 68.429 | 20.157 | 1.00 | 11.84 | C |
| ATOM | 1291 | CB | ASP | A | 176 | 48.935 | 69.767 | 20.323 | 1.00 | 13.37 | C |
| ATOM | 1292 | CG | ASP | A | 176 | 49.875 | 70.965 | 20.549 | 1.00 | 13.44 | C |
| ATOM | 1293 | OD1 | ASP | A | 176 | 51.093 | 70.805 | 20.750 | 1.00 | 12.54 | O |
| ATOM | 1294 | OD2 | ASP | A | 176 | 49.363 | 72.102 | 20.571 | 1.00 | 15.40 | O |
| ATOM | 1295 | C | ASP | A | 176 | 50.460 | 68.026 | 21.387 | 1.00 | 12.02 | C |
| ATOM | 1296 | O | ASP | A | 176 | 50.057 | 68.314 | 22.500 | 1.00 | 12.00 | O |
| ATOM | 1297 | N | GLN | A | 177 | 51.591 | 67.349 | 21.188 | 1.00 | 10.47 | N |
| ATOM | 1298 | CA | GLN | A | 177 | 52.286 | 66.701 | 22.328 | 1.00 | 10.73 | C |
| ATOM | 1299 | CB | GLN | A | 177 | 53.374 | 65.735 | 21.845 | 1.00 | 10.09 | C |
| ATOM | 1300 | CG | GLN | A | 177 | 52.861 | 64.538 | 21.071 | 1.00 | 8.92 | C |
| ATOM | 1301 | CD | GLN | A | 177 | 53.962 | 63.574 | 20.714 | 1.00 | 8.65 | C |
| ATOM | 1302 | OE1 | GLN | A | 177 | 54.292 | 63.396 | 19.525 | 1.00 | 8.24 | O |
| ATOM | 1303 | NE2 | GLN | A | 177 | 54.555 | 62.936 | 21.744 | 1.00 | 7.40 | N |
| ATOM | 1304 | C | GLN | A | 177 | 52.918 | 67.748 | 23.254 | 1.00 | 10.04 | C |
| ATOM | 1305 | O | GLN | A | 177 | 53.416 | 68.776 | 22.780 | 1.00 | 8.99 | O |
| ATOM | 1306 | N | PRO | A | 178 | 52.925 | 67.480 | 24.564 | 1.00 | 10.85 | N |
| ATOM | 1307 | CA | PRO | A | 178 | 53.580 | 68.402 | 25.484 | 1.00 | 11.80 | C |
| ATOM | 1308 | CB | PRO | A | 178 | 53.253 | 67.810 | 26.860 | 1.00 | 13.58 | C |
| ATOM | 1309 | CG | PRO | A | 178 | 53.050 | 66.344 | 26.589 | 1.00 | 14.37 | C |
| ATOM | 1310 | CD | PRO | A | 178 | 52.284 | 66.367 | 25.294 | 1.00 | 13.22 | C |
| ATOM | 1311 | C | PRO | A | 178 | 55.100 | 68.471 | 25.223 | 1.00 | 11.27 | C |
| ATOM | 1312 | O | PRO | A | 178 | 55.711 | 67.511 | 24.724 | 1.00 | 8.31 | O |
| ATOM | 1313 | N | LYS | A | 179 | 55.669 | 69.631 | 25.524 | 1.00 | 11.67 | N |
| ATOM | 1314 | CA | LYS | A | 179 | 57.099 | 69.949 | 25.295 | 1.00 | 11.52 | C |
| ATOM | 1315 | CB | LYS | A | 179 | 58.041 | 69.051 | 26.142 | 1.00 | 12.11 | C |
| ATOM | 1316 | CG | LYS | A | 179 | 57.918 | 69.337 | 27.632 | 1.00 | 14.00 | C |
| ATOM | 1317 | CD | LYS | A | 179 | 59.064 | 68.746 | 28.457 | 1.00 | 15.10 | C |
| ATOM | 1318 | CE | LYS | A | 179 | 59.028 | 69.283 | 29.879 | 1.00 | 13.97 | C |
| ATOM | 1319 | NZ | LYS | A | 179 | 60.185 | 68.786 | 30.671 | 1.00 | 11.58 | N |
| ATOM | 1320 | C | LYS | A | 179 | 57.569 | 70.059 | 23.831 | 1.00 | 10.28 | C |
| ATOM | 1321 | O | LYS | A | 179 | 58.251 | 71.039 | 23.490 | 1.00 | 10.46 | O |
| ATOM | 1322 | N | PHE | A | 180 | 57.226 | 69.099 | 22.977 | 1.00 | 8.82 | N |
| ATOM | 1323 | CA | PHE | A | 180 | 57.513 | 69.221 | 21.557 | 1.00 | 8.16 | C |
| ATOM | 1324 | CB | PHE | A | 180 | 56.907 | 68.055 | 20.771 | 1.00 | 8.23 | C |
| ATOM | 1325 | CG | PHE | A | 180 | 57.679 | 66.775 | 20.905 | 1.00 | 7.35 | C |
| ATOM | 1326 | CD1 | PHE | A | 180 | 58.850 | 66.573 | 20.189 | 1.00 | 7.39 | C |
| ATOM | 1327 | CE1 | PHE | A | 180 | 59.571 | 65.398 | 20.303 | 1.00 | 7.17 | C |
| ATOM | 1328 | CZ | PHE | A | 180 | 59.124 | 64.405 | 21.162 | 1.00 | 8.06 | C |
| ATOM | 1329 | CE2 | PHE | A | 180 | 57.961 | 64.597 | 21.890 | 1.00 | 7.86 | C |
| ATOM | 1330 | CD2 | PHE | A | 180 | 57.252 | 65.780 | 21.769 | 1.00 | 7.55 | C |
| ATOM | 1331 | C | PHE | A | 180 | 56.983 | 70.550 | 21.006 | 1.00 | 8.91 | C |
| ATOM | 1332 | O | PHE | A | 180 | 55.851 | 70.962 | 21.298 | 1.00 | 8.93 | O |
| ATOM | 1333 | N | GLY | A | 181 | 57.811 | 71.209 | 20.209 | 1.00 | 7.53 | N |
| ATOM | 1334 | CA | GLY | A | 181 | 57.482 | 72.482 | 19.604 | 1.00 | 8.62 | C |
| ATOM | 1335 | C | GLY | A | 181 | 57.420 | 73.673 | 20.541 | 1.00 | 9.53 | C |
| ATOM | 1336 | O | GLY | A | 181 | 56.997 | 74.763 | 20.130 | 1.00 | 11.40 | O |
| ATOM | 1337 | N | LYS | A | 182 | 57.833 | 73.489 | 21.794 | 1.00 | 9.58 | N |
| ATOM | 1338 | CA | LYS | A | 182 | 57.624 | 74.521 | 22.815 | 1.00 | 9.35 | C |
| ATOM | 1339 | CB | LYS | A | 182 | 56.674 | 74.002 | 23.912 | 1.00 | 10.05 | C |
| ATOM | 1340 | CG | LYS | A | 182 | 55.212 | 73.865 | 23.448 | 1.00 | 10.48 | C |
| ATOM | 1341 | CD | LYS | A | 182 | 54.618 | 72.529 | 23.903 | 1.00 | 12.92 | C |
| ATOM | 1342 | CE | LYS | A | 182 | 53.209 | 72.283 | 23.374 | 1.00 | 12.16 | C |
| ATOM | 1343 | NZ | LYS | A | 182 | 53.207 | 72.193 | 21.891 | 1.00 | 11.08 | N |
| ATOM | 1344 | C | LYS | A | 182 | 58.952 | 74.952 | 23.408 | 1.00 | 8.81 | C |
| ATOM | 1345 | O | LYS | A | 182 | 59.846 | 74.140 | 23.565 | 1.00 | 9.49 | O |
| ATOM | 1346 | N | ILE | A | 183 | 59.077 | 76.241 | 23.709 | 1.00 | 8.75 | N |
| ATOM | 1347 | CA | ILE | A | 183 | 60.255 | 76.777 | 24.346 | 1.00 | 9.45 | C |
| ATOM | 1348 | CB | ILE | A | 183 | 61.083 | 77.623 | 23.357 | 1.00 | 12.55 | C |
| ATOM | 1349 | CG1 | ILE | A | 183 | 61.710 | 76.659 | 22.331 | 1.00 | 15.65 | C |
| ATOM | 1350 | CD1 | ILE | A | 183 | 62.529 | 77.286 | 21.266 | 1.00 | 22.71 | C |
| ATOM | 1351 | CG2 | ILE | A | 183 | 62.171 | 78.408 | 24.090 | 1.00 | 10.48 | C |
| ATOM | 1352 | C | ILE | A | 183 | 59.853 | 77.545 | 25.605 | 1.00 | 9.68 | C |
| ATOM | 1353 | O | ILE | A | 183 | 58.910 | 78.353 | 25.586 | 1.00 | 9.02 | O |
| ATOM | 1354 | N | TYR | A | 184 | 60.577 | 77.257 | 26.692 | 1.00 | 8.94 | N |
| ATOM | 1355 | CA | TYR | A | 184 | 60.332 | 77.841 | 27.983 | 1.00 | 10.44 | C |
| ATOM | 1356 | CB | TYR | A | 184 | 59.983 | 76.733 | 29.003 | 1.00 | 10.93 | C |
| ATOM | 1357 | CG | TYR | A | 184 | 58.704 | 75.963 | 28.685 | 1.00 | 10.76 | C |
| ATOM | 1358 | CD1 | TYR | A | 184 | 58.718 | 74.862 | 27.841 | 1.00 | 11.50 | C |
| ATOM | 1359 | CE1 | TYR | A | 184 | 57.567 | 74.145 | 27.562 | 1.00 | 10.91 | C |
| ATOM | 1360 | CZ | TYR | A | 184 | 56.363 | 74.538 | 28.126 | 1.00 | 12.16 | C |
| ATOM | 1361 | OH | TYR | A | 184 | 55.193 | 73.849 | 27.840 | 1.00 | 15.56 | O |
| ATOM | 1362 | CE2 | TYR | A | 184 | 56.317 | 75.621 | 28.978 | 1.00 | 12.75 | C |
| ATOM | 1363 | CD2 | TYR | A | 184 | 57.486 | 76.325 | 29.256 | 1.00 | 12.17 | C |
| ATOM | 1364 | C | TYR | A | 184 | 61.548 | 78.611 | 28.467 | 1.00 | 10.26 | C |
| ATOM | 1365 | O | TYR | A | 184 | 62.674 | 78.217 | 28.213 | 1.00 | 9.38 | O |
| ATOM | 1366 | N | ASP | A | 185 | 61.318 | 79.683 | 29.229 | 1.00 | 14.31 | N |
| ATOM | 1367 | CA | ASP | A | 185 | 62.446 | 80.446 | 29.803 | 1.00 | 18.13 | C |
| ATOM | 1368 | CB | ASP | A | 185 | 62.165 | 81.960 | 29.817 | 1.00 | 22.31 | C |
| ATOM | 1369 | CG | ASP | A | 185 | 61.126 | 82.381 | 30.826 | 1.00 | 27.88 | C |
| ATOM | 1370 | OD1 | ASP | A | 185 | 60.775 | 81.621 | 31.779 | 1.00 | 27.23 | O |
| ATOM | 1371 | OD2 | ASP | A | 185 | 60.649 | 83.518 | 30.638 | 1.00 | 39.07 | O |
| ATOM | 1372 | C | ASP | A | 185 | 62.897 | 79.907 | 31.149 | 1.00 | 19.78 | C |
| ATOM | 1373 | O | ASP | A | 185 | 62.370 | 78.883 | 31.635 | 1.00 | 15.62 | O |
| ATOM | 1374 | N | LYS | A | 186 | 63.923 | 80.538 | 31.720 | 1.00 | 26.05 | N |
| ATOM | 1375 | CA | LYS | A | 186 | 64.474 | 80.086 | 33.009 | 1.00 | 28.37 | C |
| ATOM | 1376 | CB | LYS | A | 186 | 65.671 | 80.947 | 33.458 | 1.00 | 37.38 | C |
| ATOM | 1377 | CG | LYS | A | 186 | 65.436 | 82.462 | 33.452 | 1.00 | 50.77 | C |
| ATOM | 1378 | CD | LYS | A | 186 | 66.739 | 83.253 | 33.329 | 1.00 | 52.62 | C |
| ATOM | 1379 | CE | LYS | A | 186 | 66.503 | 84.664 | 32.793 | 1.00 | 54.21 | C |
| ATOM | 1380 | NZ | LYS | A | 186 | 67.681 | 85.166 | 32.024 | 1.00 | 56.86 | N |
| ATOM | 1381 | C | LYS | A | 186 | 63.376 | 80.052 | 34.091 | 1.00 | 31.29 | C |
| ATOM | 1382 | O | LYS | A | 186 | 63.306 | 79.088 | 34.838 | 1.00 | 29.02 | O |
| ATOM | 1383 | N | ASP | A | 187 | 62.486 | 81.053 | 34.111 | 1.00 | 29.44 | N |
| ATOM | 1384 | CA | ASP | A | 187 | 61.306 | 81.057 | 35.044 | 1.00 | 32.56 | C |
| ATOM | 1385 | CB | ASP | A | 187 | 60.494 | 82.354 | 34.913 | 1.00 | 33.92 | C |
| ATOM | 1386 | CG | ASP | A | 187 | 61.298 | 83.577 | 35.190 | 1.00 | 40.40 | C |
| ATOM | 1387 | OD1 | ASP | A | 187 | 62.181 | 83.493 | 36.066 | 1.00 | 39.07 | O |
| ATOM | 1388 | OD2 | ASP | A | 187 | 61.048 | 84.617 | 34.527 | 1.00 | 50.00 | O |
| ATOM | 1389 | C | ASP | A | 187 | 60.290 | 79.935 | 34.839 | 1.00 | 29.49 | C |
| ATOM | 1390 | O | ASP | A | 187 | 59.374 | 79.807 | 35.625 | 1.00 | 37.65 | O |
| ATOM | 1391 | N | GLY | A | 188 | 60.404 | 79.164 | 33.763 | 1.00 | 25.14 | N |
| ATOM | 1392 | CA | GLY | A | 188 | 59.374 | 78.211 | 33.389 | 1.00 | 17.75 | C |
| ATOM | 1393 | C | GLY | A | 188 | 58.222 | 78.783 | 32.561 | 1.00 | 16.77 | C |
| ATOM | 1394 | O | GLY | A | 188 | 57.274 | 78.068 | 32.279 | 1.00 | 17.77 | O |
| ATOM | 1395 | N | THR | A | 189 | 58.312 | 80.039 | 32.130 | 1.00 | 16.40 | N |
| ATOM | 1396 | CA | THR | A | 189 | 57.295 | 80.646 | 31.277 | 1.00 | 17.50 | C |
| ATOM | 1397 | CB | THR | A | 189 | 57.446 | 82.185 | 31.246 | 1.00 | 19.57 | C |
| ATOM | 1398 | OG1 | THR | A | 189 | 57.549 | 82.670 | 32.586 | 1.00 | 21.09 | O |
| ATOM | 1399 | CG2 | THR | A | 189 | 56.249 | 82.862 | 30.543 | 1.00 | 18.14 | C |
| ATOM | 1400 | C | THR | A | 189 | 57.374 | 80.134 | 29.841 | 1.00 | 16.32 | C |
| ATOM | 1401 | O | THR | A | 189 | 58.469 | 80.021 | 29.272 | 1.00 | 14.35 | O |
| ATOM | 1402 | N | LEU | A | 190 | 56.211 | 79.833 | 29.262 | 1.00 | 13.94 | N |
| ATOM | 1403 | CA | LEU | A | 190 | 56.107 | 79.440 | 27.863 | 1.00 | 13.04 | C |
| ATOM | 1404 | CB | LEU | A | 190 | 54.701 | 78.935 | 27.550 | 1.00 | 12.37 | C |
| ATOM | 1405 | CG | LEU | A | 190 | 54.466 | 78.433 | 26.121 | 1.00 | 12.62 | C |
| ATOM | 1406 | CD1 | LEU | A | 190 | 55.259 | 77.176 | 25.810 | 1.00 | 10.45 | C |
| ATOM | 1407 | CD2 | LEU | A | 190 | 52.954 | 78.196 | 25.942 | 1.00 | 14.52 | C |
| ATOM | 1408 | C | LEU | A | 190 | 56.359 | 80.656 | 27.018 | 1.00 | 13.62 | C |
| ATOM | 1409 | O | LEU | A | 190 | 55.620 | 81.614 | 27.103 | 1.00 | 11.26 | O |
| ATOM | 1410 | N | ILE | A | 191 | 57.377 | 80.619 | 26.171 | 1.00 | 12.52 | N |
| ATOM | 1411 | CA | ILE | A | 191 | 57.625 | 81.744 | 25.309 | 1.00 | 12.76 | C |
| ATOM | 1412 | CB | ILE | A | 191 | 58.966 | 82.411 | 25.643 | 1.00 | 14.33 | C |
| ATOM | 1413 | CG1 | ILE | A | 191 | 60.124 | 81.455 | 25.382 | 1.00 | 13.90 | C |
| ATOM | 1414 | CD1 | ILE | A | 191 | 61.495 | 82.098 | 25.563 | 1.00 | 13.42 | C |
| ATOM | 1415 | CG2 | ILE | A | 191 | 58.952 | 82.952 | 27.090 | 1.00 | 14.41 | C |
| ATOM | 1416 | C | ILE | A | 191 | 57.519 | 81.415 | 23.804 | 1.00 | 11.52 | C |
| ATOM | 1417 | O | ILE | A | 191 | 57.552 | 82.331 | 22.974 | 1.00 | 13.36 | O |
| ATOM | 1418 | N | ALA | A | 192 | 57.401 | 80.138 | 23.433 | 1.00 | 11.55 | N |
| ATOM | 1419 | CA | ALA | A | 192 | 57.132 | 79.779 | 22.010 | 1.00 | 9.87 | C |
| ATOM | 1420 | CB | ALA | A | 192 | 58.435 | 79.657 | 21.198 | 1.00 | 8.37 | C |
| ATOM | 1421 | C | ALA | A | 192 | 56.356 | 78.483 | 21.973 | 1.00 | 10.48 | C |
| ATOM | 1422 | O | ALA | A | 192 | 56.614 | 77.605 | 22.785 | 1.00 | 11.24 | O |
| ATOM | 1423 | N | ASP | A | 193 | 55.386 | 78.370 | 21.058 | 1.00 | 9.96 | N |
| ATOM | 1424 | CA | ASP | A | 193 | 54.649 | 77.122 | 20.892 | 1.00 | 9.77 | C |
| ATOM | 1425 | CB | ASP | A | 193 | 53.413 | 77.120 | 21.804 | 1.00 | 10.43 | C |
| ATOM | 1426 | CG | ASP | A | 193 | 52.706 | 75.778 | 21.851 | 1.00 | 11.12 | C |
| ATOM | 1427 | OD1 | ASP | A | 193 | 53.020 | 74.840 | 21.061 | 1.00 | 10.11 | O |
| ATOM | 1428 | OD2 | ASP | A | 193 | 51.810 | 75.656 | 22.710 | 1.00 | 10.10 | O |
| ATOM | 1429 | C | ASP | A | 193 | 54.235 | 76.941 | 19.436 | 1.00 | 9.82 | C |
| ATOM | 1430 | O | ASP | A | 193 | 53.372 | 77.659 | 18.946 | 1.00 | 8.98 | O |
| ATOM | 1431 | N | HIS | A | 194 | 54.844 | 75.954 | 18.776 | 1.00 | 9.00 | N |
| ATOM | 1432 | CA | HIS | A | 194 | 54.589 | 75.627 | 17.367 | 1.00 | 9.00 | C |
| ATOM | 1433 | CB | HIS | A | 194 | 55.705 | 74.662 | 16.876 | 1.00 | 7.91 | C |
| ATOM | 1434 | CG | HIS | A | 194 | 56.261 | 74.949 | 15.488 | 1.00 | 8.06 | C |
| ATOM | 1435 | ND1 | HIS | A | 194 | 55.640 | 74.557 | 14.337 | 1.00 | 8.17 | N |
| ATOM | 1436 | CE1 | HIS | A | 194 | 56.421 | 74.870 | 13.288 | 1.00 | 7.83 | C |
| ATOM | 1437 | NE2 | HIS | A | 194 | 57.551 | 75.418 | 13.754 | 1.00 | 7.69 | N |
| ATOM | 1438 | CD2 | HIS | A | 194 | 57.483 | 75.492 | 15.099 | 1.00 | 7.85 | C |
| ATOM | 1439 | C | HIS | A | 194 | 53.219 | 74.975 | 17.173 | 1.00 | 9.75 | C |
| ATOM | 1440 | O | HIS | A | 194 | 52.753 | 74.819 | 16.023 | 1.00 | 8.97 | O |
| ATOM | 1441 | N | GLN | A | 195 | 52.578 | 74.573 | 18.279 | 1.00 | 9.77 | N |
| ATOM | 1442 | CA | GLN | A | 195 | 51.189 | 74.077 | 18.293 | 1.00 | 10.69 | C |
| ATOM | 1443 | CB | GLN | A | 195 | 50.215 | 75.248 | 18.106 | 1.00 | 11.53 | C |
| ATOM | 1444 | CG | GLN | A | 195 | 50.263 | 76.249 | 19.232 | 1.00 | 12.30 | C |
| ATOM | 1445 | CD | GLN | A | 195 | 49.330 | 77.417 | 18.999 | 1.00 | 17.08 | C |
| ATOM | 1446 | OE1 | GLN | A | 195 | 49.424 | 78.121 | 17.999 | 1.00 | 14.58 | O |
| ATOM | 1447 | NE2 | GLN | A | 195 | 48.425 | 77.632 | 19.943 | 1.00 | 19.44 | N |
| ATOM | 1448 | C | GLN | A | 195 | 50.893 | 72.973 | 17.259 | 1.00 | 10.35 | C |
| ATOM | 1449 | O | GLN | A | 195 | 49.801 | 72.906 | 16.699 | 1.00 | 10.47 | O |
| ATOM | 1450 | N | ASN | A | 196 | 51.887 | 72.142 | 16.968 | 1.00 | 9.63 | N |
| ATOM | 1451 | CA | ASN | A | 196 | 51.719 | 71.044 | 16.024 | 1.00 | 10.05 | C |
| ATOM | 1452 | CB | ASN | A | 196 | 50.759 | 69.975 | 16.593 | 1.00 | 9.67 | C |
| ATOM | 1453 | CG | ASN | A | 196 | 50.842 | 68.643 | 15.861 | 1.00 | 9.67 | C |
| ATOM | 1454 | OD1 | ASN | A | 196 | 51.900 | 68.263 | 15.393 | 1.00 | 9.77 | O |
| ATOM | 1455 | ND2 | ASN | A | 196 | 49.725 | 67.892 | 15.827 | 1.00 | 7.83 | N |
| ATOM | 1456 | C | ASN | A | 196 | 51.242 | 71.539 | 14.668 | 1.00 | 9.69 | C |
| ATOM | 1457 | O | ASN | A | 196 | 50.549 | 70.824 | 13.975 | 1.00 | 10.02 | O |
| ATOM | 1458 | N | LEU | A | 197 | 51.639 | 72.760 | 14.299 | 1.00 | 10.69 | N |
| ATOM | 1459 | CA | LEU | A | 197 | 51.375 | 73.325 | 12.978 | 1.00 | 10.53 | C |
| ATOM | 1460 | CB | LEU | A | 197 | 50.912 | 74.779 | 13.079 | 1.00 | 10.60 | C |
| ATOM | 1461 | CG | LEU | A | 197 | 49.576 | 75.044 | 13.780 | 1.00 | 12.87 | C |
| ATOM | 1462 | CD1 | LEU | A | 197 | 49.349 | 76.556 | 14.059 | 1.00 | 11.44 | C |
| ATOM | 1463 | CD2 | LEU | A | 197 | 48.412 | 74.409 | 13.000 | 1.00 | 11.47 | C |
| ATOM | 1464 | C | LEU | A | 197 | 52.642 | 73.290 | 12.134 | 1.00 | 10.25 | C |
| ATOM | 1465 | O | LEU | A | 197 | 53.721 | 73.555 | 12.634 | 1.00 | 10.13 | O |
| ATOM | 1466 | N | PRO | A | 198 | 52.519 | 72.944 | 10.845 | 1.00 | 11.95 | N |
| ATOM | 1467 | CA | PRO | A | 198 | 53.680 | 73.161 | 9.967 | 1.00 | 12.95 | C |
| ATOM | 1468 | CB | PRO | A | 198 | 53.197 | 72.721 | 8.572 | 1.00 | 15.22 | C |
| ATOM | 1469 | CG | PRO | A | 198 | 51.746 | 72.443 | 8.688 | 1.00 | 17.25 | C |
| ATOM | 1470 | CD | PRO | A | 198 | 51.293 | 72.566 | 10.124 | 1.00 | 14.07 | C |
| ATOM | 1471 | C | PRO | A | 198 | 54.091 | 74.651 | 9.970 | 1.00 | 11.84 | C |
| ATOM | 1472 | O | PRO | A | 198 | 53.246 | 75.521 | 10.237 | 1.00 | 8.86 | O |
| ATOM | 1473 | N | PRO | A | 199 | 55.379 | 74.944 | 9.694 | 1.00 | 11.71 | N |
| ATOM | 1474 | CA | PRO | A | 199 | 55.835 | 76.339 | 9.814 | 1.00 | 11.73 | C |
| ATOM | 1475 | CB | PRO | A | 199 | 57.299 | 76.270 | 9.342 | 1.00 | 11.77 | C |
| ATOM | 1476 | CG | PRO | A | 199 | 57.716 | 74.844 | 9.631 | 1.00 | 11.53 | C |
| ATOM | 1477 | CD | PRO | A | 199 | 56.482 | 74.040 | 9.304 | 1.00 | 11.79 | C |
| ATOM | 1478 | C | PRO | A | 199 | 55.025 | 77.334 | 8.969 | 1.00 | 10.05 | C |
| ATOM | 1479 | O | PRO | A | 199 | 54.747 | 78.446 | 9.418 | 1.00 | 11.19 | O |
| ATOM | 1480 | N | GLN | A | 200 | 54.603 | 76.904 | 7.791 | 1.00 | 9.70 | N |
| ATOM | 1481 | CA | GLN | A | 200 | 53.879 | 77.769 | 6.872 | 1.00 | 11.05 | C |
| ATOM | 1482 | CB | GLN | A | 200 | 53.707 | 77.093 | 5.498 | 1.00 | 12.75 | C |
| ATOM | 1483 | CG | GLN | A | 200 | 55.007 | 76.653 | 4.831 | 1.00 | 14.55 | C |
| ATOM | 1484 | CD | GLN | A | 200 | 55.345 | 75.174 | 5.079 | 1.00 | 18.94 | C |
| ATOM | 1485 | OE1 | GLN | A | 200 | 55.220 | 74.657 | 6.199 | 1.00 | 15.92 | O |
| ATOM | 1486 | NE2 | GLN | A | 200 | 55.787 | 74.495 | 4.025 | 1.00 | 17.31 | N |
| ATOM | 1487 | C | GLN | A | 200 | 52.513 | 78.141 | 7.419 | 1.00 | 10.65 | C |
| ATOM | 1488 | O | GLN | A | 200 | 51.916 | 79.067 | 6.934 | 1.00 | 11.06 | O |
| ATOM | 1489 | N | GLN | A | 201 | 52.005 | 77.405 | 8.399 | 1.00 | 9.86 | N |
| ATOM | 1490 | CA | GLN | A | 201 | 50.724 | 77.740 | 9.034 | 1.00 | 11.01 | C |
| ATOM | 1491 | CB | GLN | A | 201 | 49.902 | 76.471 | 9.311 | 1.00 | 11.55 | C |
| ATOM | 1492 | CG | GLN | A | 201 | 49.381 | 75.828 | 8.015 | 1.00 | 14.28 | C |
| ATOM | 1493 | CD | GLN | A | 201 | 48.673 | 74.499 | 8.267 | 1.00 | 16.74 | C |
| ATOM | 1494 | OE1 | GLN | A | 201 | 47.979 | 74.334 | 9.252 | 1.00 | 18.13 | O |
| ATOM | 1495 | NE2 | GLN | A | 201 | 48.831 | 73.573 | 7.358 | 1.00 | 18.73 | N |
| ATOM | 1496 | C | GLN | A | 201 | 50.851 | 78.568 | 10.306 | 1.00 | 11.88 | C |
| ATOM | 1497 | O | GLN | A | 201 | 49.840 | 78.931 | 10.899 | 1.00 | 11.87 | O |
| ATOM | 1498 | N | LEU | A | 202 | 52.069 | 78.886 | 10.738 | 1.00 | 11.95 | N |
| ATOM | 1499 | CA | LEU | A | 202 | 52.211 | 79.762 | 11.907 | 1.00 | 11.03 | C |
| ATOM | 1500 | CB | LEU | A | 202 | 53.628 | 79.742 | 12.446 | 1.00 | 10.09 | C |
| ATOM | 1501 | CG | LEU | A | 202 | 54.211 | 78.382 | 12.856 | 1.00 | 9.78 | C |
| ATOM | 1502 | CD1 | LEU | A | 202 | 55.682 | 78.615 | 13.183 | 1.00 | 8.27 | C |
| ATOM | 1503 | CD2 | LEU | A | 202 | 53.411 | 77.789 | 14.029 | 1.00 | 8.33 | C |
| ATOM | 1504 | C | LEU | A | 202 | 51.829 | 81.200 | 11.527 | 1.00 | 12.76 | C |
| ATOM | 1505 | O | LEU | A | 202 | 51.837 | 81.545 | 10.374 | 1.00 | 11.37 | O |
| ATOM | 1506 | N | ASP | A | 203 | 51.531 | 82.032 | 12.519 | 1.00 | 13.21 | N |
| ATOM | 1507 | CA | ASP | A | 203 | 51.093 | 83.420 | 12.303 | 1.00 | 14.72 | C |
| ATOM | 1508 | CB | ASP | A | 203 | 49.574 | 83.457 | 12.540 | 1.00 | 17.99 | C |
| ATOM | 1509 | CG | ASP | A | 203 | 48.937 | 84.831 | 12.271 | 1.00 | 22.41 | C |
| ATOM | 1510 | OD1 | ASP | A | 203 | 49.585 | 85.761 | 11.748 | 1.00 | 21.46 | O |
| ATOM | 1511 | OD2 | ASP | A | 203 | 47.760 | 84.946 | 12.610 | 1.00 | 24.92 | O |
| ATOM | 1512 | C | ASP | A | 203 | 51.844 | 84.366 | 13.276 | 1.00 | 14.34 | C |
| ATOM | 1513 | O | ASP | A | 203 | 51.250 | 84.909 | 14.183 | 1.00 | 14.43 | O |
| ATOM | 1514 | N | PRO | A | 204 | 53.168 | 84.519 | 13.118 | 1.00 | 13.08 | N |
| ATOM | 1515 | CA | PRO | A | 204 | 53.934 | 85.355 | 14.056 | 1.00 | 15.31 | C |
| ATOM | 1516 | CB | PRO | A | 204 | 55.368 | 85.309 | 13.517 | 1.00 | 16.29 | C |
| ATOM | 1517 | CG | PRO | A | 204 | 55.287 | 84.726 | 12.134 | 1.00 | 14.61 | C |
| ATOM | 1518 | CD | PRO | A | 204 | 53.999 | 83.940 | 12.062 | 1.00 | 14.12 | C |
| ATOM | 1519 | C | PRO | A | 204 | 53.457 | 86.801 | 14.135 | 1.00 | 19.16 | C |
| ATOM | 1520 | O | PRO | A | 204 | 53.633 | 87.432 | 15.161 | 1.00 | 20.94 | O |
| ATOM | 1521 | N | GLU | A | 205 | 52.850 | 87.317 | 13.077 | 1.00 | 19.31 | N |
| ATOM | 1522 | CA | GLU | A | 205 | 52.336 | 88.683 | 13.112 | 1.00 | 23.12 | C |
| ATOM | 1523 | CB | GLU | A | 205 | 51.748 | 89.103 | 11.755 | 1.00 | 26.07 | C |
| ATOM | 1524 | CG | GLU | A | 205 | 51.411 | 90.595 | 11.702 | 1.00 | 37.73 | C |
| ATOM | 1525 | CD | GLU | A | 205 | 50.472 | 90.976 | 10.562 | 1.00 | 48.65 | C |
| ATOM | 1526 | OE1 | GLU | A | 205 | 50.573 | 90.397 | 9.460 | 1.00 | 50.75 | O |
| ATOM | 1527 | OE2 | GLU | A | 205 | 49.629 | 91.872 | 10.768 | 1.00 | 61.08 | O |
| ATOM | 1528 | C | GLU | A | 205 | 51.286 | 88.836 | 14.210 | 1.00 | 21.84 | C |
| ATOM | 1529 | O | GLU | A | 205 | 51.289 | 89.818 | 14.920 | 1.00 | 22.08 | O |
| ATOM | 1530 | N | HIS | A | 206 | 50.412 | 87.857 | 14.390 | 1.00 | 21.55 | N |
| ATOM | 1531 | CA | HIS | A | 206 | 49.335 | 87.998 | 15.374 | 1.00 | 22.21 | C |
| ATOM | 1532 | CB | HIS | A | 206 | 47.983 | 87.660 | 14.747 | 1.00 | 23.84 | C |
| ATOM | 1533 | CG | HIS | A | 206 | 47.680 | 88.471 | 13.501 | 1.00 | 32.48 | C |
| ATOM | 1534 | ND1 | HIS | A | 206 | 47.743 | 87.949 | 12.258 | 1.00 | 33.64 | N |
| ATOM | 1535 | CE1 | HIS | A | 206 | 47.441 | 88.906 | 11.353 | 1.00 | 35.01 | C |
| ATOM | 1536 | NE2 | HIS | A | 206 | 47.192 | 90.050 | 12.022 | 1.00 | 34.14 | N |
| ATOM | 1537 | CD2 | HIS | A | 206 | 47.330 | 89.822 | 13.344 | 1.00 | 33.21 | C |
| ATOM | 1538 | C | HIS | A | 206 | 49.536 | 87.202 | 16.633 | 1.00 | 27.52 | C |
| ATOM | 1539 | O | HIS | A | 206 | 48.958 | 87.554 | 17.653 | 1.00 | 24.65 | O |
| ATOM | 1540 | N | ASN | A | 207 | 50.342 | 86.136 | 16.580 | 1.00 | 20.01 | N |
| ATOM | 1541 | CA | ASN | A | 207 | 50.517 | 85.228 | 17.710 | 1.00 | 17.23 | C |
| ATOM | 1542 | CB | ASN | A | 207 | 50.225 | 83.799 | 17.280 | 1.00 | 17.75 | C |
| ATOM | 1543 | CG | ASN | A | 207 | 50.035 | 82.879 | 18.449 | 1.00 | 21.33 | C |
| ATOM | 1544 | OD1 | ASN | A | 207 | 50.565 | 83.111 | 19.566 | 1.00 | 21.62 | O |
| ATOM | 1545 | ND2 | ASN | A | 207 | 49.243 | 81.842 | 18.230 | 1.00 | 20.26 | N |
| ATOM | 1546 | C | ASN | A | 207 | 51.934 | 85.321 | 18.256 | 1.00 | 16.81 | C |
| ATOM | 1547 | O | ASN | A | 207 | 52.878 | 84.831 | 17.625 | 1.00 | 14.76 | O |
| ATOM | 1548 | N | PRO | A | 208 | 52.092 | 85.955 | 19.426 | 1.00 | 17.46 | N |
| ATOM | 1549 | CA | PRO | A | 208 | 53.398 | 86.096 | 20.062 | 1.00 | 16.37 | C |
| ATOM | 1550 | CB | PRO | A | 208 | 53.059 | 86.721 | 21.430 | 1.00 | 17.56 | C |
| ATOM | 1551 | CG | PRO | A | 208 | 51.778 | 87.451 | 21.201 | 1.00 | 19.70 | C |
| ATOM | 1552 | CD | PRO | A | 208 | 51.018 | 86.569 | 20.249 | 1.00 | 19.67 | C |
| ATOM | 1553 | C | PRO | A | 208 | 54.148 | 84.783 | 20.260 | 1.00 | 14.70 | C |
| ATOM | 1554 | O | PRO | A | 208 | 55.377 | 84.790 | 20.212 | 1.00 | 13.89 | O |
| ATOM | 1555 | N | LEU | A | 209 | 53.420 | 83.682 | 20.488 | 1.00 | 12.12 | N |
| ATOM | 1556 | CA | LEU | A | 209 | 54.036 | 82.365 | 20.685 | 1.00 | 13.53 | C |
| ATOM | 1557 | CB | LEU | A | 209 | 53.064 | 81.391 | 21.376 | 1.00 | 14.74 | C |
| ATOM | 1558 | CG | LEU | A | 209 | 52.626 | 81.776 | 22.798 | 1.00 | 16.44 | C |
| ATOM | 1559 | CD1 | LEU | A | 209 | 51.472 | 80.887 | 23.283 | 1.00 | 15.18 | C |
| ATOM | 1560 | CD2 | LEU | A | 209 | 53.821 | 81.684 | 23.738 | 1.00 | 15.92 | C |
| ATOM | 1561 | C | LEU | A | 209 | 54.607 | 81.731 | 19.399 | 1.00 | 10.98 | C |
| ATOM | 1562 | O | LEU | A | 209 | 55.238 | 80.671 | 19.468 | 1.00 | 9.53 | O |
| ATOM | 1563 | N | HIS | A | 210 | 54.429 | 82.387 | 18.254 | 1.00 | 9.36 | N |
| ATOM | 1564 | CA | HIS | A | 210 | 55.040 | 81.921 | 16.998 | 1.00 | 10.30 | C |
| ATOM | 1565 | CB | HIS | A | 210 | 54.016 | 81.943 | 15.859 | 1.00 | 10.35 | C |
| ATOM | 1566 | CG | HIS | A | 210 | 52.830 | 81.038 | 16.068 | 1.00 | 9.77 | C |
| ATOM | 1567 | ND1 | HIS | A | 210 | 51.738 | 81.062 | 15.241 | 1.00 | 8.73 | N |
| ATOM | 1568 | CE1 | HIS | A | 210 | 50.851 | 80.135 | 15.660 | 1.00 | 9.01 | C |
| ATOM | 1569 | NE2 | HIS | A | 210 | 51.343 | 79.547 | 16.755 | 1.00 | 10.12 | N |
| ATOM | 1570 | CD2 | HIS | A | 210 | 52.563 | 80.072 | 17.037 | 1.00 | 9.06 | C |
| ATOM | 1571 | C | HIS | A | 210 | 56.260 | 82.706 | 16.581 | 1.00 | 9.75 | C |
| ATOM | 1572 | O | HIS | A | 210 | 57.000 | 82.278 | 15.713 | 1.00 | 8.60 | O |
| ATOM | 1573 | N | ARG | A | 211 | 56.525 | 83.834 | 17.223 | 1.00 | 11.42 | N |
| ATOM | 1574 | CA | ARG | A | 211 | 57.590 | 84.752 | 16.792 | 1.00 | 10.78 | C |
| ATOM | 1575 | CB | ARG | A | 211 | 57.474 | 86.090 | 17.544 | 1.00 | 13.67 | C |
| ATOM | 1576 | CG | ARG | A | 211 | 56.265 | 86.842 | 17.093 | 1.00 | 19.18 | C |
| ATOM | 1577 | CD | ARG | A | 211 | 55.967 | 88.144 | 17.845 | 1.00 | 23.33 | C |
| ATOM | 1578 | NE | ARG | A | 211 | 54.614 | 88.564 | 17.454 | 1.00 | 26.69 | N |
| ATOM | 1579 | CZ | ARG | A | 211 | 53.811 | 89.382 | 18.138 | 1.00 | 30.08 | C |
| ATOM | 1580 | NH1 | ARG | A | 211 | 54.185 | 89.935 | 19.293 | 1.00 | 25.05 | N |
| ATOM | 1581 | NH2 | ARG | A | 211 | 52.607 | 89.639 | 17.655 | 1.00 | 29.36 | N |
| ATOM | 1582 | C | ARG | A | 211 | 59.013 | 84.238 | 16.936 | 1.00 | 9.58 | C |
| ATOM | 1583 | O | ARG | A | 211 | 59.919 | 84.750 | 16.294 | 1.00 | 9.05 | O |
| ATOM | 1584 | N | PHE | A | 212 | 59.216 | 83.243 | 17.785 | 1.00 | 8.15 | N |
| ATOM | 1585 | CA | PHE | A | 212 | 60.494 | 82.589 | 17.875 | 1.00 | 8.77 | C |
| ATOM | 1586 | CB | PHE | A | 212 | 60.556 | 81.688 | 19.124 | 1.00 | 9.63 | C |
| ATOM | 1587 | CG | PHE | A | 212 | 61.284 | 82.307 | 20.272 | 1.00 | 11.98 | C |
| ATOM | 1588 | CD1 | PHE | A | 212 | 60.969 | 83.593 | 20.694 | 1.00 | 14.73 | C |
| ATOM | 1589 | CE1 | PHE | A | 212 | 61.628 | 84.176 | 21.772 | 1.00 | 18.02 | C |
| ATOM | 1590 | CZ | PHE | A | 212 | 62.630 | 83.476 | 22.414 | 1.00 | 15.52 | C |
| ATOM | 1591 | CE2 | PHE | A | 212 | 62.960 | 82.206 | 21.997 | 1.00 | 14.74 | C |
| ATOM | 1592 | CD2 | PHE | A | 212 | 62.293 | 81.619 | 20.926 | 1.00 | 15.43 | C |
| ATOM | 1593 | C | PHE | A | 212 | 60.831 | 81.767 | 16.638 | 1.00 | 7.80 | C |
| ATOM | 1594 | O | PHE | A | 212 | 61.956 | 81.358 | 16.470 | 1.00 | 7.92 | O |
| ATOM | 1595 | N | TYR | A | 213 | 59.873 | 81.544 | 15.761 | 1.00 | 7.63 | N |
| ATOM | 1596 | CA | TYR | A | 213 | 60.067 | 80.552 | 14.714 | 1.00 | 7.71 | C |
| ATOM | 1597 | CB | TYR | A | 213 | 58.986 | 79.492 | 14.842 | 1.00 | 7.51 | C |
| ATOM | 1598 | CG | TYR | A | 213 | 58.994 | 78.744 | 16.148 | 1.00 | 7.14 | C |
| ATOM | 1599 | CD1 | TYR | A | 213 | 60.172 | 78.236 | 16.683 | 1.00 | 7.13 | C |
| ATOM | 1600 | CE1 | TYR | A | 213 | 60.172 | 77.513 | 17.868 | 1.00 | 8.13 | C |
| ATOM | 1601 | CZ | TYR | A | 213 | 58.972 | 77.293 | 18.533 | 1.00 | 7.47 | C |
| ATOM | 1602 | OH | TYR | A | 213 | 58.958 | 76.587 | 19.709 | 1.00 | 6.99 | O |
| ATOM | 1603 | CE2 | TYR | A | 213 | 57.789 | 77.796 | 18.018 | 1.00 | 6.83 | C |
| ATOM | 1604 | CD2 | TYR | A | 213 | 57.803 | 78.511 | 16.844 | 1.00 | 7.07 | C |
| ATOM | 1605 | C | TYR | A | 213 | 60.031 | 81.127 | 13.307 | 1.00 | 8.50 | C |
| ATOM | 1606 | O | TYR | A | 213 | 59.257 | 82.038 | 13.008 | 1.00 | 7.98 | O |
| ATOM | 1607 | N | ASN | A | 214 | 60.852 | 80.552 | 12.428 | 1.00 | 7.88 | N |
| ATOM | 1608 | CA | ASN | A | 214 | 60.750 | 80.821 | 10.997 | 1.00 | 7.95 | C |
| ATOM | 1609 | CB | ASN | A | 214 | 62.053 | 80.428 | 10.277 | 1.00 | 7.75 | C |
| ATOM | 1610 | CG | ASN | A | 214 | 63.168 | 81.377 | 10.587 | 1.00 | 7.21 | C |
| ATOM | 1611 | OD1 | ASN | A | 214 | 62.932 | 82.588 | 10.795 | 1.00 | 6.20 | O |
| ATOM | 1612 | ND2 | ASN | A | 214 | 64.390 | 80.856 | 10.613 | 1.00 | 6.54 | N |
| ATOM | 1613 | C | ASN | A | 214 | 59.553 | 80.050 | 10.429 | 1.00 | 8.37 | C |
| ATOM | 1614 | O | ASN | A | 214 | 59.093 | 79.068 | 11.005 | 1.00 | 6.32 | O |
| ATOM | 1615 | N | THR | A | 215 | 59.020 | 80.549 | 9.320 | 1.00 | 8.85 | N |
| ATOM | 1616 | CA | THR | A | 215 | 57.782 | 80.021 | 8.767 | 1.00 | 9.29 | C |
| ATOM | 1617 | CB | THR | A | 215 | 56.681 | 81.100 | 8.798 | 1.00 | 10.96 | C |
| ATOM | 1618 | OG1 | THR | A | 215 | 57.057 | 82.131 | 7.918 | 1.00 | 10.00 | O |
| ATOM | 1619 | CG2 | THR | A | 215 | 56.493 | 81.694 | 10.197 | 1.00 | 10.48 | C |
| ATOM | 1620 | C | THR | A | 215 | 57.960 | 79.522 | 7.335 | 1.00 | 10.13 | C |
| ATOM | 1621 | O | THR | A | 215 | 56.984 | 79.432 | 6.578 | 1.00 | 11.44 | O |
| ATOM | 1622 | N | VAL | A | 216 | 59.176 | 79.146 | 6.963 | 1.00 | 10.50 | N |
| ATOM | 1623 | CA | VAL | A | 216 | 59.468 | 78.725 | 5.569 | 1.00 | 12.11 | C |
| ATOM | 1624 | CB | VAL | A | 216 | 60.973 | 78.912 | 5.202 | 1.00 | 13.76 | C |
| ATOM | 1625 | CG1 | VAL | A | 216 | 61.295 | 78.346 | 3.811 | 1.00 | 12.99 | C |
| ATOM | 1626 | CG2 | VAL | A | 216 | 61.364 | 80.405 | 5.287 | 1.00 | 14.35 | C |
| ATOM | 1627 | C | VAL | A | 216 | 59.032 | 77.279 | 5.310 | 1.00 | 11.49 | C |
| ATOM | 1628 | O | VAL | A | 216 | 58.495 | 76.983 | 4.253 | 1.00 | 13.28 | O |
| ATOM | 1629 | N | GLY | A | 217 | 59.235 | 76.396 | 6.286 | 1.00 | 11.73 | N |
| ATOM | 1630 | CA | GLY | A | 217 | 59.054 | 74.956 | 6.112 | 1.00 | 11.01 | C |
| ATOM | 1631 | C | GLY | A | 217 | 60.278 | 74.341 | 5.441 | 1.00 | 10.70 | C |
| ATOM | 1632 | O | GLY | A | 217 | 61.156 | 75.050 | 5.041 | 1.00 | 10.37 | O |
| ATOM | 1633 | N | PRO | A | 218 | 60.323 | 73.017 | 5.297 | 1.00 | 13.52 | N |
| ATOM | 1634 | CA | PRO | A | 218 | 61.419 | 72.322 | 4.616 | 1.00 | 12.51 | C |
| ATOM | 1635 | CB | PRO | A | 218 | 60.961 | 70.862 | 4.609 | 1.00 | 16.88 | C |
| ATOM | 1636 | CG | PRO | A | 218 | 60.072 | 70.755 | 5.814 | 1.00 | 17.49 | C |
| ATOM | 1637 | CD | PRO | A | 218 | 59.319 | 72.067 | 5.822 | 1.00 | 16.29 | C |
| ATOM | 1638 | C | PRO | A | 218 | 61.554 | 72.828 | 3.207 | 1.00 | 11.94 | C |
| ATOM | 1639 | O | PRO | A | 218 | 60.555 | 73.139 | 2.573 | 1.00 | 10.11 | O |
| ATOM | 1640 | N | VAL | A | 219 | 62.781 | 72.985 | 2.741 | 1.00 | 11.40 | N |
| ATOM | 1641 | CA | VAL | A | 219 | 63.032 | 73.494 | 1.395 | 1.00 | 11.36 | C |
| ATOM | 1642 | CB | VAL | A | 219 | 63.920 | 74.756 | 1.431 | 1.00 | 11.22 | C |
| ATOM | 1643 | CG1 | VAL | A | 219 | 64.409 | 75.122 | 0.040 | 1.00 | 9.95 | C |
| ATOM | 1644 | CG2 | VAL | A | 219 | 63.154 | 75.931 | 2.060 | 1.00 | 8.78 | C |
| ATOM | 1645 | C | VAL | A | 219 | 63.683 | 72.367 | 0.611 | 1.00 | 11.48 | C |
| ATOM | 1646 | O | VAL | A | 219 | 64.702 | 71.824 | 1.020 | 1.00 | 12.35 | O |
| ATOM | 1647 | N | ASP | A | 220 | 63.065 | 72.003 | −0.499 | 1.00 | 12.70 | N |
| ATOM | 1648 | CA | ASP | A | 220 | 63.620 | 71.004 | −1.444 | 1.00 | 14.09 | C |
| ATOM | 1649 | CB | ASP | A | 220 | 62.564 | 70.651 | −2.512 | 1.00 | 19.09 | C |
| ATOM | 1650 | CG | ASP | A | 220 | 63.013 | 69.504 | −3.422 | 1.00 | 23.00 | C |
| ATOM | 1651 | OD1 | ASP | A | 220 | 62.514 | 68.395 | −3.228 | 1.00 | 33.98 | O |
| ATOM | 1652 | OD2 | ASP | A | 220 | 63.882 | 69.697 | −4.301 | 1.00 | 19.58 | O |
| ATOM | 1653 | C | ASP | A | 220 | 64.853 | 71.583 | −2.137 | 1.00 | 12.59 | C |
| ATOM | 1654 | O | ASP | A | 220 | 64.809 | 72.725 | −2.623 | 1.00 | 12.92 | O |
| ATOM | 1655 | N | GLY | A | 221 | 65.940 | 70.810 | −2.179 | 1.00 | 12.37 | N |
| ATOM | 1656 | CA | GLY | A | 221 | 67.205 | 71.221 | −2.785 | 1.00 | 11.61 | C |
| ATOM | 1657 | C | GLY | A | 221 | 67.180 | 71.643 | −4.249 | 1.00 | 14.90 | C |
| ATOM | 1658 | O | GLY | A | 221 | 68.077 | 72.339 | −4.724 | 1.00 | 12.92 | O |
| ATOM | 1659 | N | ALA | A | 222 | 66.144 | 71.221 | −4.970 | 1.00 | 15.04 | N |
| ATOM | 1660 | CA | ALA | A | 222 | 65.919 | 71.649 | −6.343 | 1.00 | 16.06 | C |
| ATOM | 1661 | CB | ALA | A | 222 | 65.042 | 70.606 | −7.053 | 1.00 | 14.37 | C |
| ATOM | 1662 | C | ALA | A | 222 | 65.259 | 73.033 | −6.437 | 1.00 | 15.61 | C |
| ATOM | 1663 | O | ALA | A | 222 | 65.190 | 73.613 | −7.528 | 1.00 | 18.27 | O |
| ATOM | 1664 | N | LYS | A | 223 | 64.720 | 73.535 | −5.326 | 1.00 | 14.15 | N |
| ATOM | 1665 | CA | LYS | A | 223 | 63.855 | 74.738 | −5.347 | 1.00 | 14.84 | C |
| ATOM | 1666 | CB | LYS | A | 223 | 62.384 | 74.339 | −5.086 | 1.00 | 17.77 | C |
| ATOM | 1667 | CG | LYS | A | 223 | 61.779 | 73.313 | −6.073 | 1.00 | 19.77 | C |
| ATOM | 1668 | CD | LYS | A | 223 | 61.871 | 73.726 | −7.547 | 1.00 | 20.13 | C |
| ATOM | 1669 | CE | LYS | A | 223 | 61.187 | 72.711 | −8.493 | 1.00 | 23.78 | C |
| ATOM | 1670 | NZ | LYS | A | 223 | 61.352 | 73.102 | −9.924 | 1.00 | 19.55 | N |
| ATOM | 1671 | C | LYS | A | 223 | 64.285 | 75.799 | −4.299 | 1.00 | 12.23 | C |
| ATOM | 1672 | O | LYS | A | 223 | 63.473 | 76.600 | −3.831 | 1.00 | 14.57 | O |
| ATOM | 1673 | N | GLY | A | 224 | 65.547 | 75.793 | −3.916 | 1.00 | 9.38 | N |
| ATOM | 1674 | CA | GLY | A | 224 | 66.077 | 76.781 | −2.953 | 1.00 | 8.00 | C |
| ATOM | 1675 | C | GLY | A | 224 | 67.222 | 76.129 | −2.212 | 1.00 | 9.36 | C |
| ATOM | 1676 | O | GLY | A | 224 | 67.511 | 74.930 | −2.420 | 1.00 | 8.77 | O |
| ATOM | 1677 | N | SER | A | 225 | 67.886 | 76.883 | −1.348 | 1.00 | 8.68 | N |
| ATOM | 1678 | CA | SER | A | 225 | 68.874 | 76.286 | −0.461 | 1.00 | 7.59 | C |
| ATOM | 1679 | CB | SER | A | 225 | 69.738 | 77.379 | 0.190 | 1.00 | 7.43 | C |
| ATOM | 1680 | OG | SER | A | 225 | 70.619 | 76.789 | 1.142 | 1.00 | 6.53 | O |
| ATOM | 1681 | C | SER | A | 225 | 68.146 | 75.534 | 0.641 | 1.00 | 8.56 | C |
| ATOM | 1682 | O | SER | A | 225 | 67.248 | 76.101 | 1.286 | 1.00 | 8.06 | O |
| ATOM | 1683 | N | ILE | A | 226 | 68.550 | 74.300 | 0.928 | 1.00 | 7.50 | N |
| ATOM | 1684 | CA | ILE | A | 226 | 67.912 | 73.585 | 2.042 | 1.00 | 7.85 | C |
| ATOM | 1685 | CB | ILE | A | 226 | 68.368 | 72.105 | 2.161 | 1.00 | 9.15 | C |
| ATOM | 1686 | CG1 | ILE | A | 226 | 69.816 | 71.994 | 2.658 | 1.00 | 10.46 | C |
| ATOM | 1687 | CD1 | ILE | A | 226 | 70.309 | 70.556 | 2.877 | 1.00 | 9.97 | C |
| ATOM | 1688 | CG2 | ILE | A | 226 | 68.147 | 71.364 | 0.826 | 1.00 | 8.05 | C |
| ATOM | 1689 | C | ILE | A | 226 | 68.100 | 74.330 | 3.366 | 1.00 | 8.37 | C |
| ATOM | 1690 | O | ILE | A | 226 | 67.236 | 74.287 | 4.236 | 1.00 | 7.38 | O |
| ATOM | 1691 | N | PHE | A | 227 | 69.187 | 75.099 | 3.472 | 1.00 | 8.65 | N |
| ATOM | 1692 | CA | PHE | A | 227 | 69.505 | 75.797 | 4.708 | 1.00 | 9.48 | C |
| ATOM | 1693 | CB | PHE | A | 227 | 70.965 | 76.273 | 4.707 | 1.00 | 9.08 | C |
| ATOM | 1694 | CG | PHE | A | 227 | 71.922 | 75.176 | 4.443 | 1.00 | 8.38 | C |
| ATOM | 1695 | CD1 | PHE | A | 227 | 71.943 | 74.077 | 5.252 | 1.00 | 8.71 | C |
| ATOM | 1696 | CE1 | PHE | A | 227 | 72.813 | 73.038 | 5.018 | 1.00 | 9.21 | C |
| ATOM | 1697 | CZ | PHE | A | 227 | 73.664 | 73.090 | 3.938 | 1.00 | 9.94 | C |
| ATOM | 1698 | CE2 | PHE | A | 227 | 73.644 | 74.190 | 3.120 | 1.00 | 10.56 | C |
| ATOM | 1699 | CD2 | PHE | A | 227 | 72.771 | 75.225 | 3.370 | 1.00 | 8.98 | C |
| ATOM | 1700 | C | PHE | A | 227 | 68.595 | 76.940 | 5.044 | 1.00 | 9.68 | C |
| ATOM | 1701 | O | PHE | A | 227 | 68.628 | 77.417 | 6.180 | 1.00 | 9.62 | O |
| ATOM | 1702 | N | ASP | A | 228 | 67.765 | 77.360 | 4.092 | 1.00 | 9.83 | N |
| ATOM | 1703 | CA | ASP | A | 228 | 66.748 | 78.367 | 4.328 | 1.00 | 10.19 | C |
| ATOM | 1704 | CB | ASP | A | 228 | 66.376 | 79.108 | 3.015 | 1.00 | 11.95 | C |
| ATOM | 1705 | CG | ASP | A | 228 | 67.518 | 79.985 | 2.481 | 1.00 | 15.69 | C |
| ATOM | 1706 | OD1 | ASP | A | 228 | 68.495 | 80.196 | 3.224 | 1.00 | 15.19 | O |
| ATOM | 1707 | OD2 | ASP | A | 228 | 67.453 | 80.449 | 1.318 | 1.00 | 17.15 | O |
| ATOM | 1708 | C | ASP | A | 228 | 65.489 | 77.796 | 4.948 | 1.00 | 9.82 | C |
| ATOM | 1709 | O | ASP | A | 228 | 64.611 | 78.559 | 5.382 | 1.00 | 9.11 | O |
| ATOM | 1710 | N | GLY | A | 229 | 65.395 | 76.468 | 5.033 | 1.00 | 8.99 | N |
| ATOM | 1711 | CA | GLY | A | 229 | 64.217 | 75.831 | 5.589 | 1.00 | 8.16 | C |
| ATOM | 1712 | C | GLY | A | 229 | 64.504 | 75.012 | 6.812 | 1.00 | 8.52 | C |
| ATOM | 1713 | O | GLY | A | 229 | 65.634 | 74.940 | 7.273 | 1.00 | 8.41 | O |
| ATOM | 1714 | N | ASN | A | 230 | 63.456 | 74.399 | 7.351 | 1.00 | 8.65 | N |
| ATOM | 1715 | CA | ASN | A | 230 | 63.588 | 73.541 | 8.519 | 1.00 | 8.57 | C |
| ATOM | 1716 | CB | ASN | A | 230 | 62.218 | 73.058 | 9.001 | 1.00 | 9.59 | C |
| ATOM | 1717 | CG | ASN | A | 230 | 61.289 | 74.185 | 9.351 | 1.00 | 7.92 | C |
| ATOM | 1718 | OD1 | ASN | A | 230 | 60.844 | 74.916 | 8.444 | 1.00 | 7.22 | O |
| ATOM | 1719 | ND2 | ASN | A | 230 | 60.981 | 74.360 | 10.674 | 1.00 | 7.00 | N |
| ATOM | 1720 | C | ASN | A | 230 | 64.449 | 72.320 | 8.230 | 1.00 | 9.27 | C |
| ATOM | 1721 | O | ASN | A | 230 | 64.451 | 71.807 | 7.112 | 1.00 | 8.03 | O |
| ATOM | 1722 | N | LEU | A | 231 | 65.184 | 71.872 | 9.241 | 1.00 | 9.54 | N |
| ATOM | 1723 | CA | LEU | A | 231 | 65.806 | 70.562 | 9.229 | 1.00 | 9.90 | C |
| ATOM | 1724 | CB | LEU | A | 231 | 67.043 | 70.567 | 10.122 | 1.00 | 9.26 | C |
| ATOM | 1725 | CG | LEU | A | 231 | 67.814 | 69.251 | 10.327 | 1.00 | 10.74 | C |
| ATOM | 1726 | CD1 | LEU | A | 231 | 68.277 | 68.661 | 8.991 | 1.00 | 9.80 | C |
| ATOM | 1727 | CD2 | LEU | A | 231 | 68.981 | 69.494 | 11.291 | 1.00 | 10.19 | C |
| ATOM | 1728 | C | LEU | A | 231 | 64.804 | 69.515 | 9.734 | 1.00 | 10.16 | C |
| ATOM | 1729 | O | LEU | A | 231 | 64.239 | 69.699 | 10.801 | 1.00 | 9.71 | O |
| ATOM | 1730 | N | ALA | A | 232 | 64.596 | 68.435 | 8.971 | 1.00 | 10.65 | N |
| ATOM | 1731 | CA | ALA | A | 232 | 63.612 | 67.394 | 9.295 | 1.00 | 11.25 | C |
| ATOM | 1732 | CB | ALA | A | 232 | 64.144 | 66.460 | 10.397 | 1.00 | 11.26 | C |
| ATOM | 1733 | C | ALA | A | 232 | 62.311 | 68.067 | 9.720 | 1.00 | 10.60 | C |
| ATOM | 1734 | O | ALA | A | 232 | 61.822 | 68.893 | 8.990 | 1.00 | 11.68 | O |
| ATOM | 1735 | N | GLN | A | 233 | 61.748 | 67.733 | 10.886 | 1.00 | 10.40 | N |
| ATOM | 1736 | CA | GLN | A | 233 | 60.548 | 68.426 | 11.361 | 1.00 | 11.70 | C |
| ATOM | 1737 | CB | GLN | A | 233 | 59.371 | 67.471 | 11.600 | 1.00 | 13.09 | C |
| ATOM | 1738 | CG | GLN | A | 233 | 59.068 | 66.550 | 10.399 | 1.00 | 13.83 | C |
| ATOM | 1739 | CD | GLN | A | 233 | 60.063 | 65.419 | 10.209 | 1.00 | 17.23 | C |
| ATOM | 1740 | OE1 | GLN | A | 233 | 60.507 | 64.750 | 11.188 | 1.00 | 18.61 | O |
| ATOM | 1741 | NE2 | GLN | A | 233 | 60.446 | 65.188 | 8.943 | 1.00 | 14.32 | N |
| ATOM | 1742 | C | GLN | A | 233 | 60.800 | 69.278 | 12.595 | 1.00 | 10.17 | C |
| ATOM | 1743 | O | GLN | A | 233 | 59.869 | 69.575 | 13.362 | 1.00 | 10.82 | O |
| ATOM | 1744 | N | LEU | A | 234 | 62.031 | 69.735 | 12.728 | 1.00 | 8.49 | N |
| ATOM | 1745 | CA | LEU | A | 234 | 62.459 | 70.529 | 13.869 | 1.00 | 8.01 | C |
| ATOM | 1746 | CB | LEU | A | 234 | 63.982 | 70.517 | 13.986 | 1.00 | 7.26 | C |
| ATOM | 1747 | CG | LEU | A | 234 | 64.645 | 69.146 | 14.226 | 1.00 | 7.55 | C |
| ATOM | 1748 | CD1 | LEU | A | 234 | 66.147 | 69.240 | 13.920 | 1.00 | 6.15 | C |
| ATOM | 1749 | CD2 | LEU | A | 234 | 64.347 | 68.643 | 15.646 | 1.00 | 6.17 | C |
| ATOM | 1750 | C | LEU | A | 234 | 62.008 | 71.949 | 13.707 | 1.00 | 8.63 | C |
| ATOM | 1751 | O | LEU | A | 234 | 62.103 | 72.508 | 12.615 | 1.00 | 10.74 | O |
| ATOM | 1752 | N | SER | A | 235 | 61.529 | 72.547 | 14.794 | 1.00 | 7.87 | N |
| ATOM | 1753 | CA | SER | A | 235 | 61.231 | 73.982 | 14.819 | 1.00 | 9.14 | C |
| ATOM | 1754 | CB | SER | A | 235 | 60.806 | 74.421 | 16.222 | 1.00 | 9.04 | C |
| ATOM | 1755 | OG | SER | A | 235 | 61.847 | 74.248 | 17.190 | 1.00 | 7.50 | O |
| ATOM | 1756 | C | SER | A | 235 | 62.479 | 74.751 | 14.361 | 1.00 | 10.95 | C |
| ATOM | 1757 | O | SER | A | 235 | 63.584 | 74.394 | 14.739 | 1.00 | 13.76 | O |
| ATOM | 1758 | N | ASP | A | 236 | 62.309 | 75.765 | 13.518 | 1.00 | 9.21 | N |
| ATOM | 1759 | CA | ASP | A | 236 | 63.437 | 76.550 | 13.094 | 1.00 | 8.91 | C |
| ATOM | 1760 | CB | ASP | A | 236 | 63.436 | 76.728 | 11.578 | 1.00 | 10.02 | C |
| ATOM | 1761 | CG | ASP | A | 236 | 64.838 | 76.978 | 11.019 | 1.00 | 10.03 | C |
| ATOM | 1762 | OD1 | ASP | A | 236 | 65.716 | 76.086 | 11.130 | 1.00 | 9.94 | O |
| ATOM | 1763 | OD2 | ASP | A | 236 | 65.060 | 78.058 | 10.476 | 1.00 | 10.02 | O |
| ATOM | 1764 | C | ASP | A | 236 | 63.463 | 77.916 | 13.794 | 1.00 | 9.38 | C |
| ATOM | 1765 | O | ASP | A | 236 | 62.599 | 78.763 | 13.561 | 1.00 | 9.80 | O |
| ATOM | 1766 | N | LEU | A | 237 | 64.460 | 78.134 | 14.644 | 1.00 | 7.95 | N |
| ATOM | 1767 | CA | LEU | A | 237 | 64.514 | 79.368 | 15.403 | 1.00 | 8.32 | C |
| ATOM | 1768 | CB | LEU | A | 237 | 65.517 | 79.257 | 16.557 | 1.00 | 8.30 | C |
| ATOM | 1769 | CG | LEU | A | 237 | 65.179 | 78.200 | 17.623 | 1.00 | 9.05 | C |
| ATOM | 1770 | CD1 | LEU | A | 237 | 66.160 | 78.296 | 18.787 | 1.00 | 7.94 | C |
| ATOM | 1771 | CD2 | LEU | A | 237 | 63.744 | 78.343 | 18.139 | 1.00 | 9.53 | C |
| ATOM | 1772 | C | LEU | A | 237 | 64.831 | 80.558 | 14.480 | 1.00 | 8.89 | C |
| ATOM | 1773 | O | LEU | A | 237 | 65.658 | 80.467 | 13.556 | 1.00 | 8.00 | O |
| ATOM | 1774 | N | ASN | A | 238 | 64.130 | 81.663 | 14.724 | 1.00 | 9.05 | N |
| ATOM | 1775 | CA | ASN | A | 238 | 64.217 | 82.851 | 13.856 | 1.00 | 8.98 | C |
| ATOM | 1776 | CB | ASN | A | 238 | 62.946 | 83.700 | 13.976 | 1.00 | 9.29 | C |
| ATOM | 1777 | CG | ASN | A | 238 | 63.008 | 84.967 | 13.146 | 1.00 | 9.11 | C |
| ATOM | 1778 | OD1 | ASN | A | 238 | 63.990 | 85.223 | 12.433 | 1.00 | 9.95 | O |
| ATOM | 1779 | ND2 | ASN | A | 238 | 61.937 | 85.731 | 13.178 | 1.00 | 8.67 | N |
| ATOM | 1780 | C | ASN | A | 238 | 65.439 | 83.672 | 14.224 | 1.00 | 8.67 | C |
| ATOM | 1781 | O | ASN | A | 238 | 65.413 | 84.447 | 15.183 | 1.00 | 8.71 | O |
| ATOM | 1782 | N | GLU | A | 239 | 66.515 | 83.455 | 13.470 | 1.00 | 9.46 | N |
| ATOM | 1783 | CA | GLU | A | 239 | 67.788 | 84.146 | 13.678 | 1.00 | 9.95 | C |
| ATOM | 1784 | CB | GLU | A | 239 | 68.805 | 83.725 | 12.609 | 1.00 | 9.75 | C |
| ATOM | 1785 | CG | GLU | A | 239 | 68.472 | 84.174 | 11.178 | 1.00 | 10.21 | C |
| ATOM | 1786 | CD | GLU | A | 239 | 67.484 | 83.271 | 10.427 | 1.00 | 12.25 | C |
| ATOM | 1787 | OE1 | GLU | A | 239 | 66.965 | 82.242 | 10.962 | 1.00 | 11.92 | O |
| ATOM | 1788 | OE2 | GLU | A | 239 | 67.177 | 83.629 | 9.270 | 1.00 | 12.19 | O |
| ATOM | 1789 | C | GLU | A | 239 | 67.685 | 85.670 | 13.643 | 1.00 | 9.26 | C |
| ATOM | 1790 | O | GLU | A | 239 | 68.587 | 86.346 | 14.119 | 1.00 | 9.01 | O |
| ATOM | 1791 | N | ARG | A | 240 | 66.628 | 86.202 | 13.041 | 1.00 | 9.48 | N |
| ATOM | 1792 | CA | ARG | A | 240 | 66.453 | 87.658 | 12.934 | 1.00 | 10.29 | C |
| ATOM | 1793 | CB | ARG | A | 240 | 65.680 | 88.024 | 11.642 | 1.00 | 9.90 | C |
| ATOM | 1794 | CG | ARG | A | 240 | 66.375 | 87.568 | 10.355 | 1.00 | 10.51 | C |
| ATOM | 1795 | CD | ARG | A | 240 | 65.548 | 87.841 | 9.095 | 1.00 | 10.04 | C |
| ATOM | 1796 | NE | ARG | A | 240 | 64.229 | 87.222 | 9.204 | 1.00 | 11.65 | N |
| ATOM | 1797 | CZ | ARG | A | 240 | 63.049 | 87.839 | 9.156 | 1.00 | 14.40 | C |
| ATOM | 1798 | NH1 | ARG | A | 240 | 62.939 | 89.143 | 8.960 | 1.00 | 17.97 | N |
| ATOM | 1799 | NH2 | ARG | A | 240 | 61.949 | 87.131 | 9.287 | 1.00 | 13.77 | N |
| ATOM | 1800 | C | ARG | A | 240 | 65.730 | 88.262 | 14.120 | 1.00 | 10.21 | C |
| ATOM | 1801 | O | ARG | A | 240 | 65.625 | 89.477 | 14.220 | 1.00 | 10.26 | O |
| ATOM | 1802 | N | ASN | A | 241 | 65.225 | 87.430 | 15.018 | 1.00 | 9.85 | N |
| ATOM | 1803 | CA | ASN | A | 241 | 64.440 | 87.930 | 16.138 | 1.00 | 9.04 | C |
| ATOM | 1804 | CB | ASN | A | 241 | 63.365 | 86.886 | 16.527 | 1.00 | 8.60 | C |
| ATOM | 1805 | CG | ASN | A | 241 | 62.496 | 87.320 | 17.703 | 1.00 | 11.12 | C |
| ATOM | 1806 | OD1 | ASN | A | 241 | 62.865 | 88.212 | 18.479 | 1.00 | 8.46 | O |
| ATOM | 1807 | ND2 | ASN | A | 241 | 61.340 | 86.657 | 17.857 | 1.00 | 8.55 | N |
| ATOM | 1808 | C | ASN | A | 241 | 65.416 | 88.167 | 17.262 | 1.00 | 7.66 | C |
| ATOM | 1809 | O | ASN | A | 241 | 66.020 | 87.223 | 17.760 | 1.00 | 7.50 | O |
| ATOM | 1810 | N | PRO | A | 242 | 65.550 | 89.412 | 17.716 | 1.00 | 7.70 | N |
| ATOM | 1811 | CA | PRO | A | 242 | 66.588 | 89.627 | 18.728 | 1.00 | 8.44 | C |
| ATOM | 1812 | CB | PRO | A | 242 | 66.637 | 91.155 | 18.869 | 1.00 | 8.57 | C |
| ATOM | 1813 | CG | PRO | A | 242 | 65.261 | 91.603 | 18.501 | 1.00 | 9.52 | C |
| ATOM | 1814 | CD | PRO | A | 242 | 64.789 | 90.646 | 17.421 | 1.00 | 8.80 | C |
| ATOM | 1815 | C | PRO | A | 242 | 66.298 | 88.945 | 20.062 | 1.00 | 8.57 | C |
| ATOM | 1816 | O | PRO | A | 242 | 67.230 | 88.685 | 20.834 | 1.00 | 9.95 | O |
| ATOM | 1817 | N | ASP | A | 243 | 65.041 | 88.622 | 20.330 | 1.00 | 9.65 | N |
| ATOM | 1818 | CA | ASP | A | 243 | 64.699 | 87.818 | 21.513 | 1.00 | 10.05 | C |
| ATOM | 1819 | CB | ASP | A | 243 | 63.207 | 87.645 | 21.654 | 1.00 | 10.85 | C |
| ATOM | 1820 | CG | ASP | A | 243 | 62.491 | 88.910 | 22.053 | 1.00 | 12.95 | C |
| ATOM | 1821 | OD1 | ASP | A | 243 | 63.098 | 89.879 | 22.552 | 1.00 | 14.17 | O |
| ATOM | 1822 | OD2 | ASP | A | 243 | 61.265 | 88.884 | 21.906 | 1.00 | 14.33 | O |
| ATOM | 1823 | C | ASP | A | 243 | 65.317 | 86.408 | 21.443 | 1.00 | 10.25 | C |
| ATOM | 1824 | O | ASP | A | 243 | 65.688 | 85.843 | 22.485 | 1.00 | 8.57 | O |
| ATOM | 1825 | N | VAL | A | 244 | 65.394 | 85.841 | 20.232 | 1.00 | 9.35 | N |
| ATOM | 1826 | CA | VAL | A | 244 | 66.012 | 84.510 | 20.027 | 1.00 | 8.85 | C |
| ATOM | 1827 | CB | VAL | A | 244 | 65.807 | 83.992 | 18.584 | 1.00 | 8.65 | C |
| ATOM | 1828 | CG1 | VAL | A | 244 | 66.668 | 82.777 | 18.279 | 1.00 | 6.72 | C |
| ATOM | 1829 | CG2 | VAL | A | 244 | 64.332 | 83.715 | 18.338 | 1.00 | 7.73 | C |
| ATOM | 1830 | C | VAL | A | 244 | 67.491 | 84.556 | 20.372 | 1.00 | 9.96 | C |
| ATOM | 1831 | O | VAL | A | 244 | 68.000 | 83.725 | 21.140 | 1.00 | 8.97 | O |
| ATOM | 1832 | N | LEU | A | 245 | 68.194 | 85.554 | 19.852 | 1.00 | 8.99 | N |
| ATOM | 1833 | CA | LEU | A | 245 | 69.607 | 85.664 | 20.140 | 1.00 | 8.92 | C |
| ATOM | 1834 | CB | LEU | A | 245 | 70.253 | 86.795 | 19.296 | 1.00 | 9.87 | C |
| ATOM | 1835 | CG | LEU | A | 245 | 71.749 | 87.033 | 19.518 | 1.00 | 11.93 | C |
| ATOM | 1836 | CD1 | LEU | A | 245 | 72.556 | 85.781 | 19.201 | 1.00 | 10.23 | C |
| ATOM | 1837 | CD2 | LEU | A | 245 | 72.230 | 88.246 | 18.685 | 1.00 | 12.15 | C |
| ATOM | 1838 | C | LEU | A | 245 | 69.806 | 85.887 | 21.642 | 1.00 | 8.71 | C |
| ATOM | 1839 | O | LEU | A | 245 | 70.660 | 85.240 | 22.252 | 1.00 | 7.81 | O |
| ATOM | 1840 | N | ASP | A | 246 | 68.991 | 86.767 | 22.236 | 1.00 | 8.57 | N |
| ATOM | 1841 | CA | ASP | A | 246 | 69.076 | 87.047 | 23.676 | 1.00 | 8.50 | C |
| ATOM | 1842 | CB | ASP | A | 246 | 68.036 | 88.071 | 24.127 | 1.00 | 8.41 | C |
| ATOM | 1843 | CG | ASP | A | 246 | 68.344 | 89.478 | 23.683 | 1.00 | 9.39 | C |
| ATOM | 1844 | OD1 | ASP | A | 246 | 69.427 | 89.766 | 23.094 | 1.00 | 11.58 | O |
| ATOM | 1845 | OD2 | ASP | A | 246 | 67.467 | 90.314 | 23.934 | 1.00 | 9.12 | O |
| ATOM | 1846 | C | ASP | A | 246 | 68.848 | 85.768 | 24.467 | 1.00 | 8.86 | C |
| ATOM | 1847 | O | ASP | A | 246 | 69.564 | 85.504 | 25.434 | 1.00 | 8.72 | O |
| ATOM | 1848 | N | TYR | A | 247 | 67.840 | 84.988 | 24.071 | 1.00 | 7.85 | N |
| ATOM | 1849 | CA | TYR | A | 247 | 67.528 | 83.743 | 24.778 | 1.00 | 8.07 | C |
| ATOM | 1850 | CB | TYR | A | 247 | 66.283 | 83.109 | 24.176 | 1.00 | 8.64 | C |
| ATOM | 1851 | CG | TYR | A | 247 | 65.903 | 81.792 | 24.786 | 1.00 | 8.20 | C |
| ATOM | 1852 | CD1 | TYR | A | 247 | 66.344 | 80.609 | 24.241 | 1.00 | 8.56 | C |
| ATOM | 1853 | CE1 | TYR | A | 247 | 65.985 | 79.393 | 24.796 | 1.00 | 8.28 | C |
| ATOM | 1854 | CZ | TYR | A | 247 | 65.167 | 79.365 | 25.895 | 1.00 | 7.93 | C |
| ATOM | 1855 | OH | TYR | A | 247 | 64.830 | 78.149 | 26.438 | 1.00 | 8.17 | O |
| ATOM | 1856 | CE2 | TYR | A | 247 | 64.706 | 80.523 | 26.448 | 1.00 | 8.24 | C |
| ATOM | 1857 | CD2 | TYR | A | 247 | 65.051 | 81.735 | 25.888 | 1.00 | 8.63 | C |
| ATOM | 1858 | C | TYR | A | 247 | 68.729 | 82.752 | 24.748 | 1.00 | 8.02 | C |
| ATOM | 1859 | O | TYR | A | 247 | 69.188 | 82.258 | 25.802 | 1.00 | 8.17 | O |
| ATOM | 1860 | N | LEU | A | 248 | 69.267 | 82.524 | 23.567 | 1.00 | 7.77 | N |
| ATOM | 1861 | CA | LEU | A | 248 | 70.357 | 81.561 | 23.396 | 1.00 | 7.94 | C |
| ATOM | 1862 | CB | LEU | A | 248 | 70.538 | 81.235 | 21.924 | 1.00 | 8.51 | C |
| ATOM | 1863 | CG | LEU | A | 248 | 69.348 | 80.538 | 21.227 | 1.00 | 8.35 | C |
| ATOM | 1864 | CD1 | LEU | A | 248 | 69.495 | 80.612 | 19.705 | 1.00 | 7.39 | C |
| ATOM | 1865 | CD2 | LEU | A | 248 | 69.262 | 79.111 | 21.735 | 1.00 | 7.29 | C |
| ATOM | 1866 | C | LEU | A | 248 | 71.681 | 82.054 | 24.016 | 1.00 | 7.97 | C |
| ATOM | 1867 | O | LEU | A | 248 | 72.395 | 81.284 | 24.641 | 1.00 | 8.18 | O |
| ATOM | 1868 | N | VAL | A | 249 | 72.005 | 83.326 | 23.848 | 1.00 | 7.89 | N |
| ATOM | 1869 | CA | VAL | A | 249 | 73.208 | 83.868 | 24.460 | 1.00 | 8.40 | C |
| ATOM | 1870 | CB | VAL | A | 249 | 73.460 | 85.339 | 24.076 | 1.00 | 8.93 | C |
| ATOM | 1871 | CG1 | VAL | A | 249 | 74.480 | 85.991 | 25.022 | 1.00 | 8.60 | C |
| ATOM | 1872 | CG2 | VAL | A | 249 | 73.907 | 85.462 | 22.627 | 1.00 | 8.39 | C |
| ATOM | 1873 | C | VAL | A | 249 | 73.125 | 83.699 | 25.978 | 1.00 | 8.41 | C |
| ATOM | 1874 | O | VAL | A | 249 | 74.070 | 83.252 | 26.595 | 1.00 | 9.01 | O |
| ATOM | 1875 | N | GLY | A | 250 | 71.983 | 84.008 | 26.573 | 1.00 | 8.12 | N |
| ATOM | 1876 | CA | GLY | A | 250 | 71.854 | 83.924 | 28.026 | 1.00 | 8.21 | C |
| ATOM | 1877 | C | GLY | A | 250 | 71.981 | 82.487 | 28.514 | 1.00 | 7.99 | C |
| ATOM | 1878 | O | GLY | A | 250 | 72.579 | 82.221 | 29.545 | 1.00 | 7.72 | O |
| ATOM | 1879 | N | ALA | A | 251 | 71.432 | 81.550 | 27.759 | 1.00 | 8.09 | N |
| ATOM | 1880 | CA | ALA | A | 251 | 71.494 | 80.131 | 28.121 | 1.00 | 8.04 | C |
| ATOM | 1881 | CB | ALA | A | 251 | 70.635 | 79.295 | 27.169 | 1.00 | 7.87 | C |
| ATOM | 1882 | C | ALA | A | 251 | 72.939 | 79.662 | 28.100 | 1.00 | 8.94 | C |
| ATOM | 1883 | O | ALA | A | 251 | 73.433 | 79.079 | 29.069 | 1.00 | 8.37 | O |
| ATOM | 1884 | N | TYR | A | 252 | 73.644 | 79.941 | 27.002 | 1.00 | 9.53 | N |
| ATOM | 1885 | CA | TYR | A | 252 | 75.003 | 79.460 | 26.874 | 1.00 | 9.09 | C |
| ATOM | 1886 | CB | TYR | A | 252 | 75.396 | 79.343 | 25.395 | 1.00 | 9.00 | C |
| ATOM | 1887 | CG | TYR | A | 252 | 74.508 | 78.284 | 24.726 | 1.00 | 9.23 | C |
| ATOM | 1888 | CD1 | TYR | A | 252 | 74.482 | 76.976 | 25.209 | 1.00 | 9.03 | C |
| ATOM | 1889 | CE1 | TYR | A | 252 | 73.653 | 76.026 | 24.660 | 1.00 | 8.33 | C |
| ATOM | 1890 | CZ | TYR | A | 252 | 72.830 | 76.371 | 23.604 | 1.00 | 8.72 | C |
| ATOM | 1891 | OH | TYR | A | 252 | 71.999 | 75.425 | 23.038 | 1.00 | 7.85 | O |
| ATOM | 1892 | CE2 | TYR | A | 252 | 72.853 | 77.657 | 23.103 | 1.00 | 8.90 | C |
| ATOM | 1893 | CD2 | TYR | A | 252 | 73.674 | 78.601 | 23.672 | 1.00 | 8.75 | C |
| ATOM | 1894 | C | TYR | A | 252 | 76.019 | 80.177 | 27.777 | 1.00 | 9.99 | C |
| ATOM | 1895 | O | TYR | A | 252 | 76.988 | 79.541 | 28.247 | 1.00 | 10.36 | O |
| ATOM | 1896 | N | LEU | A | 253 | 75.769 | 81.449 | 28.084 | 1.00 | 9.12 | N |
| ATOM | 1897 | CA | LEU | A | 253 | 76.558 | 82.146 | 29.107 | 1.00 | 9.45 | C |
| ATOM | 1898 | CB | LEU | A | 253 | 76.254 | 83.656 | 29.161 | 1.00 | 8.84 | C |
| ATOM | 1899 | CG | LEU | A | 253 | 76.794 | 84.547 | 28.022 | 1.00 | 9.73 | C |
| ATOM | 1900 | CD1 | LEU | A | 253 | 76.288 | 85.995 | 28.233 | 1.00 | 8.83 | C |
| ATOM | 1901 | CD2 | LEU | A | 253 | 78.328 | 84.535 | 27.930 | 1.00 | 8.02 | C |
| ATOM | 1902 | C | LEU | A | 253 | 76.332 | 81.501 | 30.482 | 1.00 | 8.81 | C |
| ATOM | 1903 | O | LEU | A | 253 | 77.262 | 81.386 | 31.274 | 1.00 | 9.64 | O |
| ATOM | 1904 | N | GLN | A | 254 | 75.116 | 81.077 | 30.772 | 1.00 | 8.17 | N |
| ATOM | 1905 | CA | GLN | A | 254 | 74.864 | 80.349 | 32.026 | 1.00 | 8.63 | C |
| ATOM | 1906 | CB | GLN | A | 254 | 73.393 | 79.954 | 32.166 | 1.00 | 9.69 | C |
| ATOM | 1907 | CG | GLN | A | 254 | 73.048 | 79.230 | 33.460 | 1.00 | 9.28 | C |
| ATOM | 1908 | CD | GLN | A | 254 | 71.595 | 78.842 | 33.503 | 1.00 | 9.80 | C |
| ATOM | 1909 | OE1 | GLN | A | 254 | 70.769 | 79.682 | 33.718 | 1.00 | 10.05 | O |
| ATOM | 1910 | NE2 | GLN | A | 254 | 71.279 | 77.561 | 33.263 | 1.00 | 9.63 | N |
| ATOM | 1911 | C | GLN | A | 254 | 75.723 | 79.086 | 32.108 | 1.00 | 7.72 | C |
| ATOM | 1912 | O | GLN | A | 254 | 76.364 | 78.817 | 33.127 | 1.00 | 7.05 | O |
| ATOM | 1913 | N | TRP | A | 255 | 75.742 | 78.314 | 31.039 | 1.00 | 7.22 | N |
| ATOM | 1914 | CA | TRP | A | 255 | 76.489 | 77.064 | 31.059 | 1.00 | 7.92 | C |
| ATOM | 1915 | CB | TRP | A | 255 | 76.096 | 76.135 | 29.909 | 1.00 | 7.81 | C |
| ATOM | 1916 | CG | TRP | A | 255 | 74.630 | 75.821 | 29.890 | 1.00 | 8.61 | C |
| ATOM | 1917 | CD1 | TRP | A | 255 | 73.755 | 76.010 | 28.839 | 1.00 | 8.34 | C |
| ATOM | 1918 | NE1 | TRP | A | 255 | 72.494 | 75.637 | 29.180 | 1.00 | 9.61 | N |
| ATOM | 1919 | CE2 | TRP | A | 255 | 72.442 | 75.205 | 30.449 | 1.00 | 9.34 | C |
| ATOM | 1920 | CD2 | TRP | A | 255 | 73.789 | 75.314 | 30.993 | 1.00 | 9.16 | C |
| ATOM | 1921 | CE3 | TRP | A | 255 | 74.013 | 74.938 | 32.308 | 1.00 | 9.42 | C |
| ATOM | 1922 | CZ3 | TRP | A | 255 | 72.922 | 74.497 | 33.071 | 1.00 | 9.28 | C |
| ATOM | 1923 | CH2 | TRP | A | 255 | 71.641 | 74.410 | 32.522 | 1.00 | 10.00 | C |
| ATOM | 1924 | CZ2 | TRP | A | 255 | 71.378 | 74.781 | 31.205 | 1.00 | 9.11 | C |
| ATOM | 1925 | C | TRP | A | 255 | 77.974 | 77.271 | 31.119 | 1.00 | 7.83 | C |
| ATOM | 1926 | O | TRP | A | 255 | 78.687 | 76.457 | 31.736 | 1.00 | 7.86 | O |
| ATOM | 1927 | N | ILE | A | 256 | 78.474 | 78.343 | 30.506 | 1.00 | 8.00 | N |
| ATOM | 1928 | CA | ILE | A | 256 | 79.860 | 78.725 | 30.758 | 1.00 | 8.64 | C |
| ATOM | 1929 | CB | ILE | A | 256 | 80.337 | 79.860 | 29.848 | 1.00 | 9.07 | C |
| ATOM | 1930 | CG1 | ILE | A | 256 | 80.362 | 79.386 | 28.396 | 1.00 | 9.62 | C |
| ATOM | 1931 | CD1 | ILE | A | 256 | 80.391 | 80.512 | 27.374 | 1.00 | 9.62 | C |
| ATOM | 1932 | CG2 | ILE | A | 256 | 81.729 | 80.327 | 30.274 | 1.00 | 9.03 | C |
| ATOM | 1933 | C | ILE | A | 256 | 80.092 | 79.115 | 32.224 | 1.00 | 9.08 | C |
| ATOM | 1934 | O | ILE | A | 256 | 81.085 | 78.699 | 32.820 | 1.00 | 9.19 | O |
| ATOM | 1935 | N | ASP | A | 257 | 79.209 | 79.914 | 32.813 | 1.00 | 11.89 | N |
| ATOM | 1936 | CA | ASP | A | 257 | 79.341 | 80.243 | 34.230 | 1.00 | 12.12 | C |
| ATOM | 1937 | CB | ASP | A | 257 | 78.161 | 81.043 | 34.791 | 1.00 | 16.27 | C |
| ATOM | 1938 | CG | ASP | A | 257 | 78.065 | 82.429 | 34.226 | 1.00 | 25.30 | C |
| ATOM | 1939 | OD1 | ASP | A | 257 | 79.053 | 82.920 | 33.631 | 1.00 | 31.80 | O |
| ATOM | 1940 | OD2 | ASP | A | 257 | 76.973 | 83.024 | 34.362 | 1.00 | 34.61 | O |
| ATOM | 1941 | C | ASP | A | 257 | 79.434 | 78.975 | 35.054 | 1.00 | 11.56 | C |
| ATOM | 1942 | O | ASP | A | 257 | 80.121 | 78.973 | 36.041 | 1.00 | 11.65 | O |
| ATOM | 1943 | N | GLN | A | 258 | 78.715 | 77.928 | 34.666 | 1.00 | 10.15 | N |
| ATOM | 1944 | CA | GLN | A | 258 | 78.734 | 76.662 | 35.397 | 1.00 | 9.64 | C |
| ATOM | 1945 | CB | GLN | A | 258 | 77.416 | 75.915 | 35.191 | 1.00 | 9.24 | C |
| ATOM | 1946 | CG | GLN | A | 258 | 76.260 | 76.704 | 35.767 | 1.00 | 9.14 | C |
| ATOM | 1947 | CD | GLN | A | 258 | 74.975 | 75.917 | 35.872 | 1.00 | 11.25 | C |
| ATOM | 1948 | OE1 | GLN | A | 258 | 74.984 | 74.690 | 35.785 | 1.00 | 11.49 | O |
| ATOM | 1949 | NE2 | GLN | A | 258 | 73.852 | 76.631 | 36.071 | 1.00 | 9.01 | N |
| ATOM | 1950 | C | GLN | A | 258 | 79.911 | 75.757 | 35.124 | 1.00 | 10.42 | C |
| ATOM | 1951 | O | GLN | A | 258 | 80.042 | 74.736 | 35.802 | 1.00 | 10.37 | O |
| ATOM | 1952 | N | GLY | A | 259 | 80.780 | 76.111 | 34.173 | 1.00 | 9.71 | N |
| ATOM | 1953 | CA | GLY | A | 259 | 82.029 | 75.373 | 33.972 | 1.00 | 9.16 | C |
| ATOM | 1954 | C | GLY | A | 259 | 82.350 | 74.824 | 32.593 | 1.00 | 9.89 | C |
| ATOM | 1955 | O | GLY | A | 259 | 83.379 | 74.180 | 32.436 | 1.00 | 9.39 | O |
| ATOM | 1956 | N | ALA | A | 260 | 81.526 | 75.110 | 31.580 | 1.00 | 9.37 | N |
| ATOM | 1957 | CA | ALA | A | 260 | 81.819 | 74.691 | 30.215 | 1.00 | 9.37 | C |
| ATOM | 1958 | CB | ALA | A | 260 | 80.634 | 74.971 | 29.287 | 1.00 | 8.87 | C |
| ATOM | 1959 | C | ALA | A | 260 | 83.061 | 75.431 | 29.711 | 1.00 | 10.05 | C |
| ATOM | 1960 | O | ALA | A | 260 | 83.136 | 76.656 | 29.839 | 1.00 | 10.90 | O |
| ATOM | 1961 | N | ASP | A | 261 | 84.012 | 74.685 | 29.146 | 1.00 | 8.84 | N |
| ATOM | 1962 | CA | ASP | A | 261 | 85.298 | 75.218 | 28.699 | 1.00 | 10.61 | C |
| ATOM | 1963 | CB | ASP | A | 261 | 86.396 | 74.238 | 29.069 | 1.00 | 11.23 | C |
| ATOM | 1964 | CG | ASP | A | 261 | 86.511 | 74.070 | 30.560 | 1.00 | 13.42 | C |
| ATOM | 1965 | OD1 | ASP | A | 261 | 86.750 | 75.108 | 31.212 | 1.00 | 12.25 | O |
| ATOM | 1966 | OD2 | ASP | A | 261 | 86.296 | 72.942 | 31.091 | 1.00 | 12.74 | O |
| ATOM | 1967 | C | ASP | A | 261 | 85.393 | 75.562 | 27.209 | 1.00 | 11.02 | C |
| ATOM | 1968 | O | ASP | A | 261 | 86.235 | 76.329 | 26.811 | 1.00 | 12.22 | O |
| ATOM | 1969 | N | ALA | A | 262 | 84.512 | 74.987 | 26.404 | 1.00 | 9.84 | N |
| ATOM | 1970 | CA | ALA | A | 262 | 84.473 | 75.175 | 24.968 | 1.00 | 8.24 | C |
| ATOM | 1971 | CB | ALA | A | 262 | 85.608 | 74.384 | 24.317 | 1.00 | 6.61 | C |
| ATOM | 1972 | C | ALA | A | 262 | 83.116 | 74.648 | 24.493 | 1.00 | 8.23 | C |
| ATOM | 1973 | O | ALA | A | 262 | 82.416 | 73.922 | 25.240 | 1.00 | 8.59 | O |
| ATOM | 1974 | N | PHE | A | 263 | 82.717 | 75.035 | 23.284 | 1.00 | 7.94 | N |
| ATOM | 1975 | CA | PHE | A | 263 | 81.500 | 74.519 | 22.654 | 1.00 | 7.83 | C |
| ATOM | 1976 | CB | PHE | A | 263 | 80.557 | 75.651 | 22.247 | 1.00 | 8.04 | C |
| ATOM | 1977 | CG | PHE | A | 263 | 80.028 | 76.476 | 23.375 | 1.00 | 7.66 | C |
| ATOM | 1978 | CD1 | PHE | A | 263 | 79.780 | 75.934 | 24.625 | 1.00 | 7.82 | C |
| ATOM | 1979 | CE1 | PHE | A | 263 | 79.236 | 76.709 | 25.641 | 1.00 | 7.86 | C |
| ATOM | 1980 | CZ | PHE | A | 263 | 78.902 | 78.033 | 25.404 | 1.00 | 8.25 | C |
| ATOM | 1981 | CE2 | PHE | A | 263 | 79.119 | 78.585 | 24.158 | 1.00 | 7.88 | C |
| ATOM | 1982 | CD2 | PHE | A | 263 | 79.694 | 77.802 | 23.147 | 1.00 | 8.19 | C |
| ATOM | 1983 | C | PHE | A | 263 | 81.793 | 73.767 | 21.364 | 1.00 | 8.11 | C |
| ATOM | 1984 | O | PHE | A | 263 | 82.657 | 74.157 | 20.539 | 1.00 | 8.29 | O |
| ATOM | 1985 | N | ARG | A | 264 | 81.044 | 72.704 | 21.186 | 1.00 | 8.17 | N |
| ATOM | 1986 | CA | ARG | A | 264 | 80.885 | 72.100 | 19.879 | 1.00 | 8.28 | C |
| ATOM | 1987 | CB | ARG | A | 264 | 80.966 | 70.574 | 19.960 | 1.00 | 8.04 | C |
| ATOM | 1988 | CG | ARG | A | 264 | 80.730 | 69.848 | 18.629 | 1.00 | 9.74 | C |
| ATOM | 1989 | CD | ARG | A | 264 | 79.253 | 69.554 | 18.350 | 1.00 | 10.19 | C |
| ATOM | 1990 | NE | ARG | A | 264 | 79.056 | 68.614 | 17.241 | 1.00 | 10.60 | N |
| ATOM | 1991 | CZ | ARG | A | 264 | 77.870 | 68.172 | 16.818 | 1.00 | 9.18 | C |
| ATOM | 1992 | NH1 | ARG | A | 264 | 76.739 | 68.575 | 17.374 | 1.00 | 8.78 | N |
| ATOM | 1993 | NH2 | ARG | A | 264 | 77.815 | 67.334 | 15.810 | 1.00 | 11.27 | N |
| ATOM | 1994 | C | ARG | A | 264 | 79.532 | 72.589 | 19.401 | 1.00 | 8.33 | C |
| ATOM | 1995 | O | ARG | A | 264 | 78.503 | 72.354 | 20.088 | 1.00 | 7.37 | O |
| ATOM | 1996 | N | ILE | A | 265 | 79.517 | 73.294 | 18.254 | 1.00 | 7.99 | N |
| ATOM | 1997 | CA | ILE | A | 265 | 78.281 | 73.894 | 17.724 | 1.00 | 8.22 | C |
| ATOM | 1998 | CB | ILE | A | 265 | 78.541 | 75.265 | 17.060 | 1.00 | 9.13 | C |
| ATOM | 1999 | CG1 | ILE | A | 265 | 79.226 | 76.245 | 18.029 | 1.00 | 10.88 | C |
| ATOM | 2000 | CD1 | ILE | A | 265 | 79.666 | 77.548 | 17.385 | 1.00 | 11.69 | C |
| ATOM | 2001 | CG2 | ILE | A | 265 | 77.239 | 75.892 | 16.556 | 1.00 | 8.65 | C |
| ATOM | 2002 | C | ILE | A | 265 | 77.580 | 72.987 | 16.711 | 1.00 | 9.45 | C |
| ATOM | 2003 | O | ILE | A | 265 | 78.026 | 72.828 | 15.570 | 1.00 | 9.58 | O |
| ATOM | 2004 | N | ASP | A | 266 | 76.476 | 72.405 | 17.132 | 1.00 | 8.42 | N |
| ATOM | 2005 | CA | ASP | A | 266 | 75.616 | 71.606 | 16.267 | 1.00 | 8.74 | C |
| ATOM | 2006 | CB | ASP | A | 266 | 74.428 | 71.066 | 17.084 | 1.00 | 9.76 | C |
| ATOM | 2007 | CG | ASP | A | 266 | 73.624 | 70.019 | 16.338 | 1.00 | 10.48 | C |
| ATOM | 2008 | OD1 | ASP | A | 266 | 74.174 | 68.910 | 16.102 | 1.00 | 10.90 | O |
| ATOM | 2009 | OD2 | ASP | A | 266 | 72.445 | 70.298 | 15.975 | 1.00 | 11.82 | O |
| ATOM | 2010 | C | ASP | A | 266 | 75.063 | 72.447 | 15.116 | 1.00 | 8.79 | C |
| ATOM | 2011 | O | ASP | A | 266 | 74.866 | 73.657 | 15.262 | 1.00 | 7.60 | O |
| ATOM | 2012 | N | THR | A | 267 | 74.835 | 71.767 | 13.991 | 1.00 | 8.41 | N |
| ATOM | 2013 | CA | THR | A | 267 | 74.082 | 72.254 | 12.846 | 1.00 | 8.27 | C |
| ATOM | 2014 | CB | THR | A | 267 | 72.568 | 72.065 | 13.060 | 1.00 | 7.93 | C |
| ATOM | 2015 | OG1 | THR | A | 267 | 72.233 | 72.475 | 14.401 | 1.00 | 7.29 | O |
| ATOM | 2016 | CG2 | THR | A | 267 | 72.156 | 70.559 | 12.815 | 1.00 | 8.83 | C |
| ATOM | 2017 | C | THR | A | 267 | 74.423 | 73.683 | 12.453 | 1.00 | 8.67 | C |
| ATOM | 2018 | O | THR | A | 267 | 73.542 | 74.476 | 12.203 | 1.00 | 9.33 | O |
| ATOM | 2019 | N | ILE | A | 268 | 75.715 | 73.975 | 12.314 | 1.00 | 9.47 | N |
| ATOM | 2020 | CA | ILE | A | 268 | 76.171 | 75.349 | 12.125 | 1.00 | 8.75 | C |
| ATOM | 2021 | CB | ILE | A | 268 | 77.710 | 75.496 | 12.374 | 1.00 | 8.74 | C |
| ATOM | 2022 | CG1 | ILE | A | 268 | 78.075 | 76.957 | 12.691 | 1.00 | 7.85 | C |
| ATOM | 2023 | CD1 | ILE | A | 268 | 79.502 | 77.162 | 13.177 | 1.00 | 8.43 | C |
| ATOM | 2024 | CG2 | ILE | A | 268 | 78.516 | 74.876 | 11.222 | 1.00 | 7.16 | C |
| ATOM | 2025 | C | ILE | A | 268 | 75.731 | 75.905 | 10.780 | 1.00 | 8.85 | C |
| ATOM | 2026 | O | ILE | A | 268 | 75.455 | 77.084 | 10.677 | 1.00 | 8.54 | O |
| ATOM | 2027 | N | ALA | A | 269 | 75.582 | 75.049 | 9.771 | 1.00 | 8.14 | N |
| ATOM | 2028 | CA | ALA | A | 269 | 75.172 | 75.505 | 8.431 | 1.00 | 8.02 | C |
| ATOM | 2029 | CB | ALA | A | 269 | 75.413 | 74.392 | 7.413 | 1.00 | 8.12 | C |
| ATOM | 2030 | C | ALA | A | 269 | 73.715 | 75.917 | 8.380 | 1.00 | 8.11 | C |
| ATOM | 2031 | O | ALA | A | 269 | 73.276 | 76.556 | 7.406 | 1.00 | 8.13 | O |
| ATOM | 2032 | N | TRP | A | 270 | 72.952 | 75.540 | 9.405 | 1.00 | 6.32 | N |
| ATOM | 2033 | CA | TRP | A | 270 | 71.513 | 75.759 | 9.377 | 1.00 | 7.80 | C |
| ATOM | 2034 | CB | TRP | A | 270 | 70.799 | 74.603 | 10.099 | 1.00 | 7.06 | C |
| ATOM | 2035 | CG | TRP | A | 270 | 70.941 | 73.347 | 9.295 | 1.00 | 7.18 | C |
| ATOM | 2036 | CD1 | TRP | A | 270 | 72.021 | 72.457 | 9.247 | 1.00 | 7.64 | C |
| ATOM | 2037 | NE1 | TRP | A | 270 | 71.786 | 71.467 | 8.298 | 1.00 | 7.96 | N |
| ATOM | 2038 | CE2 | TRP | A | 270 | 70.579 | 71.658 | 7.709 | 1.00 | 7.74 | C |
| ATOM | 2039 | CD2 | TRP | A | 270 | 69.995 | 72.862 | 8.290 | 1.00 | 7.30 | C |
| ATOM | 2040 | CE3 | TRP | A | 270 | 68.757 | 73.284 | 7.853 | 1.00 | 8.03 | C |
| ATOM | 2041 | CZ3 | TRP | A | 270 | 68.102 | 72.541 | 6.875 | 1.00 | 8.01 | C |
| ATOM | 2042 | CH2 | TRP | A | 270 | 68.673 | 71.402 | 6.327 | 1.00 | 8.56 | C |
| ATOM | 2043 | CZ2 | TRP | A | 270 | 69.924 | 70.932 | 6.739 | 1.00 | 8.33 | C |
| ATOM | 2044 | C | TRP | A | 270 | 71.055 | 77.132 | 9.874 | 1.00 | 8.35 | C |
| ATOM | 2045 | O | TRP | A | 270 | 69.861 | 77.425 | 9.819 | 1.00 | 7.81 | O |
| ATOM | 2046 | N | MET | A | 271 | 71.993 | 77.966 | 10.346 | 1.00 | 8.38 | N |
| ATOM | 2047 | CA | MET | A | 271 | 71.730 | 79.389 | 10.564 | 1.00 | 10.00 | C |
| ATOM | 2048 | CB | MET | A | 271 | 71.844 | 79.731 | 12.052 | 1.00 | 10.84 | C |
| ATOM | 2049 | CG | MET | A | 271 | 70.960 | 78.861 | 12.947 | 1.00 | 12.10 | C |
| ATOM | 2050 | SD | MET | A | 271 | 71.071 | 79.290 | 14.704 | 1.00 | 13.63 | S |
| ATOM | 2051 | CE | MET | A | 271 | 69.680 | 80.438 | 14.835 | 1.00 | 11.73 | C |
| ATOM | 2052 | C | MET | A | 271 | 72.749 | 80.197 | 9.767 | 1.00 | 10.23 | C |
| ATOM | 2053 | O | MET | A | 271 | 73.867 | 79.736 | 9.544 | 1.00 | 8.46 | O |
| ATOM | 2054 | N | PRO | A | 272 | 72.406 | 81.435 | 9.386 | 1.00 | 12.06 | N |
| ATOM | 2055 | CA | PRO | A | 272 | 73.403 | 82.176 | 8.610 | 1.00 | 10.33 | C |
| ATOM | 2056 | CB | PRO | A | 272 | 72.674 | 83.481 | 8.232 | 1.00 | 12.08 | C |
| ATOM | 2057 | CG | PRO | A | 272 | 71.208 | 83.140 | 8.369 | 1.00 | 13.71 | C |
| ATOM | 2058 | CD | PRO | A | 272 | 71.146 | 82.177 | 9.527 | 1.00 | 12.40 | C |
| ATOM | 2059 | C | PRO | A | 272 | 74.668 | 82.496 | 9.405 | 1.00 | 10.38 | C |
| ATOM | 2060 | O | PRO | A | 272 | 74.607 | 82.645 | 10.637 | 1.00 | 9.16 | O |
| ATOM | 2061 | N | ASP | A | 273 | 75.783 | 82.639 | 8.690 | 1.00 | 9.25 | N |
| ATOM | 2062 | CA | ASP | A | 273 | 77.058 | 83.069 | 9.275 | 1.00 | 11.03 | C |
| ATOM | 2063 | CB | ASP | A | 273 | 78.048 | 83.421 | 8.181 | 1.00 | 12.34 | C |
| ATOM | 2064 | CG | ASP | A | 273 | 78.514 | 82.236 | 7.394 | 1.00 | 14.80 | C |
| ATOM | 2065 | OD1 | ASP | A | 273 | 78.096 | 81.089 | 7.645 | 1.00 | 17.36 | O |
| ATOM | 2066 | OD2 | ASP | A | 273 | 79.332 | 82.476 | 6.485 | 1.00 | 17.41 | O |
| ATOM | 2067 | C | ASP | A | 273 | 76.906 | 84.340 | 10.141 | 1.00 | 9.98 | C |
| ATOM | 2068 | O | ASP | A | 273 | 77.574 | 84.486 | 11.158 | 1.00 | 9.36 | O |
| ATOM | 2069 | N | SER | A | 274 | 76.055 | 85.261 | 9.720 | 1.00 | 9.01 | N |
| ATOM | 2070 | CA | SER | A | 274 | 75.896 | 86.527 | 10.445 | 1.00 | 10.29 | C |
| ATOM | 2071 | CB | SER | A | 274 | 75.022 | 87.489 | 9.650 | 1.00 | 12.00 | C |
| ATOM | 2072 | OG | SER | A | 274 | 73.760 | 86.886 | 9.349 | 1.00 | 12.83 | O |
| ATOM | 2073 | C | SER | A | 274 | 75.279 | 86.285 | 11.823 | 1.00 | 11.00 | C |
| ATOM | 2074 | O | SER | A | 274 | 75.561 | 87.019 | 12.766 | 1.00 | 8.84 | O |
| ATOM | 2075 | N | PHE | A | 275 | 74.426 | 85.263 | 11.944 | 1.00 | 9.61 | N |
| ATOM | 2076 | CA | PHE | A | 275 | 73.928 | 84.896 | 13.266 | 1.00 | 8.95 | C |
| ATOM | 2077 | CB | PHE | A | 275 | 72.783 | 83.868 | 13.211 | 1.00 | 9.22 | C |
| ATOM | 2078 | CG | PHE | A | 275 | 72.324 | 83.441 | 14.578 | 1.00 | 9.15 | C |
| ATOM | 2079 | CD1 | PHE | A | 275 | 72.929 | 82.362 | 15.217 | 1.00 | 11.17 | C |
| ATOM | 2080 | CE1 | PHE | A | 275 | 72.545 | 81.984 | 16.486 | 1.00 | 11.39 | C |
| ATOM | 2081 | CZ | PHE | A | 275 | 71.523 | 82.657 | 17.116 | 1.00 | 11.35 | C |
| ATOM | 2082 | CE2 | PHE | A | 275 | 70.904 | 83.723 | 16.483 | 1.00 | 11.89 | C |
| ATOM | 2083 | CD2 | PHE | A | 275 | 71.324 | 84.123 | 15.232 | 1.00 | 9.81 | C |
| ATOM | 2084 | C | PHE | A | 275 | 75.044 | 84.370 | 14.154 | 1.00 | 8.45 | C |
| ATOM | 2085 | O | PHE | A | 275 | 75.152 | 84.767 | 15.315 | 1.00 | 9.57 | O |
| ATOM | 2086 | N | TRP | A | 276 | 75.857 | 83.447 | 13.647 | 1.00 | 7.89 | N |
| ATOM | 2087 | CA | TRP | A | 276 | 76.933 | 82.921 | 14.457 | 1.00 | 8.10 | C |
| ATOM | 2088 | CB | TRP | A | 276 | 77.629 | 81.750 | 13.759 | 1.00 | 7.97 | C |
| ATOM | 2089 | CG | TRP | A | 276 | 76.691 | 80.593 | 13.606 | 1.00 | 8.55 | C |
| ATOM | 2090 | CD1 | TRP | A | 276 | 76.202 | 80.043 | 12.440 | 1.00 | 8.47 | C |
| ATOM | 2091 | NE1 | TRP | A | 276 | 75.342 | 79.028 | 12.715 | 1.00 | 9.25 | N |
| ATOM | 2092 | CE2 | TRP | A | 276 | 75.213 | 78.859 | 14.054 | 1.00 | 9.45 | C |
| ATOM | 2093 | CD2 | TRP | A | 276 | 76.039 | 79.856 | 14.686 | 1.00 | 9.04 | C |
| ATOM | 2094 | CE3 | TRP | A | 276 | 76.107 | 79.895 | 16.077 | 1.00 | 10.69 | C |
| ATOM | 2095 | CZ3 | TRP | A | 276 | 75.332 | 78.990 | 16.809 | 1.00 | 8.96 | C |
| ATOM | 2096 | CH2 | TRP | A | 276 | 74.555 | 78.028 | 16.167 | 1.00 | 8.94 | C |
| ATOM | 2097 | CZ2 | TRP | A | 276 | 74.483 | 77.938 | 14.789 | 1.00 | 8.47 | C |
| ATOM | 2098 | C | TRP | A | 276 | 77.892 | 84.005 | 14.854 | 1.00 | 8.26 | C |
| ATOM | 2099 | O | TRP | A | 276 | 78.393 | 84.003 | 15.962 | 1.00 | 9.13 | O |
| ATOM | 2100 | N | GLN | A | 277 | 78.180 | 84.941 | 13.960 | 1.00 | 8.58 | N |
| ATOM | 2101 | CA | GLN | A | 277 | 79.079 | 86.062 | 14.308 | 1.00 | 9.13 | C |
| ATOM | 2102 | CB | GLN | A | 277 | 79.352 | 86.934 | 13.071 | 1.00 | 9.92 | C |
| ATOM | 2103 | CG | GLN | A | 277 | 80.263 | 88.139 | 13.335 | 1.00 | 8.79 | C |
| ATOM | 2104 | CD | GLN | A | 277 | 81.686 | 87.732 | 13.527 | 1.00 | 8.57 | C |
| ATOM | 2105 | OE1 | GLN | A | 277 | 82.090 | 86.651 | 13.139 | 1.00 | 9.25 | O |
| ATOM | 2106 | NE2 | GLN | A | 277 | 82.463 | 88.590 | 14.171 | 1.00 | 10.36 | N |
| ATOM | 2107 | C | GLN | A | 277 | 78.522 | 86.943 | 15.434 | 1.00 | 9.78 | C |
| ATOM | 2108 | O | GLN | A | 277 | 79.256 | 87.310 | 16.355 | 1.00 | 10.07 | O |
| ATOM | 2109 | N | ALA | A | 278 | 77.239 | 87.285 | 15.370 | 1.00 | 10.30 | N |
| ATOM | 2110 | CA | ALA | A | 278 | 76.564 | 87.992 | 16.499 | 1.00 | 11.00 | C |
| ATOM | 2111 | CB | ALA | A | 278 | 75.102 | 88.337 | 16.144 | 1.00 | 9.15 | C |
| ATOM | 2112 | C | ALA | A | 278 | 76.631 | 87.147 | 17.804 | 1.00 | 10.57 | C |
| ATOM | 2113 | O | ALA | A | 278 | 76.927 | 87.675 | 18.892 | 1.00 | 11.24 | O |
| ATOM | 2114 | N | PHE | A | 279 | 76.417 | 85.846 | 17.692 | 1.00 | 10.59 | N |
| ATOM | 2115 | CA | PHE | A | 279 | 76.454 | 84.946 | 18.867 | 1.00 | 9.81 | C |
| ATOM | 2116 | CB | PHE | A | 279 | 75.912 | 83.557 | 18.507 | 1.00 | 9.84 | C |
| ATOM | 2117 | CG | PHE | A | 279 | 76.025 | 82.534 | 19.623 | 1.00 | 8.63 | C |
| ATOM | 2118 | CD1 | PHE | A | 279 | 75.045 | 82.437 | 20.606 | 1.00 | 9.28 | C |
| ATOM | 2119 | CE1 | PHE | A | 279 | 75.157 | 81.481 | 21.635 | 1.00 | 10.65 | C |
| ATOM | 2120 | CZ | PHE | A | 279 | 76.227 | 80.602 | 21.654 | 1.00 | 9.20 | C |
| ATOM | 2121 | CE2 | PHE | A | 279 | 77.194 | 80.681 | 20.661 | 1.00 | 8.93 | C |
| ATOM | 2122 | CD2 | PHE | A | 279 | 77.083 | 81.632 | 19.651 | 1.00 | 8.26 | C |
| ATOM | 2123 | C | PHE | A | 279 | 77.860 | 84.824 | 19.483 | 1.00 | 10.19 | C |
| ATOM | 2124 | O | PHE | A | 279 | 78.035 | 85.053 | 20.694 | 1.00 | 10.52 | O |
| ATOM | 2125 | N | THR | A | 280 | 78.861 | 84.476 | 18.675 | 1.00 | 9.66 | N |
| ATOM | 2126 | CA | THR | A | 280 | 80.201 | 84.331 | 19.221 | 1.00 | 8.43 | C |
| ATOM | 2127 | CB | THR | A | 280 | 81.186 | 83.689 | 18.227 | 1.00 | 8.87 | C |
| ATOM | 2128 | OG1 | THR | A | 280 | 81.401 | 84.562 | 17.105 | 1.00 | 6.74 | O |
| ATOM | 2129 | CG2 | THR | A | 280 | 80.643 | 82.319 | 17.770 | 1.00 | 8.64 | C |
| ATOM | 2130 | C | THR | A | 280 | 80.770 | 85.648 | 19.742 | 1.00 | 9.37 | C |
| ATOM | 2131 | O | THR | A | 280 | 81.550 | 85.636 | 20.686 | 1.00 | 8.49 | O |
| ATOM | 2132 | N | THR | A | 281 | 80.413 | 86.774 | 19.137 | 1.00 | 9.37 | N |
| ATOM | 2133 | CA | THR | A | 281 | 80.868 | 88.082 | 19.646 | 1.00 | 9.96 | C |
| ATOM | 2134 | CB | THR | A | 281 | 80.388 | 89.234 | 18.732 | 1.00 | 10.41 | C |
| ATOM | 2135 | OG1 | THR | A | 281 | 81.007 | 89.085 | 17.461 | 1.00 | 10.47 | O |
| ATOM | 2136 | CG2 | THR | A | 281 | 80.728 | 90.647 | 19.324 | 1.00 | 10.46 | C |
| ATOM | 2137 | C | THR | A | 281 | 80.344 | 88.312 | 21.068 | 1.00 | 10.67 | C |
| ATOM | 2138 | O | THR | A | 281 | 81.082 | 88.710 | 21.965 | 1.00 | 10.13 | O |
| ATOM | 2139 | N | ARG | A | 282 | 79.059 | 88.042 | 21.255 | 1.00 | 11.38 | N |
| ATOM | 2140 | CA | ARG | A | 282 | 78.415 | 88.222 | 22.544 | 1.00 | 12.07 | C |
| ATOM | 2141 | CB | ARG | A | 282 | 76.888 | 88.182 | 22.392 | 1.00 | 13.37 | C |
| ATOM | 2142 | CG | ARG | A | 282 | 76.439 | 89.573 | 21.990 | 1.00 | 16.82 | C |
| ATOM | 2143 | CD | ARG | A | 282 | 74.982 | 89.693 | 21.745 | 1.00 | 21.18 | C |
| ATOM | 2144 | NE | ARG | A | 282 | 74.212 | 89.471 | 22.962 | 1.00 | 22.09 | N |
| ATOM | 2145 | CZ | ARG | A | 282 | 72.896 | 89.567 | 22.991 | 1.00 | 18.56 | C |
| ATOM | 2146 | NH1 | ARG | A | 282 | 72.248 | 89.899 | 21.891 | 1.00 | 21.11 | N |
| ATOM | 2147 | NH2 | ARG | A | 282 | 72.233 | 89.301 | 24.103 | 1.00 | 22.68 | N |
| ATOM | 2148 | C | ARG | A | 282 | 78.926 | 87.256 | 23.597 | 1.00 | 9.91 | C |
| ATOM | 2149 | O | ARG | A | 282 | 79.157 | 87.666 | 24.732 | 1.00 | 9.98 | O |
| ATOM | 2150 | N | ILE | A | 283 | 79.186 | 86.008 | 23.211 | 1.00 | 8.83 | N |
| ATOM | 2151 | CA | ILE | A | 283 | 79.809 | 85.034 | 24.144 | 1.00 | 8.69 | C |
| ATOM | 2152 | CB | ILE | A | 283 | 79.914 | 83.612 | 23.560 | 1.00 | 8.33 | C |
| ATOM | 2153 | CG1 | ILE | A | 283 | 78.512 | 83.012 | 23.332 | 1.00 | 8.21 | C |
| ATOM | 2154 | CD1 | ILE | A | 283 | 77.723 | 82.744 | 24.600 | 1.00 | 7.73 | C |
| ATOM | 2155 | CG2 | ILE | A | 283 | 80.757 | 82.724 | 24.461 | 1.00 | 7.70 | C |
| ATOM | 2156 | C | ILE | A | 283 | 81.200 | 85.522 | 24.533 | 1.00 | 9.40 | C |
| ATOM | 2157 | O | ILE | A | 283 | 81.547 | 85.519 | 25.708 | 1.00 | 8.82 | O |
| ATOM | 2158 | N | ARG | A | 284 | 81.986 | 85.988 | 23.555 | 1.00 | 9.27 | N |
| ATOM | 2159 | CA | ARG | A | 284 | 83.368 | 86.323 | 23.810 | 1.00 | 9.40 | C |
| ATOM | 2160 | CB | ARG | A | 284 | 84.219 | 86.135 | 22.570 | 1.00 | 9.54 | C |
| ATOM | 2161 | CG | ARG | A | 284 | 84.283 | 84.672 | 22.149 | 1.00 | 9.85 | C |
| ATOM | 2162 | CD | ARG | A | 284 | 84.867 | 84.517 | 20.762 | 1.00 | 11.05 | C |
| ATOM | 2163 | NE | ARG | A | 284 | 85.155 | 83.130 | 20.424 | 1.00 | 9.94 | N |
| ATOM | 2164 | CZ | ARG | A | 284 | 85.190 | 82.648 | 19.192 | 1.00 | 10.48 | C |
| ATOM | 2165 | NH1 | ARG | A | 284 | 84.932 | 83.437 | 18.158 | 1.00 | 11.07 | N |
| ATOM | 2166 | NH2 | ARG | A | 284 | 85.506 | 81.370 | 18.982 | 1.00 | 9.33 | N |
| ATOM | 2167 | C | ARG | A | 284 | 83.561 | 87.712 | 24.418 | 1.00 | 10.50 | C |
| ATOM | 2168 | O | ARG | A | 284 | 84.616 | 87.981 | 24.964 | 1.00 | 9.41 | O |
| ATOM | 2169 | N | ALA | A | 285 | 82.542 | 88.558 | 24.397 | 1.00 | 12.56 | N |
| ATOM | 2170 | CA | ALA | A | 285 | 82.618 | 89.842 | 25.131 | 1.00 | 14.16 | C |
| ATOM | 2171 | CB | ALA | A | 285 | 81.335 | 90.649 | 24.977 | 1.00 | 13.34 | C |
| ATOM | 2172 | C | ALA | A | 285 | 82.890 | 89.560 | 26.603 | 1.00 | 16.76 | C |
| ATOM | 2173 | O | ALA | A | 285 | 83.639 | 90.278 | 27.241 | 1.00 | 14.83 | O |
| ATOM | 2174 | N | LYS | A | 286 | 82.289 | 88.491 | 27.123 | 1.00 | 17.44 | N |
| ATOM | 2175 | CA | LYS | A | 286 | 82.434 | 88.068 | 28.517 | 1.00 | 21.02 | C |
| ATOM | 2176 | CB | LYS | A | 286 | 81.126 | 87.360 | 28.937 | 1.00 | 27.53 | C |
| ATOM | 2177 | CG | LYS | A | 286 | 79.855 | 88.212 | 28.846 | 1.00 | 36.52 | C |
| ATOM | 2178 | CD | LYS | A | 286 | 79.645 | 89.078 | 30.082 | 1.00 | 41.07 | C |
| ATOM | 2179 | CE | LYS | A | 286 | 78.341 | 89.872 | 29.999 | 1.00 | 48.98 | C |
| ATOM | 2180 | NZ | LYS | A | 286 | 77.157 | 89.108 | 30.506 | 1.00 | 43.98 | N |
| ATOM | 2181 | C | LYS | A | 286 | 83.606 | 87.089 | 28.704 | 1.00 | 17.36 | C |
| ATOM | 2182 | O | LYS | A | 286 | 84.221 | 87.044 | 29.750 | 1.00 | 16.20 | O |
| ATOM | 2183 | N | HIS | A | 287 | 83.890 | 86.297 | 27.671 | 1.00 | 16.25 | N |
| ATOM | 2184 | CA | HIS | A | 287 | 84.897 | 85.226 | 27.723 | 1.00 | 11.67 | C |
| ATOM | 2185 | CB | HIS | A | 287 | 84.176 | 83.887 | 27.787 | 1.00 | 10.16 | C |
| ATOM | 2186 | CG | HIS | A | 287 | 83.236 | 83.803 | 28.965 | 1.00 | 11.21 | C |
| ATOM | 2187 | ND1 | HIS | A | 287 | 83.671 | 83.554 | 30.231 | 1.00 | 10.60 | N |
| ATOM | 2188 | CE1 | HIS | A | 287 | 82.627 | 83.636 | 31.088 | 1.00 | 11.11 | C |
| ATOM | 2189 | NE2 | HIS | A | 287 | 81.513 | 83.926 | 30.365 | 1.00 | 11.59 | N |
| ATOM | 2190 | CD2 | HIS | A | 287 | 81.867 | 84.063 | 29.063 | 1.00 | 12.14 | C |
| ATOM | 2191 | C | HIS | A | 287 | 85.807 | 85.319 | 26.542 | 1.00 | 11.38 | C |
| ATOM | 2192 | O | HIS | A | 287 | 85.665 | 84.559 | 25.601 | 1.00 | 10.48 | O |
| ATOM | 2193 | N | PRO | A | 288 | 86.754 | 86.268 | 26.568 | 1.00 | 11.91 | N |
| ATOM | 2194 | CA | PRO | A | 288 | 87.639 | 86.448 | 25.430 | 1.00 | 13.13 | C |
| ATOM | 2195 | CB | PRO | A | 288 | 88.691 | 87.450 | 25.951 | 1.00 | 16.17 | C |
| ATOM | 2196 | CG | PRO | A | 288 | 88.016 | 88.184 | 27.054 | 1.00 | 15.44 | C |
| ATOM | 2197 | CD | PRO | A | 288 | 87.025 | 87.239 | 27.652 | 1.00 | 14.38 | C |
| ATOM | 2198 | C | PRO | A | 288 | 88.294 | 85.147 | 24.969 | 1.00 | 12.37 | C |
| ATOM | 2199 | O | PRO | A | 288 | 88.768 | 84.379 | 25.775 | 1.00 | 11.70 | O |
| ATOM | 2200 | N | GLY | A | 289 | 88.301 | 84.912 | 23.666 | 1.00 | 12.99 | N |
| ATOM | 2201 | CA | GLY | A | 289 | 88.974 | 83.756 | 23.096 | 1.00 | 14.02 | C |
| ATOM | 2202 | C | GLY | A | 289 | 88.232 | 82.435 | 23.283 | 1.00 | 15.06 | C |
| ATOM | 2203 | O | GLY | A | 289 | 88.771 | 81.411 | 22.938 | 1.00 | 12.37 | O |
| ATOM | 2204 | N | PHE | A | 290 | 86.999 | 82.444 | 23.809 | 1.00 | 11.44 | N |
| ATOM | 2205 | CA | PHE | A | 290 | 86.307 | 81.192 | 24.102 | 1.00 | 10.22 | C |
| ATOM | 2206 | CB | PHE | A | 290 | 84.875 | 81.445 | 24.620 | 1.00 | 9.87 | C |
| ATOM | 2207 | CG | PHE | A | 290 | 84.238 | 80.258 | 25.257 | 1.00 | 8.59 | C |
| ATOM | 2208 | CD1 | PHE | A | 290 | 84.499 | 79.942 | 26.580 | 1.00 | 8.96 | C |
| ATOM | 2209 | CE1 | PHE | A | 290 | 83.913 | 78.832 | 27.166 | 1.00 | 8.20 | C |
| ATOM | 2210 | CZ | PHE | A | 290 | 83.079 | 78.023 | 26.439 | 1.00 | 9.40 | C |
| ATOM | 2211 | CE2 | PHE | A | 290 | 82.790 | 78.343 | 25.125 | 1.00 | 8.96 | C |
| ATOM | 2212 | CD2 | PHE | A | 290 | 83.392 | 79.452 | 24.543 | 1.00 | 8.91 | C |
| ATOM | 2213 | C | PHE | A | 290 | 86.316 | 80.317 | 22.836 | 1.00 | 9.94 | C |
| ATOM | 2214 | O | PHE | A | 290 | 85.907 | 80.755 | 21.740 | 1.00 | 10.10 | O |
| ATOM | 2215 | N | PHE | A | 291 | 86.825 | 79.103 | 22.976 | 1.00 | 9.05 | N |
| ATOM | 2216 | CA | PHE | A | 291 | 87.013 | 78.210 | 21.814 | 1.00 | 9.15 | C |
| ATOM | 2217 | CB | PHE | A | 291 | 88.145 | 77.220 | 22.076 | 1.00 | 9.25 | C |
| ATOM | 2218 | CG | PHE | A | 291 | 88.474 | 76.347 | 20.871 | 1.00 | 9.99 | C |
| ATOM | 2219 | CD1 | PHE | A | 291 | 89.121 | 76.887 | 19.754 | 1.00 | 10.58 | C |
| ATOM | 2220 | CE1 | PHE | A | 291 | 89.433 | 76.081 | 18.662 | 1.00 | 12.19 | C |
| ATOM | 2221 | CZ | PHE | A | 291 | 89.133 | 74.731 | 18.695 | 1.00 | 11.62 | C |
| ATOM | 2222 | CE2 | PHE | A | 291 | 88.477 | 74.205 | 19.785 | 1.00 | 11.64 | C |
| ATOM | 2223 | CD2 | PHE | A | 291 | 88.166 | 75.019 | 20.866 | 1.00 | 10.63 | C |
| ATOM | 2224 | C | PHE | A | 291 | 85.735 | 77.434 | 21.441 | 1.00 | 9.18 | C |
| ATOM | 2225 | O | PHE | A | 291 | 85.036 | 76.887 | 22.309 | 1.00 | 8.66 | O |
| ATOM | 2226 | N | MET | A | 292 | 85.433 | 77.413 | 20.152 | 1.00 | 7.88 | N |
| ATOM | 2227 | CA | MET | A | 292 | 84.284 | 76.716 | 19.632 | 1.00 | 9.77 | C |
| ATOM | 2228 | CB | MET | A | 292 | 83.108 | 77.659 | 19.387 | 1.00 | 10.35 | C |
| ATOM | 2229 | CG | MET | A | 292 | 82.811 | 78.590 | 20.549 | 1.00 | 11.54 | C |
| ATOM | 2230 | SD | MET | A | 292 | 81.378 | 79.612 | 20.212 | 1.00 | 14.13 | S |
| ATOM | 2231 | CE | MET | A | 292 | 81.483 | 80.824 | 21.522 | 1.00 | 8.87 | C |
| ATOM | 2232 | C | MET | A | 292 | 84.632 | 76.044 | 18.318 | 1.00 | 9.69 | C |
| ATOM | 2233 | O | MET | A | 292 | 85.452 | 76.554 | 17.556 | 1.00 | 9.85 | O |
| ATOM | 2234 | N | PHE | A | 293 | 84.023 | 74.887 | 18.082 | 1.00 | 9.21 | N |
| ATOM | 2235 | CA | PHE | A | 293 | 84.140 | 74.214 | 16.792 | 1.00 | 9.16 | C |
| ATOM | 2236 | CB | PHE | A | 293 | 85.195 | 73.102 | 16.794 | 1.00 | 10.12 | C |
| ATOM | 2237 | CG | PHE | A | 293 | 84.996 | 72.012 | 17.819 | 1.00 | 11.55 | C |
| ATOM | 2238 | CD1 | PHE | A | 293 | 85.522 | 72.138 | 19.099 | 1.00 | 11.47 | C |
| ATOM | 2239 | CE1 | PHE | A | 293 | 85.374 | 71.115 | 20.041 | 1.00 | 12.63 | C |
| ATOM | 2240 | CZ | PHE | A | 293 | 84.740 | 69.935 | 19.688 | 1.00 | 12.04 | C |
| ATOM | 2241 | CE2 | PHE | A | 293 | 84.237 | 69.786 | 18.398 | 1.00 | 12.95 | C |
| ATOM | 2242 | CD2 | PHE | A | 293 | 84.400 | 70.808 | 17.459 | 1.00 | 10.73 | C |
| ATOM | 2243 | C | PHE | A | 293 | 82.777 | 73.754 | 16.279 | 1.00 | 9.55 | C |
| ATOM | 2244 | O | PHE | A | 293 | 81.991 | 73.147 | 16.992 | 1.00 | 9.18 | O |
| ATOM | 2245 | N | GLY | A | 294 | 82.496 | 74.053 | 15.021 | 1.00 | 9.20 | N |
| ATOM | 2246 | CA | GLY | A | 294 | 81.203 | 73.738 | 14.477 | 1.00 | 9.79 | C |
| ATOM | 2247 | C | GLY | A | 294 | 81.115 | 72.512 | 13.588 | 1.00 | 10.37 | C |
| ATOM | 2248 | O | GLY | A | 294 | 82.108 | 72.110 | 12.940 | 1.00 | 8.84 | O |
| ATOM | 2249 | N | GLU | A | 295 | 79.908 | 71.946 | 13.560 | 1.00 | 9.56 | N |
| ATOM | 2250 | CA | GLU | A | 295 | 79.588 | 70.801 | 12.739 | 1.00 | 10.45 | C |
| ATOM | 2251 | CB | GLU | A | 295 | 78.672 | 69.815 | 13.450 | 1.00 | 10.41 | C |
| ATOM | 2252 | CG | GLU | A | 295 | 78.214 | 68.681 | 12.515 | 1.00 | 9.73 | C |
| ATOM | 2253 | CD | GLU | A | 295 | 76.714 | 68.373 | 12.552 | 1.00 | 12.79 | C |
| ATOM | 2254 | OE1 | GLU | A | 295 | 75.900 | 69.107 | 13.171 | 1.00 | 12.40 | O |
| ATOM | 2255 | OE2 | GLU | A | 295 | 76.325 | 67.362 | 11.909 | 1.00 | 12.93 | O |
| ATOM | 2256 | C | GLU | A | 295 | 78.901 | 71.281 | 11.467 | 1.00 | 9.52 | C |
| ATOM | 2257 | O | GLU | A | 295 | 77.705 | 71.599 | 11.487 | 1.00 | 9.34 | O |
| ATOM | 2258 | N | ALA | A | 296 | 79.692 | 71.360 | 10.393 | 1.00 | 8.43 | N |
| ATOM | 2259 | CA | ALA | A | 296 | 79.210 | 71.570 | 9.030 | 1.00 | 10.11 | C |
| ATOM | 2260 | CB | ALA | A | 296 | 79.970 | 72.714 | 8.353 | 1.00 | 9.64 | C |
| ATOM | 2261 | C | ALA | A | 296 | 79.409 | 70.251 | 8.284 | 1.00 | 9.91 | C |
| ATOM | 2262 | O | ALA | A | 296 | 80.539 | 69.874 | 7.984 | 1.00 | 11.06 | O |
| ATOM | 2263 | N | PHE | A | 297 | 78.309 | 69.545 | 8.019 | 1.00 | 10.07 | N |
| ATOM | 2264 | CA | PHE | A | 297 | 78.357 | 68.157 | 7.504 | 1.00 | 10.08 | C |
| ATOM | 2265 | CB | PHE | A | 297 | 77.140 | 67.342 | 7.961 | 1.00 | 10.16 | C |
| ATOM | 2266 | CG | PHE | A | 297 | 77.341 | 65.851 | 7.936 | 1.00 | 11.39 | C |
| ATOM | 2267 | CD1 | PHE | A | 297 | 77.927 | 65.211 | 6.851 | 1.00 | 11.69 | C |
| ATOM | 2268 | CE1 | PHE | A | 297 | 78.076 | 63.835 | 6.822 | 1.00 | 12.87 | C |
| ATOM | 2269 | CZ | PHE | A | 297 | 77.593 | 63.081 | 7.863 | 1.00 | 11.34 | C |
| ATOM | 2270 | CE2 | PHE | A | 297 | 77.009 | 63.700 | 8.936 | 1.00 | 11.87 | C |
| ATOM | 2271 | CD2 | PHE | A | 297 | 76.879 | 65.078 | 8.975 | 1.00 | 12.63 | C |
| ATOM | 2272 | C | PHE | A | 297 | 78.479 | 68.253 | 6.000 | 1.00 | 9.35 | C |
| ATOM | 2273 | O | PHE | A | 297 | 77.481 | 68.241 | 5.255 | 1.00 | 7.99 | O |
| ATOM | 2274 | N | ASP | A | 298 | 79.734 | 68.362 | 5.561 | 1.00 | 9.67 | N |
| ATOM | 2275 | CA | ASP | A | 298 | 80.038 | 68.660 | 4.167 | 1.00 | 9.68 | C |
| ATOM | 2276 | CB | ASP | A | 298 | 79.778 | 70.150 | 3.934 | 1.00 | 10.36 | C |
| ATOM | 2277 | CG | ASP | A | 298 | 79.952 | 70.597 | 2.484 | 1.00 | 13.00 | C |
| ATOM | 2278 | OD1 | ASP | A | 298 | 80.669 | 69.928 | 1.694 | 1.00 | 14.93 | O |
| ATOM | 2279 | OD2 | ASP | A | 298 | 79.395 | 71.666 | 2.141 | 1.00 | 12.78 | O |
| ATOM | 2280 | C | ASP | A | 298 | 81.511 | 68.282 | 3.964 | 1.00 | 9.95 | C |
| ATOM | 2281 | O | ASP | A | 298 | 82.378 | 68.683 | 4.741 | 1.00 | 10.40 | O |
| ATOM | 2282 | N | TYR | A | 299 | 81.779 | 67.466 | 2.950 | 1.00 | 9.57 | N |
| ATOM | 2283 | CA | TYR | A | 299 | 83.139 | 66.995 | 2.653 | 1.00 | 11.03 | C |
| ATOM | 2284 | CB | TYR | A | 299 | 83.053 | 65.591 | 2.031 | 1.00 | 12.44 | C |
| ATOM | 2285 | CG | TYR | A | 299 | 82.544 | 64.590 | 3.060 | 1.00 | 11.99 | C |
| ATOM | 2286 | CD1 | TYR | A | 299 | 83.360 | 64.170 | 4.084 | 1.00 | 12.22 | C |
| ATOM | 2287 | CE1 | TYR | A | 299 | 82.914 | 63.285 | 5.045 | 1.00 | 11.45 | C |
| ATOM | 2288 | CZ | TYR | A | 299 | 81.626 | 62.803 | 5.006 | 1.00 | 14.21 | C |
| ATOM | 2289 | OH | TYR | A | 299 | 81.241 | 61.901 | 5.984 | 1.00 | 17.09 | O |
| ATOM | 2290 | CE2 | TYR | A | 299 | 80.768 | 63.207 | 3.997 | 1.00 | 13.87 | C |
| ATOM | 2291 | CD2 | TYR | A | 299 | 81.237 | 64.107 | 3.026 | 1.00 | 13.67 | C |
| ATOM | 2292 | C | TYR | A | 299 | 83.995 | 67.938 | 1.809 | 1.00 | 11.50 | C |
| ATOM | 2293 | O | TYR | A | 299 | 85.140 | 67.619 | 1.474 | 1.00 | 11.92 | O |
| ATOM | 2294 | N | ASP | A | 300 | 83.457 | 69.121 | 1.527 | 1.00 | 11.83 | N |
| ATOM | 2295 | CA | ASP | A | 300 | 84.159 | 70.163 | 0.798 | 1.00 | 9.72 | C |
| ATOM | 2296 | CB | ASP | A | 300 | 83.194 | 70.903 | −0.112 | 1.00 | 10.78 | C |
| ATOM | 2297 | CG | ASP | A | 300 | 83.924 | 71.912 | −0.992 | 1.00 | 15.25 | C |
| ATOM | 2298 | OD1 | ASP | A | 300 | 84.592 | 71.447 | −1.917 | 1.00 | 17.17 | O |
| ATOM | 2299 | OD2 | ASP | A | 300 | 83.918 | 73.135 | −0.703 | 1.00 | 16.54 | O |
| ATOM | 2300 | C | ASP | A | 300 | 84.822 | 71.191 | 1.719 | 1.00 | 9.38 | C |
| ATOM | 2301 | O | ASP | A | 300 | 84.124 | 71.947 | 2.455 | 1.00 | 8.06 | O |
| ATOM | 2302 | N | ALA | A | 301 | 86.152 | 71.269 | 1.652 | 1.00 | 8.38 | N |
| ATOM | 2303 | CA | ALA | A | 301 | 86.925 | 72.136 | 2.569 | 1.00 | 8.68 | C |
| ATOM | 2304 | CB | ALA | A | 301 | 88.423 | 71.973 | 2.308 | 1.00 | 8.56 | C |
| ATOM | 2305 | C | ALA | A | 301 | 86.546 | 73.617 | 2.480 | 1.00 | 9.46 | C |
| ATOM | 2306 | O | ALA | A | 301 | 86.364 | 74.303 | 3.499 | 1.00 | 9.71 | O |
| ATOM | 2307 | N | ALA | A | 302 | 86.467 | 74.127 | 1.270 | 1.00 | 11.18 | N |
| ATOM | 2308 | CA | ALA | A | 302 | 86.122 | 75.529 | 1.036 | 1.00 | 11.02 | C |
| ATOM | 2309 | CB | ALA | A | 302 | 86.191 | 75.844 | −0.462 | 1.00 | 10.72 | C |
| ATOM | 2310 | C | ALA | A | 302 | 84.739 | 75.900 | 1.608 | 1.00 | 12.21 | C |
| ATOM | 2311 | O | ALA | A | 302 | 84.576 | 76.947 | 2.212 | 1.00 | 11.22 | O |
| ATOM | 2312 | N | ARG | A | 303 | 83.731 | 75.055 | 1.412 | 1.00 | 13.60 | N |
| ATOM | 2313 | CA | ARG | A | 303 | 82.399 | 75.356 | 1.965 | 1.00 | 12.42 | C |
| ATOM | 2314 | CB | ARG | A | 303 | 81.305 | 74.425 | 1.397 | 1.00 | 13.39 | C |
| ATOM | 2315 | CG | ARG | A | 303 | 80.699 | 74.895 | 0.083 | 1.00 | 16.07 | C |
| ATOM | 2316 | CD | ARG | A | 303 | 79.416 | 74.164 | −0.301 | 1.00 | 14.66 | C |
| ATOM | 2317 | NE | ARG | A | 303 | 79.670 | 72.733 | −0.348 | 1.00 | 16.46 | N |
| ATOM | 2318 | CZ | ARG | A | 303 | 79.876 | 71.991 | −1.440 | 1.00 | 14.22 | C |
| ATOM | 2319 | NH1 | ARG | A | 303 | 79.811 | 72.505 | −2.665 | 1.00 | 15.11 | N |
| ATOM | 2320 | NH2 | ARG | A | 303 | 80.145 | 70.702 | −1.294 | 1.00 | 12.11 | N |
| ATOM | 2321 | C | ARG | A | 303 | 82.376 | 75.344 | 3.504 | 1.00 | 11.66 | C |
| ATOM | 2322 | O | ARG | A | 303 | 81.738 | 76.190 | 4.091 | 1.00 | 12.84 | O |
| ATOM | 2323 | N | ILE | A | 304 | 83.035 | 74.399 | 4.170 | 1.00 | 9.76 | N |
| ATOM | 2324 | CA | ILE | A | 304 | 83.008 | 74.409 | 5.624 | 1.00 | 9.85 | C |
| ATOM | 2325 | CB | ILE | A | 304 | 83.284 | 73.014 | 6.263 | 1.00 | 9.85 | C |
| ATOM | 2326 | CG1 | ILE | A | 304 | 84.706 | 72.493 | 6.013 | 1.00 | 9.03 | C |
| ATOM | 2327 | CD1 | ILE | A | 304 | 85.056 | 71.286 | 6.873 | 1.00 | 7.52 | C |
| ATOM | 2328 | CG2 | ILE | A | 304 | 82.282 | 71.965 | 5.745 | 1.00 | 9.80 | C |
| ATOM | 2329 | C | ILE | A | 304 | 83.945 | 75.488 | 6.195 | 1.00 | 10.95 | C |
| ATOM | 2330 | O | ILE | A | 304 | 83.711 | 76.000 | 7.314 | 1.00 | 9.86 | O |
| ATOM | 2331 | N | ALA | A | 305 | 84.961 | 75.883 | 5.406 | 1.00 | 9.86 | N |
| ATOM | 2332 | CA | ALA | A | 305 | 85.950 | 76.848 | 5.879 | 1.00 | 9.62 | C |
| ATOM | 2333 | CB | ALA | A | 305 | 87.108 | 76.978 | 4.878 | 1.00 | 8.53 | C |
| ATOM | 2334 | C | ALA | A | 305 | 85.361 | 78.232 | 6.187 | 1.00 | 8.63 | C |
| ATOM | 2335 | O | ALA | A | 305 | 85.946 | 79.000 | 6.948 | 1.00 | 7.54 | O |
| ATOM | 2336 | N | THR | A | 306 | 84.252 | 78.574 | 5.548 | 1.00 | 9.00 | N |
| ATOM | 2337 | CA | THR | A | 306 | 83.642 | 79.902 | 5.669 | 1.00 | 10.22 | C |
| ATOM | 2338 | CB | THR | A | 306 | 82.323 | 79.974 | 4.860 | 1.00 | 14.38 | C |
| ATOM | 2339 | OG1 | THR | A | 306 | 81.466 | 78.879 | 5.265 | 1.00 | 21.19 | O |
| ATOM | 2340 | CG2 | THR | A | 306 | 82.580 | 79.887 | 3.361 | 1.00 | 11.24 | C |
| ATOM | 2341 | C | THR | A | 306 | 83.358 | 80.293 | 7.132 | 1.00 | 10.35 | C |
| ATOM | 2342 | O | THR | A | 306 | 83.494 | 81.456 | 7.516 | 1.00 | 9.68 | O |
| ATOM | 2343 | N | HIS | A | 307 | 83.002 | 79.300 | 7.947 | 1.00 | 8.78 | N |
| ATOM | 2344 | CA | HIS | A | 307 | 82.683 | 79.498 | 9.351 | 1.00 | 9.49 | C |
| ATOM | 2345 | CB | HIS | A | 307 | 82.091 | 78.195 | 9.919 | 1.00 | 8.87 | C |
| ATOM | 2346 | CG | HIS | A | 307 | 80.878 | 77.723 | 9.189 | 1.00 | 8.30 | C |
| ATOM | 2347 | ND1 | HIS | A | 307 | 79.697 | 78.362 | 9.266 | 1.00 | 8.68 | N |
| ATOM | 2348 | CE1 | HIS | A | 307 | 78.783 | 77.731 | 8.498 | 1.00 | 7.50 | C |
| ATOM | 2349 | NE2 | HIS | A | 307 | 79.384 | 76.691 | 7.898 | 1.00 | 8.42 | N |
| ATOM | 2350 | CD2 | HIS | A | 307 | 80.685 | 76.646 | 8.309 | 1.00 | 9.11 | C |
| ATOM | 2351 | C | HIS | A | 307 | 83.871 | 79.903 | 10.166 | 1.00 | 9.45 | C |
| ATOM | 2352 | O | HIS | A | 307 | 83.705 | 80.474 | 11.242 | 1.00 | 11.07 | O |
| ATOM | 2353 | N | THR | A | 308 | 85.081 | 79.646 | 9.652 | 1.00 | 9.41 | N |
| ATOM | 2354 | CA | THR | A | 308 | 86.318 | 79.958 | 10.351 | 1.00 | 9.31 | C |
| ATOM | 2355 | CB | THR | A | 308 | 87.501 | 79.029 | 9.955 | 1.00 | 9.37 | C |
| ATOM | 2356 | OG1 | THR | A | 308 | 87.961 | 79.325 | 8.630 | 1.00 | 7.62 | O |
| ATOM | 2357 | CG2 | THR | A | 308 | 87.113 | 77.525 | 10.036 | 1.00 | 6.91 | C |
| ATOM | 2358 | C | THR | A | 308 | 86.752 | 81.411 | 10.137 | 1.00 | 10.18 | C |
| ATOM | 2359 | O | THR | A | 308 | 87.598 | 81.896 | 10.855 | 1.00 | 8.94 | O |
| ATOM | 2360 | N | LEU | A | 309 | 86.170 | 82.093 | 9.162 | 1.00 | 10.55 | N |
| ATOM | 2361 | CA | LEU | A | 309 | 86.743 | 83.343 | 8.688 | 1.00 | 11.94 | C |
| ATOM | 2362 | CB | LEU | A | 309 | 86.366 | 83.554 | 7.222 | 1.00 | 12.52 | C |
| ATOM | 2363 | CG | LEU | A | 309 | 86.850 | 82.471 | 6.250 | 1.00 | 15.75 | C |
| ATOM | 2364 | CD1 | LEU | A | 309 | 86.375 | 82.827 | 4.826 | 1.00 | 16.29 | C |
| ATOM | 2365 | CD2 | LEU | A | 309 | 88.371 | 82.260 | 6.365 | 1.00 | 12.11 | C |
| ATOM | 2366 | C | LEU | A | 309 | 86.256 | 84.547 | 9.488 | 1.00 | 13.55 | C |
| ATOM | 2367 | O | LEU | A | 309 | 85.178 | 84.488 | 10.098 | 1.00 | 11.41 | O |
| ATOM | 2368 | N | PRO | A | 310 | 87.032 | 85.658 | 9.464 | 1.00 | 14.42 | N |
| ATOM | 2369 | CA | PRO | A | 310 | 86.597 | 86.860 | 10.179 | 1.00 | 13.98 | C |
| ATOM | 2370 | CB | PRO | A | 310 | 87.711 | 87.881 | 9.887 | 1.00 | 16.41 | C |
| ATOM | 2371 | CG | PRO | A | 310 | 88.922 | 87.061 | 9.574 | 1.00 | 16.94 | C |
| ATOM | 2372 | CD | PRO | A | 310 | 88.389 | 85.810 | 8.893 | 1.00 | 16.27 | C |
| ATOM | 2373 | C | PRO | A | 310 | 85.244 | 87.355 | 9.688 | 1.00 | 12.93 | C |
| ATOM | 2374 | O | PRO | A | 310 | 85.000 | 87.407 | 8.480 | 1.00 | 11.94 | O |
| ATOM | 2375 | N | GLY | A | 311 | 84.358 | 87.675 | 10.625 | 1.00 | 12.00 | N |
| ATOM | 2376 | CA | GLY | A | 311 | 83.024 | 88.172 | 10.296 | 1.00 | 11.69 | C |
| ATOM | 2377 | C | GLY | A | 311 | 82.001 | 87.082 | 10.013 | 1.00 | 12.30 | C |
| ATOM | 2378 | O | GLY | A | 311 | 80.854 | 87.384 | 9.832 | 1.00 | 12.37 | O |
| ATOM | 2379 | N | HIS | A | 312 | 82.412 | 85.812 | 10.002 | 1.00 | 11.89 | N |
| ATOM | 2380 | CA | HIS | A | 312 | 81.531 | 84.718 | 9.611 | 1.00 | 12.94 | C |
| ATOM | 2381 | CB | HIS | A | 312 | 82.092 | 84.023 | 8.373 | 1.00 | 12.91 | C |
| ATOM | 2382 | CG | HIS | A | 312 | 81.940 | 84.839 | 7.129 | 1.00 | 17.78 | C |
| ATOM | 2383 | ND1 | HIS | A | 312 | 80.922 | 84.649 | 6.259 | 1.00 | 19.08 | N |
| ATOM | 2384 | CE1 | HIS | A | 312 | 81.031 | 85.530 | 5.238 | 1.00 | 21.50 | C |
| ATOM | 2385 | NE2 | HIS | A | 312 | 82.108 | 86.312 | 5.478 | 1.00 | 24.25 | N |
| ATOM | 2386 | CD2 | HIS | A | 312 | 82.693 | 85.905 | 6.636 | 1.00 | 18.49 | C |
| ATOM | 2387 | C | HIS | A | 312 | 81.280 | 83.729 | 10.729 | 1.00 | 11.50 | C |
| ATOM | 2388 | O | HIS | A | 312 | 80.817 | 82.621 | 10.477 | 1.00 | 11.13 | O |
| ATOM | 2389 | N | GLY | A | 313 | 81.528 | 84.172 | 11.963 | 1.00 | 10.71 | N |
| ATOM | 2390 | CA | GLY | A | 313 | 81.391 | 83.368 | 13.158 | 1.00 | 11.15 | C |
| ATOM | 2391 | C | GLY | A | 313 | 82.693 | 83.135 | 13.905 | 1.00 | 10.67 | C |
| ATOM | 2392 | O | GLY | A | 313 | 82.700 | 83.087 | 15.125 | 1.00 | 11.53 | O |
| ATOM | 2393 | N | GLU | A | 314 | 83.785 | 82.962 | 13.175 | 1.00 | 10.32 | N |
| ATOM | 2394 | CA | GLU | A | 314 | 85.125 | 82.880 | 13.757 | 1.00 | 12.38 | C |
| ATOM | 2395 | CB | GLU | A | 314 | 85.483 | 84.221 | 14.445 | 1.00 | 14.49 | C |
| ATOM | 2396 | CG | GLU | A | 314 | 85.600 | 85.376 | 13.446 | 1.00 | 17.46 | C |
| ATOM | 2397 | CD | GLU | A | 314 | 85.534 | 86.784 | 14.058 | 1.00 | 20.63 | C |
| ATOM | 2398 | OE1 | GLU | A | 314 | 85.541 | 86.931 | 15.290 | 1.00 | 24.13 | O |
| ATOM | 2399 | OE2 | GLU | A | 314 | 85.442 | 87.759 | 13.289 | 1.00 | 15.75 | O |
| ATOM | 2400 | C | GLU | A | 314 | 85.251 | 81.690 | 14.700 | 1.00 | 10.85 | C |
| ATOM | 2401 | O | GLU | A | 314 | 85.640 | 81.833 | 15.842 | 1.00 | 10.16 | O |
| ATOM | 2402 | N | THR | A | 315 | 84.842 | 80.515 | 14.233 | 1.00 | 10.17 | N |
| ATOM | 2403 | CA | THR | A | 315 | 84.970 | 79.309 | 15.032 | 1.00 | 10.01 | C |
| ATOM | 2404 | CB | THR | A | 315 | 83.609 | 78.646 | 15.383 | 1.00 | 10.24 | C |
| ATOM | 2405 | OG1 | THR | A | 315 | 82.962 | 78.136 | 14.202 | 1.00 | 11.04 | O |
| ATOM | 2406 | CG2 | THR | A | 315 | 82.663 | 79.635 | 16.098 | 1.00 | 9.54 | C |
| ATOM | 2407 | C | THR | A | 315 | 85.846 | 78.365 | 14.214 | 1.00 | 11.39 | C |
| ATOM | 2408 | O | THR | A | 315 | 86.026 | 78.550 | 13.002 | 1.00 | 11.64 | O |
| ATOM | 2409 | N | SER | A | 316 | 86.380 | 77.356 | 14.876 | 1.00 | 9.87 | N |
| ATOM | 2410 | CA | SER | A | 316 | 86.924 | 76.215 | 14.178 | 1.00 | 9.57 | C |
| ATOM | 2411 | CB | SER | A | 316 | 87.825 | 75.437 | 15.130 | 1.00 | 9.99 | C |
| ATOM | 2412 | OG | SER | A | 316 | 88.293 | 74.240 | 14.532 | 1.00 | 13.22 | O |
| ATOM | 2413 | C | SER | A | 316 | 85.753 | 75.372 | 13.631 | 1.00 | 8.57 | C |
| ATOM | 2414 | O | SER | A | 316 | 84.574 | 75.694 | 13.846 | 1.00 | 7.97 | O |
| ATOM | 2415 | N | VAL | A | 317 | 86.073 | 74.347 | 12.857 | 1.00 | 8.01 | N |
| ATOM | 2416 | CA | VAL | A | 317 | 85.083 | 73.392 | 12.378 | 1.00 | 8.11 | C |
| ATOM | 2417 | CB | VAL | A | 317 | 84.653 | 73.640 | 10.913 | 1.00 | 8.46 | C |
| ATOM | 2418 | CG1 | VAL | A | 317 | 83.855 | 74.952 | 10.742 | 1.00 | 6.89 | C |
| ATOM | 2419 | CG2 | VAL | A | 317 | 85.853 | 73.616 | 9.961 | 1.00 | 7.09 | C |
| ATOM | 2420 | C | VAL | A | 317 | 85.635 | 71.955 | 12.523 | 1.00 | 9.18 | C |
| ATOM | 2421 | O | VAL | A | 317 | 86.853 | 71.742 | 12.629 | 1.00 | 8.08 | O |
| ATOM | 2422 | N | LEU | A | 318 | 84.721 | 70.982 | 12.515 | 1.00 | 9.42 | N |
| ATOM | 2423 | CA | LEU | A | 318 | 85.064 | 69.589 | 12.452 | 1.00 | 8.66 | C |
| ATOM | 2424 | CB | LEU | A | 318 | 83.865 | 68.745 | 12.843 | 1.00 | 9.25 | C |
| ATOM | 2425 | CG | LEU | A | 318 | 83.636 | 68.814 | 14.347 | 1.00 | 9.01 | C |
| ATOM | 2426 | CD1 | LEU | A | 318 | 82.147 | 68.709 | 14.755 | 1.00 | 8.12 | C |
| ATOM | 2427 | CD2 | LEU | A | 318 | 84.499 | 67.734 | 14.991 | 1.00 | 7.86 | C |
| ATOM | 2428 | C | LEU | A | 318 | 85.561 | 69.242 | 11.040 | 1.00 | 9.13 | C |
| ATOM | 2429 | O | LEU | A | 318 | 84.936 | 69.608 | 10.032 | 1.00 | 8.59 | O |
| ATOM | 2430 | N | ASP | A | 319 | 86.688 | 68.547 | 10.970 | 1.00 | 9.15 | N |
| ATOM | 2431 | CA | ASP | A | 319 | 87.386 | 68.329 | 9.709 | 1.00 | 9.72 | C |
| ATOM | 2432 | CB | ASP | A | 319 | 88.877 | 68.105 | 9.989 | 1.00 | 10.73 | C |
| ATOM | 2433 | CG | ASP | A | 319 | 89.750 | 68.169 | 8.730 | 1.00 | 11.92 | C |
| ATOM | 2434 | OD1 | ASP | A | 319 | 89.197 | 68.122 | 7.596 | 1.00 | 10.68 | O |
| ATOM | 2435 | OD2 | ASP | A | 319 | 91.003 | 68.292 | 8.894 | 1.00 | 11.40 | O |
| ATOM | 2436 | C | ASP | A | 319 | 86.780 | 67.147 | 8.942 | 1.00 | 10.86 | C |
| ATOM | 2437 | O | ASP | A | 319 | 87.364 | 66.057 | 8.884 | 1.00 | 11.13 | O |
| ATOM | 2438 | N | PHE | A | 320 | 85.638 | 67.383 | 8.293 | 1.00 | 10.86 | N |
| ATOM | 2439 | CA | PHE | A | 320 | 85.007 | 66.332 | 7.492 | 1.00 | 9.39 | C |
| ATOM | 2440 | CB | PHE | A | 320 | 83.595 | 66.739 | 7.045 | 1.00 | 10.08 | C |
| ATOM | 2441 | CG | PHE | A | 320 | 82.541 | 66.502 | 8.101 | 1.00 | 9.82 | C |
| ATOM | 2442 | CD1 | PHE | A | 320 | 82.409 | 67.376 | 9.169 | 1.00 | 10.08 | C |
| ATOM | 2443 | CE1 | PHE | A | 320 | 81.465 | 67.155 | 10.163 | 1.00 | 10.37 | C |
| ATOM | 2444 | CZ | PHE | A | 320 | 80.620 | 66.064 | 10.093 | 1.00 | 10.49 | C |
| ATOM | 2445 | CE2 | PHE | A | 320 | 80.715 | 65.184 | 9.007 | 1.00 | 10.69 | C |
| ATOM | 2446 | CD2 | PHE | A | 320 | 81.689 | 65.409 | 8.026 | 1.00 | 10.64 | C |
| ATOM | 2447 | C | PHE | A | 320 | 85.874 | 65.825 | 6.333 | 1.00 | 10.54 | C |
| ATOM | 2448 | O | PHE | A | 320 | 85.950 | 64.625 | 6.125 | 1.00 | 11.15 | O |
| ATOM | 2449 | N | PRO | A | 321 | 86.558 | 66.728 | 5.598 | 1.00 | 10.61 | N |
| ATOM | 2450 | CA | PRO | A | 321 | 87.447 | 66.257 | 4.533 | 1.00 | 9.85 | C |
| ATOM | 2451 | CB | PRO | A | 321 | 88.059 | 67.547 | 3.996 | 1.00 | 9.33 | C |
| ATOM | 2452 | CG | PRO | A | 321 | 87.001 | 68.576 | 4.260 | 1.00 | 10.73 | C |
| ATOM | 2453 | CD | PRO | A | 321 | 86.466 | 68.204 | 5.620 | 1.00 | 9.17 | C |
| ATOM | 2454 | C | PRO | A | 321 | 88.527 | 65.269 | 4.989 | 1.00 | 10.60 | C |
| ATOM | 2455 | O | PRO | A | 321 | 88.766 | 64.270 | 4.334 | 1.00 | 9.09 | O |
| ATOM | 2456 | N | MET | A | 322 | 89.144 | 65.523 | 6.133 | 1.00 | 10.53 | N |
| ATOM | 2457 | CA | MET | A | 322 | 90.122 | 64.605 | 6.663 | 1.00 | 9.54 | C |
| ATOM | 2458 | CB | MET | A | 322 | 90.867 | 65.253 | 7.824 | 1.00 | 9.97 | C |
| ATOM | 2459 | CG | MET | A | 322 | 91.833 | 64.361 | 8.565 | 1.00 | 14.30 | C |
| ATOM | 2460 | SD | MET | A | 322 | 93.096 | 63.690 | 7.471 | 1.00 | 18.22 | S |
| ATOM | 2461 | CE | MET | A | 322 | 94.130 | 65.154 | 7.388 | 1.00 | 19.25 | C |
| ATOM | 2462 | C | MET | A | 322 | 89.438 | 63.332 | 7.112 | 1.00 | 10.68 | C |
| ATOM | 2463 | O | MET | A | 322 | 90.005 | 62.250 | 6.978 | 1.00 | 9.25 | O |
| ATOM | 2464 | N | LYS | A | 323 | 88.212 | 63.427 | 7.627 | 1.00 | 9.54 | N |
| ATOM | 2465 | CA | LYS | A | 323 | 87.533 | 62.205 | 8.003 | 1.00 | 9.37 | C |
| ATOM | 2466 | CB | LYS | A | 323 | 86.192 | 62.493 | 8.674 | 1.00 | 10.72 | C |
| ATOM | 2467 | CG | LYS | A | 323 | 85.409 | 61.244 | 9.004 | 1.00 | 12.09 | C |
| ATOM | 2468 | CD | LYS | A | 323 | 84.308 | 60.996 | 7.996 | 1.00 | 13.58 | C |
| ATOM | 2469 | CE | LYS | A | 323 | 84.082 | 59.527 | 7.716 | 1.00 | 17.41 | C |
| ATOM | 2470 | NZ | LYS | A | 323 | 82.924 | 59.399 | 6.788 | 1.00 | 15.20 | N |
| ATOM | 2471 | C | LYS | A | 323 | 87.344 | 61.281 | 6.807 | 1.00 | 8.95 | C |
| ATOM | 2472 | O | LYS | A | 323 | 87.502 | 60.062 | 6.937 | 1.00 | 6.69 | O |
| ATOM | 2473 | N | GLN | A | 324 | 86.982 | 61.839 | 5.655 | 1.00 | 9.41 | N |
| ATOM | 2474 | CA | GLN | A | 324 | 86.791 | 61.018 | 4.437 | 1.00 | 10.60 | C |
| ATOM | 2475 | CB | GLN | A | 324 | 86.238 | 61.873 | 3.290 | 1.00 | 13.02 | C |
| ATOM | 2476 | CG | GLN | A | 324 | 85.877 | 61.138 | 1.997 | 1.00 | 18.08 | C |
| ATOM | 2477 | CD | GLN | A | 324 | 85.349 | 62.120 | 0.935 | 1.00 | 27.45 | C |
| ATOM | 2478 | OE1 | GLN | A | 324 | 86.076 | 63.007 | 0.482 | 1.00 | 34.72 | O |
| ATOM | 2479 | NE2 | GLN | A | 324 | 84.091 | 61.979 | 0.557 | 1.00 | 31.23 | N |
| ATOM | 2480 | C | GLN | A | 324 | 88.106 | 60.390 | 4.008 | 1.00 | 9.18 | C |
| ATOM | 2481 | O | GLN | A | 324 | 88.135 | 59.235 | 3.578 | 1.00 | 9.76 | O |
| ATOM | 2482 | N | ALA | A | 325 | 89.188 | 61.152 | 4.120 | 1.00 | 8.67 | N |
| ATOM | 2483 | CA | ALA | A | 325 | 90.533 | 60.633 | 3.831 | 1.00 | 9.60 | C |
| ATOM | 2484 | CB | ALA | A | 325 | 91.597 | 61.752 | 3.919 | 1.00 | 7.32 | C |
| ATOM | 2485 | C | ALA | A | 325 | 90.863 | 59.474 | 4.775 | 1.00 | 8.16 | C |
| ATOM | 2486 | O | ALA | A | 325 | 91.298 | 58.404 | 4.333 | 1.00 | 7.96 | O |
| ATOM | 2487 | N | MET | A | 326 | 90.599 | 59.663 | 6.059 | 1.00 | 9.11 | N |
| ATOM | 2488 | CA | MET | A | 326 | 90.811 | 58.592 | 7.059 | 1.00 | 9.47 | C |
| ATOM | 2489 | CB | MET | A | 326 | 90.625 | 59.102 | 8.477 | 1.00 | 9.63 | C |
| ATOM | 2490 | CG | MET | A | 326 | 91.741 | 60.034 | 8.883 | 1.00 | 10.31 | C |
| ATOM | 2491 | SD | MET | A | 326 | 91.694 | 60.589 | 10.598 | 1.00 | 11.08 | S |
| ATOM | 2492 | CE | MET | A | 326 | 92.299 | 59.086 | 11.400 | 1.00 | 8.51 | C |
| ATOM | 2493 | C | MET | A | 326 | 89.982 | 57.343 | 6.817 | 1.00 | 9.92 | C |
| ATOM | 2494 | O | MET | A | 326 | 90.504 | 56.216 | 6.914 | 1.00 | 9.99 | O |
| ATOM | 2495 | N | GLU | A | 327 | 88.727 | 57.521 | 6.425 | 1.00 | 11.43 | N |
| ATOM | 2496 | CA | GLU | A | 327 | 87.881 | 56.387 | 6.075 | 1.00 | 12.28 | C |
| ATOM | 2497 | CB | GLU | A | 327 | 86.449 | 56.841 | 5.753 | 1.00 | 14.88 | C |
| ATOM | 2498 | CG | GLU | A | 327 | 85.539 | 55.675 | 5.407 | 1.00 | 22.11 | C |
| ATOM | 2499 | CD | GLU | A | 327 | 84.143 | 56.113 | 5.048 | 1.00 | 29.70 | C |
| ATOM | 2500 | OE1 | GLU | A | 327 | 83.661 | 57.127 | 5.597 | 1.00 | 30.92 | O |
| ATOM | 2501 | OE2 | GLU | A | 327 | 83.538 | 55.448 | 4.193 | 1.00 | 46.65 | O |
| ATOM | 2502 | C | GLU | A | 327 | 88.461 | 55.578 | 4.892 | 1.00 | 12.13 | C |
| ATOM | 2503 | O | GLU | A | 327 | 88.486 | 54.347 | 4.926 | 1.00 | 11.15 | O |
| ATOM | 2504 | N | GLU | A | 328 | 88.907 | 56.268 | 3.854 | 1.00 | 10.41 | N |
| ATOM | 2505 | CA | GLU | A | 328 | 89.497 | 55.607 | 2.705 | 1.00 | 11.59 | C |
| ATOM | 2506 | CB | GLU | A | 328 | 89.628 | 56.596 | 1.548 | 1.00 | 13.52 | C |
| ATOM | 2507 | CG | GLU | A | 328 | 88.243 | 57.057 | 1.092 | 1.00 | 17.77 | C |
| ATOM | 2508 | CD | GLU | A | 328 | 88.255 | 57.986 | −0.107 | 1.00 | 22.00 | C |
| ATOM | 2509 | OE1 | GLU | A | 328 | 89.326 | 58.226 | −0.674 | 1.00 | 21.70 | O |
| ATOM | 2510 | OE2 | GLU | A | 328 | 87.175 | 58.497 | −0.467 | 1.00 | 27.21 | O |
| ATOM | 2511 | C | GLU | A | 328 | 90.837 | 54.924 | 3.024 | 1.00 | 10.91 | C |
| ATOM | 2512 | O | GLU | A | 328 | 91.116 | 53.811 | 2.544 | 1.00 | 9.33 | O |
| ATOM | 2513 | N | VAL | A | 329 | 91.636 | 55.559 | 3.872 | 1.00 | 10.66 | N |
| ATOM | 2514 | CA | VAL | A | 329 | 92.971 | 55.067 | 4.172 | 1.00 | 9.49 | C |
| ATOM | 2515 | CB | VAL | A | 329 | 93.865 | 56.225 | 4.666 | 1.00 | 9.29 | C |
| ATOM | 2516 | CG1 | VAL | A | 329 | 95.176 | 55.693 | 5.242 | 1.00 | 8.33 | C |
| ATOM | 2517 | CG2 | VAL | A | 329 | 94.116 | 57.210 | 3.515 | 1.00 | 8.07 | C |
| ATOM | 2518 | C | VAL | A | 329 | 92.999 | 53.892 | 5.166 | 1.00 | 10.93 | C |
| ATOM | 2519 | O | VAL | A | 329 | 93.745 | 52.937 | 4.975 | 1.00 | 13.97 | O |
| ATOM | 2520 | N | PHE | A | 330 | 92.216 | 53.993 | 6.235 | 1.00 | 10.30 | N |
| ATOM | 2521 | CA | PHE | A | 330 | 92.216 | 53.007 | 7.301 | 1.00 | 10.50 | C |
| ATOM | 2522 | CB | PHE | A | 330 | 92.300 | 53.736 | 8.663 | 1.00 | 10.10 | C |
| ATOM | 2523 | CG | PHE | A | 330 | 93.515 | 54.616 | 8.780 | 1.00 | 9.79 | C |
| ATOM | 2524 | CD1 | PHE | A | 330 | 94.783 | 54.057 | 8.743 | 1.00 | 9.81 | C |
| ATOM | 2525 | CE1 | PHE | A | 330 | 95.923 | 54.840 | 8.827 | 1.00 | 8.98 | C |
| ATOM | 2526 | CZ | PHE | A | 330 | 95.791 | 56.201 | 8.941 | 1.00 | 9.58 | C |
| ATOM | 2527 | CE2 | PHE | A | 330 | 94.535 | 56.770 | 8.980 | 1.00 | 9.73 | C |
| ATOM | 2528 | CD2 | PHE | A | 330 | 93.401 | 55.981 | 8.893 | 1.00 | 9.05 | C |
| ATOM | 2529 | C | PHE | A | 330 | 91.018 | 52.064 | 7.279 | 1.00 | 12.26 | C |
| ATOM | 2530 | O | PHE | A | 330 | 91.043 | 51.042 | 7.954 | 1.00 | 13.04 | O |
| ATOM | 2531 | N | GLY | A | 331 | 89.977 | 52.427 | 6.531 | 1.00 | 12.02 | N |
| ATOM | 2532 | CA | GLY | A | 331 | 88.811 | 51.589 | 6.370 | 1.00 | 13.60 | C |
| ATOM | 2533 | C | GLY | A | 331 | 89.013 | 50.578 | 5.263 | 1.00 | 15.34 | C |
| ATOM | 2534 | O | GLY | A | 331 | 90.153 | 50.314 | 4.815 | 1.00 | 16.11 | O |
| ATOM | 2535 | N | ARG | A | 332 | 87.899 | 50.022 | 4.807 | 1.00 | 16.06 | N |
| ATOM | 2536 | CA | ARG | A | 332 | 87.940 | 48.910 | 3.877 | 1.00 | 18.06 | C |
| ATOM | 2537 | CB | ARG | A | 332 | 86.544 | 48.342 | 3.630 | 1.00 | 24.83 | C |
| ATOM | 2538 | CG | ARG | A | 332 | 86.143 | 47.309 | 4.669 | 1.00 | 37.10 | C |
| ATOM | 2539 | CD | ARG | A | 332 | 84.849 | 46.605 | 4.285 | 1.00 | 48.85 | C |
| ATOM | 2540 | NE | ARG | A | 332 | 84.707 | 45.379 | 5.064 | 1.00 | 59.60 | N |
| ATOM | 2541 | CZ | ARG | A | 332 | 85.244 | 44.210 | 4.726 | 1.00 | 69.59 | C |
| ATOM | 2542 | NH1 | ARG | A | 332 | 85.950 | 44.078 | 3.602 | 1.00 | 78.32 | N |
| ATOM | 2543 | NH2 | ARG | A | 332 | 85.070 | 43.158 | 5.512 | 1.00 | 75.92 | N |
| ATOM | 2544 | C | ARG | A | 332 | 88.597 | 49.241 | 2.548 | 1.00 | 15.39 | C |
| ATOM | 2545 | O | ARG | A | 332 | 89.214 | 48.388 | 1.976 | 1.00 | 13.38 | O |
| ATOM | 2546 | N | LYS | A | 333 | 88.475 | 50.464 | 2.048 | 1.00 | 14.25 | N |
| ATOM | 2547 | CA | LYS | A | 333 | 89.137 | 50.798 | 0.780 | 1.00 | 16.66 | C |
| ATOM | 2548 | CB | LYS | A | 333 | 88.773 | 52.214 | 0.364 | 1.00 | 20.03 | C |
| ATOM | 2549 | CG | LYS | A | 333 | 89.086 | 52.561 | −1.079 | 1.00 | 30.01 | C |
| ATOM | 2550 | CD | LYS | A | 333 | 88.438 | 53.900 | −1.449 | 1.00 | 43.02 | C |
| ATOM | 2551 | CE | LYS | A | 333 | 88.714 | 54.319 | −2.889 | 1.00 | 44.84 | C |
| ATOM | 2552 | NZ | LYS | A | 333 | 90.177 | 54.396 | −3.107 | 1.00 | 44.14 | N |
| ATOM | 2553 | C | LYS | A | 333 | 90.676 | 50.616 | 0.857 | 1.00 | 13.92 | C |
| ATOM | 2554 | O | LYS | A | 333 | 91.304 | 50.272 | −0.117 | 1.00 | 11.72 | O |
| ATOM | 2555 | N | GLN | A | 334 | 91.266 | 50.808 | 2.029 | 1.00 | 10.94 | N |
| ATOM | 2556 | CA | GLN | A | 334 | 92.710 | 50.663 | 2.204 | 1.00 | 12.50 | C |
| ATOM | 2557 | CB | GLN | A | 334 | 93.165 | 49.183 | 2.116 | 1.00 | 14.64 | C |
| ATOM | 2558 | CG | GLN | A | 334 | 92.677 | 48.272 | 3.251 | 1.00 | 14.64 | C |
| ATOM | 2559 | CD | GLN | A | 334 | 93.307 | 48.623 | 4.579 | 1.00 | 15.76 | C |
| ATOM | 2560 | OE1 | GLN | A | 334 | 94.497 | 48.398 | 4.780 | 1.00 | 15.21 | O |
| ATOM | 2561 | NE2 | GLN | A | 334 | 92.509 | 49.191 | 5.494 | 1.00 | 14.54 | N |
| ATOM | 2562 | C | GLN | A | 334 | 93.491 | 51.529 | 1.192 | 1.00 | 10.81 | C |
| ATOM | 2563 | O | GLN | A | 334 | 94.433 | 51.069 | 0.572 | 1.00 | 9.71 | O |
| ATOM | 2564 | N | ALA | A | 335 | 93.103 | 52.776 | 1.032 | 1.00 | 10.60 | N |
| ATOM | 2565 | CA | ALA | A | 335 | 93.894 | 53.690 | 0.186 | 1.00 | 11.76 | C |
| ATOM | 2566 | CB | ALA | A | 335 | 93.136 | 55.015 | −0.060 | 1.00 | 12.19 | C |
| ATOM | 2567 | C | ALA | A | 335 | 95.275 | 53.937 | 0.832 | 1.00 | 9.79 | C |
| ATOM | 2568 | O | ALA | A | 335 | 95.458 | 53.776 | 2.039 | 1.00 | 9.13 | O |
| ATOM | 2569 | N | GLY | A | 336 | 96.267 | 54.247 | 0.011 | 1.00 | 9.47 | N |
| ATOM | 2570 | CA | GLY | A | 336 | 97.589 | 54.612 | 0.521 | 1.00 | 9.15 | C |
| ATOM | 2571 | C | GLY | A | 336 | 97.549 | 55.985 | 1.166 | 1.00 | 8.00 | C |
| ATOM | 2572 | O | GLY | A | 336 | 96.621 | 56.775 | 0.931 | 1.00 | 8.04 | O |
| ATOM | 2573 | N | PHE | A | 337 | 98.585 | 56.278 | 1.948 | 1.00 | 7.05 | N |
| ATOM | 2574 | CA | PHE | A | 337 | 98.704 | 57.540 | 2.647 | 1.00 | 7.59 | C |
| ATOM | 2575 | CB | PHE | A | 337 | 99.975 | 57.594 | 3.487 | 1.00 | 7.16 | C |
| ATOM | 2576 | CG | PHE | A | 337 | 99.808 | 57.104 | 4.865 | 1.00 | 7.21 | C |
| ATOM | 2577 | CD1 | PHE | A | 337 | 99.003 | 57.795 | 5.761 | 1.00 | 7.38 | C |
| ATOM | 2578 | CE1 | PHE | A | 337 | 98.864 | 57.374 | 7.066 | 1.00 | 8.33 | C |
| ATOM | 2579 | CZ | PHE | A | 337 | 99.567 | 56.259 | 7.505 | 1.00 | 8.60 | C |
| ATOM | 2580 | CE2 | PHE | A | 337 | 100.398 | 55.564 | 6.619 | 1.00 | 8.77 | C |
| ATOM | 2581 | CD2 | PHE | A | 337 | 100.508 | 55.990 | 5.308 | 1.00 | 8.62 | C |
| ATOM | 2582 | C | PHE | A | 337 | 98.696 | 58.776 | 1.757 | 1.00 | 7.99 | C |
| ATOM | 2583 | O | PHE | A | 337 | 98.430 | 59.861 | 2.268 | 1.00 | 7.70 | O |
| ATOM | 2584 | N | GLU | A | 338 | 98.992 | 58.618 | 0.467 | 1.00 | 8.15 | N |
| ATOM | 2585 | CA | GLU | A | 338 | 98.899 | 59.729 | −0.491 | 1.00 | 9.74 | C |
| ATOM | 2586 | CB | GLU | A | 338 | 99.244 | 59.294 | −1.947 | 1.00 | 9.63 | C |
| ATOM | 2587 | CG | GLU | A | 338 | 98.362 | 58.191 | −2.537 | 1.00 | 9.65 | C |
| ATOM | 2588 | CD | GLU | A | 338 | 98.856 | 56.762 | −2.257 | 1.00 | 10.53 | C |
| ATOM | 2589 | OE1 | GLU | A | 338 | 99.721 | 56.570 | −1.362 | 1.00 | 10.81 | O |
| ATOM | 2590 | OE2 | GLU | A | 338 | 98.390 | 55.817 | −2.944 | 1.00 | 10.79 | O |
| ATOM | 2591 | C | GLU | A | 338 | 97.513 | 60.415 | −0.436 | 1.00 | 10.37 | C |
| ATOM | 2592 | O | GLU | A | 338 | 97.409 | 61.601 | −0.684 | 1.00 | 10.60 | O |
| ATOM | 2593 | N | ARG | A | 339 | 96.468 | 59.666 | −0.110 | 1.00 | 10.40 | N |
| ATOM | 2594 | CA | ARG | A | 339 | 95.116 | 60.199 | 0.001 | 1.00 | 10.06 | C |
| ATOM | 2595 | CB | ARG | A | 339 | 94.153 | 59.043 | 0.312 | 1.00 | 11.83 | C |
| ATOM | 2596 | CG | ARG | A | 339 | 92.681 | 59.396 | 0.462 | 1.00 | 13.18 | C |
| ATOM | 2597 | CD | ARG | A | 339 | 92.134 | 60.106 | −0.771 | 1.00 | 14.10 | C |
| ATOM | 2598 | NE | ARG | A | 339 | 90.753 | 60.596 | −0.578 | 1.00 | 13.98 | N |
| ATOM | 2599 | CZ | ARG | A | 339 | 90.417 | 61.759 | −0.005 | 1.00 | 13.41 | C |
| ATOM | 2600 | NH1 | ARG | A | 339 | 91.332 | 62.597 | 0.458 | 1.00 | 13.50 | N |
| ATOM | 2601 | NH2 | ARG | A | 339 | 89.139 | 62.104 | 0.075 | 1.00 | 14.68 | N |
| ATOM | 2602 | C | ARG | A | 339 | 95.008 | 61.304 | 1.078 | 1.00 | 11.01 | C |
| ATOM | 2603 | O | ARG | A | 339 | 94.088 | 62.127 | 1.045 | 1.00 | 10.77 | O |
| ATOM | 2604 | N | MET | A | 340 | 95.966 | 61.358 | 2.006 | 1.00 | 10.31 | N |
| ATOM | 2605 | CA | MET | A | 340 | 96.020 | 62.457 | 2.963 | 1.00 | 9.26 | C |
| ATOM | 2606 | CB | MET | A | 340 | 96.993 | 62.146 | 4.099 | 1.00 | 9.11 | C |
| ATOM | 2607 | CG | MET | A | 340 | 96.646 | 60.889 | 4.879 | 1.00 | 10.01 | C |
| ATOM | 2608 | SD | MET | A | 340 | 94.987 | 60.980 | 5.573 | 1.00 | 12.13 | S |
| ATOM | 2609 | CE | MET | A | 340 | 95.008 | 59.506 | 6.613 | 1.00 | 10.09 | C |
| ATOM | 2610 | C | MET | A | 340 | 96.427 | 63.803 | 2.338 | 1.00 | 9.36 | C |
| ATOM | 2611 | O | MET | A | 340 | 96.074 | 64.848 | 2.842 | 1.00 | 8.38 | O |
| ATOM | 2612 | N | ILE | A | 341 | 97.157 | 63.781 | 1.235 | 1.00 | 11.09 | N |
| ATOM | 2613 | CA | ILE | A | 341 | 97.748 | 65.000 | 0.686 | 1.00 | 10.39 | C |
| ATOM | 2614 | CB | ILE | A | 341 | 98.657 | 64.687 | −0.527 | 1.00 | 10.79 | C |
| ATOM | 2615 | CG1 | ILE | A | 341 | 99.852 | 63.876 | −0.052 | 1.00 | 11.35 | C |
| ATOM | 2616 | CD1 | ILE | A | 341 | 100.648 | 63.174 | −1.145 | 1.00 | 11.56 | C |
| ATOM | 2617 | CG2 | ILE | A | 341 | 99.130 | 65.981 | −1.238 | 1.00 | 10.01 | C |
| ATOM | 2618 | C | ILE | A | 341 | 96.722 | 66.097 | 0.380 | 1.00 | 10.90 | C |
| ATOM | 2619 | O | ILE | A | 341 | 96.892 | 67.243 | 0.799 | 1.00 | 12.31 | O |
| ATOM | 2620 | N | PRO | A | 342 | 95.629 | 65.756 | −0.302 | 1.00 | 12.28 | N |
| ATOM | 2621 | CA | PRO | A | 342 | 94.676 | 66.832 | −0.590 | 1.00 | 12.47 | C |
| ATOM | 2622 | CB | PRO | A | 342 | 93.843 | 66.256 | −1.741 | 1.00 | 14.17 | C |
| ATOM | 2623 | CG | PRO | A | 342 | 93.910 | 64.782 | −1.539 | 1.00 | 13.44 | C |
| ATOM | 2624 | CD | PRO | A | 342 | 95.301 | 64.519 | −1.033 | 1.00 | 12.37 | C |
| ATOM | 2625 | C | PRO | A | 342 | 93.771 | 67.220 | 0.601 | 1.00 | 11.11 | C |
| ATOM | 2626 | O | PRO | A | 342 | 93.086 | 68.223 | 0.518 | 1.00 | 9.19 | O |
| ATOM | 2627 | N | ALA | A | 343 | 93.799 | 66.462 | 1.705 | 1.00 | 10.31 | N |
| ATOM | 2628 | CA | ALA | A | 343 | 92.963 | 66.766 | 2.872 | 1.00 | 9.59 | C |
| ATOM | 2629 | CB | ALA | A | 343 | 92.467 | 65.479 | 3.543 | 1.00 | 8.88 | C |
| ATOM | 2630 | C | ALA | A | 343 | 93.700 | 67.643 | 3.886 | 1.00 | 10.02 | C |
| ATOM | 2631 | O | ALA | A | 343 | 93.063 | 68.311 | 4.708 | 1.00 | 10.02 | O |
| ATOM | 2632 | N | LEU | A | 344 | 95.034 | 67.664 | 3.865 | 1.00 | 9.70 | N |
| ATOM | 2633 | CA | LEU | A | 344 | 95.772 | 68.381 | 4.934 | 1.00 | 8.81 | C |
| ATOM | 2634 | CB | LEU | A | 344 | 97.255 | 67.973 | 4.998 | 1.00 | 8.59 | C |
| ATOM | 2635 | CG | LEU | A | 344 | 97.518 | 66.577 | 5.587 | 1.00 | 8.97 | C |
| ATOM | 2636 | CD1 | LEU | A | 344 | 98.847 | 66.032 | 5.058 | 1.00 | 8.21 | C |
| ATOM | 2637 | CD2 | LEU | A | 344 | 97.450 | 66.546 | 7.108 | 1.00 | 7.43 | C |
| ATOM | 2638 | C | LEU | A | 344 | 95.657 | 69.916 | 4.874 | 1.00 | 9.98 | C |
| ATOM | 2639 | O | LEU | A | 344 | 95.549 | 70.562 | 5.911 | 1.00 | 8.75 | O |
| ATOM | 2640 | N | HIS | A | 345 | 95.711 | 70.505 | 3.678 | 1.00 | 9.35 | N |
| ATOM | 2641 | CA | HIS | A | 345 | 95.587 | 71.942 | 3.535 | 1.00 | 9.92 | C |
| ATOM | 2642 | CB | HIS | A | 345 | 94.129 | 72.401 | 3.757 | 1.00 | 10.02 | C |
| ATOM | 2643 | CG | HIS | A | 345 | 93.179 | 71.894 | 2.699 | 1.00 | 11.67 | C |
| ATOM | 2644 | ND1 | HIS | A | 345 | 93.205 | 72.350 | 1.424 | 1.00 | 12.62 | N |
| ATOM | 2645 | CE1 | HIS | A | 345 | 92.256 | 71.709 | 0.692 | 1.00 | 13.33 | C |
| ATOM | 2646 | NE2 | HIS | A | 345 | 91.648 | 70.814 | 1.496 | 1.00 | 14.96 | N |
| ATOM | 2647 | CD2 | HIS | A | 345 | 92.202 | 70.899 | 2.742 | 1.00 | 12.33 | C |
| ATOM | 2648 | C | HIS | A | 345 | 96.551 | 72.703 | 4.428 | 1.00 | 9.82 | C |
| ATOM | 2649 | O | HIS | A | 345 | 96.178 | 73.689 | 5.069 | 1.00 | 8.43 | O |
| ATOM | 2650 | N | LEU | A | 346 | 97.816 | 72.289 | 4.423 | 1.00 | 9.30 | N |
| ATOM | 2651 | CA | LEU | A | 346 | 98.818 | 72.912 | 5.300 | 1.00 | 10.80 | C |
| ATOM | 2652 | CB | LEU | A | 346 | 100.117 | 72.093 | 5.298 | 1.00 | 10.45 | C |
| ATOM | 2653 | CG | LEU | A | 346 | 99.976 | 70.661 | 5.826 | 1.00 | 11.00 | C |
| ATOM | 2654 | CD1 | LEU | A | 346 | 101.354 | 70.006 | 5.726 | 1.00 | 8.88 | C |
| ATOM | 2655 | CD2 | LEU | A | 346 | 99.373 | 70.612 | 7.255 | 1.00 | 8.53 | C |
| ATOM | 2656 | C | LEU | A | 346 | 99.139 | 74.368 | 4.902 | 1.00 | 11.13 | C |
| ATOM | 2657 | O | LEU | A | 346 | 99.466 | 75.186 | 5.758 | 1.00 | 12.08 | O |
| ATOM | 2658 | N | THR | A | 347 | 99.068 | 74.668 | 3.610 | 1.00 | 10.80 | N |
| ATOM | 2659 | CA | THR | A | 347 | 99.282 | 76.010 | 3.112 | 1.00 | 11.97 | C |
| ATOM | 2660 | CB | THR | A | 347 | 100.670 | 76.166 | 2.491 | 1.00 | 13.64 | C |
| ATOM | 2661 | OG1 | THR | A | 347 | 100.818 | 75.232 | 1.414 | 1.00 | 13.71 | O |
| ATOM | 2662 | CG2 | THR | A | 347 | 101.794 | 75.970 | 3.528 | 1.00 | 11.95 | C |
| ATOM | 2663 | C | THR | A | 347 | 98.234 | 76.304 | 2.045 | 1.00 | 13.99 | C |
| ATOM | 2664 | O | THR | A | 347 | 97.624 | 75.386 | 1.488 | 1.00 | 12.36 | O |
| ATOM | 2665 | N | GLY | A | 348 | 97.988 | 77.590 | 1.800 | 1.00 | 15.02 | N |
| ATOM | 2666 | CA | GLY | A | 348 | 97.083 | 78.010 | 0.745 | 1.00 | 15.65 | C |
| ATOM | 2667 | C | GLY | A | 348 | 95.579 | 77.924 | 1.069 | 1.00 | 19.22 | C |
| ATOM | 2668 | O | GLY | A | 348 | 94.776 | 78.001 | 0.171 | 1.00 | 17.83 | O |
| ATOM | 2669 | N | GLY | A | 349 | 95.173 | 77.792 | 2.333 | 1.00 | 18.00 | N |
| ATOM | 2670 | CA | GLY | A | 349 | 93.734 | 77.951 | 2.676 | 1.00 | 14.01 | C |
| ATOM | 2671 | C | GLY | A | 349 | 93.073 | 76.615 | 2.480 | 1.00 | 14.38 | C |
| ATOM | 2672 | O | GLY | A | 349 | 93.752 | 75.667 | 2.099 | 1.00 | 17.92 | O |
| ATOM | 2673 | N | PRO | A | 350 | 91.737 | 76.537 | 2.633 | 1.00 | 12.78 | N |
| ATOM | 2674 | CA | PRO | A | 350 | 90.787 | 77.659 | 2.748 | 1.00 | 11.55 | C |
| ATOM | 2675 | CB | PRO | A | 350 | 89.556 | 77.130 | 2.031 | 1.00 | 12.62 | C |
| ATOM | 2676 | CG | PRO | A | 350 | 89.586 | 75.643 | 2.346 | 1.00 | 13.72 | C |
| ATOM | 2677 | CD | PRO | A | 350 | 91.061 | 75.279 | 2.260 | 1.00 | 13.72 | C |
| ATOM | 2678 | C | PRO | A | 350 | 90.440 | 78.055 | 4.177 | 1.00 | 10.05 | C |
| ATOM | 2679 | O | PRO | A | 350 | 89.797 | 79.087 | 4.398 | 1.00 | 9.27 | O |
| ATOM | 2680 | N | TYR | A | 351 | 90.862 | 77.244 | 5.135 | 1.00 | 9.71 | N |
| ATOM | 2681 | CA | TYR | A | 351 | 90.567 | 77.499 | 6.539 | 1.00 | 10.28 | C |
| ATOM | 2682 | CB | TYR | A | 351 | 90.875 | 76.267 | 7.384 | 1.00 | 10.43 | C |
| ATOM | 2683 | CG | TYR | A | 351 | 90.321 | 74.951 | 6.885 | 1.00 | 11.04 | C |
| ATOM | 2684 | CD1 | TYR | A | 351 | 89.002 | 74.613 | 7.113 | 1.00 | 10.28 | C |
| ATOM | 2685 | CE1 | TYR | A | 351 | 88.485 | 73.407 | 6.679 | 1.00 | 10.77 | C |
| ATOM | 2686 | CZ | TYR | A | 351 | 89.287 | 72.508 | 6.017 | 1.00 | 11.60 | C |
| ATOM | 2687 | OH | TYR | A | 351 | 88.757 | 71.315 | 5.623 | 1.00 | 10.91 | O |
| ATOM | 2688 | CE2 | TYR | A | 351 | 90.616 | 72.818 | 5.757 | 1.00 | 11.66 | C |
| ATOM | 2689 | CD2 | TYR | A | 351 | 91.122 | 74.042 | 6.190 | 1.00 | 10.88 | C |
| ATOM | 2690 | C | TYR | A | 351 | 91.384 | 78.653 | 7.097 | 1.00 | 10.89 | C |
| ATOM | 2691 | O | TYR | A | 351 | 92.554 | 78.838 | 6.698 | 1.00 | 12.30 | O |
| ATOM | 2692 | N | ALA | A | 352 | 90.819 | 79.392 | 8.057 | 1.00 | 9.49 | N |
| ATOM | 2693 | CA | ALA | A | 352 | 91.595 | 80.444 | 8.729 | 1.00 | 10.18 | C |
| ATOM | 2694 | CB | ALA | A | 352 | 90.810 | 81.124 | 9.847 | 1.00 | 8.72 | C |
| ATOM | 2695 | C | ALA | A | 352 | 92.863 | 79.803 | 9.282 | 1.00 | 11.77 | C |
| ATOM | 2696 | O | ALA | A | 352 | 93.935 | 80.370 | 9.171 | 1.00 | 11.42 | O |
| ATOM | 2697 | N | ASN | A | 353 | 92.752 | 78.613 | 9.878 | 1.00 | 10.81 | N |
| ATOM | 2698 | CA | ASN | A | 353 | 93.946 | 77.953 | 10.423 | 1.00 | 9.81 | C |
| ATOM | 2699 | CB | ASN | A | 353 | 94.346 | 78.587 | 11.759 | 1.00 | 10.38 | C |
| ATOM | 2700 | CG | ASN | A | 353 | 95.666 | 78.059 | 12.290 | 1.00 | 10.94 | C |
| ATOM | 2701 | OD1 | ASN | A | 353 | 95.939 | 76.857 | 12.206 | 1.00 | 10.93 | O |
| ATOM | 2702 | ND2 | ASN | A | 353 | 96.494 | 78.954 | 12.874 | 1.00 | 8.59 | N |
| ATOM | 2703 | C | ASN | A | 353 | 93.701 | 76.475 | 10.528 | 1.00 | 8.44 | C |
| ATOM | 2704 | O | ASN | A | 353 | 92.907 | 76.019 | 11.361 | 1.00 | 10.00 | O |
| ATOM | 2705 | N | PRO | A | 354 | 94.354 | 75.697 | 9.661 | 1.00 | 8.94 | N |
| ATOM | 2706 | CA | PRO | A | 354 | 94.054 | 74.251 | 9.635 | 1.00 | 8.55 | C |
| ATOM | 2707 | CB | PRO | A | 354 | 94.814 | 73.777 | 8.414 | 1.00 | 8.43 | C |
| ATOM | 2708 | CG | PRO | A | 354 | 96.007 | 74.682 | 8.355 | 1.00 | 8.58 | C |
| ATOM | 2709 | CD | PRO | A | 354 | 95.475 | 76.039 | 8.758 | 1.00 | 8.26 | C |
| ATOM | 2710 | C | PRO | A | 354 | 94.518 | 73.521 | 10.889 | 1.00 | 8.60 | C |
| ATOM | 2711 | O | PRO | A | 354 | 94.028 | 72.431 | 11.192 | 1.00 | 8.91 | O |
| ATOM | 2712 | N | TYR | A | 355 | 95.429 | 74.128 | 11.645 | 1.00 | 8.49 | N |
| ATOM | 2713 | CA | TYR | A | 355 | 95.945 | 73.520 | 12.889 | 1.00 | 7.59 | C |
| ATOM | 2714 | CB | TYR | A | 355 | 97.283 | 74.172 | 13.277 | 1.00 | 7.82 | C |
| ATOM | 2715 | CG | TYR | A | 355 | 98.365 | 73.932 | 12.243 | 1.00 | 7.26 | C |
| ATOM | 2716 | CD1 | TYR | A | 355 | 99.166 | 72.802 | 12.299 | 1.00 | 8.70 | C |
| ATOM | 2717 | CE1 | TYR | A | 355 | 100.141 | 72.558 | 11.356 | 1.00 | 8.41 | C |
| ATOM | 2718 | CZ | TYR | A | 355 | 100.321 | 73.451 | 10.338 | 1.00 | 8.87 | C |
| ATOM | 2719 | OH | TYR | A | 355 | 101.272 | 73.201 | 9.390 | 1.00 | 8.82 | O |
| ATOM | 2720 | CE2 | TYR | A | 355 | 99.522 | 74.584 | 10.250 | 1.00 | 8.37 | C |
| ATOM | 2721 | CD2 | TYR | A | 355 | 98.554 | 74.801 | 11.207 | 1.00 | 7.94 | C |
| ATOM | 2722 | C | TYR | A | 355 | 94.928 | 73.573 | 14.058 | 1.00 | 8.97 | C |
| ATOM | 2723 | O | TYR | A | 355 | 95.127 | 72.918 | 15.095 | 1.00 | 8.72 | O |
| ATOM | 2724 | N | GLU | A | 356 | 93.824 | 74.298 | 13.866 | 1.00 | 9.38 | N |
| ATOM | 2725 | CA | GLU | A | 356 | 92.786 | 74.426 | 14.891 | 1.00 | 10.40 | C |
| ATOM | 2726 | CB | GLU | A | 356 | 92.372 | 75.889 | 15.031 | 1.00 | 11.74 | C |
| ATOM | 2727 | CG | GLU | A | 356 | 93.511 | 76.789 | 15.465 | 1.00 | 14.48 | C |
| ATOM | 2728 | CD | GLU | A | 356 | 93.179 | 78.287 | 15.344 | 1.00 | 17.27 | C |
| ATOM | 2729 | OE1 | GLU | A | 356 | 92.031 | 78.661 | 14.974 | 1.00 | 18.33 | O |
| ATOM | 2730 | OE2 | GLU | A | 356 | 94.097 | 79.084 | 15.586 | 1.00 | 14.75 | O |
| ATOM | 2731 | C | GLU | A | 356 | 91.545 | 73.584 | 14.592 | 1.00 | 9.45 | C |
| ATOM | 2732 | O | GLU | A | 356 | 90.577 | 73.580 | 15.362 | 1.00 | 10.71 | O |
| ATOM | 2733 | N | LEU | A | 357 | 91.567 | 72.856 | 13.488 | 1.00 | 9.76 | N |
| ATOM | 2734 | CA | LEU | A | 357 | 90.419 | 72.017 | 13.127 | 1.00 | 8.67 | C |
| ATOM | 2735 | CB | LEU | A | 357 | 90.563 | 71.482 | 11.717 | 1.00 | 8.92 | C |
| ATOM | 2736 | CG | LEU | A | 357 | 90.652 | 72.544 | 10.624 | 1.00 | 9.86 | C |
| ATOM | 2737 | CD1 | LEU | A | 357 | 90.900 | 71.810 | 9.301 | 1.00 | 10.02 | C |
| ATOM | 2738 | CD2 | LEU | A | 357 | 89.411 | 73.427 | 10.599 | 1.00 | 9.32 | C |
| ATOM | 2739 | C | LEU | A | 357 | 90.306 | 70.837 | 14.078 | 1.00 | 9.42 | C |
| ATOM | 2740 | O | LEU | A | 357 | 91.325 | 70.301 | 14.551 | 1.00 | 8.12 | O |
| ATOM | 2741 | N | ALA | A | 358 | 89.063 | 70.443 | 14.359 | 1.00 | 9.05 | N |
| ATOM | 2742 | CA | ALA | A | 358 | 88.783 | 69.275 | 15.207 | 1.00 | 8.40 | C |
| ATOM | 2743 | CB | ALA | A | 358 | 87.441 | 69.448 | 15.877 | 1.00 | 8.38 | C |
| ATOM | 2744 | C | ALA | A | 358 | 88.776 | 68.079 | 14.272 | 1.00 | 9.28 | C |
| ATOM | 2745 | O | ALA | A | 358 | 87.914 | 67.989 | 13.357 | 1.00 | 9.07 | O |
| ATOM | 2746 | N | THR | A | 359 | 89.745 | 67.186 | 14.445 | 1.00 | 9.68 | N |
| ATOM | 2747 | CA | THR | A | 359 | 89.871 | 66.034 | 13.554 | 1.00 | 8.81 | C |
| ATOM | 2748 | CB | THR | A | 359 | 91.347 | 65.763 | 13.184 | 1.00 | 8.14 | C |
| ATOM | 2749 | OG1 | THR | A | 359 | 92.120 | 65.685 | 14.377 | 1.00 | 8.36 | O |
| ATOM | 2750 | CG2 | THR | A | 359 | 91.922 | 66.886 | 12.295 | 1.00 | 8.45 | C |
| ATOM | 2751 | C | THR | A | 359 | 89.215 | 64.776 | 14.152 | 1.00 | 8.41 | C |
| ATOM | 2752 | O | THR | A | 359 | 89.174 | 64.575 | 15.377 | 1.00 | 9.75 | O |
| ATOM | 2753 | N | PHE | A | 360 | 88.696 | 63.931 | 13.272 | 1.00 | 8.08 | N |
| ATOM | 2754 | CA | PHE | A | 360 | 87.965 | 62.713 | 13.634 | 1.00 | 7.27 | C |
| ATOM | 2755 | CB | PHE | A | 360 | 86.542 | 63.070 | 14.107 | 1.00 | 7.53 | C |
| ATOM | 2756 | CG | PHE | A | 360 | 85.627 | 63.576 | 13.005 | 1.00 | 8.76 | C |
| ATOM | 2757 | CD1 | PHE | A | 360 | 85.772 | 64.853 | 12.472 | 1.00 | 9.54 | C |
| ATOM | 2758 | CE1 | PHE | A | 360 | 84.940 | 65.302 | 11.459 | 1.00 | 9.36 | C |
| ATOM | 2759 | CZ | PHE | A | 360 | 83.921 | 64.491 | 10.976 | 1.00 | 9.69 | C |
| ATOM | 2760 | CE2 | PHE | A | 360 | 83.752 | 63.231 | 11.506 | 1.00 | 9.12 | C |
| ATOM | 2761 | CD2 | PHE | A | 360 | 84.598 | 62.787 | 12.523 | 1.00 | 9.65 | C |
| ATOM | 2762 | C | PHE | A | 360 | 87.922 | 61.769 | 12.425 | 1.00 | 7.67 | C |
| ATOM | 2763 | O | PHE | A | 360 | 88.170 | 62.194 | 11.276 | 1.00 | 7.78 | O |
| ATOM | 2764 | N | TYR | A | 361 | 87.609 | 60.498 | 12.668 | 1.00 | 7.49 | N |
| ATOM | 2765 | CA | TYR | A | 361 | 87.405 | 59.524 | 11.599 | 1.00 | 7.70 | C |
| ATOM | 2766 | CB | TYR | A | 361 | 88.494 | 58.425 | 11.591 | 1.00 | 8.98 | C |
| ATOM | 2767 | CG | TYR | A | 361 | 88.562 | 57.653 | 12.867 | 1.00 | 9.92 | C |
| ATOM | 2768 | CD1 | TYR | A | 361 | 87.638 | 56.658 | 13.140 | 1.00 | 9.78 | C |
| ATOM | 2769 | CE1 | TYR | A | 361 | 87.670 | 55.964 | 14.326 | 1.00 | 9.23 | C |
| ATOM | 2770 | CZ | TYR | A | 361 | 88.631 | 56.253 | 15.252 | 1.00 | 11.11 | C |
| ATOM | 2771 | OH | TYR | A | 361 | 88.674 | 55.559 | 16.424 | 1.00 | 9.75 | O |
| ATOM | 2772 | CE2 | TYR | A | 361 | 89.580 | 57.240 | 15.015 | 1.00 | 10.68 | C |
| ATOM | 2773 | CD2 | TYR | A | 361 | 89.540 | 57.930 | 13.820 | 1.00 | 10.41 | C |
| ATOM | 2774 | C | TYR | A | 361 | 86.008 | 58.900 | 11.611 | 1.00 | 8.19 | C |
| ATOM | 2775 | O | TYR | A | 361 | 85.647 | 58.252 | 10.666 | 1.00 | 7.67 | O |
| ATOM | 2776 | N | ASP | A | 362 | 85.234 | 59.057 | 12.686 | 1.00 | 8.73 | N |
| ATOM | 2777 | CA | ASP | A | 362 | 83.824 | 58.657 | 12.646 | 1.00 | 8.35 | C |
| ATOM | 2778 | CB | ASP | A | 362 | 83.653 | 57.128 | 12.753 | 1.00 | 8.07 | C |
| ATOM | 2779 | CG | ASP | A | 362 | 84.086 | 56.555 | 14.113 | 1.00 | 8.16 | C |
| ATOM | 2780 | OD1 | ASP | A | 362 | 84.234 | 57.344 | 15.073 | 1.00 | 9.60 | O |
| ATOM | 2781 | OD2 | ASP | A | 362 | 84.268 | 55.295 | 14.204 | 1.00 | 7.55 | O |
| ATOM | 2782 | C | ASP | A | 362 | 83.094 | 59.398 | 13.755 | 1.00 | 8.22 | C |
| ATOM | 2783 | O | ASP | A | 362 | 83.690 | 60.184 | 14.497 | 1.00 | 8.44 | O |
| ATOM | 2784 | N | ASN | A | 363 | 81.797 | 59.191 | 13.826 | 1.00 | 8.18 | N |
| ATOM | 2785 | CA | ASN | A | 363 | 80.986 | 59.838 | 14.845 | 1.00 | 9.19 | C |
| ATOM | 2786 | CB | ASN | A | 363 | 80.909 | 61.380 | 14.651 | 1.00 | 8.52 | C |
| ATOM | 2787 | CG | ASN | A | 363 | 80.095 | 61.816 | 13.415 | 1.00 | 9.77 | C |
| ATOM | 2788 | OD1 | ASN | A | 363 | 79.273 | 61.067 | 12.890 | 1.00 | 9.07 | O |
| ATOM | 2789 | ND2 | ASN | A | 363 | 80.352 | 63.063 | 12.944 | 1.00 | 9.67 | N |
| ATOM | 2790 | C | ASN | A | 363 | 79.624 | 59.146 | 14.955 | 1.00 | 7.68 | C |
| ATOM | 2791 | O | ASN | A | 363 | 79.439 | 58.048 | 14.427 | 1.00 | 8.21 | O |
| ATOM | 2792 | N | HIS | A | 364 | 78.701 | 59.804 | 15.637 | 1.00 | 8.34 | N |
| ATOM | 2793 | CA | HIS | A | 364 | 77.354 | 59.308 | 15.913 | 1.00 | 8.10 | C |
| ATOM | 2794 | CB | HIS | A | 364 | 76.786 | 59.985 | 17.176 | 1.00 | 7.63 | C |
| ATOM | 2795 | CG | HIS | A | 364 | 76.772 | 61.502 | 17.124 | 1.00 | 8.17 | C |
| ATOM | 2796 | ND1 | HIS | A | 364 | 77.735 | 62.229 | 16.507 | 1.00 | 8.37 | N |
| ATOM | 2797 | CE1 | HIS | A | 364 | 77.454 | 63.538 | 16.646 | 1.00 | 8.63 | C |
| ATOM | 2798 | NE2 | HIS | A | 364 | 76.343 | 63.653 | 17.371 | 1.00 | 7.82 | N |
| ATOM | 2799 | CD2 | HIS | A | 364 | 75.886 | 62.415 | 17.664 | 1.00 | 8.46 | C |
| ATOM | 2800 | C | HIS | A | 364 | 76.371 | 59.448 | 14.799 | 1.00 | 8.51 | C |
| ATOM | 2801 | O | HIS | A | 364 | 75.255 | 58.965 | 14.915 | 1.00 | 9.11 | O |
| ATOM | 2802 | N | ASP | A | 365 | 76.741 | 60.115 | 13.712 | 1.00 | 9.46 | N |
| ATOM | 2803 | CA | ASP | A | 365 | 75.817 | 60.414 | 12.593 | 1.00 | 9.11 | C |
| ATOM | 2804 | CB | ASP | A | 365 | 75.830 | 61.915 | 12.323 | 1.00 | 10.88 | C |
| ATOM | 2805 | CG | ASP | A | 365 | 75.312 | 62.724 | 13.500 | 1.00 | 10.65 | C |
| ATOM | 2806 | OD1 | ASP | A | 365 | 74.346 | 62.279 | 14.153 | 1.00 | 9.96 | O |
| ATOM | 2807 | OD2 | ASP | A | 365 | 75.844 | 63.818 | 13.718 | 1.00 | 11.29 | O |
| ATOM | 2808 | C | ASP | A | 365 | 76.192 | 59.734 | 11.274 | 1.00 | 9.75 | C |
| ATOM | 2809 | O | ASP | A | 365 | 75.760 | 60.176 | 10.189 | 1.00 | 11.96 | O |
| ATOM | 2810 | N | MET | A | 366 | 77.034 | 58.714 | 11.372 | 1.00 | 9.99 | N |
| ATOM | 2811 | CA | MET | A | 366 | 77.556 | 57.964 | 10.237 | 1.00 | 10.18 | C |
| ATOM | 2812 | CB | MET | A | 366 | 78.713 | 58.741 | 9.578 | 1.00 | 9.75 | C |
| ATOM | 2813 | CG | MET | A | 366 | 79.959 | 58.872 | 10.461 | 1.00 | 11.62 | C |
| ATOM | 2814 | SD | MET | A | 366 | 81.295 | 59.914 | 9.803 | 1.00 | 13.99 | S |
| ATOM | 2815 | CE | MET | A | 366 | 80.479 | 61.530 | 9.682 | 1.00 | 8.71 | C |
| ATOM | 2816 | C | MET | A | 366 | 78.049 | 56.598 | 10.762 | 1.00 | 9.78 | C |
| ATOM | 2817 | O | MET | A | 366 | 78.186 | 56.414 | 11.992 | 1.00 | 12.65 | O |
| ATOM | 2818 | N | PRO | A | 367 | 78.325 | 55.653 | 9.862 | 1.00 | 9.51 | N |
| ATOM | 2819 | CA | PRO | A | 367 | 78.852 | 54.387 | 10.374 | 1.00 | 10.78 | C |
| ATOM | 2820 | CB | PRO | A | 367 | 79.045 | 53.527 | 9.101 | 1.00 | 12.65 | C |
| ATOM | 2821 | CG | PRO | A | 367 | 78.001 | 54.079 | 8.125 | 1.00 | 13.10 | C |
| ATOM | 2822 | CD | PRO | A | 367 | 77.978 | 55.576 | 8.420 | 1.00 | 10.75 | C |
| ATOM | 2823 | C | PRO | A | 367 | 80.160 | 54.582 | 11.122 | 1.00 | 11.09 | C |
| ATOM | 2824 | O | PRO | A | 367 | 80.975 | 55.408 | 10.731 | 1.00 | 10.60 | O |
| ATOM | 2825 | N | ARG | A | 368 | 80.349 | 53.859 | 12.216 | 1.00 | 9.20 | N |
| ATOM | 2826 | CA | ARG | A | 368 | 81.663 | 53.831 | 12.827 | 1.00 | 9.49 | C |
| ATOM | 2827 | CB | ARG | A | 368 | 81.643 | 53.025 | 14.110 | 1.00 | 9.30 | C |
| ATOM | 2828 | CG | ARG | A | 368 | 81.025 | 53.784 | 15.274 | 1.00 | 10.19 | C |
| ATOM | 2829 | CD | ARG | A | 368 | 80.882 | 52.871 | 16.469 | 1.00 | 10.27 | C |
| ATOM | 2830 | NE | ARG | A | 368 | 80.243 | 53.454 | 17.672 | 1.00 | 9.66 | N |
| ATOM | 2831 | CZ | ARG | A | 368 | 80.903 | 53.947 | 18.717 | 1.00 | 9.05 | C |
| ATOM | 2832 | NH1 | ARG | A | 368 | 82.245 | 54.025 | 18.713 | 1.00 | 10.32 | N |
| ATOM | 2833 | NH2 | ARG | A | 368 | 80.231 | 54.339 | 19.787 | 1.00 | 8.12 | N |
| ATOM | 2834 | C | ARG | A | 368 | 82.663 | 53.249 | 11.845 | 1.00 | 11.61 | C |
| ATOM | 2835 | O | ARG | A | 368 | 82.279 | 52.520 | 10.912 | 1.00 | 11.13 | O |
| ATOM | 2836 | N | LEU | A | 369 | 83.943 | 53.540 | 12.074 | 1.00 | 13.78 | N |
| ATOM | 2837 | CA | LEU | A | 369 | 85.014 | 53.049 | 11.196 | 1.00 | 12.69 | C |
| ATOM | 2838 | CB | LEU | A | 369 | 86.382 | 53.395 | 11.783 | 1.00 | 10.93 | C |
| ATOM | 2839 | CG | LEU | A | 369 | 87.618 | 53.010 | 10.941 | 1.00 | 12.41 | C |
| ATOM | 2840 | CD1 | LEU | A | 369 | 87.665 | 53.862 | 9.662 | 1.00 | 11.24 | C |
| ATOM | 2841 | CD2 | LEU | A | 369 | 88.881 | 53.165 | 11.794 | 1.00 | 10.18 | C |
| ATOM | 2842 | C | LEU | A | 369 | 84.897 | 51.539 | 11.041 | 1.00 | 13.30 | C |
| ATOM | 2843 | O | LEU | A | 369 | 84.695 | 50.826 | 12.030 | 1.00 | 12.35 | O |
| ATOM | 2844 | N | ASP | A | 370 | 84.991 | 51.063 | 9.800 | 1.00 | 12.59 | N |
| ATOM | 2845 | CA | ASP | A | 370 | 84.883 | 49.637 | 9.504 | 1.00 | 12.43 | C |
| ATOM | 2846 | CB | ASP | A | 370 | 83.975 | 49.453 | 8.284 | 1.00 | 13.60 | C |
| ATOM | 2847 | CG | ASP | A | 370 | 83.785 | 47.969 | 7.870 | 1.00 | 17.03 | C |
| ATOM | 2848 | OD1 | ASP | A | 370 | 84.111 | 47.032 | 8.611 | 1.00 | 17.52 | O |
| ATOM | 2849 | OD2 | ASP | A | 370 | 83.322 | 47.754 | 6.752 | 1.00 | 19.77 | O |
| ATOM | 2850 | C | ASP | A | 370 | 86.314 | 49.112 | 9.286 | 1.00 | 13.54 | C |
| ATOM | 2851 | O | ASP | A | 370 | 86.820 | 49.088 | 8.175 | 1.00 | 11.76 | O |
| ATOM | 2852 | N | ALA | A | 371 | 86.967 | 48.720 | 10.375 | 1.00 | 13.86 | N |
| ATOM | 2853 | CA | ALA | A | 371 | 88.379 | 48.397 | 10.352 | 1.00 | 13.56 | C |
| ATOM | 2854 | CB | ALA | A | 371 | 89.208 | 49.607 | 10.786 | 1.00 | 15.22 | C |
| ATOM | 2855 | C | ALA | A | 371 | 88.647 | 47.237 | 11.284 | 1.00 | 13.93 | C |
| ATOM | 2856 | O | ALA | A | 371 | 87.898 | 47.002 | 12.234 | 1.00 | 12.26 | O |
| ATOM | 2857 | N | SER | A | 372 | 89.720 | 46.514 | 10.984 | 1.00 | 12.45 | N |
| ATOM | 2858 | CA | SER | A | 372 | 90.307 | 45.555 | 11.892 | 1.00 | 11.87 | C |
| ATOM | 2859 | CB | SER | A | 372 | 91.462 | 44.823 | 11.180 | 1.00 | 11.70 | C |
| ATOM | 2860 | OG | SER | A | 372 | 92.501 | 45.791 | 10.948 | 1.00 | 11.05 | O |
| ATOM | 2861 | C | SER | A | 372 | 90.908 | 46.311 | 13.099 | 1.00 | 12.28 | C |
| ATOM | 2862 | O | SER | A | 372 | 90.999 | 47.544 | 13.102 | 1.00 | 10.61 | O |
| ATOM | 2863 | N | ASP | A | 373 | 91.346 | 45.560 | 14.108 | 1.00 | 12.03 | N |
| ATOM | 2864 | CA | ASP | A | 373 | 91.992 | 46.153 | 15.276 | 1.00 | 13.25 | C |
| ATOM | 2865 | CB | ASP | A | 373 | 92.475 | 45.078 | 16.247 | 1.00 | 13.20 | C |
| ATOM | 2866 | CG | ASP | A | 373 | 91.370 | 44.539 | 17.148 | 1.00 | 14.89 | C |
| ATOM | 2867 | OD1 | ASP | A | 373 | 90.166 | 44.703 | 16.853 | 1.00 | 12.62 | O |
| ATOM | 2868 | OD2 | ASP | A | 373 | 91.736 | 43.978 | 18.190 | 1.00 | 13.86 | O |
| ATOM | 2869 | C | ASP | A | 373 | 93.198 | 46.988 | 14.842 | 1.00 | 12.53 | C |
| ATOM | 2870 | O | ASP | A | 373 | 93.423 | 48.093 | 15.337 | 1.00 | 10.18 | O |
| ATOM | 2871 | N | GLU | A | 374 | 93.959 | 46.427 | 13.921 | 1.00 | 11.93 | N |
| ATOM | 2872 | CA | GLU | A | 374 | 95.127 | 47.082 | 13.354 | 1.00 | 11.52 | C |
| ATOM | 2873 | CB | GLU | A | 374 | 95.789 | 46.152 | 12.336 | 1.00 | 14.06 | C |
| ATOM | 2874 | CG | GLU | A | 374 | 96.440 | 44.888 | 12.889 | 1.00 | 17.40 | C |
| ATOM | 2875 | CD | GLU | A | 374 | 95.456 | 43.815 | 13.375 | 1.00 | 23.88 | C |
| ATOM | 2876 | OE1 | GLU | A | 374 | 94.275 | 43.768 | 12.949 | 1.00 | 21.21 | O |
| ATOM | 2877 | OE2 | GLU | A | 374 | 95.874 | 43.018 | 14.222 | 1.00 | 29.19 | O |
| ATOM | 2878 | C | GLU | A | 374 | 94.777 | 48.408 | 12.683 | 1.00 | 11.68 | C |
| ATOM | 2879 | O | GLU | A | 374 | 95.514 | 49.379 | 12.804 | 1.00 | 13.01 | O |
| ATOM | 2880 | N | GLY | A | 375 | 93.653 | 48.458 | 11.971 | 1.00 | 9.99 | N |
| ATOM | 2881 | CA | GLY | A | 375 | 93.163 | 49.687 | 11.351 | 1.00 | 10.09 | C |
| ATOM | 2882 | C | GLY | A | 375 | 92.830 | 50.784 | 12.365 | 1.00 | 10.49 | C |
| ATOM | 2883 | O | GLY | A | 375 | 93.176 | 51.939 | 12.163 | 1.00 | 10.04 | O |
| ATOM | 2884 | N | PHE | A | 376 | 92.158 | 50.427 | 13.452 | 1.00 | 10.02 | N |
| ATOM | 2885 | CA | PHE | A | 376 | 91.932 | 51.386 | 14.536 | 1.00 | 10.75 | C |
| ATOM | 2886 | CB | PHE | A | 376 | 91.070 | 50.792 | 15.656 | 1.00 | 8.72 | C |
| ATOM | 2887 | CG | PHE | A | 376 | 89.603 | 50.807 | 15.347 | 1.00 | 8.79 | C |
| ATOM | 2888 | CD1 | PHE | A | 376 | 88.999 | 49.749 | 14.705 | 1.00 | 9.04 | C |
| ATOM | 2889 | CE1 | PHE | A | 376 | 87.652 | 49.776 | 14.390 | 1.00 | 9.80 | C |
| ATOM | 2890 | CZ | PHE | A | 376 | 86.894 | 50.874 | 14.744 | 1.00 | 10.46 | C |
| ATOM | 2891 | CE2 | PHE | A | 376 | 87.487 | 51.922 | 15.413 | 1.00 | 9.77 | C |
| ATOM | 2892 | CD2 | PHE | A | 376 | 88.831 | 51.886 | 15.703 | 1.00 | 8.64 | C |
| ATOM | 2893 | C | PHE | A | 376 | 93.267 | 51.884 | 15.092 | 1.00 | 8.86 | C |
| ATOM | 2894 | O | PHE | A | 376 | 93.442 | 53.081 | 15.344 | 1.00 | 9.11 | O |
| ATOM | 2895 | N | ILE | A | 377 | 94.204 | 50.971 | 15.298 | 1.00 | 8.28 | N |
| ATOM | 2896 | CA | ILE | A | 377 | 95.513 | 51.362 | 15.839 | 1.00 | 9.19 | C |
| ATOM | 2897 | CB | ILE | A | 377 | 96.411 | 50.113 | 16.082 | 1.00 | 8.69 | C |
| ATOM | 2898 | CG1 | ILE | A | 377 | 95.916 | 49.352 | 17.324 | 1.00 | 9.02 | C |
| ATOM | 2899 | CD1 | ILE | A | 377 | 96.440 | 47.927 | 17.475 | 1.00 | 9.66 | C |
| ATOM | 2900 | CG2 | ILE | A | 377 | 97.866 | 50.532 | 16.255 | 1.00 | 8.80 | C |
| ATOM | 2901 | C | ILE | A | 377 | 96.200 | 52.391 | 14.910 | 1.00 | 9.14 | C |
| ATOM | 2902 | O | ILE | A | 377 | 96.706 | 53.421 | 15.366 | 1.00 | 9.95 | O |
| ATOM | 2903 | N | ASP | A | 378 | 96.205 | 52.098 | 13.611 | 1.00 | 9.15 | N |
| ATOM | 2904 | CA | ASP | A | 378 | 96.789 | 52.992 | 12.601 | 1.00 | 10.39 | C |
| ATOM | 2905 | CB | ASP | A | 378 | 96.760 | 52.329 | 11.205 | 1.00 | 10.75 | C |
| ATOM | 2906 | CG | ASP | A | 378 | 97.653 | 51.067 | 11.113 | 1.00 | 11.43 | C |
| ATOM | 2907 | OD1 | ASP | A | 378 | 98.628 | 50.988 | 11.883 | 1.00 | 9.90 | O |
| ATOM | 2908 | OD2 | ASP | A | 378 | 97.370 | 50.167 | 10.263 | 1.00 | 12.74 | O |
| ATOM | 2909 | C | ASP | A | 378 | 96.065 | 54.361 | 12.564 | 1.00 | 10.57 | C |
| ATOM | 2910 | O | ASP | A | 378 | 96.704 | 55.399 | 12.441 | 1.00 | 9.95 | O |
| ATOM | 2911 | N | ALA | A | 379 | 94.735 | 54.346 | 12.693 | 1.00 | 9.88 | N |
| ATOM | 2912 | CA | ALA | A | 379 | 93.926 | 55.555 | 12.618 | 1.00 | 8.95 | C |
| ATOM | 2913 | CB | ALA | A | 379 | 92.416 | 55.217 | 12.669 | 1.00 | 8.20 | C |
| ATOM | 2914 | C | ALA | A | 379 | 94.300 | 56.446 | 13.773 | 1.00 | 8.88 | C |
| ATOM | 2915 | O | ALA | A | 379 | 94.473 | 57.640 | 13.601 | 1.00 | 9.55 | O |
| ATOM | 2916 | N | HIS | A | 380 | 94.404 | 55.870 | 14.967 | 1.00 | 8.76 | N |
| ATOM | 2917 | CA | HIS | A | 380 | 94.764 | 56.664 | 16.139 | 1.00 | 9.21 | C |
| ATOM | 2918 | CB | HIS | A | 380 | 94.550 | 55.894 | 17.423 | 1.00 | 8.88 | C |
| ATOM | 2919 | CG | HIS | A | 380 | 93.110 | 55.551 | 17.684 | 1.00 | 9.44 | C |
| ATOM | 2920 | ND1 | HIS | A | 380 | 92.734 | 54.366 | 18.184 | 1.00 | 8.74 | N |
| ATOM | 2921 | CE1 | HIS | A | 380 | 91.374 | 54.332 | 18.275 | 1.00 | 10.92 | C |
| ATOM | 2922 | NE2 | HIS | A | 380 | 90.894 | 55.509 | 17.846 | 1.00 | 10.69 | N |
| ATOM | 2923 | CD2 | HIS | A | 380 | 91.941 | 56.283 | 17.464 | 1.00 | 9.96 | C |
| ATOM | 2924 | C | HIS | A | 380 | 96.188 | 57.175 | 16.044 | 1.00 | 8.44 | C |
| ATOM | 2925 | O | HIS | A | 380 | 96.453 | 58.294 | 16.454 | 1.00 | 9.30 | O |
| ATOM | 2926 | N | ASN | A | 381 | 97.097 | 56.384 | 15.495 | 1.00 | 7.47 | N |
| ATOM | 2927 | CA | ASN | A | 381 | 98.466 | 56.834 | 15.340 | 1.00 | 8.52 | C |
| ATOM | 2928 | CB | ASN | A | 381 | 99.370 | 55.705 | 14.773 | 1.00 | 8.35 | C |
| ATOM | 2929 | CG | ASN | A | 381 | 99.708 | 54.645 | 15.818 | 1.00 | 8.34 | C |
| ATOM | 2930 | OD1 | ASN | A | 381 | 99.577 | 54.900 | 17.015 | 1.00 | 8.06 | O |
| ATOM | 2931 | ND2 | ASN | A | 381 | 100.163 | 53.449 | 15.374 | 1.00 | 7.82 | N |
| ATOM | 2932 | C | ASN | A | 381 | 98.520 | 58.093 | 14.459 | 1.00 | 8.41 | C |
| ATOM | 2933 | O | ASN | A | 381 | 99.241 | 59.036 | 14.745 | 1.00 | 8.56 | O |
| ATOM | 2934 | N | TRP | A | 382 | 97.741 | 58.109 | 13.385 | 1.00 | 9.15 | N |
| ATOM | 2935 | CA | TRP | A | 382 | 97.636 | 59.315 | 12.526 | 1.00 | 8.80 | C |
| ATOM | 2936 | CB | TRP | A | 382 | 96.884 | 58.963 | 11.248 | 1.00 | 8.39 | C |
| ATOM | 2937 | CG | TRP | A | 382 | 96.581 | 60.147 | 10.375 | 1.00 | 8.03 | C |
| ATOM | 2938 | CD1 | TRP | A | 382 | 95.403 | 60.848 | 10.284 | 1.00 | 9.01 | C |
| ATOM | 2939 | NE1 | TRP | A | 382 | 95.516 | 61.892 | 9.374 | 1.00 | 8.56 | N |
| ATOM | 2940 | CE2 | TRP | A | 382 | 96.754 | 61.914 | 8.845 | 1.00 | 8.18 | C |
| ATOM | 2941 | CD2 | TRP | A | 382 | 97.500 | 60.815 | 9.452 | 1.00 | 8.04 | C |
| ATOM | 2942 | CE3 | TRP | A | 382 | 98.818 | 60.608 | 9.074 | 1.00 | 8.37 | C |
| ATOM | 2943 | CZ3 | TRP | A | 382 | 99.390 | 61.482 | 8.143 | 1.00 | 8.26 | C |
| ATOM | 2944 | CH2 | TRP | A | 382 | 98.647 | 62.518 | 7.551 | 1.00 | 7.81 | C |
| ATOM | 2945 | CZ2 | TRP | A | 382 | 97.326 | 62.757 | 7.894 | 1.00 | 8.30 | C |
| ATOM | 2946 | C | TRP | A | 382 | 96.945 | 60.481 | 13.227 | 1.00 | 8.48 | C |
| ATOM | 2947 | O | TRP | A | 382 | 97.400 | 61.622 | 13.177 | 1.00 | 8.06 | O |
| ATOM | 2948 | N | LEU | A | 383 | 95.807 | 60.208 | 13.852 | 1.00 | 8.56 | N |
| ATOM | 2949 | CA | LEU | A | 383 | 94.972 | 61.257 | 14.448 | 1.00 | 8.01 | C |
| ATOM | 2950 | CB | LEU | A | 383 | 93.716 | 60.621 | 15.076 | 1.00 | 7.16 | C |
| ATOM | 2951 | CG | LEU | A | 383 | 92.510 | 61.501 | 15.385 | 1.00 | 8.24 | C |
| ATOM | 2952 | CD1 | LEU | A | 383 | 91.842 | 62.030 | 14.103 | 1.00 | 7.28 | C |
| ATOM | 2953 | CD2 | LEU | A | 383 | 91.496 | 60.788 | 16.288 | 1.00 | 7.85 | C |
| ATOM | 2954 | C | LEU | A | 383 | 95.748 | 62.039 | 15.513 | 1.00 | 8.74 | C |
| ATOM | 2955 | O | LEU | A | 383 | 95.588 | 63.255 | 15.631 | 1.00 | 9.24 | O |
| ATOM | 2956 | N | PHE | A | 384 | 96.605 | 61.332 | 16.258 | 1.00 | 7.95 | N |
| ATOM | 2957 | CA | PHE | A | 384 | 97.345 | 61.920 | 17.384 | 1.00 | 8.10 | C |
| ATOM | 2958 | CB | PHE | A | 384 | 97.443 | 60.918 | 18.555 | 1.00 | 7.93 | C |
| ATOM | 2959 | CG | PHE | A | 384 | 96.180 | 60.831 | 19.337 | 1.00 | 8.31 | C |
| ATOM | 2960 | CD1 | PHE | A | 384 | 95.173 | 59.968 | 18.951 | 1.00 | 8.85 | C |
| ATOM | 2961 | CE1 | PHE | A | 384 | 93.963 | 59.940 | 19.650 | 1.00 | 9.55 | C |
| ATOM | 2962 | CZ | PHE | A | 384 | 93.766 | 60.776 | 20.724 | 1.00 | 8.55 | C |
| ATOM | 2963 | CE2 | PHE | A | 384 | 94.770 | 61.640 | 21.113 | 1.00 | 8.59 | C |
| ATOM | 2964 | CD2 | PHE | A | 384 | 95.956 | 61.674 | 20.415 | 1.00 | 8.78 | C |
| ATOM | 2965 | C | PHE | A | 384 | 98.705 | 62.486 | 17.004 | 1.00 | 8.54 | C |
| ATOM | 2966 | O | PHE | A | 384 | 99.399 | 63.021 | 17.872 | 1.00 | 8.69 | O |
| ATOM | 2967 | N | THR | A | 385 | 99.063 | 62.415 | 15.716 | 1.00 | 8.38 | N |
| ATOM | 2968 | CA | THR | A | 385 | 100.333 | 62.984 | 15.243 | 1.00 | 7.85 | C |
| ATOM | 2969 | CB | THR | A | 385 | 101.325 | 61.911 | 14.740 | 1.00 | 7.61 | C |
| ATOM | 2970 | OG1 | THR | A | 385 | 100.730 | 61.155 | 13.682 | 1.00 | 8.05 | O |
| ATOM | 2971 | CG2 | THR | A | 385 | 101.765 | 60.997 | 15.869 | 1.00 | 6.91 | C |
| ATOM | 2972 | C | THR | A | 385 | 100.211 | 64.042 | 14.146 | 1.00 | 8.32 | C |
| ATOM | 2973 | O | THR | A | 385 | 101.047 | 64.951 | 14.085 | 1.00 | 8.72 | O |
| ATOM | 2974 | N | ALA | A | 386 | 99.204 | 63.910 | 13.285 | 1.00 | 8.50 | N |
| ATOM | 2975 | CA | ALA | A | 386 | 98.920 | 64.854 | 12.205 | 1.00 | 8.68 | C |
| ATOM | 2976 | CB | ALA | A | 386 | 98.028 | 64.195 | 11.144 | 1.00 | 7.78 | C |
| ATOM | 2977 | C | ALA | A | 386 | 98.217 | 66.098 | 12.741 | 1.00 | 8.58 | C |
| ATOM | 2978 | O | ALA | A | 386 | 97.604 | 66.057 | 13.797 | 1.00 | 9.11 | O |
| ATOM | 2979 | N | ARG | A | 387 | 98.256 | 67.170 | 11.958 | 1.00 | 7.88 | N |
| ATOM | 2980 | CA | ARG | A | 387 | 97.813 | 68.483 | 12.414 | 1.00 | 8.17 | C |
| ATOM | 2981 | CB | ARG | A | 387 | 98.098 | 69.506 | 11.322 | 1.00 | 9.24 | C |
| ATOM | 2982 | CG | ARG | A | 387 | 97.347 | 69.207 | 10.029 | 1.00 | 11.67 | C |
| ATOM | 2983 | CD | ARG | A | 387 | 96.192 | 70.133 | 9.906 | 1.00 | 11.85 | C |
| ATOM | 2984 | NE | ARG | A | 387 | 95.311 | 69.859 | 8.768 | 1.00 | 11.11 | N |
| ATOM | 2985 | CZ | ARG | A | 387 | 94.113 | 69.285 | 8.840 | 1.00 | 10.57 | C |
| ATOM | 2986 | NH1 | ARG | A | 387 | 93.655 | 68.759 | 9.982 | 1.00 | 9.60 | N |
| ATOM | 2987 | NH2 | ARG | A | 387 | 93.364 | 69.211 | 7.751 | 1.00 | 10.59 | N |
| ATOM | 2988 | C | ARG | A | 387 | 96.325 | 68.495 | 12.797 | 1.00 | 7.95 | C |
| ATOM | 2989 | O | ARG | A | 387 | 95.512 | 67.835 | 12.171 | 1.00 | 7.47 | O |
| ATOM | 2990 | N | GLY | A | 388 | 95.983 | 69.278 | 13.818 | 1.00 | 7.52 | N |
| ATOM | 2991 | CA | GLY | A | 388 | 94.614 | 69.385 | 14.283 | 1.00 | 7.75 | C |
| ATOM | 2992 | C | GLY | A | 388 | 94.475 | 68.905 | 15.720 | 1.00 | 8.33 | C |
| ATOM | 2993 | O | GLY | A | 388 | 95.469 | 68.534 | 16.369 | 1.00 | 10.59 | O |
| ATOM | 2994 | N | ILE | A | 389 | 93.230 | 68.903 | 16.188 | 1.00 | 7.79 | N |
| ATOM | 2995 | CA | ILE | A | 389 | 92.862 | 68.622 | 17.562 | 1.00 | 7.45 | C |
| ATOM | 2996 | CB | ILE | A | 389 | 92.061 | 69.790 | 18.178 | 1.00 | 7.55 | C |
| ATOM | 2997 | CG1 | ILE | A | 389 | 92.809 | 71.138 | 17.985 | 1.00 | 8.63 | C |
| ATOM | 2998 | CD1 | ILE | A | 389 | 92.093 | 72.405 | 18.531 | 1.00 | 6.90 | C |
| ATOM | 2999 | CG2 | ILE | A | 389 | 91.710 | 69.462 | 19.643 | 1.00 | 8.04 | C |
| ATOM | 3000 | C | ILE | A | 389 | 92.014 | 67.367 | 17.524 | 1.00 | 7.61 | C |
| ATOM | 3001 | O | ILE | A | 389 | 90.896 | 67.387 | 16.999 | 1.00 | 8.70 | O |
| ATOM | 3002 | N | PRO | A | 390 | 92.541 | 66.249 | 18.026 | 1.00 | 8.53 | N |
| ATOM | 3003 | CA | PRO | A | 390 | 91.890 | 64.949 | 17.796 | 1.00 | 8.74 | C |
| ATOM | 3004 | CB | PRO | A | 390 | 92.970 | 63.945 | 18.193 | 1.00 | 9.38 | C |
| ATOM | 3005 | CG | PRO | A | 390 | 93.792 | 64.682 | 19.194 | 1.00 | 9.41 | C |
| ATOM | 3006 | CD | PRO | A | 390 | 93.839 | 66.097 | 18.692 | 1.00 | 8.91 | C |
| ATOM | 3007 | C | PRO | A | 390 | 90.653 | 64.742 | 18.668 | 1.00 | 8.66 | C |
| ATOM | 3008 | O | PRO | A | 390 | 90.660 | 65.121 | 19.813 | 1.00 | 7.91 | O |
| ATOM | 3009 | N | VAL | A | 391 | 89.625 | 64.133 | 18.085 | 1.00 | 9.23 | N |
| ATOM | 3010 | CA | VAL | A | 391 | 88.380 | 63.770 | 18.742 | 1.00 | 8.84 | C |
| ATOM | 3011 | CB | VAL | A | 391 | 87.182 | 64.547 | 18.206 | 1.00 | 9.48 | C |
| ATOM | 3012 | CG1 | VAL | A | 391 | 85.918 | 64.148 | 19.026 | 1.00 | 8.02 | C |
| ATOM | 3013 | CG2 | VAL | A | 391 | 87.473 | 66.063 | 18.250 | 1.00 | 9.25 | C |
| ATOM | 3014 | C | VAL | A | 391 | 88.129 | 62.293 | 18.503 | 1.00 | 9.69 | C |
| ATOM | 3015 | O | VAL | A | 391 | 88.115 | 61.851 | 17.345 | 1.00 | 10.62 | O |
| ATOM | 3016 | N | VAL | A | 392 | 87.953 | 61.523 | 19.583 | 1.00 | 8.65 | N |
| ATOM | 3017 | CA | VAL | A | 392 | 87.690 | 60.084 | 19.472 | 1.00 | 8.29 | C |
| ATOM | 3018 | CB | VAL | A | 392 | 88.797 | 59.266 | 20.178 | 1.00 | 8.70 | C |
| ATOM | 3019 | CG1 | VAL | A | 392 | 88.464 | 57.773 | 20.224 | 1.00 | 7.24 | C |
| ATOM | 3020 | CG2 | VAL | A | 392 | 90.160 | 59.487 | 19.464 | 1.00 | 7.11 | C |
| ATOM | 3021 | C | VAL | A | 392 | 86.318 | 59.801 | 20.076 | 1.00 | 8.78 | C |
| ATOM | 3022 | O | VAL | A | 392 | 86.003 | 60.260 | 21.176 | 1.00 | 6.91 | O |
| ATOM | 3023 | N | TYR | A | 393 | 85.505 | 59.072 | 19.325 | 1.00 | 7.24 | N |
| ATOM | 3024 | CA | TYR | A | 393 | 84.132 | 58.760 | 19.703 | 1.00 | 6.81 | C |
| ATOM | 3025 | CB | TYR | A | 393 | 83.357 | 58.425 | 18.420 | 1.00 | 6.91 | C |
| ATOM | 3026 | CG | TYR | A | 393 | 81.884 | 58.134 | 18.514 | 1.00 | 7.60 | C |
| ATOM | 3027 | CD1 | TYR | A | 393 | 81.064 | 58.829 | 19.374 | 1.00 | 7.48 | C |
| ATOM | 3028 | CE1 | TYR | A | 393 | 79.708 | 58.555 | 19.447 | 1.00 | 7.52 | C |
| ATOM | 3029 | CZ | TYR | A | 393 | 79.162 | 57.600 | 18.604 | 1.00 | 8.60 | C |
| ATOM | 3030 | OH | TYR | A | 393 | 77.816 | 57.317 | 18.677 | 1.00 | 8.86 | O |
| ATOM | 3031 | CE2 | TYR | A | 393 | 79.958 | 56.909 | 17.721 | 1.00 | 8.31 | C |
| ATOM | 3032 | CD2 | TYR | A | 393 | 81.301 | 57.167 | 17.676 | 1.00 | 7.64 | C |
| ATOM | 3033 | C | TYR | A | 393 | 84.153 | 57.586 | 20.671 | 1.00 | 7.01 | C |
| ATOM | 3034 | O | TYR | A | 393 | 84.895 | 56.602 | 20.461 | 1.00 | 7.64 | O |
| ATOM | 3035 | N | TYR | A | 394 | 83.352 | 57.677 | 21.736 | 1.00 | 6.60 | N |
| ATOM | 3036 | CA | TYR | A | 394 | 83.415 | 56.677 | 22.835 | 1.00 | 7.13 | C |
| ATOM | 3037 | CB | TYR | A | 394 | 82.275 | 56.888 | 23.858 | 1.00 | 7.04 | C |
| ATOM | 3038 | CG | TYR | A | 394 | 80.931 | 56.373 | 23.424 | 1.00 | 7.20 | C |
| ATOM | 3039 | CD1 | TYR | A | 394 | 80.135 | 57.102 | 22.546 | 1.00 | 7.20 | C |
| ATOM | 3040 | CE1 | TYR | A | 394 | 78.912 | 56.619 | 22.135 | 1.00 | 7.49 | C |
| ATOM | 3041 | CZ | TYR | A | 394 | 78.459 | 55.388 | 22.589 | 1.00 | 7.57 | C |
| ATOM | 3042 | OH | TYR | A | 394 | 77.224 | 54.883 | 22.155 | 1.00 | 7.40 | O |
| ATOM | 3043 | CE2 | TYR | A | 394 | 79.229 | 54.645 | 23.479 | 1.00 | 7.52 | C |
| ATOM | 3044 | CD2 | TYR | A | 394 | 80.458 | 55.143 | 23.885 | 1.00 | 8.12 | C |
| ATOM | 3045 | C | TYR | A | 394 | 83.391 | 55.264 | 22.307 | 1.00 | 6.88 | C |
| ATOM | 3046 | O | TYR | A | 394 | 82.624 | 54.944 | 21.408 | 1.00 | 6.13 | O |
| ATOM | 3047 | N | GLY | A | 395 | 84.237 | 54.419 | 22.866 | 1.00 | 7.42 | N |
| ATOM | 3048 | CA | GLY | A | 395 | 84.254 | 53.018 | 22.479 | 1.00 | 8.26 | C |
| ATOM | 3049 | C | GLY | A | 395 | 85.251 | 52.654 | 21.398 | 1.00 | 8.44 | C |
| ATOM | 3050 | O | GLY | A | 395 | 85.594 | 51.475 | 21.215 | 1.00 | 8.15 | O |
| ATOM | 3051 | N | SER | A | 396 | 85.711 | 53.658 | 20.662 | 1.00 | 9.10 | N |
| ATOM | 3052 | CA | SER | A | 396 | 86.706 | 53.441 | 19.629 | 1.00 | 9.33 | C |
| ATOM | 3053 | CB | SER | A | 396 | 87.121 | 54.760 | 19.022 | 1.00 | 8.64 | C |
| ATOM | 3054 | OG | SER | A | 396 | 86.037 | 55.357 | 18.348 | 1.00 | 9.74 | O |
| ATOM | 3055 | C | SER | A | 396 | 87.961 | 52.742 | 20.155 | 1.00 | 9.75 | C |
| ATOM | 3056 | O | SER | A | 396 | 88.654 | 52.066 | 19.411 | 1.00 | 9.65 | O |
| ATOM | 3057 | N | GLU | A | 397 | 88.258 | 52.961 | 21.424 | 1.00 | 10.33 | N |
| ATOM | 3058 | CA | GLU | A | 397 | 89.432 | 52.388 | 22.073 | 1.00 | 11.40 | C |
| ATOM | 3059 | CB | GLU | A | 397 | 89.641 | 53.011 | 23.474 | 1.00 | 11.29 | C |
| ATOM | 3060 | CG | GLU | A | 397 | 88.551 | 52.770 | 24.523 | 1.00 | 12.39 | C |
| ATOM | 3061 | CD | GLU | A | 397 | 87.385 | 53.748 | 24.508 | 1.00 | 11.96 | C |
| ATOM | 3062 | OE1 | GLU | A | 397 | 87.086 | 54.341 | 23.452 | 1.00 | 13.51 | O |
| ATOM | 3063 | OE2 | GLU | A | 397 | 86.726 | 53.892 | 25.570 | 1.00 | 13.49 | O |
| ATOM | 3064 | C | GLU | A | 397 | 89.403 | 50.844 | 22.125 | 1.00 | 11.34 | C |
| ATOM | 3065 | O | GLU | A | 397 | 90.374 | 50.224 | 22.572 | 1.00 | 11.52 | O |
| ATOM | 3066 | N | MET | A | 398 | 88.288 | 50.245 | 21.712 | 1.00 | 10.06 | N |
| ATOM | 3067 | CA | MET | A | 398 | 88.184 | 48.796 | 21.499 | 1.00 | 10.02 | C |
| ATOM | 3068 | CB | MET | A | 398 | 87.437 | 48.128 | 22.672 | 1.00 | 12.71 | C |
| ATOM | 3069 | CG | MET | A | 398 | 85.964 | 48.528 | 22.786 | 1.00 | 12.53 | C |
| ATOM | 3070 | SD | MET | A | 398 | 85.029 | 47.544 | 23.997 | 1.00 | 16.23 | S |
| ATOM | 3071 | CE | MET | A | 398 | 84.659 | 46.047 | 23.032 | 1.00 | 14.44 | C |
| ATOM | 3072 | C | MET | A | 398 | 87.455 | 48.473 | 20.207 | 1.00 | 9.47 | C |
| ATOM | 3073 | O | MET | A | 398 | 87.041 | 47.335 | 19.997 | 1.00 | 8.75 | O |
| ATOM | 3074 | N | GLY | A | 399 | 87.271 | 49.465 | 19.333 | 1.00 | 9.52 | N |
| ATOM | 3075 | CA | GLY | A | 399 | 86.505 | 49.261 | 18.110 | 1.00 | 9.21 | C |
| ATOM | 3076 | C | GLY | A | 399 | 85.090 | 48.803 | 18.401 | 1.00 | 7.96 | C |
| ATOM | 3077 | O | GLY | A | 399 | 84.568 | 47.957 | 17.694 | 1.00 | 8.09 | O |
| ATOM | 3078 | N | PHE | A | 400 | 84.483 | 49.386 | 19.440 | 1.00 | 7.58 | N |
| ATOM | 3079 | CA | PHE | A | 400 | 83.107 | 49.084 | 19.861 | 1.00 | 8.56 | C |
| ATOM | 3080 | CB | PHE | A | 400 | 82.767 | 49.963 | 21.061 | 1.00 | 9.59 | C |
| ATOM | 3081 | CG | PHE | A | 400 | 81.358 | 49.810 | 21.604 | 1.00 | 9.31 | C |
| ATOM | 3082 | CD1 | PHE | A | 400 | 80.966 | 48.659 | 22.265 | 1.00 | 11.80 | C |
| ATOM | 3083 | CE1 | PHE | A | 400 | 79.695 | 48.552 | 22.820 | 1.00 | 11.61 | C |
| ATOM | 3084 | CZ | PHE | A | 400 | 78.810 | 49.615 | 22.714 | 1.00 | 11.48 | C |
| ATOM | 3085 | CE2 | PHE | A | 400 | 79.188 | 50.765 | 22.071 | 1.00 | 11.37 | C |
| ATOM | 3086 | CD2 | PHE | A | 400 | 80.464 | 50.861 | 21.530 | 1.00 | 10.30 | C |
| ATOM | 3087 | C | PHE | A | 400 | 82.153 | 49.387 | 18.722 | 1.00 | 9.36 | C |
| ATOM | 3088 | O | PHE | A | 400 | 82.151 | 50.511 | 18.218 | 1.00 | 8.65 | O |
| ATOM | 3089 | N | MET | A | 401 | 81.350 | 48.397 | 18.319 | 1.00 | 9.03 | N |
| ATOM | 3090 | CA | MET | A | 401 | 80.299 | 48.602 | 17.296 | 1.00 | 9.83 | C |
| ATOM | 3091 | CB | MET | A | 401 | 79.216 | 49.567 | 17.806 | 1.00 | 11.64 | C |
| ATOM | 3092 | CG | MET | A | 401 | 78.416 | 49.024 | 18.991 | 1.00 | 13.59 | C |
| ATOM | 3093 | SD | MET | A | 401 | 77.307 | 47.646 | 18.569 | 1.00 | 17.07 | S |
| ATOM | 3094 | CE | MET | A | 401 | 78.174 | 46.251 | 19.273 | 1.00 | 21.13 | C |
| ATOM | 3095 | C | MET | A | 401 | 80.905 | 49.108 | 15.988 | 1.00 | 9.89 | C |
| ATOM | 3096 | O | MET | A | 401 | 80.275 | 49.872 | 15.216 | 1.00 | 9.34 | O |
| ATOM | 3097 | N | ARG | A | 402 | 82.128 | 48.656 | 15.716 | 1.00 | 9.32 | N |
| ATOM | 3098 | CA | ARG | A | 402 | 82.829 | 49.061 | 14.503 | 1.00 | 9.24 | C |
| ATOM | 3099 | CB | ARG | A | 402 | 84.162 | 48.336 | 14.382 | 1.00 | 9.99 | C |
| ATOM | 3100 | CG | ARG | A | 402 | 84.055 | 46.823 | 14.315 | 1.00 | 10.89 | C |
| ATOM | 3101 | CD | ARG | A | 402 | 85.424 | 46.171 | 14.380 | 1.00 | 10.30 | C |
| ATOM | 3102 | NE | ARG | A | 402 | 85.985 | 46.226 | 15.733 | 1.00 | 10.74 | N |
| ATOM | 3103 | CZ | ARG | A | 402 | 87.217 | 45.848 | 16.064 | 1.00 | 12.31 | C |
| ATOM | 3104 | NH1 | ARG | A | 402 | 88.111 | 45.453 | 15.137 | 1.00 | 14.64 | N |
| ATOM | 3105 | NH2 | ARG | A | 402 | 87.580 | 45.897 | 17.330 | 1.00 | 12.67 | N |
| ATOM | 3106 | C | ARG | A | 402 | 81.962 | 48.772 | 13.300 | 1.00 | 8.71 | C |
| ATOM | 3107 | O | ARG | A | 402 | 81.260 | 47.770 | 13.255 | 1.00 | 10.20 | O |
| ATOM | 3108 | N | GLY | A | 403 | 81.980 | 49.662 | 12.331 | 1.00 | 8.92 | N |
| ATOM | 3109 | CA | GLY | A | 403 | 81.199 | 49.458 | 11.162 | 1.00 | 9.36 | C |
| ATOM | 3110 | C | GLY | A | 403 | 79.717 | 49.743 | 11.273 | 1.00 | 9.18 | C |
| ATOM | 3111 | O | GLY | A | 403 | 79.048 | 49.718 | 10.265 | 1.00 | 10.53 | O |
| ATOM | 3112 | N | ARG | A | 404 | 79.177 | 50.032 | 12.450 | 1.00 | 11.12 | N |
| ATOM | 3113 | CA | ARG | A | 404 | 77.711 | 50.091 | 12.601 | 1.00 | 11.58 | C |
| ATOM | 3114 | CB | ARG | A | 404 | 77.268 | 49.569 | 13.951 | 1.00 | 10.97 | C |
| ATOM | 3115 | CG | ARG | A | 404 | 77.811 | 48.205 | 14.320 | 1.00 | 14.19 | C |
| ATOM | 3116 | CD | ARG | A | 404 | 77.059 | 47.080 | 13.693 | 1.00 | 15.25 | C |
| ATOM | 3117 | NE | ARG | A | 404 | 77.652 | 45.769 | 13.974 | 1.00 | 17.42 | N |
| ATOM | 3118 | CZ | ARG | A | 404 | 77.110 | 44.616 | 13.569 | 1.00 | 16.10 | C |
| ATOM | 3119 | NH1 | ARG | A | 404 | 75.943 | 44.593 | 12.937 | 1.00 | 15.16 | N |
| ATOM | 3120 | NH2 | ARG | A | 404 | 77.735 | 43.479 | 13.794 | 1.00 | 14.11 | N |
| ATOM | 3121 | C | ARG | A | 404 | 77.156 | 51.490 | 12.419 | 1.00 | 13.21 | C |
| ATOM | 3122 | O | ARG | A | 404 | 77.733 | 52.441 | 12.927 | 1.00 | 11.53 | O |
| ATOM | 3123 | N | PRO | A | 405 | 76.001 | 51.611 | 11.730 | 1.00 | 12.40 | N |
| ATOM | 3124 | CA | PRO | A | 405 | 75.357 | 52.898 | 11.519 | 1.00 | 12.75 | C |
| ATOM | 3125 | CB | PRO | A | 405 | 74.593 | 52.660 | 10.208 | 1.00 | 13.94 | C |
| ATOM | 3126 | CG | PRO | A | 405 | 74.145 | 51.261 | 10.328 | 1.00 | 15.91 | C |
| ATOM | 3127 | CD | PRO | A | 405 | 75.258 | 50.526 | 11.060 | 1.00 | 13.75 | C |
| ATOM | 3128 | C | PRO | A | 405 | 74.373 | 53.284 | 12.629 | 1.00 | 11.38 | C |
| ATOM | 3129 | O | PRO | A | 405 | 73.994 | 52.445 | 13.492 | 1.00 | 9.44 | O |
| ATOM | 3130 | N | GLU | A | 406 | 73.962 | 54.540 | 12.646 | 1.00 | 11.11 | N |
| ATOM | 3131 | CA | GLU | A | 406 | 72.835 | 54.921 | 13.495 | 1.00 | 11.92 | C |
| ATOM | 3132 | CB | GLU | A | 406 | 72.447 | 56.398 | 13.339 | 1.00 | 11.86 | C |
| ATOM | 3133 | CG | GLU | A | 406 | 71.359 | 56.796 | 14.344 | 1.00 | 13.35 | C |
| ATOM | 3134 | CD | GLU | A | 406 | 70.896 | 58.242 | 14.262 | 1.00 | 17.58 | C |
| ATOM | 3135 | OE1 | GLU | A | 406 | 71.185 | 58.921 | 13.258 | 1.00 | 18.39 | O |
| ATOM | 3136 | OE2 | GLU | A | 406 | 70.248 | 58.705 | 15.230 | 1.00 | 14.63 | O |
| ATOM | 3137 | C | GLU | A | 406 | 71.636 | 54.026 | 13.143 | 1.00 | 10.51 | C |
| ATOM | 3138 | O | GLU | A | 406 | 71.390 | 53.760 | 11.972 | 1.00 | 9.65 | O |
| ATOM | 3139 | N | HIS | A | 407 | 70.909 | 53.600 | 14.176 | 1.00 | 11.75 | N |
| ATOM | 3140 | CA | HIS | A | 407 | 69.770 | 52.662 | 14.105 | 1.00 | 10.58 | C |
| ATOM | 3141 | CB | HIS | A | 407 | 68.723 | 53.082 | 13.058 | 1.00 | 10.81 | C |
| ATOM | 3142 | CG | HIS | A | 407 | 68.307 | 54.536 | 13.170 | 1.00 | 11.70 | C |
| ATOM | 3143 | ND1 | HIS | A | 407 | 68.332 | 55.385 | 12.116 | 1.00 | 13.69 | N |
| ATOM | 3144 | CE1 | HIS | A | 407 | 67.968 | 56.616 | 12.521 | 1.00 | 13.15 | C |
| ATOM | 3145 | NE2 | HIS | A | 407 | 67.697 | 56.560 | 13.831 | 1.00 | 12.12 | N |
| ATOM | 3146 | CD2 | HIS | A | 407 | 67.907 | 55.289 | 14.265 | 1.00 | 12.01 | C |
| ATOM | 3147 | C | HIS | A | 407 | 70.207 | 51.235 | 13.911 | 1.00 | 11.19 | C |
| ATOM | 3148 | O | HIS | A | 407 | 69.374 | 50.349 | 13.786 | 1.00 | 11.04 | O |
| ATOM | 3149 | N | GLY | A | 408 | 71.516 | 50.990 | 13.910 | 1.00 | 9.43 | N |
| ATOM | 3150 | CA | GLY | A | 408 | 72.052 | 49.635 | 13.812 | 1.00 | 9.69 | C |
| ATOM | 3151 | C | GLY | A | 408 | 73.146 | 49.337 | 14.829 | 1.00 | 9.07 | C |
| ATOM | 3152 | O | GLY | A | 408 | 73.943 | 48.396 | 14.651 | 1.00 | 8.47 | O |
| ATOM | 3153 | N | GLY | A | 409 | 73.225 | 50.156 | 15.879 | 1.00 | 9.25 | N |
| ATOM | 3154 | CA | GLY | A | 409 | 74.146 | 49.869 | 16.983 | 1.00 | 9.82 | C |
| ATOM | 3155 | C | GLY | A | 409 | 75.266 | 50.867 | 17.219 | 1.00 | 9.83 | C |
| ATOM | 3156 | O | GLY | A | 409 | 75.963 | 50.763 | 18.219 | 1.00 | 11.53 | O |
| ATOM | 3157 | N | ASN | A | 410 | 75.431 | 51.836 | 16.319 | 1.00 | 9.21 | N |
| ATOM | 3158 | CA | ASN | A | 410 | 76.487 | 52.863 | 16.431 | 1.00 | 9.27 | C |
| ATOM | 3159 | CB | ASN | A | 410 | 76.248 | 53.961 | 15.375 | 1.00 | 8.73 | C |
| ATOM | 3160 | CG | ASN | A | 410 | 77.062 | 55.217 | 15.614 | 1.00 | 10.22 | C |
| ATOM | 3161 | OD1 | ASN | A | 410 | 76.945 | 55.853 | 16.674 | 1.00 | 10.70 | O |
| ATOM | 3162 | ND2 | ASN | A | 410 | 77.864 | 55.630 | 14.595 | 1.00 | 9.57 | N |
| ATOM | 3163 | C | ASN | A | 410 | 76.507 | 53.459 | 17.823 | 1.00 | 9.80 | C |
| ATOM | 3164 | O | ASN | A | 410 | 77.570 | 53.617 | 18.437 | 1.00 | 9.69 | O |
| ATOM | 3165 | N | ARG | A | 411 | 75.313 | 53.769 | 18.323 | 1.00 | 8.55 | N |
| ATOM | 3166 | CA | ARG | A | 411 | 75.152 | 54.507 | 19.587 | 1.00 | 9.67 | C |
| ATOM | 3167 | CB | ARG | A | 411 | 74.069 | 55.560 | 19.401 | 1.00 | 10.14 | C |
| ATOM | 3168 | CG | ARG | A | 411 | 74.368 | 56.565 | 18.305 | 1.00 | 9.41 | C |
| ATOM | 3169 | CD | ARG | A | 411 | 73.169 | 57.449 | 18.073 | 1.00 | 9.64 | C |
| ATOM | 3170 | NE | ARG | A | 411 | 73.414 | 58.495 | 17.074 | 1.00 | 8.16 | N |
| ATOM | 3171 | CZ | ARG | A | 411 | 72.728 | 59.634 | 17.005 | 1.00 | 8.73 | C |
| ATOM | 3172 | NH1 | ARG | A | 411 | 71.739 | 59.888 | 17.850 | 1.00 | 8.48 | N |
| ATOM | 3173 | NH2 | ARG | A | 411 | 73.014 | 60.532 | 16.079 | 1.00 | 9.18 | N |
| ATOM | 3174 | C | ARG | A | 411 | 74.797 | 53.621 | 20.801 | 1.00 | 8.76 | C |
| ATOM | 3175 | O | ARG | A | 411 | 74.301 | 54.097 | 21.818 | 1.00 | 8.13 | O |
| ATOM | 3176 | N | ASN | A | 412 | 75.067 | 52.336 | 20.712 | 1.00 | 8.92 | N |
| ATOM | 3177 | CA | ASN | A | 412 | 74.708 | 51.429 | 21.798 | 1.00 | 9.01 | C |
| ATOM | 3178 | CB | ASN | A | 412 | 74.914 | 49.949 | 21.388 | 1.00 | 9.99 | C |
| ATOM | 3179 | CG | ASN | A | 412 | 73.736 | 49.390 | 20.568 | 1.00 | 10.79 | C |
| ATOM | 3180 | OD1 | ASN | A | 412 | 72.828 | 50.126 | 20.188 | 1.00 | 10.43 | O |
| ATOM | 3181 | ND2 | ASN | A | 412 | 73.742 | 48.073 | 20.329 | 1.00 | 10.06 | N |
| ATOM | 3182 | C | ASN | A | 412 | 75.428 | 51.771 | 23.113 | 1.00 | 9.19 | C |
| ATOM | 3183 | O | ASN | A | 412 | 76.485 | 52.412 | 23.118 | 1.00 | 8.19 | O |
| ATOM | 3184 | N | TYR | A | 413 | 74.786 | 51.377 | 24.218 | 1.00 | 9.36 | N |
| ATOM | 3185 | CA | TYR | A | 413 | 75.240 | 51.660 | 25.564 | 1.00 | 9.55 | C |
| ATOM | 3186 | CB | TYR | A | 413 | 74.164 | 51.225 | 26.583 | 1.00 | 10.00 | C |
| ATOM | 3187 | CG | TYR | A | 413 | 74.551 | 51.420 | 28.013 | 1.00 | 10.43 | C |
| ATOM | 3188 | CD1 | TYR | A | 413 | 74.396 | 52.644 | 28.634 | 1.00 | 10.31 | C |
| ATOM | 3189 | CE1 | TYR | A | 413 | 74.767 | 52.825 | 29.955 | 1.00 | 10.24 | C |
| ATOM | 3190 | CZ | TYR | A | 413 | 75.292 | 51.769 | 30.667 | 1.00 | 10.83 | C |
| ATOM | 3191 | OH | TYR | A | 413 | 75.686 | 51.949 | 31.966 | 1.00 | 9.63 | O |
| ATOM | 3192 | CE2 | TYR | A | 413 | 75.461 | 50.545 | 30.069 | 1.00 | 11.66 | C |
| ATOM | 3193 | CD2 | TYR | A | 413 | 75.093 | 50.370 | 28.751 | 1.00 | 11.76 | C |
| ATOM | 3194 | C | TYR | A | 413 | 76.541 | 50.937 | 25.823 | 1.00 | 8.78 | C |
| ATOM | 3195 | O | TYR | A | 413 | 76.666 | 49.767 | 25.526 | 1.00 | 8.26 | O |
| ATOM | 3196 | N | PHE | A | 414 | 77.525 | 51.643 | 26.374 | 1.00 | 9.51 | N |
| ATOM | 3197 | CA | PHE | A | 414 | 78.899 | 51.112 | 26.523 | 1.00 | 8.44 | C |
| ATOM | 3198 | CB | PHE | A | 414 | 79.878 | 52.264 | 26.303 | 1.00 | 8.41 | C |
| ATOM | 3199 | CG | PHE | A | 414 | 81.332 | 51.867 | 26.226 | 1.00 | 7.71 | C |
| ATOM | 3200 | CD1 | PHE | A | 414 | 81.790 | 51.031 | 25.238 | 1.00 | 8.09 | C |
| ATOM | 3201 | CE1 | PHE | A | 414 | 83.137 | 50.696 | 25.155 | 1.00 | 8.79 | C |
| ATOM | 3202 | CZ | PHE | A | 414 | 84.043 | 51.211 | 26.046 | 1.00 | 8.29 | C |
| ATOM | 3203 | CE2 | PHE | A | 414 | 83.599 | 52.059 | 27.046 | 1.00 | 9.45 | C |
| ATOM | 3204 | CD2 | PHE | A | 414 | 82.244 | 52.383 | 27.128 | 1.00 | 8.59 | C |
| ATOM | 3205 | C | PHE | A | 414 | 79.053 | 50.487 | 27.899 | 1.00 | 8.90 | C |
| ATOM | 3206 | O | PHE | A | 414 | 79.303 | 49.293 | 28.038 | 1.00 | 8.60 | O |
| ATOM | 3207 | N | GLY | A | 415 | 78.864 | 51.292 | 28.939 | 1.00 | 10.68 | N |
| ATOM | 3208 | CA | GLY | A | 415 | 78.755 | 50.759 | 30.307 | 1.00 | 11.20 | C |
| ATOM | 3209 | C | GLY | A | 415 | 80.077 | 50.315 | 30.904 | 1.00 | 11.49 | C |
| ATOM | 3210 | O | GLY | A | 415 | 81.134 | 50.334 | 30.244 | 1.00 | 9.66 | O |
| ATOM | 3211 | N | THR | A | 416 | 80.011 | 49.889 | 32.157 | 1.00 | 12.37 | N |
| ATOM | 3212 | CA | THR | A | 416 | 81.176 | 49.297 | 32.815 | 1.00 | 12.28 | C |
| ATOM | 3213 | CB | THR | A | 416 | 80.869 | 48.975 | 34.282 | 1.00 | 12.76 | C |
| ATOM | 3214 | OG1 | THR | A | 416 | 79.677 | 48.206 | 34.307 | 1.00 | 11.49 | O |
| ATOM | 3215 | CG2 | THR | A | 416 | 80.666 | 50.284 | 35.113 | 1.00 | 12.14 | C |
| ATOM | 3216 | C | THR | A | 416 | 81.610 | 48.015 | 32.106 | 1.00 | 12.64 | C |
| ATOM | 3217 | O | THR | A | 416 | 82.780 | 47.681 | 32.124 | 1.00 | 13.25 | O |
| ATOM | 3218 | N | GLU | A | 417 | 80.693 | 47.318 | 31.445 | 1.00 | 12.00 | N |
| ATOM | 3219 | CA | GLU | A | 417 | 81.080 | 46.178 | 30.594 | 1.00 | 15.75 | C |
| ATOM | 3220 | CB | GLU | A | 417 | 79.853 | 45.442 | 30.043 | 1.00 | 21.97 | C |
| ATOM | 3221 | CG | GLU | A | 417 | 79.130 | 44.614 | 31.097 | 1.00 | 30.92 | C |
| ATOM | 3222 | CD | GLU | A | 417 | 78.104 | 43.632 | 30.520 | 1.00 | 41.78 | C |
| ATOM | 3223 | OE1 | GLU | A | 417 | 77.862 | 43.612 | 29.286 | 1.00 | 41.73 | O |
| ATOM | 3224 | OE2 | GLU | A | 417 | 77.530 | 42.864 | 31.324 | 1.00 | 54.62 | O |
| ATOM | 3225 | C | GLU | A | 417 | 81.959 | 46.579 | 29.418 | 1.00 | 12.75 | C |
| ATOM | 3226 | O | GLU | A | 417 | 82.930 | 45.894 | 29.104 | 1.00 | 12.24 | O |
| ATOM | 3227 | N | GLY | A | 418 | 81.603 | 47.667 | 28.730 | 1.00 | 10.82 | N |
| ATOM | 3228 | CA | GLY | A | 418 | 82.473 | 48.196 | 27.668 | 1.00 | 10.01 | C |
| ATOM | 3229 | C | GLY | A | 418 | 83.852 | 48.571 | 28.189 | 1.00 | 10.14 | C |
| ATOM | 3230 | O | GLY | A | 418 | 84.883 | 48.261 | 27.564 | 1.00 | 11.11 | O |
| ATOM | 3231 | N | ILE | A | 419 | 83.873 | 49.257 | 29.329 | 1.00 | 10.51 | N |
| ATOM | 3232 | CA | ILE | A | 419 | 85.131 | 49.682 | 29.955 | 1.00 | 11.23 | C |
| ATOM | 3233 | CB | ILE | A | 419 | 84.879 | 50.478 | 31.241 | 1.00 | 12.65 | C |
| ATOM | 3234 | CG1 | ILE | A | 419 | 84.409 | 51.890 | 30.885 | 1.00 | 13.62 | C |
| ATOM | 3235 | CD1 | ILE | A | 419 | 83.646 | 52.570 | 31.993 | 1.00 | 14.41 | C |
| ATOM | 3236 | CG2 | ILE | A | 419 | 86.141 | 50.538 | 32.127 | 1.00 | 11.69 | C |
| ATOM | 3237 | C | ILE | A | 419 | 86.035 | 48.488 | 30.232 | 1.00 | 12.04 | C |
| ATOM | 3238 | O | ILE | A | 419 | 87.226 | 48.547 | 29.924 | 1.00 | 9.88 | O |
| ATOM | 3239 | N | ALA | A | 420 | 85.454 | 47.406 | 30.768 | 1.00 | 12.10 | N |
| ATOM | 3240 | CA | ALA | A | 420 | 86.203 | 46.175 | 31.100 | 1.00 | 14.98 | C |
| ATOM | 3241 | CB | ALA | A | 420 | 85.335 | 45.171 | 31.892 | 1.00 | 13.23 | C |
| ATOM | 3242 | C | ALA | A | 420 | 86.735 | 45.541 | 29.827 | 1.00 | 14.21 | C |
| ATOM | 3243 | O | ALA | A | 420 | 87.912 | 45.168 | 29.737 | 1.00 | 13.95 | O |
| ATOM | 3244 | N | ALA | A | 421 | 85.878 | 45.462 | 28.820 | 1.00 | 14.06 | N |
| ATOM | 3245 | CA | ALA | A | 421 | 86.292 | 44.905 | 27.541 | 1.00 | 13.64 | C |
| ATOM | 3246 | CB | ALA | A | 421 | 85.087 | 44.736 | 26.612 | 1.00 | 13.35 | C |
| ATOM | 3247 | C | ALA | A | 421 | 87.401 | 45.760 | 26.903 | 1.00 | 14.05 | C |
| ATOM | 3248 | O | ALA | A | 421 | 88.346 | 45.227 | 26.345 | 1.00 | 15.44 | O |
| ATOM | 3249 | N | ALA | A | 422 | 87.319 | 47.082 | 27.036 | 1.00 | 14.54 | N |
| ATOM | 3250 | CA | ALA | A | 422 | 88.318 | 47.973 | 26.427 | 1.00 | 14.27 | C |
| ATOM | 3251 | CB | ALA | A | 422 | 87.825 | 49.423 | 26.405 | 1.00 | 10.31 | C |
| ATOM | 3252 | C | ALA | A | 422 | 89.682 | 47.872 | 27.112 | 1.00 | 14.26 | C |
| ATOM | 3253 | O | ALA | A | 422 | 90.720 | 47.955 | 26.458 | 1.00 | 13.78 | O |
| ATOM | 3254 | N | LYS | A | 423 | 89.686 | 47.668 | 28.422 | 1.00 | 16.41 | N |
| ATOM | 3255 | CA | LYS | A | 423 | 90.952 | 47.552 | 29.158 | 1.00 | 17.57 | C |
| ATOM | 3256 | CB | LYS | A | 423 | 90.733 | 47.489 | 30.673 | 1.00 | 18.04 | C |
| ATOM | 3257 | CG | LYS | A | 423 | 90.364 | 48.829 | 31.291 | 1.00 | 20.96 | C |
| ATOM | 3258 | CD | LYS | A | 423 | 90.145 | 48.702 | 32.782 | 1.00 | 22.90 | C |
| ATOM | 3259 | CE | LYS | A | 423 | 89.713 | 50.010 | 33.396 | 1.00 | 29.43 | C |
| ATOM | 3260 | NZ | LYS | A | 423 | 89.573 | 49.913 | 34.876 | 1.00 | 35.53 | N |
| ATOM | 3261 | C | LYS | A | 423 | 91.729 | 46.325 | 28.717 | 1.00 | 17.71 | C |
| ATOM | 3262 | O | LYS | A | 423 | 92.921 | 46.244 | 28.948 | 1.00 | 18.10 | O |
| ATOM | 3263 | N | ALA | A | 424 | 91.055 | 45.370 | 28.090 | 1.00 | 18.81 | N |
| ATOM | 3264 | CA | ALA | A | 424 | 91.709 | 44.156 | 27.629 | 1.00 | 17.13 | C |
| ATOM | 3265 | CB | ALA | A | 424 | 90.792 | 42.946 | 27.888 | 1.00 | 17.01 | C |
| ATOM | 3266 | C | ALA | A | 424 | 92.084 | 44.241 | 26.152 | 1.00 | 19.84 | C |
| ATOM | 3267 | O | ALA | A | 424 | 92.621 | 43.292 | 25.613 | 1.00 | 19.77 | O |
| ATOM | 3268 | N | SER | A | 425 | 91.775 | 45.359 | 25.485 | 1.00 | 17.11 | N |
| ATOM | 3269 | CA | SER | A | 425 | 92.008 | 45.493 | 24.047 | 1.00 | 15.39 | C |
| ATOM | 3270 | CB | SER | A | 425 | 90.935 | 46.401 | 23.432 | 1.00 | 17.94 | C |
| ATOM | 3271 | OG | SER | A | 425 | 91.178 | 46.615 | 22.057 | 1.00 | 14.67 | O |
| ATOM | 3272 | C | SER | A | 425 | 93.375 | 46.088 | 23.701 | 1.00 | 12.91 | C |
| ATOM | 3273 | O | SER | A | 425 | 93.788 | 47.099 | 24.289 | 1.00 | 12.79 | O |
| ATOM | 3274 | N | PRO | A | 426 | 94.073 | 45.498 | 22.723 | 1.00 | 14.11 | N |
| ATOM | 3275 | CA | PRO | A | 426 | 95.335 | 46.127 | 22.274 | 1.00 | 14.44 | C |
| ATOM | 3276 | CB | PRO | A | 426 | 95.945 | 45.104 | 21.293 | 1.00 | 16.99 | C |
| ATOM | 3277 | CG | PRO | A | 426 | 94.858 | 44.108 | 20.994 | 1.00 | 24.47 | C |
| ATOM | 3278 | CD | PRO | A | 426 | 93.852 | 44.158 | 22.129 | 1.00 | 18.07 | C |
| ATOM | 3279 | C | PRO | A | 426 | 95.153 | 47.485 | 21.590 | 1.00 | 12.29 | C |
| ATOM | 3280 | O | PRO | A | 426 | 96.106 | 48.262 | 21.517 | 1.00 | 10.83 | O |
| ATOM | 3281 | N | ILE | A | 427 | 93.947 | 47.777 | 21.093 | 1.00 | 10.87 | N |
| ATOM | 3282 | CA | ILE | A | 427 | 93.651 | 49.111 | 20.544 | 1.00 | 9.18 | C |
| ATOM | 3283 | CB | ILE | A | 427 | 92.228 | 49.216 | 19.927 | 1.00 | 8.07 | C |
| ATOM | 3284 | CG1 | ILE | A | 427 | 92.028 | 48.155 | 18.839 | 1.00 | 8.24 | C |
| ATOM | 3285 | CD1 | ILE | A | 427 | 90.563 | 47.951 | 18.430 | 1.00 | 8.25 | C |
| ATOM | 3286 | CG2 | ILE | A | 427 | 91.976 | 50.608 | 19.376 | 1.00 | 6.93 | C |
| ATOM | 3287 | C | ILE | A | 427 | 93.813 | 50.155 | 21.634 | 1.00 | 8.85 | C |
| ATOM | 3288 | O | ILE | A | 427 | 94.454 | 51.173 | 21.434 | 1.00 | 9.48 | O |
| ATOM | 3289 | N | ARG | A | 428 | 93.294 | 49.886 | 22.823 | 1.00 | 8.60 | N |
| ATOM | 3290 | CA | ARG | A | 428 | 93.400 | 50.866 | 23.894 | 1.00 | 8.95 | C |
| ATOM | 3291 | CB | ARG | A | 428 | 92.500 | 50.479 | 25.056 | 1.00 | 10.81 | C |
| ATOM | 3292 | CG | ARG | A | 428 | 92.422 | 51.559 | 26.136 | 1.00 | 11.59 | C |
| ATOM | 3293 | CD | ARG | A | 428 | 91.447 | 51.164 | 27.240 | 1.00 | 12.02 | C |
| ATOM | 3294 | NE | ARG | A | 428 | 91.350 | 52.213 | 28.252 | 1.00 | 12.91 | N |
| ATOM | 3295 | CZ | ARG | A | 428 | 92.020 | 52.236 | 29.398 | 1.00 | 13.84 | C |
| ATOM | 3296 | NH1 | ARG | A | 428 | 92.856 | 51.263 | 29.698 | 1.00 | 14.05 | N |
| ATOM | 3297 | NH2 | ARG | A | 428 | 91.835 | 53.234 | 30.258 | 1.00 | 14.78 | N |
| ATOM | 3298 | C | ARG | A | 428 | 94.849 | 51.122 | 24.363 | 1.00 | 9.27 | C |
| ATOM | 3299 | O | ARG | A | 428 | 95.228 | 52.266 | 24.663 | 1.00 | 8.53 | O |
| ATOM | 3300 | N | ALA | A | 429 | 95.663 | 50.069 | 24.391 | 1.00 | 9.95 | N |
| ATOM | 3301 | CA | ALA | A | 429 | 97.056 | 50.169 | 24.807 | 1.00 | 11.02 | C |
| ATOM | 3302 | CB | ALA | A | 429 | 97.705 | 48.757 | 24.946 | 1.00 | 11.56 | C |
| ATOM | 3303 | C | ALA | A | 429 | 97.837 | 51.028 | 23.809 | 1.00 | 10.12 | C |
| ATOM | 3304 | O | ALA | A | 429 | 98.608 | 51.895 | 24.214 | 1.00 | 9.00 | O |
| ATOM | 3305 | N | ALA | A | 430 | 97.619 | 50.792 | 22.512 | 1.00 | 8.74 | N |
| ATOM | 3306 | CA | ALA | A | 430 | 98.286 | 51.579 | 21.459 | 1.00 | 9.32 | C |
| ATOM | 3307 | CB | ALA | A | 430 | 98.092 | 50.932 | 20.081 | 1.00 | 7.90 | C |
| ATOM | 3308 | C | ALA | A | 430 | 97.794 | 53.037 | 21.466 | 1.00 | 9.63 | C |
| ATOM | 3309 | O | ALA | A | 430 | 98.571 | 53.965 | 21.245 | 1.00 | 10.12 | O |
| ATOM | 3310 | N | LEU | A | 431 | 96.505 | 53.239 | 21.720 | 1.00 | 9.43 | N |
| ATOM | 3311 | CA | LEU | A | 431 | 95.941 | 54.610 | 21.769 | 1.00 | 9.39 | C |
| ATOM | 3312 | CB | LEU | A | 431 | 94.422 | 54.577 | 21.980 | 1.00 | 8.79 | C |
| ATOM | 3313 | CG | LEU | A | 431 | 93.674 | 55.877 | 22.294 | 1.00 | 10.37 | C |
| ATOM | 3314 | CD1 | LEU | A | 431 | 93.855 | 56.860 | 21.155 | 1.00 | 10.80 | C |
| ATOM | 3315 | CD2 | LEU | A | 431 | 92.191 | 55.572 | 22.517 | 1.00 | 10.19 | C |
| ATOM | 3316 | C | LEU | A | 431 | 96.590 | 55.393 | 22.902 | 1.00 | 9.89 | C |
| ATOM | 3317 | O | LEU | A | 431 | 97.041 | 56.529 | 22.713 | 1.00 | 8.92 | O |
| ATOM | 3318 | N | THR | A | 432 | 96.634 | 54.765 | 24.075 | 1.00 | 9.59 | N |
| ATOM | 3319 | CA | THR | A | 432 | 97.269 | 55.343 | 25.242 | 1.00 | 9.93 | C |
| ATOM | 3320 | CB | THR | A | 432 | 97.247 | 54.365 | 26.429 | 1.00 | 10.14 | C |
| ATOM | 3321 | OG1 | THR | A | 432 | 95.881 | 54.127 | 26.769 | 1.00 | 8.71 | O |
| ATOM | 3322 | CG2 | THR | A | 432 | 98.019 | 54.934 | 27.635 | 1.00 | 10.34 | C |
| ATOM | 3323 | C | THR | A | 432 | 98.696 | 55.768 | 24.965 | 1.00 | 10.79 | C |
| ATOM | 3324 | O | THR | A | 432 | 99.088 | 56.864 | 25.351 | 1.00 | 10.03 | O |
| ATOM | 3325 | N | ARG | A | 433 | 99.468 | 54.916 | 24.300 | 1.00 | 11.71 | N |
| ATOM | 3326 | CA | ARG | A | 433 | 100.872 | 55.225 | 24.053 | 1.00 | 12.68 | C |
| ATOM | 3327 | CB | ARG | A | 433 | 101.629 | 54.062 | 23.396 | 1.00 | 16.53 | C |
| ATOM | 3328 | CG | ARG | A | 433 | 102.155 | 53.002 | 24.374 | 1.00 | 23.81 | C |
| ATOM | 3329 | CD | ARG | A | 433 | 102.820 | 51.822 | 23.651 | 1.00 | 27.35 | C |
| ATOM | 3330 | NE | ARG | A | 433 | 101.846 | 50.739 | 23.405 | 1.00 | 40.17 | N |
| ATOM | 3331 | CZ | ARG | A | 433 | 101.725 | 50.034 | 22.279 | 1.00 | 38.69 | C |
| ATOM | 3332 | NH1 | ARG | A | 433 | 102.512 | 50.262 | 21.224 | 1.00 | 54.11 | N |
| ATOM | 3333 | NH2 | ARG | A | 433 | 100.800 | 49.089 | 22.199 | 1.00 | 29.01 | N |
| ATOM | 3334 | C | ARG | A | 433 | 101.003 | 56.474 | 23.188 | 1.00 | 11.20 | C |
| ATOM | 3335 | O | ARG | A | 433 | 101.728 | 57.383 | 23.547 | 1.00 | 10.17 | O |
| ATOM | 3336 | N | ILE | A | 434 | 100.320 | 56.514 | 22.042 | 1.00 | 10.54 | N |
| ATOM | 3337 | CA | ILE | A | 434 | 100.474 | 57.664 | 21.136 | 1.00 | 9.22 | C |
| ATOM | 3338 | CB | ILE | A | 434 | 100.008 | 57.353 | 19.680 | 1.00 | 8.85 | C |
| ATOM | 3339 | CG1 | ILE | A | 434 | 100.583 | 58.386 | 18.710 | 1.00 | 9.47 | C |
| ATOM | 3340 | CD1 | ILE | A | 434 | 102.113 | 58.485 | 18.712 | 1.00 | 8.53 | C |
| ATOM | 3341 | CG2 | ILE | A | 434 | 98.482 | 57.213 | 19.569 | 1.00 | 8.39 | C |
| ATOM | 3342 | C | ILE | A | 434 | 99.815 | 58.935 | 21.711 | 1.00 | 10.10 | C |
| ATOM | 3343 | O | ILE | A | 434 | 100.351 | 60.047 | 21.564 | 1.00 | 10.27 | O |
| ATOM | 3344 | N | ALA | A | 435 | 98.665 | 58.784 | 22.377 | 1.00 | 9.56 | N |
| ATOM | 3345 | CA | ALA | A | 435 | 97.962 | 59.938 | 22.968 | 1.00 | 8.34 | C |
| ATOM | 3346 | CB | ALA | A | 435 | 96.551 | 59.546 | 23.477 | 1.00 | 7.35 | C |
| ATOM | 3347 | C | ALA | A | 435 | 98.779 | 60.590 | 24.056 | 1.00 | 8.51 | C |
| ATOM | 3348 | O | ALA | A | 435 | 98.835 | 61.827 | 24.143 | 1.00 | 9.31 | O |
| ATOM | 3349 | N | GLN | A | 436 | 99.441 | 59.790 | 24.879 | 1.00 | 9.61 | N |
| ATOM | 3350 | CA | GLN | A | 436 | 100.296 | 60.347 | 25.919 | 1.00 | 11.37 | C |
| ATOM | 3351 | CB | GLN | A | 436 | 100.699 | 59.282 | 26.971 | 1.00 | 14.19 | C |
| ATOM | 3352 | CG | GLN | A | 436 | 99.515 | 58.768 | 27.816 | 1.00 | 16.35 | C |
| ATOM | 3353 | CD | GLN | A | 436 | 98.955 | 59.800 | 28.825 | 1.00 | 22.66 | C |
| ATOM | 3354 | OE1 | GLN | A | 436 | 99.375 | 60.978 | 28.861 | 1.00 | 25.03 | O |
| ATOM | 3355 | NE2 | GLN | A | 436 | 97.980 | 59.366 | 29.636 | 1.00 | 24.58 | N |
| ATOM | 3356 | C | GLN | A | 436 | 101.546 | 61.037 | 25.307 | 1.00 | 12.28 | C |
| ATOM | 3357 | O | GLN | A | 436 | 102.025 | 62.050 | 25.850 | 1.00 | 11.97 | O |
| ATOM | 3358 | N | VAL | A | 437 | 102.059 | 60.513 | 24.192 | 1.00 | 10.31 | N |
| ATOM | 3359 | CA | VAL | A | 437 | 103.124 | 61.222 | 23.447 | 1.00 | 9.13 | C |
| ATOM | 3360 | CB | VAL | A | 437 | 103.704 | 60.387 | 22.288 | 1.00 | 10.04 | C |
| ATOM | 3361 | CG1 | VAL | A | 437 | 104.592 | 61.244 | 21.390 | 1.00 | 9.42 | C |
| ATOM | 3362 | CG2 | VAL | A | 437 | 104.483 | 59.182 | 22.836 | 1.00 | 10.83 | C |
| ATOM | 3363 | C | VAL | A | 437 | 102.639 | 62.605 | 22.973 | 1.00 | 8.86 | C |
| ATOM | 3364 | O | VAL | A | 437 | 103.320 | 63.602 | 23.212 | 1.00 | 8.87 | O |
| ATOM | 3365 | N | ARG | A | 438 | 101.441 | 62.702 | 22.379 | 1.00 | 8.39 | N |
| ATOM | 3366 | CA | ARG | A | 438 | 100.914 | 64.002 | 21.944 | 1.00 | 8.28 | C |
| ATOM | 3367 | CB | ARG | A | 438 | 99.553 | 63.855 | 21.250 | 1.00 | 8.00 | C |
| ATOM | 3368 | CG | ARG | A | 438 | 99.075 | 65.152 | 20.619 | 1.00 | 9.42 | C |
| ATOM | 3369 | CD | ARG | A | 438 | 97.660 | 65.039 | 20.072 | 1.00 | 8.50 | C |
| ATOM | 3370 | NE | ARG | A | 438 | 97.240 | 66.241 | 19.353 | 1.00 | 8.96 | N |
| ATOM | 3371 | CZ | ARG | A | 438 | 96.840 | 67.380 | 19.917 | 1.00 | 8.98 | C |
| ATOM | 3372 | NH1 | ARG | A | 438 | 96.806 | 67.526 | 21.232 | 1.00 | 11.49 | N |
| ATOM | 3373 | NH2 | ARG | A | 438 | 96.445 | 68.379 | 19.162 | 1.00 | 9.76 | N |
| ATOM | 3374 | C | ARG | A | 438 | 100.762 | 64.940 | 23.154 | 1.00 | 9.49 | C |
| ATOM | 3375 | O | ARG | A | 438 | 101.134 | 66.103 | 23.108 | 1.00 | 7.66 | O |
| ATOM | 3376 | N | ALA | A | 439 | 100.202 | 64.405 | 24.243 | 1.00 | 9.79 | N |
| ATOM | 3377 | CA | ALA | A | 439 | 99.893 | 65.231 | 25.406 | 1.00 | 10.72 | C |
| ATOM | 3378 | CB | ALA | A | 439 | 99.141 | 64.419 | 26.476 | 1.00 | 10.49 | C |
| ATOM | 3379 | C | ALA | A | 439 | 101.166 | 65.845 | 25.991 | 1.00 | 11.63 | C |
| ATOM | 3380 | O | ALA | A | 439 | 101.091 | 66.875 | 26.602 | 1.00 | 9.51 | O |
| ATOM | 3381 | N | ALA | A | 440 | 102.326 | 65.218 | 25.794 | 1.00 | 10.51 | N |
| ATOM | 3382 | CA | ALA | A | 440 | 103.597 | 65.766 | 26.279 | 1.00 | 11.02 | C |
| ATOM | 3383 | CB | ALA | A | 440 | 104.458 | 64.652 | 26.889 | 1.00 | 10.64 | C |
| ATOM | 3384 | C | ALA | A | 440 | 104.416 | 66.526 | 25.216 | 1.00 | 10.53 | C |
| ATOM | 3385 | O | ALA | A | 440 | 105.501 | 66.993 | 25.519 | 1.00 | 10.60 | O |
| ATOM | 3386 | N | SER | A | 441 | 103.906 | 66.683 | 23.998 | 1.00 | 10.76 | N |
| ATOM | 3387 | CA | SER | A | 441 | 104.695 | 67.246 | 22.879 | 1.00 | 10.42 | C |
| ATOM | 3388 | CB | SER | A | 441 | 104.815 | 66.223 | 21.732 | 1.00 | 11.33 | C |
| ATOM | 3389 | OG | SER | A | 441 | 105.390 | 66.801 | 20.538 | 1.00 | 9.86 | O |
| ATOM | 3390 | C | SER | A | 441 | 104.028 | 68.493 | 22.321 | 1.00 | 10.28 | C |
| ATOM | 3391 | O | SER | A | 441 | 102.993 | 68.395 | 21.663 | 1.00 | 9.49 | O |
| ATOM | 3392 | N | PRO | A | 442 | 104.631 | 69.664 | 22.547 | 1.00 | 11.42 | N |
| ATOM | 3393 | CA | PRO | A | 442 | 104.121 | 70.887 | 21.926 | 1.00 | 11.27 | C |
| ATOM | 3394 | CB | PRO | A | 442 | 105.070 | 71.968 | 22.430 | 1.00 | 13.81 | C |
| ATOM | 3395 | CG | PRO | A | 442 | 105.690 | 71.414 | 23.661 | 1.00 | 16.52 | C |
| ATOM | 3396 | CD | PRO | A | 442 | 105.736 | 69.929 | 23.484 | 1.00 | 14.07 | C |
| ATOM | 3397 | C | PRO | A | 442 | 104.142 | 70.839 | 20.426 | 1.00 | 9.13 | C |
| ATOM | 3398 | O | PRO | A | 442 | 103.254 | 71.388 | 19.787 | 1.00 | 9.98 | O |
| ATOM | 3399 | N | ALA | A | 443 | 105.163 | 70.202 | 19.856 | 1.00 | 8.71 | N |
| ATOM | 3400 | CA | ALA | A | 443 | 105.228 | 70.013 | 18.402 | 1.00 | 8.33 | C |
| ATOM | 3401 | CB | ALA | A | 443 | 106.525 | 69.325 | 18.013 | 1.00 | 7.21 | C |
| ATOM | 3402 | C | ALA | A | 443 | 104.034 | 69.251 | 17.860 | 1.00 | 7.70 | C |
| ATOM | 3403 | O | ALA | A | 443 | 103.437 | 69.661 | 16.878 | 1.00 | 6.78 | O |
| ATOM | 3404 | N | LEU | A | 444 | 103.687 | 68.125 | 18.483 | 1.00 | 7.83 | N |
| ATOM | 3405 | CA | LEU | A | 444 | 102.546 | 67.318 | 18.001 | 1.00 | 8.98 | C |
| ATOM | 3406 | CB | LEU | A | 444 | 102.543 | 65.917 | 18.631 | 1.00 | 9.75 | C |
| ATOM | 3407 | CG | LEU | A | 444 | 103.683 | 64.943 | 18.311 | 1.00 | 9.68 | C |
| ATOM | 3408 | CD1 | LEU | A | 444 | 103.319 | 63.530 | 18.766 | 1.00 | 8.68 | C |
| ATOM | 3409 | CD2 | LEU | A | 444 | 103.926 | 64.924 | 16.809 | 1.00 | 7.72 | C |
| ATOM | 3410 | C | LEU | A | 444 | 101.201 | 68.010 | 18.296 | 1.00 | 10.38 | C |
| ATOM | 3411 | O | LEU | A | 444 | 100.196 | 67.754 | 17.621 | 1.00 | 10.11 | O |
| ATOM | 3412 | N | GLN | A | 445 | 101.177 | 68.889 | 19.292 | 1.00 | 9.45 | N |
| ATOM | 3413 | CA | GLN | A | 445 | 99.938 | 69.615 | 19.622 | 1.00 | 9.74 | C |
| ATOM | 3414 | CB | GLN | A | 445 | 100.027 | 70.197 | 21.040 | 1.00 | 10.47 | C |
| ATOM | 3415 | CG | GLN | A | 445 | 99.956 | 69.140 | 22.147 | 1.00 | 11.24 | C |
| ATOM | 3416 | CD | GLN | A | 445 | 100.441 | 69.657 | 23.483 | 1.00 | 11.90 | C |
| ATOM | 3417 | OE1 | GLN | A | 445 | 100.443 | 70.857 | 23.716 | 1.00 | 11.93 | O |
| ATOM | 3418 | NE2 | GLN | A | 445 | 100.841 | 68.750 | 24.376 | 1.00 | 11.74 | N |
| ATOM | 3419 | C | GLN | A | 445 | 99.647 | 70.741 | 18.626 | 1.00 | 9.80 | C |
| ATOM | 3420 | O | GLN | A | 445 | 98.516 | 70.888 | 18.142 | 1.00 | 10.73 | O |
| ATOM | 3421 | N | ARG | A | 446 | 100.680 | 71.502 | 18.292 | 1.00 | 8.91 | N |
| ATOM | 3422 | CA | ARG | A | 446 | 100.507 | 72.783 | 17.632 | 1.00 | 8.84 | C |
| ATOM | 3423 | CB | ARG | A | 446 | 100.285 | 73.874 | 18.676 | 1.00 | 8.99 | C |
| ATOM | 3424 | CG | ARG | A | 446 | 101.481 | 74.092 | 19.587 | 1.00 | 12.37 | C |
| ATOM | 3425 | CD | ARG | A | 446 | 101.339 | 75.297 | 20.550 | 1.00 | 13.03 | C |
| ATOM | 3426 | NE | ARG | A | 446 | 100.342 | 75.023 | 21.586 | 1.00 | 10.64 | N |
| ATOM | 3427 | CZ | ARG | A | 446 | 99.090 | 75.447 | 21.581 | 1.00 | 12.57 | C |
| ATOM | 3428 | NH1 | ARG | A | 446 | 98.618 | 76.178 | 20.593 | 1.00 | 13.47 | N |
| ATOM | 3429 | NH2 | ARG | A | 446 | 98.273 | 75.111 | 22.579 | 1.00 | 16.12 | N |
| ATOM | 3430 | C | ARG | A | 446 | 101.648 | 73.214 | 16.704 | 1.00 | 8.88 | C |
| ATOM | 3431 | O | ARG | A | 446 | 101.640 | 74.350 | 16.216 | 1.00 | 8.87 | O |
| ATOM | 3432 | N | GLY | A | 447 | 102.584 | 72.319 | 16.415 | 1.00 | 8.14 | N |
| ATOM | 3433 | CA | GLY | A | 447 | 103.690 | 72.651 | 15.559 | 1.00 | 9.16 | C |
| ATOM | 3434 | C | GLY | A | 447 | 103.279 | 72.667 | 14.105 | 1.00 | 8.98 | C |
| ATOM | 3435 | O | GLY | A | 447 | 102.307 | 72.011 | 13.721 | 1.00 | 8.78 | O |
| ATOM | 3436 | N | LEU | A | 448 | 104.020 | 73.423 | 13.300 | 1.00 | 8.43 | N |
| ATOM | 3437 | CA | LEU | A | 448 | 103.860 | 73.373 | 11.856 | 1.00 | 9.00 | C |
| ATOM | 3438 | CB | LEU | A | 448 | 104.858 | 74.315 | 11.165 | 1.00 | 8.58 | C |
| ATOM | 3439 | CG | LEU | A | 448 | 104.576 | 75.795 | 11.379 | 1.00 | 9.35 | C |
| ATOM | 3440 | CD1 | LEU | A | 448 | 105.818 | 76.659 | 11.023 | 1.00 | 7.31 | C |
| ATOM | 3441 | CD2 | LEU | A | 448 | 103.334 | 76.189 | 10.581 | 1.00 | 6.84 | C |
| ATOM | 3442 | C | LEU | A | 448 | 104.124 | 71.928 | 11.378 | 1.00 | 8.70 | C |
| ATOM | 3443 | O | LEU | A | 448 | 104.971 | 71.223 | 11.942 | 1.00 | 9.89 | O |
| ATOM | 3444 | N | GLN | A | 449 | 103.399 | 71.494 | 10.362 | 1.00 | 8.17 | N |
| ATOM | 3445 | CA | GLN | A | 449 | 103.636 | 70.205 | 9.729 | 1.00 | 7.61 | C |
| ATOM | 3446 | CB | GLN | A | 449 | 102.329 | 69.446 | 9.527 | 1.00 | 7.18 | C |
| ATOM | 3447 | CG | GLN | A | 449 | 102.507 | 68.040 | 8.926 | 1.00 | 6.89 | C |
| ATOM | 3448 | CD | GLN | A | 449 | 101.274 | 67.190 | 9.103 | 1.00 | 7.36 | C |
| ATOM | 3449 | OE1 | GLN | A | 449 | 100.531 | 67.327 | 10.121 | 1.00 | 8.33 | O |
| ATOM | 3450 | NE2 | GLN | A | 449 | 101.034 | 66.284 | 8.150 | 1.00 | 7.23 | N |
| ATOM | 3451 | C | GLN | A | 449 | 104.356 | 70.369 | 8.397 | 1.00 | 7.73 | C |
| ATOM | 3452 | O | GLN | A | 449 | 103.955 | 71.193 | 7.558 | 1.00 | 10.39 | O |
| ATOM | 3453 | N | LEU | A | 450 | 105.424 | 69.601 | 8.213 | 1.00 | 7.59 | N |
| ATOM | 3454 | CA | LEU | A | 450 | 106.077 | 69.487 | 6.897 | 1.00 | 8.52 | C |
| ATOM | 3455 | CB | LEU | A | 450 | 107.499 | 70.024 | 6.941 | 1.00 | 8.31 | C |
| ATOM | 3456 | CG | LEU | A | 450 | 107.671 | 71.496 | 7.301 | 1.00 | 8.49 | C |
| ATOM | 3457 | CD1 | LEU | A | 450 | 109.155 | 71.752 | 7.571 | 1.00 | 7.66 | C |
| ATOM | 3458 | CD2 | LEU | A | 450 | 107.091 | 72.398 | 6.211 | 1.00 | 8.71 | C |
| ATOM | 3459 | C | LEU | A | 450 | 106.087 | 68.028 | 6.452 | 1.00 | 8.12 | C |
| ATOM | 3460 | O | LEU | A | 450 | 106.672 | 67.176 | 7.119 | 1.00 | 8.15 | O |
| ATOM | 3461 | N | ASN | A | 451 | 105.422 | 67.740 | 5.343 | 1.00 | 8.05 | N |
| ATOM | 3462 | CA | ASN | A | 451 | 105.323 | 66.383 | 4.835 | 1.00 | 8.39 | C |
| ATOM | 3463 | CB | ASN | A | 451 | 104.189 | 66.272 | 3.804 | 1.00 | 8.84 | C |
| ATOM | 3464 | CG | ASN | A | 451 | 102.826 | 66.555 | 4.397 | 1.00 | 8.85 | C |
| ATOM | 3465 | OD1 | ASN | A | 451 | 102.612 | 66.390 | 5.602 | 1.00 | 10.53 | O |
| ATOM | 3466 | ND2 | ASN | A | 451 | 101.893 | 67.002 | 3.555 | 1.00 | 8.66 | N |
| ATOM | 3467 | C | ASN | A | 451 | 106.617 | 65.923 | 4.168 | 1.00 | 9.39 | C |
| ATOM | 3468 | O | ASN | A | 451 | 107.200 | 66.659 | 3.399 | 1.00 | 7.88 | O |
| ATOM | 3469 | N | LEU | A | 452 | 107.040 | 64.691 | 4.457 | 1.00 | 8.89 | N |
| ATOM | 3470 | CA | LEU | A | 452 | 108.214 | 64.099 | 3.842 | 1.00 | 10.73 | C |
| ATOM | 3471 | CB | LEU | A | 452 | 109.203 | 63.640 | 4.900 | 1.00 | 9.84 | C |
| ATOM | 3472 | CG | LEU | A | 452 | 109.802 | 64.737 | 5.795 | 1.00 | 12.35 | C |
| ATOM | 3473 | CD1 | LEU | A | 452 | 110.541 | 64.105 | 6.968 | 1.00 | 12.21 | C |
| ATOM | 3474 | CD2 | LEU | A | 452 | 110.732 | 65.628 | 4.984 | 1.00 | 9.86 | C |
| ATOM | 3475 | C | LEU | A | 452 | 107.838 | 62.928 | 2.942 | 1.00 | 10.79 | C |
| ATOM | 3476 | O | LEU | A | 452 | 108.383 | 62.794 | 1.843 | 1.00 | 11.51 | O |
| ATOM | 3477 | N | GLU | A | 453 | 106.938 | 62.070 | 3.416 | 1.00 | 8.44 | N |
| ATOM | 3478 | CA | GLU | A | 453 | 106.483 | 60.925 | 2.630 | 1.00 | 8.57 | C |
| ATOM | 3479 | CB | GLU | A | 453 | 107.396 | 59.702 | 2.844 | 1.00 | 8.80 | C |
| ATOM | 3480 | CG | GLU | A | 453 | 106.949 | 58.484 | 2.047 | 1.00 | 9.90 | C |
| ATOM | 3481 | CD | GLU | A | 453 | 107.862 | 57.254 | 2.128 | 1.00 | 10.82 | C |
| ATOM | 3482 | OE1 | GLU | A | 453 | 108.834 | 57.219 | 2.904 | 1.00 | 12.72 | O |
| ATOM | 3483 | OE2 | GLU | A | 453 | 107.576 | 56.293 | 1.377 | 1.00 | 15.14 | O |
| ATOM | 3484 | C | GLU | A | 453 | 105.066 | 60.577 | 3.051 | 1.00 | 7.83 | C |
| ATOM | 3485 | O | GLU | A | 453 | 104.782 | 60.470 | 4.232 | 1.00 | 7.09 | O |
| ATOM | 3486 | N | LEU | A | 454 | 104.178 | 60.443 | 2.081 | 1.00 | 8.64 | N |
| ATOM | 3487 | CA | LEU | A | 454 | 102.808 | 59.973 | 2.310 | 1.00 | 9.02 | C |
| ATOM | 3488 | CB | LEU | A | 454 | 101.852 | 61.162 | 2.398 | 1.00 | 9.84 | C |
| ATOM | 3489 | CG | LEU | A | 454 | 101.953 | 62.009 | 3.654 | 1.00 | 9.51 | C |
| ATOM | 3490 | CD1 | LEU | A | 454 | 101.219 | 63.340 | 3.440 | 1.00 | 11.30 | C |
| ATOM | 3491 | CD2 | LEU | A | 454 | 101.411 | 61.251 | 4.860 | 1.00 | 8.61 | C |
| ATOM | 3492 | C | LEU | A | 454 | 102.512 | 59.116 | 1.078 | 1.00 | 9.20 | C |
| ATOM | 3493 | O | LEU | A | 454 | 102.124 | 59.616 | 0.031 | 1.00 | 8.58 | O |
| ATOM | 3494 | N | GLN | A | 455 | 102.767 | 57.827 | 1.191 | 1.00 | 9.05 | N |
| ATOM | 3495 | CA | GLN | A | 455 | 102.848 | 56.968 | 0.030 | 1.00 | 9.12 | C |
| ATOM | 3496 | CB | GLN | A | 455 | 104.235 | 57.129 | −0.599 | 1.00 | 9.92 | C |
| ATOM | 3497 | CG | GLN | A | 455 | 104.454 | 56.334 | −1.868 | 1.00 | 12.29 | C |
| ATOM | 3498 | CD | GLN | A | 455 | 105.791 | 56.637 | −2.564 | 1.00 | 11.72 | C |
| ATOM | 3499 | OE1 | GLN | A | 455 | 105.850 | 56.679 | −3.784 | 1.00 | 13.34 | O |
| ATOM | 3500 | NE2 | GLN | A | 455 | 106.850 | 56.824 | −1.794 | 1.00 | 12.93 | N |
| ATOM | 3501 | C | GLN | A | 455 | 102.630 | 55.544 | 0.493 | 1.00 | 8.61 | C |
| ATOM | 3502 | O | GLN | A | 455 | 103.356 | 55.051 | 1.350 | 1.00 | 7.56 | O |
| ATOM | 3503 | N | GLY | A | 456 | 101.607 | 54.899 | −0.041 | 1.00 | 9.28 | N |
| ATOM | 3504 | CA | GLY | A | 456 | 101.355 | 53.490 | 0.267 | 1.00 | 9.96 | C |
| ATOM | 3505 | C | GLY | A | 456 | 101.128 | 53.318 | 1.753 | 1.00 | 9.93 | C |
| ATOM | 3506 | O | GLY | A | 456 | 100.357 | 54.060 | 2.369 | 1.00 | 10.32 | O |
| ATOM | 3507 | N | ASN | A | 457 | 101.840 | 52.377 | 2.337 | 1.00 | 9.51 | N |
| ATOM | 3508 | CA | ASN | A | 457 | 101.719 | 52.098 | 3.753 | 1.00 | 11.18 | C |
| ATOM | 3509 | CB | ASN | A | 457 | 102.118 | 50.640 | 4.018 | 1.00 | 11.06 | C |
| ATOM | 3510 | CG | ASN | A | 457 | 101.092 | 49.650 | 3.480 | 1.00 | 10.38 | C |
| ATOM | 3511 | OD1 | ASN | A | 457 | 101.446 | 48.646 | 2.872 | 1.00 | 13.80 | O |
| ATOM | 3512 | ND2 | ASN | A | 457 | 99.857 | 49.946 | 3.670 | 1.00 | 7.53 | N |
| ATOM | 3513 | C | ASN | A | 457 | 102.536 | 53.015 | 4.665 | 1.00 | 10.84 | C |
| ATOM | 3514 | O | ASN | A | 457 | 102.499 | 52.839 | 5.876 | 1.00 | 10.92 | O |
| ATOM | 3515 | N | ARG | A | 458 | 103.218 | 54.001 | 4.091 | 1.00 | 9.12 | N |
| ATOM | 3516 | CA | ARG | A | 458 | 104.253 | 54.747 | 4.781 | 1.00 | 9.92 | C |
| ATOM | 3517 | CB | ARG | A | 458 | 105.590 | 54.605 | 4.015 | 1.00 | 10.01 | C |
| ATOM | 3518 | CG | ARG | A | 458 | 106.094 | 53.171 | 3.862 | 1.00 | 12.11 | C |
| ATOM | 3519 | CD | ARG | A | 458 | 107.144 | 53.017 | 2.739 | 1.00 | 11.48 | C |
| ATOM | 3520 | NE | ARG | A | 458 | 108.249 | 53.944 | 2.968 | 1.00 | 10.95 | N |
| ATOM | 3521 | CZ | ARG | A | 458 | 109.373 | 53.652 | 3.616 | 1.00 | 13.00 | C |
| ATOM | 3522 | NH1 | ARG | A | 458 | 109.606 | 52.422 | 4.081 | 1.00 | 11.48 | N |
| ATOM | 3523 | NH2 | ARG | A | 458 | 110.316 | 54.593 | 3.753 | 1.00 | 12.75 | N |
| ATOM | 3524 | C | ARG | A | 458 | 103.888 | 56.231 | 4.906 | 1.00 | 9.10 | C |
| ATOM | 3525 | O | ARG | A | 458 | 103.423 | 56.834 | 3.947 | 1.00 | 10.94 | O |
| ATOM | 3526 | N | ALA | A | 459 | 104.098 | 56.798 | 6.089 | 1.00 | 8.77 | N |
| ATOM | 3527 | CA | ALA | A | 459 | 104.010 | 58.251 | 6.327 | 1.00 | 8.88 | C |
| ATOM | 3528 | CB | ALA | A | 459 | 102.760 | 58.619 | 7.089 | 1.00 | 7.78 | C |
| ATOM | 3529 | C | ALA | A | 459 | 105.241 | 58.726 | 7.096 | 1.00 | 9.20 | C |
| ATOM | 3530 | O | ALA | A | 459 | 105.692 | 58.073 | 8.044 | 1.00 | 9.88 | O |
| ATOM | 3531 | N | ALA | A | 460 | 105.779 | 59.872 | 6.695 | 1.00 | 9.71 | N |
| ATOM | 3532 | CA | ALA | A | 460 | 106.843 | 60.537 | 7.442 | 1.00 | 8.98 | C |
| ATOM | 3533 | CB | ALA | A | 460 | 108.218 | 60.141 | 6.902 | 1.00 | 7.69 | C |
| ATOM | 3534 | C | ALA | A | 460 | 106.614 | 62.060 | 7.331 | 1.00 | 9.17 | C |
| ATOM | 3535 | O | ALA | A | 460 | 106.328 | 62.573 | 6.244 | 1.00 | 8.73 | O |
| ATOM | 3536 | N | PHE | A | 461 | 106.693 | 62.767 | 8.458 | 1.00 | 7.86 | N |
| ATOM | 3537 | CA | PHE | A | 461 | 106.517 | 64.207 | 8.465 | 1.00 | 7.58 | C |
| ATOM | 3538 | CB | PHE | A | 461 | 105.049 | 64.579 | 8.296 | 1.00 | 8.14 | C |
| ATOM | 3539 | CG | PHE | A | 461 | 104.111 | 63.965 | 9.316 | 1.00 | 8.08 | C |
| ATOM | 3540 | CD1 | PHE | A | 461 | 103.459 | 62.771 | 9.063 | 1.00 | 9.21 | C |
| ATOM | 3541 | CE1 | PHE | A | 461 | 102.552 | 62.228 | 9.988 | 1.00 | 9.07 | C |
| ATOM | 3542 | CZ | PHE | A | 461 | 102.279 | 62.906 | 11.149 | 1.00 | 8.95 | C |
| ATOM | 3543 | CE2 | PHE | A | 461 | 102.917 | 64.095 | 11.420 | 1.00 | 9.15 | C |
| ATOM | 3544 | CD2 | PHE | A | 461 | 103.817 | 64.631 | 10.493 | 1.00 | 8.86 | C |
| ATOM | 3545 | C | PHE | A | 461 | 107.110 | 64.813 | 9.731 | 1.00 | 7.51 | C |
| ATOM | 3546 | O | PHE | A | 461 | 107.298 | 64.110 | 10.721 | 1.00 | 7.73 | O |
| ATOM | 3547 | N | TYR | A | 462 | 107.487 | 66.089 | 9.660 | 1.00 | 7.26 | N |
| ATOM | 3548 | CA | TYR | A | 462 | 107.939 | 66.832 | 10.828 | 1.00 | 7.73 | C |
| ATOM | 3549 | CB | TYR | A | 462 | 109.029 | 67.861 | 10.475 | 1.00 | 8.20 | C |
| ATOM | 3550 | CG | TYR | A | 462 | 110.324 | 67.288 | 9.935 | 1.00 | 8.39 | C |
| ATOM | 3551 | CD1 | TYR | A | 462 | 111.122 | 66.451 | 10.705 | 1.00 | 8.74 | C |
| ATOM | 3552 | CE1 | TYR | A | 462 | 112.308 | 65.951 | 10.208 | 1.00 | 9.16 | C |
| ATOM | 3553 | CZ | TYR | A | 462 | 112.731 | 66.300 | 8.962 | 1.00 | 9.89 | C |
| ATOM | 3554 | OH | TYR | A | 462 | 113.894 | 65.795 | 8.465 | 1.00 | 9.19 | O |
| ATOM | 3555 | CE2 | TYR | A | 462 | 111.973 | 67.146 | 8.181 | 1.00 | 9.88 | C |
| ATOM | 3556 | CD2 | TYR | A | 462 | 110.788 | 67.640 | 8.681 | 1.00 | 9.71 | C |
| ATOM | 3557 | C | TYR | A | 462 | 106.754 | 67.580 | 11.438 | 1.00 | 7.62 | C |
| ATOM | 3558 | O | TYR | A | 462 | 105.818 | 67.962 | 10.730 | 1.00 | 7.10 | O |
| ATOM | 3559 | N | ARG | A | 463 | 106.797 | 67.767 | 12.755 | 1.00 | 6.76 | N |
| ATOM | 3560 | CA | ARG | A | 463 | 106.007 | 68.772 | 13.438 | 1.00 | 6.89 | C |
| ATOM | 3561 | CB | ARG | A | 463 | 105.019 | 68.159 | 14.422 | 1.00 | 7.03 | C |
| ATOM | 3562 | CG | ARG | A | 463 | 103.964 | 67.236 | 13.814 | 1.00 | 7.68 | C |
| ATOM | 3563 | CD | ARG | A | 463 | 103.052 | 67.950 | 12.831 | 1.00 | 7.20 | C |
| ATOM | 3564 | NE | ARG | A | 463 | 102.264 | 69.023 | 13.464 | 1.00 | 7.15 | N |
| ATOM | 3565 | CZ | ARG | A | 463 | 101.142 | 68.855 | 14.151 | 1.00 | 7.38 | C |
| ATOM | 3566 | NH1 | ARG | A | 463 | 100.630 | 67.640 | 14.360 | 1.00 | 8.84 | N |
| ATOM | 3567 | NH2 | ARG | A | 463 | 100.535 | 69.908 | 14.660 | 1.00 | 7.17 | N |
| ATOM | 3568 | C | ARG | A | 463 | 107.014 | 69.643 | 14.172 | 1.00 | 6.74 | C |
| ATOM | 3569 | O | ARG | A | 463 | 107.906 | 69.115 | 14.824 | 1.00 | 7.05 | O |
| ATOM | 3570 | N | VAL | A | 464 | 106.906 | 70.958 | 14.025 | 1.00 | 6.88 | N |
| ATOM | 3571 | CA | VAL | A | 464 | 107.875 | 71.887 | 14.601 | 1.00 | 8.03 | C |
| ATOM | 3572 | CB | VAL | A | 464 | 108.817 | 72.497 | 13.536 | 1.00 | 7.08 | C |
| ATOM | 3573 | CG1 | VAL | A | 464 | 109.893 | 73.329 | 14.217 | 1.00 | 6.33 | C |
| ATOM | 3574 | CG2 | VAL | A | 464 | 109.469 | 71.395 | 12.710 | 1.00 | 7.39 | C |
| ATOM | 3575 | C | VAL | A | 464 | 107.219 | 73.028 | 15.366 | 1.00 | 8.00 | C |
| ATOM | 3576 | O | VAL | A | 464 | 106.409 | 73.742 | 14.831 | 1.00 | 7.76 | O |
| ATOM | 3577 | N | TYR | A | 465 | 107.584 | 73.165 | 16.633 | 1.00 | 8.64 | N |
| ATOM | 3578 | CA | TYR | A | 465 | 107.122 | 74.276 | 17.471 | 1.00 | 9.47 | C |
| ATOM | 3579 | CB | TYR | A | 465 | 106.258 | 73.752 | 18.600 | 1.00 | 10.60 | C |
| ATOM | 3580 | CG | TYR | A | 465 | 105.753 | 74.825 | 19.541 | 1.00 | 11.90 | C |
| ATOM | 3581 | CD1 | TYR | A | 465 | 104.732 | 75.670 | 19.151 | 1.00 | 13.84 | C |
| ATOM | 3582 | CE1 | TYR | A | 465 | 104.254 | 76.663 | 19.993 | 1.00 | 14.60 | C |
| ATOM | 3583 | CZ | TYR | A | 465 | 104.779 | 76.792 | 21.257 | 1.00 | 13.31 | C |
| ATOM | 3584 | OH | TYR | A | 465 | 104.254 | 77.782 | 22.030 | 1.00 | 16.79 | O |
| ATOM | 3585 | CE2 | TYR | A | 465 | 105.806 | 75.977 | 21.684 | 1.00 | 11.86 | C |
| ATOM | 3586 | CD2 | TYR | A | 465 | 106.295 | 74.991 | 20.825 | 1.00 | 12.53 | C |
| ATOM | 3587 | C | TYR | A | 465 | 108.320 | 74.983 | 18.079 | 1.00 | 9.77 | C |
| ATOM | 3588 | O | TYR | A | 465 | 109.147 | 74.354 | 18.740 | 1.00 | 11.56 | O |
| ATOM | 3589 | N | GLN | A | 466 | 108.391 | 76.283 | 17.871 | 1.00 | 10.07 | N |
| ATOM | 3590 | CA | GLN | A | 466 | 109.439 | 77.128 | 18.468 | 1.00 | 10.77 | C |
| ATOM | 3591 | CB | GLN | A | 466 | 110.612 | 77.330 | 17.505 | 1.00 | 10.07 | C |
| ATOM | 3592 | CG | GLN | A | 466 | 111.717 | 78.268 | 18.002 | 1.00 | 10.39 | C |
| ATOM | 3593 | CD | GLN | A | 466 | 112.976 | 78.111 | 17.142 | 1.00 | 12.11 | C |
| ATOM | 3594 | OE1 | GLN | A | 466 | 113.598 | 77.051 | 17.154 | 1.00 | 12.62 | O |
| ATOM | 3595 | NE2 | GLN | A | 466 | 113.314 | 79.136 | 16.361 | 1.00 | 10.16 | N |
| ATOM | 3596 | C | GLN | A | 466 | 108.820 | 78.484 | 18.834 | 1.00 | 10.05 | C |
| ATOM | 3597 | O | GLN | A | 466 | 108.450 | 79.264 | 17.963 | 1.00 | 9.79 | O |
| ATOM | 3598 | N | HIS | A | 467 | 108.721 | 78.749 | 20.126 | 1.00 | 11.10 | N |
| ATOM | 3599 | CA | HIS | A | 467 | 108.041 | 79.942 | 20.617 | 1.00 | 13.74 | C |
| ATOM | 3600 | CB | HIS | A | 467 | 106.521 | 79.793 | 20.415 | 1.00 | 14.41 | C |
| ATOM | 3601 | CG | HIS | A | 467 | 105.732 | 81.007 | 20.819 | 1.00 | 15.26 | C |
| ATOM | 3602 | ND1 | HIS | A | 467 | 105.904 | 82.213 | 20.230 | 1.00 | 15.80 | N |
| ATOM | 3603 | CE1 | HIS | A | 467 | 105.081 | 83.117 | 20.823 | 1.00 | 17.11 | C |
| ATOM | 3604 | NE2 | HIS | A | 467 | 104.388 | 82.486 | 21.798 | 1.00 | 15.48 | N |
| ATOM | 3605 | CD2 | HIS | A | 467 | 104.760 | 81.187 | 21.825 | 1.00 | 16.55 | C |
| ATOM | 3606 | C | HIS | A | 467 | 108.363 | 80.097 | 22.076 | 1.00 | 14.42 | C |
| ATOM | 3607 | O | HIS | A | 467 | 108.351 | 79.098 | 22.827 | 1.00 | 14.47 | O |
| ATOM | 3608 | N | ASP | A | 468 | 108.659 | 81.329 | 22.499 | 1.00 | 17.70 | N |
| ATOM | 3609 | CA | ASP | A | 468 | 108.890 | 81.657 | 23.942 | 1.00 | 22.07 | C |
| ATOM | 3610 | CB | ASP | A | 468 | 107.587 | 81.581 | 24.745 | 1.00 | 26.48 | C |
| ATOM | 3611 | CG | ASP | A | 468 | 106.657 | 82.741 | 24.468 | 1.00 | 42.65 | C |
| ATOM | 3612 | OD1 | ASP | A | 468 | 107.077 | 83.711 | 23.780 | 1.00 | 43.62 | O |
| ATOM | 3613 | OD2 | ASP | A | 468 | 105.500 | 82.676 | 24.953 | 1.00 | 44.61 | O |
| ATOM | 3614 | C | ASP | A | 468 | 109.903 | 80.749 | 24.623 | 1.00 | 20.64 | C |
| ATOM | 3615 | O | ASP | A | 468 | 109.624 | 80.195 | 25.680 | 1.00 | 26.80 | O |
| ATOM | 3616 | N | GLY | A | 469 | 111.064 | 80.569 | 24.007 | 1.00 | 19.35 | N |
| ATOM | 3617 | CA | GLY | A | 469 | 112.078 | 79.708 | 24.574 | 1.00 | 18.61 | C |
| ATOM | 3618 | C | GLY | A | 469 | 111.811 | 78.209 | 24.559 | 1.00 | 17.77 | C |
| ATOM | 3619 | O | GLY | A | 469 | 112.649 | 77.459 | 25.032 | 1.00 | 24.81 | O |
| ATOM | 3620 | N | VAL | A | 470 | 110.662 | 77.754 | 24.047 | 1.00 | 15.70 | N |
| ATOM | 3621 | CA | VAL | A | 470 | 110.409 | 76.323 | 23.886 | 1.00 | 14.21 | C |
| ATOM | 3622 | CB | VAL | A | 470 | 108.949 | 75.970 | 24.204 | 1.00 | 17.62 | C |
| ATOM | 3623 | CG1 | VAL | A | 470 | 108.680 | 74.490 | 23.966 | 1.00 | 17.53 | C |
| ATOM | 3624 | CG2 | VAL | A | 470 | 108.629 | 76.377 | 25.641 | 1.00 | 20.01 | C |
| ATOM | 3625 | C | VAL | A | 470 | 110.718 | 75.980 | 22.427 | 1.00 | 13.68 | C |
| ATOM | 3626 | O | VAL | A | 470 | 110.289 | 76.704 | 21.520 | 1.00 | 13.92 | O |
| ATOM | 3627 | N | HIS | A | 471 | 111.478 | 74.909 | 22.201 | 1.00 | 11.87 | N |
| ATOM | 3628 | CA | HIS | A | 471 | 111.801 | 74.482 | 20.838 | 1.00 | 11.99 | C |
| ATOM | 3629 | CB | HIS | A | 471 | 113.095 | 75.096 | 20.336 | 1.00 | 12.32 | C |
| ATOM | 3630 | CG | HIS | A | 471 | 114.213 | 75.114 | 21.342 | 1.00 | 13.48 | C |
| ATOM | 3631 | ND1 | HIS | A | 471 | 114.667 | 74.004 | 21.956 | 1.00 | 13.35 | N |
| ATOM | 3632 | CE1 | HIS | A | 471 | 115.671 | 74.348 | 22.782 | 1.00 | 16.30 | C |
| ATOM | 3633 | NE2 | HIS | A | 471 | 115.861 | 75.671 | 22.685 | 1.00 | 14.13 | N |
| ATOM | 3634 | CD2 | HIS | A | 471 | 114.989 | 76.171 | 21.809 | 1.00 | 15.06 | C |
| ATOM | 3635 | C | HIS | A | 471 | 111.835 | 72.995 | 20.698 | 1.00 | 12.91 | C |
| ATOM | 3636 | O | HIS | A | 471 | 112.680 | 72.312 | 21.313 | 1.00 | 12.73 | O |
| ATOM | 3637 | N | GLN | A | 472 | 110.902 | 72.465 | 19.900 | 1.00 | 9.92 | N |
| ATOM | 3638 | CA | GLN | A | 472 | 110.824 | 71.025 | 19.710 | 1.00 | 10.20 | C |
| ATOM | 3639 | CB | GLN | A | 472 | 109.733 | 70.401 | 20.602 | 1.00 | 9.95 | C |
| ATOM | 3640 | CG | GLN | A | 472 | 109.721 | 68.875 | 20.534 | 1.00 | 10.88 | C |
| ATOM | 3641 | CD | GLN | A | 472 | 108.632 | 68.239 | 21.380 | 1.00 | 10.47 | C |
| ATOM | 3642 | OE1 | GLN | A | 472 | 107.426 | 68.462 | 21.156 | 1.00 | 8.61 | O |
| ATOM | 3643 | NE2 | GLN | A | 472 | 109.046 | 67.401 | 22.331 | 1.00 | 7.85 | N |
| ATOM | 3644 | C | GLN | A | 472 | 110.607 | 70.692 | 18.240 | 1.00 | 11.45 | C |
| ATOM | 3645 | O | GLN | A | 472 | 109.686 | 71.230 | 17.602 | 1.00 | 10.08 | O |
| ATOM | 3646 | N | ILE | A | 473 | 111.473 | 69.830 | 17.701 | 1.00 | 10.13 | N |
| ATOM | 3647 | CA | ILE | A | 473 | 111.311 | 69.305 | 16.352 | 1.00 | 10.91 | C |
| ATOM | 3648 | CB | ILE | A | 473 | 112.593 | 69.498 | 15.494 | 1.00 | 12.38 | C |
| ATOM | 3649 | CG1 | ILE | A | 473 | 113.011 | 70.960 | 15.449 | 1.00 | 12.96 | C |
| ATOM | 3650 | CD1 | ILE | A | 473 | 114.480 | 71.192 | 15.089 | 1.00 | 15.54 | C |
| ATOM | 3651 | CG2 | ILE | A | 473 | 112.369 | 68.961 | 14.083 | 1.00 | 13.05 | C |
| ATOM | 3652 | C | ILE | A | 473 | 110.984 | 67.814 | 16.477 | 1.00 | 10.90 | C |
| ATOM | 3653 | O | ILE | A | 473 | 111.742 | 67.053 | 17.106 | 1.00 | 12.78 | O |
| ATOM | 3654 | N | ALA | A | 474 | 109.837 | 67.413 | 15.931 | 1.00 | 10.30 | N |
| ATOM | 3655 | CA | ALA | A | 474 | 109.392 | 66.012 | 15.953 | 1.00 | 9.91 | C |
| ATOM | 3656 | CB | ALA | A | 474 | 108.010 | 65.886 | 16.576 | 1.00 | 7.86 | C |
| ATOM | 3657 | C | ALA | A | 474 | 109.383 | 65.424 | 14.551 | 1.00 | 9.44 | C |
| ATOM | 3658 | O | ALA | A | 474 | 108.960 | 66.082 | 13.593 | 1.00 | 10.72 | O |
| ATOM | 3659 | N | LEU | A | 475 | 109.879 | 64.205 | 14.441 | 1.00 | 8.79 | N |
| ATOM | 3660 | CA | LEU | A | 475 | 109.812 | 63.405 | 13.222 | 1.00 | 7.97 | C |
| ATOM | 3661 | CB | LEU | A | 475 | 111.190 | 62.896 | 12.834 | 1.00 | 7.16 | C |
| ATOM | 3662 | CG | LEU | A | 475 | 111.248 | 61.869 | 11.682 | 1.00 | 7.43 | C |
| ATOM | 3663 | CD1 | LEU | A | 475 | 110.660 | 62.450 | 10.390 | 1.00 | 6.53 | C |
| ATOM | 3664 | CD2 | LEU | A | 475 | 112.690 | 61.417 | 11.452 | 1.00 | 6.30 | C |
| ATOM | 3665 | C | LEU | A | 475 | 108.906 | 62.230 | 13.500 | 1.00 | 8.86 | C |
| ATOM | 3666 | O | LEU | A | 475 | 109.220 | 61.394 | 14.372 | 1.00 | 9.58 | O |
| ATOM | 3667 | N | VAL | A | 476 | 107.794 | 62.150 | 12.766 | 1.00 | 9.42 | N |
| ATOM | 3668 | CA | VAL | A | 476 | 106.803 | 61.098 | 12.963 | 1.00 | 7.93 | C |
| ATOM | 3669 | CB | VAL | A | 476 | 105.379 | 61.686 | 12.999 | 1.00 | 8.69 | C |
| ATOM | 3670 | CG1 | VAL | A | 476 | 104.310 | 60.581 | 13.034 | 1.00 | 6.98 | C |
| ATOM | 3671 | CG2 | VAL | A | 476 | 105.220 | 62.653 | 14.164 | 1.00 | 7.14 | C |
| ATOM | 3672 | C | VAL | A | 476 | 106.921 | 60.140 | 11.795 | 1.00 | 9.09 | C |
| ATOM | 3673 | O | VAL | A | 476 | 106.947 | 60.580 | 10.632 | 1.00 | 9.05 | O |
| ATOM | 3674 | N | LEU | A | 477 | 107.013 | 58.842 | 12.103 | 1.00 | 8.19 | N |
| ATOM | 3675 | CA | LEU | A | 477 | 107.026 | 57.787 | 11.108 | 1.00 | 7.85 | C |
| ATOM | 3676 | CB | LEU | A | 477 | 108.360 | 57.035 | 11.113 | 1.00 | 7.10 | C |
| ATOM | 3677 | CG | LEU | A | 477 | 109.662 | 57.837 | 11.136 | 1.00 | 7.47 | C |
| ATOM | 3678 | CD1 | LEU | A | 477 | 110.856 | 56.885 | 11.282 | 1.00 | 7.28 | C |
| ATOM | 3679 | CD2 | LEU | A | 477 | 109.829 | 58.741 | 9.897 | 1.00 | 6.57 | C |
| ATOM | 3680 | C | LEU | A | 477 | 105.882 | 56.807 | 11.375 | 1.00 | 9.01 | C |
| ATOM | 3681 | O | LEU | A | 477 | 105.751 | 56.303 | 12.495 | 1.00 | 9.23 | O |
| ATOM | 3682 | N | LEU | A | 478 | 105.074 | 56.531 | 10.339 | 1.00 | 8.76 | N |
| ATOM | 3683 | CA | LEU | A | 478 | 103.941 | 55.620 | 10.444 | 1.00 | 8.70 | C |
| ATOM | 3684 | CB | LEU | A | 478 | 102.614 | 56.382 | 10.265 | 1.00 | 8.48 | C |
| ATOM | 3685 | CG | LEU | A | 478 | 102.349 | 57.590 | 11.188 | 1.00 | 9.45 | C |
| ATOM | 3686 | CD1 | LEU | A | 478 | 101.102 | 58.392 | 10.793 | 1.00 | 8.74 | C |
| ATOM | 3687 | CD2 | LEU | A | 478 | 102.188 | 57.125 | 12.614 | 1.00 | 9.75 | C |
| ATOM | 3688 | C | LEU | A | 478 | 104.055 | 54.495 | 9.410 | 1.00 | 8.94 | C |
| ATOM | 3689 | O | LEU | A | 478 | 104.468 | 54.719 | 8.255 | 1.00 | 8.93 | O |
| ATOM | 3690 | N | ASN | A | 479 | 103.660 | 53.296 | 9.826 | 1.00 | 8.49 | N |
| ATOM | 3691 | CA | ASN | A | 479 | 103.580 | 52.136 | 8.974 | 1.00 | 8.70 | C |
| ATOM | 3692 | CB | ASN | A | 479 | 104.713 | 51.185 | 9.333 | 1.00 | 10.04 | C |
| ATOM | 3693 | CG | ASN | A | 479 | 104.643 | 49.865 | 8.581 | 1.00 | 10.21 | C |
| ATOM | 3694 | OD1 | ASN | A | 479 | 103.847 | 49.701 | 7.652 | 1.00 | 8.10 | O |
| ATOM | 3695 | ND2 | ASN | A | 479 | 105.494 | 48.916 | 8.980 | 1.00 | 9.01 | N |
| ATOM | 3696 | C | ASN | A | 479 | 102.208 | 51.476 | 9.205 | 1.00 | 10.92 | C |
| ATOM | 3697 | O | ASN | A | 479 | 101.954 | 50.865 | 10.278 | 1.00 | 10.14 | O |
| ATOM | 3698 | N | LYS | A | 480 | 101.313 | 51.632 | 8.235 | 1.00 | 9.12 | N |
| ATOM | 3699 | CA | LYS | A | 480 | 99.965 | 51.066 | 8.337 | 1.00 | 9.96 | C |
| ATOM | 3700 | CB | LYS | A | 480 | 98.905 | 52.061 | 7.801 | 1.00 | 10.03 | C |
| ATOM | 3701 | CG | LYS | A | 480 | 98.904 | 52.249 | 6.286 | 1.00 | 10.64 | C |
| ATOM | 3702 | CD | LYS | A | 480 | 97.837 | 53.234 | 5.751 | 1.00 | 10.86 | C |
| ATOM | 3703 | CE | LYS | A | 480 | 97.583 | 53.015 | 4.250 | 1.00 | 11.36 | C |
| ATOM | 3704 | NZ | LYS | A | 480 | 96.412 | 52.109 | 3.973 | 1.00 | 13.64 | N |
| ATOM | 3705 | C | LYS | A | 480 | 99.852 | 49.725 | 7.601 | 1.00 | 10.86 | C |
| ATOM | 3706 | O | LYS | A | 480 | 98.736 | 49.272 | 7.333 | 1.00 | 10.55 | O |
| ATOM | 3707 | N | GLY | A | 481 | 100.987 | 49.111 | 7.254 | 1.00 | 9.20 | N |
| ATOM | 3708 | CA | GLY | A | 481 | 100.996 | 47.815 | 6.584 | 1.00 | 10.33 | C |
| ATOM | 3709 | C | GLY | A | 481 | 101.345 | 46.666 | 7.520 | 1.00 | 11.31 | C |
| ATOM | 3710 | O | GLY | A | 481 | 101.498 | 46.848 | 8.747 | 1.00 | 12.10 | O |
| ATOM | 3711 | N | ASP | A | 482 | 101.483 | 45.489 | 6.927 | 1.00 | 12.13 | N |
| ATOM | 3712 | CA | ASP | A | 482 | 101.528 | 44.225 | 7.659 | 1.00 | 14.92 | C |
| ATOM | 3713 | CB | ASP | A | 482 | 100.811 | 43.119 | 6.868 | 1.00 | 17.05 | C |
| ATOM | 3714 | CG | ASP | A | 482 | 99.280 | 43.268 | 6.895 | 1.00 | 18.25 | C |
| ATOM | 3715 | OD1 | ASP | A | 482 | 98.728 | 43.944 | 7.780 | 1.00 | 17.35 | O |
| ATOM | 3716 | OD2 | ASP | A | 482 | 98.627 | 42.699 | 6.016 | 1.00 | 21.98 | O |
| ATOM | 3717 | C | ASP | A | 482 | 102.953 | 43.774 | 7.941 | 1.00 | 15.43 | C |
| ATOM | 3718 | O | ASP | A | 482 | 103.142 | 42.827 | 8.689 | 1.00 | 13.49 | O |
| ATOM | 3719 | N | ALA | A | 483 | 103.937 | 44.424 | 7.326 | 1.00 | 15.02 | N |
| ATOM | 3720 | CA | ALA | A | 483 | 105.342 | 44.073 | 7.545 | 1.00 | 17.79 | C |
| ATOM | 3721 | CB | ALA | A | 483 | 105.964 | 43.459 | 6.279 | 1.00 | 14.25 | C |
| ATOM | 3722 | C | ALA | A | 483 | 106.091 | 45.329 | 7.971 | 1.00 | 15.42 | C |
| ATOM | 3723 | O | ALA | A | 483 | 105.643 | 46.442 | 7.687 | 1.00 | 14.23 | O |
| ATOM | 3724 | N | PRO | A | 484 | 107.237 | 45.159 | 8.638 | 1.00 | 16.34 | N |
| ATOM | 3725 | CA | PRO | A | 484 | 108.015 | 46.319 | 9.059 | 1.00 | 14.26 | C |
| ATOM | 3726 | CB | PRO | A | 484 | 109.237 | 45.692 | 9.768 | 1.00 | 15.32 | C |
| ATOM | 3727 | CG | PRO | A | 484 | 108.774 | 44.314 | 10.192 | 1.00 | 18.54 | C |
| ATOM | 3728 | CD | PRO | A | 484 | 107.864 | 43.883 | 9.068 | 1.00 | 17.52 | C |
| ATOM | 3729 | C | PRO | A | 484 | 108.493 | 47.175 | 7.875 | 1.00 | 14.36 | C |
| ATOM | 3730 | O | PRO | A | 484 | 108.743 | 46.645 | 6.808 | 1.00 | 12.69 | O |
| ATOM | 3731 | N | GLU | A | 485 | 108.632 | 48.480 | 8.082 | 1.00 | 12.15 | N |
| ATOM | 3732 | CA | GLU | A | 485 | 109.123 | 49.399 | 7.059 | 1.00 | 13.43 | C |
| ATOM | 3733 | CB | GLU | A | 485 | 108.047 | 50.429 | 6.747 | 1.00 | 13.80 | C |
| ATOM | 3734 | CG | GLU | A | 485 | 106.862 | 49.835 | 6.021 | 1.00 | 14.56 | C |
| ATOM | 3735 | CD | GLU | A | 485 | 107.147 | 49.536 | 4.564 | 1.00 | 16.23 | C |
| ATOM | 3736 | OE1 | GLU | A | 485 | 108.189 | 49.985 | 4.037 | 1.00 | 13.54 | O |
| ATOM | 3737 | OE2 | GLU | A | 485 | 106.298 | 48.863 | 3.949 | 1.00 | 20.24 | O |
| ATOM | 3738 | C | GLU | A | 485 | 110.365 | 50.132 | 7.542 | 1.00 | 12.82 | C |
| ATOM | 3739 | O | GLU | A | 485 | 110.428 | 50.513 | 8.711 | 1.00 | 13.04 | O |
| ATOM | 3740 | N | HIS | A | 486 | 111.336 | 50.300 | 6.645 | 1.00 | 12.11 | N |
| ATOM | 3741 | CA | AHIS | A | 486 | 112.585 | 50.984 | 6.945 | 0.50 | 12.85 | C |
| ATOM | 3742 | CA | BHIS | A | 486 | 112.593 | 51.005 | 6.966 | 0.50 | 12.63 | C |
| ATOM | 3743 | CB | AHIS | A | 486 | 113.742 | 50.184 | 6.352 | 0.50 | 14.74 | C |
| ATOM | 3744 | CB | BHIS | A | 486 | 113.837 | 50.256 | 6.460 | 0.50 | 14.15 | C |
| ATOM | 3745 | CG | AHIS | A | 486 | 113.795 | 48.744 | 6.826 | 0.50 | 16.29 | C |
| ATOM | 3746 | CG | BHIS | A | 486 | 115.164 | 50.829 | 6.980 | 0.50 | 15.34 | C |
| ATOM | 3747 | ND1 | AHIS | A | 486 | 113.154 | 47.740 | 6.184 | 0.50 | 17.98 | N |
| ATOM | 3748 | ND1 | BHIS | A | 486 | 115.935 | 51.678 | 6.246 | 0.50 | 15.67 | N |
| ATOM | 3749 | CE1 | AHIS | A | 486 | 113.374 | 46.583 | 6.829 | 0.50 | 17.75 | C |
| ATOM | 3750 | CE1 | BHIS | A | 486 | 117.025 | 52.030 | 6.964 | 0.50 | 14.82 | C |
| ATOM | 3751 | NE2 | AHIS | A | 486 | 114.158 | 46.841 | 7.885 | 0.50 | 19.45 | N |
| ATOM | 3752 | NE2 | BHIS | A | 486 | 116.963 | 51.392 | 8.162 | 0.50 | 16.80 | N |
| ATOM | 3753 | CD2 | AHIS | A | 486 | 114.431 | 48.164 | 7.919 | 0.50 | 17.74 | C |
| ATOM | 3754 | CD2 | BHIS | A | 486 | 115.828 | 50.657 | 8.211 | 0.50 | 14.06 | C |
| ATOM | 3755 | C | HIS | A | 486 | 112.536 | 52.389 | 6.388 | 1.00 | 12.98 | C |
| ATOM | 3756 | O | HIS | A | 486 | 112.129 | 52.586 | 5.236 | 1.00 | 12.49 | O |
| ATOM | 3757 | N | PHE | A | 487 | 112.911 | 53.376 | 7.200 | 1.00 | 11.23 | N |
| ATOM | 3758 | CA | PHE | A | 487 | 112.912 | 54.779 | 6.793 | 1.00 | 10.68 | C |
| ATOM | 3759 | CB | PHE | A | 487 | 112.022 | 55.643 | 7.725 | 1.00 | 9.60 | C |
| ATOM | 3760 | CG | PHE | A | 487 | 110.548 | 55.402 | 7.559 | 1.00 | 9.63 | C |
| ATOM | 3761 | CD1 | PHE | A | 487 | 109.929 | 54.307 | 8.168 | 1.00 | 10.45 | C |
| ATOM | 3762 | CE1 | PHE | A | 487 | 108.568 | 54.073 | 7.978 | 1.00 | 10.69 | C |
| ATOM | 3763 | CZ | PHE | A | 487 | 107.812 | 54.946 | 7.200 | 1.00 | 10.79 | C |
| ATOM | 3764 | CE2 | PHE | A | 487 | 108.415 | 56.036 | 6.611 | 1.00 | 9.55 | C |
| ATOM | 3765 | CD2 | PHE | A | 487 | 109.773 | 56.245 | 6.783 | 1.00 | 9.44 | C |
| ATOM | 3766 | C | PHE | A | 487 | 114.371 | 55.281 | 6.848 | 1.00 | 10.89 | C |
| ATOM | 3767 | O | PHE | A | 487 | 115.100 | 54.924 | 7.753 | 1.00 | 10.54 | O |
| ATOM | 3768 | N | ALA | A | 488 | 114.766 | 56.084 | 5.864 | 1.00 | 10.91 | N |
| ATOM | 3769 | CA | ALA | A | 488 | 116.055 | 56.780 | 5.855 | 1.00 | 12.54 | C |
| ATOM | 3770 | CB | ALA | A | 488 | 116.984 | 56.210 | 4.789 | 1.00 | 11.00 | C |
| ATOM | 3771 | C | ALA | A | 488 | 115.721 | 58.229 | 5.569 | 1.00 | 12.80 | C |
| ATOM | 3772 | O | ALA | A | 488 | 115.445 | 58.587 | 4.428 | 1.00 | 14.24 | O |
| ATOM | 3773 | N | VAL | A | 489 | 115.681 | 59.057 | 6.617 | 1.00 | 11.73 | N |
| ATOM | 3774 | CA | VAL | A | 489 | 115.214 | 60.438 | 6.488 | 1.00 | 11.05 | C |
| ATOM | 3775 | CB | VAL | A | 489 | 114.159 | 60.787 | 7.578 | 1.00 | 11.13 | C |
| ATOM | 3776 | CG1 | VAL | A | 489 | 113.767 | 62.266 | 7.524 | 1.00 | 9.30 | C |
| ATOM | 3777 | CG2 | VAL | A | 489 | 112.935 | 59.885 | 7.423 | 1.00 | 11.83 | C |
| ATOM | 3778 | C | VAL | A | 489 | 116.409 | 61.366 | 6.587 | 1.00 | 10.27 | C |
| ATOM | 3779 | O | VAL | A | 489 | 117.234 | 61.237 | 7.474 | 1.00 | 11.04 | O |
| ATOM | 3780 | N | GLN | A | 490 | 116.474 | 62.317 | 5.683 | 1.00 | 10.58 | N |
| ATOM | 3781 | CA | GLN | A | 490 | 117.583 | 63.256 | 5.612 | 1.00 | 10.03 | C |
| ATOM | 3782 | CB | GLN | A | 490 | 118.698 | 62.663 | 4.740 | 1.00 | 12.01 | C |
| ATOM | 3783 | CG | GLN | A | 490 | 120.005 | 63.467 | 4.833 | 1.00 | 11.32 | C |
| ATOM | 3784 | CD | GLN | A | 490 | 121.224 | 62.666 | 4.434 | 1.00 | 10.88 | C |
| ATOM | 3785 | OE1 | GLN | A | 490 | 121.163 | 61.443 | 4.255 | 1.00 | 11.57 | O |
| ATOM | 3786 | NE2 | GLN | A | 490 | 122.341 | 63.348 | 4.285 | 1.00 | 11.90 | N |
| ATOM | 3787 | C | GLN | A | 490 | 117.155 | 64.627 | 5.066 | 1.00 | 10.17 | C |
| ATOM | 3788 | O | GLN | A | 490 | 117.786 | 65.631 | 5.362 | 1.00 | 8.72 | O |
| ATOM | 3789 | N | THR | A | 491 | 116.085 | 64.699 | 4.279 | 1.00 | 9.58 | N |
| ATOM | 3790 | CA | THR | A | 491 | 115.626 | 66.000 | 3.792 | 1.00 | 11.73 | C |
| ATOM | 3791 | CB | THR | A | 491 | 114.481 | 65.858 | 2.753 | 1.00 | 13.80 | C |
| ATOM | 3792 | OG1 | THR | A | 491 | 115.010 | 65.226 | 1.570 | 1.00 | 18.48 | O |
| ATOM | 3793 | CG2 | THR | A | 491 | 113.911 | 67.234 | 2.351 | 1.00 | 12.80 | C |
| ATOM | 3794 | C | THR | A | 491 | 115.198 | 66.859 | 4.981 | 1.00 | 10.05 | C |
| ATOM | 3795 | O | THR | A | 491 | 114.442 | 66.420 | 5.827 | 1.00 | 10.42 | O |
| ATOM | 3796 | N | MET | A | 492 | 115.729 | 68.071 | 5.031 | 1.00 | 9.45 | N |
| ATOM | 3797 | CA | MET | A | 492 | 115.485 | 69.015 | 6.112 | 1.00 | 11.41 | C |
| ATOM | 3798 | CB | MET | A | 492 | 114.053 | 69.579 | 6.012 | 1.00 | 10.69 | C |
| ATOM | 3799 | CG | MET | A | 492 | 113.800 | 70.374 | 4.730 | 1.00 | 10.20 | C |
| ATOM | 3800 | SD | MET | A | 492 | 112.164 | 71.124 | 4.680 | 1.00 | 12.67 | S |
| ATOM | 3801 | CE | MET | A | 492 | 111.091 | 69.704 | 4.563 | 1.00 | 11.26 | C |
| ATOM | 3802 | C | MET | A | 492 | 115.748 | 68.465 | 7.523 | 1.00 | 10.13 | C |
| ATOM | 3803 | O | MET | A | 492 | 115.215 | 68.985 | 8.491 | 1.00 | 10.70 | O |
| ATOM | 3804 | N | LEU | A | 493 | 116.595 | 67.453 | 7.640 | 1.00 | 9.99 | N |
| ATOM | 3805 | CA | LEU | A | 493 | 116.859 | 66.830 | 8.927 | 1.00 | 10.70 | C |
| ATOM | 3806 | CB | LEU | A | 493 | 117.301 | 65.371 | 8.749 | 1.00 | 11.02 | C |
| ATOM | 3807 | CG | LEU | A | 493 | 117.551 | 64.643 | 10.085 | 1.00 | 11.96 | C |
| ATOM | 3808 | CD1 | LEU | A | 493 | 116.226 | 64.224 | 10.715 | 1.00 | 10.78 | C |
| ATOM | 3809 | CD2 | LEU | A | 493 | 118.482 | 63.443 | 9.853 | 1.00 | 13.60 | C |
| ATOM | 3810 | C | LEU | A | 493 | 117.966 | 67.606 | 9.642 | 1.00 | 11.06 | C |
| ATOM | 3811 | O | LEU | A | 493 | 119.067 | 67.744 | 9.122 | 1.00 | 10.71 | O |
| ATOM | 3812 | N | GLN | A | 494 | 117.685 | 68.095 | 10.839 | 1.00 | 9.75 | N |
| ATOM | 3813 | CA | GLN | A | 494 | 118.670 | 68.858 | 11.592 | 1.00 | 9.84 | C |
| ATOM | 3814 | CB | GLN | A | 494 | 117.982 | 69.838 | 12.523 | 1.00 | 10.66 | C |
| ATOM | 3815 | CG | GLN | A | 494 | 118.942 | 70.688 | 13.357 | 1.00 | 11.22 | C |
| ATOM | 3816 | CD | GLN | A | 494 | 118.237 | 71.903 | 13.930 | 1.00 | 11.78 | C |
| ATOM | 3817 | OE1 | GLN | A | 494 | 117.596 | 72.661 | 13.193 | 1.00 | 9.17 | O |
| ATOM | 3818 | NE2 | GLN | A | 494 | 118.373 | 72.115 | 15.241 | 1.00 | 10.94 | N |
| ATOM | 3819 | C | GLN | A | 494 | 119.533 | 67.879 | 12.405 | 1.00 | 9.56 | C |
| ATOM | 3820 | O | GLN | A | 494 | 119.001 | 67.073 | 13.177 | 1.00 | 8.81 | O |
| ATOM | 3821 | N | PRO | A | 495 | 120.856 | 67.935 | 12.223 | 1.00 | 8.63 | N |
| ATOM | 3822 | CA | PRO | A | 495 | 121.741 | 67.115 | 13.035 | 1.00 | 9.61 | C |
| ATOM | 3823 | CB | PRO | A | 495 | 123.150 | 67.572 | 12.592 | 1.00 | 9.42 | C |
| ATOM | 3824 | CG | PRO | A | 495 | 122.956 | 68.053 | 11.194 | 1.00 | 9.16 | C |
| ATOM | 3825 | CD | PRO | A | 495 | 121.616 | 68.731 | 11.230 | 1.00 | 9.72 | C |
| ATOM | 3826 | C | PRO | A | 495 | 121.548 | 67.360 | 14.510 | 1.00 | 10.17 | C |
| ATOM | 3827 | O | PRO | A | 495 | 121.250 | 68.481 | 14.914 | 1.00 | 11.25 | O |
| ATOM | 3828 | N | GLY | A | 496 | 121.702 | 66.312 | 15.308 | 1.00 | 10.49 | N |
| ATOM | 3829 | CA | GLY | A | 496 | 121.533 | 66.412 | 16.733 | 1.00 | 11.03 | C |
| ATOM | 3830 | C | GLY | A | 496 | 121.206 | 65.089 | 17.392 | 1.00 | 12.19 | C |
| ATOM | 3831 | O | GLY | A | 496 | 121.296 | 64.004 | 16.774 | 1.00 | 11.67 | O |
| ATOM | 3832 | N | ARG | A | 497 | 120.821 | 65.183 | 18.659 | 1.00 | 12.78 | N |
| ATOM | 3833 | CA | ARG | A | 497 | 120.500 | 64.001 | 19.430 | 1.00 | 14.38 | C |
| ATOM | 3834 | CB | ARG | A | 497 | 121.150 | 64.096 | 20.820 | 1.00 | 17.05 | C |
| ATOM | 3835 | CG | ARG | A | 497 | 122.660 | 64.292 | 20.741 | 1.00 | 17.85 | C |
| ATOM | 3836 | CD | ARG | A | 497 | 123.314 | 64.179 | 22.110 | 1.00 | 20.04 | C |
| ATOM | 3837 | NE | ARG | A | 497 | 124.776 | 64.253 | 22.074 | 1.00 | 18.23 | N |
| ATOM | 3838 | CZ | ARG | A | 497 | 125.496 | 65.361 | 22.285 | 1.00 | 19.00 | C |
| ATOM | 3839 | NH1 | ARG | A | 497 | 124.891 | 66.515 | 22.517 | 1.00 | 21.56 | N |
| ATOM | 3840 | NH2 | ARG | A | 497 | 126.838 | 65.329 | 22.273 | 1.00 | 15.13 | N |
| ATOM | 3841 | C | ARG | A | 497 | 119.005 | 63.833 | 19.496 | 1.00 | 13.31 | C |
| ATOM | 3842 | O | ARG | A | 497 | 118.332 | 64.522 | 20.247 | 1.00 | 17.01 | O |
| ATOM | 3843 | N | TRP | A | 498 | 118.493 | 62.910 | 18.690 | 1.00 | 12.85 | N |
| ATOM | 3844 | CA | TRP | A | 498 | 117.061 | 62.667 | 18.564 | 1.00 | 13.09 | C |
| ATOM | 3845 | CB | TRP | A | 498 | 116.732 | 62.305 | 17.139 | 1.00 | 10.29 | C |
| ATOM | 3846 | CG | TRP | A | 498 | 116.891 | 63.411 | 16.133 | 1.00 | 8.66 | C |
| ATOM | 3847 | CD1 | TRP | A | 498 | 118.063 | 63.884 | 15.559 | 1.00 | 8.76 | C |
| ATOM | 3848 | NE1 | TRP | A | 498 | 117.802 | 64.876 | 14.656 | 1.00 | 8.86 | N |
| ATOM | 3849 | CE2 | TRP | A | 498 | 116.460 | 65.087 | 14.578 | 1.00 | 8.51 | C |
| ATOM | 3850 | CD2 | TRP | A | 498 | 115.824 | 64.159 | 15.502 | 1.00 | 8.53 | C |
| ATOM | 3851 | CE3 | TRP | A | 498 | 114.443 | 64.163 | 15.619 | 1.00 | 9.16 | C |
| ATOM | 3852 | CZ3 | TRP | A | 498 | 113.720 | 65.054 | 14.845 | 1.00 | 9.25 | C |
| ATOM | 3853 | CH2 | TRP | A | 498 | 114.357 | 65.938 | 13.955 | 1.00 | 8.63 | C |
| ATOM | 3854 | CZ2 | TRP | A | 498 | 115.732 | 65.973 | 13.819 | 1.00 | 8.09 | C |
| ATOM | 3855 | C | TRP | A | 498 | 116.627 | 61.527 | 19.454 | 1.00 | 16.40 | C |
| ATOM | 3856 | O | TRP | A | 498 | 117.265 | 60.478 | 19.445 | 1.00 | 24.22 | O |
| ATOM | 3857 | N | HIS | A | 499 | 115.546 | 61.707 | 20.207 | 1.00 | 15.34 | N |
| ATOM | 3858 | CA | HIS | A | 499 | 115.081 | 60.697 | 21.196 | 1.00 | 17.77 | C |
| ATOM | 3859 | CB | HIS | A | 499 | 114.972 | 61.300 | 22.618 | 1.00 | 21.46 | C |
| ATOM | 3860 | CG | HIS | A | 499 | 116.269 | 61.968 | 23.121 | 1.00 | 36.41 | C |
| ATOM | 3861 | ND1 | HIS | A | 499 | 117.129 | 61.359 | 23.981 | 1.00 | 40.93 | N |
| ATOM | 3862 | CE1 | HIS | A | 499 | 118.172 | 62.188 | 24.239 | 1.00 | 39.20 | C |
| ATOM | 3863 | NE2 | HIS | A | 499 | 117.983 | 63.330 | 23.545 | 1.00 | 36.63 | N |
| ATOM | 3864 | CD2 | HIS | A | 499 | 116.823 | 63.236 | 22.852 | 1.00 | 34.57 | C |
| ATOM | 3865 | C | HIS | A | 499 | 113.751 | 60.145 | 20.771 | 1.00 | 15.91 | C |
| ATOM | 3866 | O | HIS | A | 499 | 112.801 | 60.901 | 20.495 | 1.00 | 13.95 | O |
| ATOM | 3867 | N | ASP | A | 500 | 113.658 | 58.826 | 20.693 | 1.00 | 17.76 | N |
| ATOM | 3868 | CA | ASP | A | 500 | 112.422 | 58.140 | 20.353 | 1.00 | 21.93 | C |
| ATOM | 3869 | CB | ASP | A | 500 | 112.692 | 56.641 | 20.136 | 1.00 | 30.24 | C |
| ATOM | 3870 | CG | ASP | A | 500 | 111.423 | 55.828 | 19.818 | 1.00 | 33.18 | C |
| ATOM | 3871 | OD1 | ASP | A | 500 | 110.286 | 56.318 | 19.959 | 1.00 | 43.09 | O |
| ATOM | 3872 | OD2 | ASP | A | 500 | 111.582 | 54.675 | 19.391 | 1.00 | 42.81 | O |
| ATOM | 3873 | C | ASP | A | 500 | 111.492 | 58.312 | 21.539 | 1.00 | 20.91 | C |
| ATOM | 3874 | O | ASP | A | 500 | 111.796 | 57.809 | 22.591 | 1.00 | 22.20 | O |
| ATOM | 3875 | N | ALA | A | 501 | 110.374 | 59.008 | 21.363 | 1.00 | 14.99 | N |
| ATOM | 3876 | CA | ALA | A | 501 | 109.471 | 59.304 | 22.477 | 1.00 | 17.10 | C |
| ATOM | 3877 | CB | ALA | A | 501 | 108.385 | 60.274 | 22.036 | 1.00 | 14.69 | C |
| ATOM | 3878 | C | ALA | A | 501 | 108.830 | 58.076 | 23.139 | 1.00 | 22.71 | C |
| ATOM | 3879 | O | ALA | A | 501 | 108.475 | 58.154 | 24.303 | 1.00 | 35.66 | O |
| ATOM | 3880 | N | ILE | A | 502 | 108.632 | 56.983 | 22.410 | 1.00 | 31.51 | N |
| ATOM | 3881 | CA | ILE | A | 502 | 107.991 | 55.756 | 22.969 | 1.00 | 43.49 | C |
| ATOM | 3882 | CB | ILE | A | 502 | 106.961 | 55.114 | 22.023 | 1.00 | 43.64 | C |
| ATOM | 3883 | CG1 | ILE | A | 502 | 105.735 | 56.028 | 21.853 | 1.00 | 44.63 | C |
| ATOM | 3884 | CD1 | ILE | A | 502 | 105.095 | 55.971 | 20.474 | 1.00 | 35.62 | C |
| ATOM | 3885 | CG2 | ILE | A | 502 | 106.552 | 53.733 | 22.560 | 1.00 | 42.82 | C |
| ATOM | 3886 | C | ILE | A | 502 | 109.023 | 54.694 | 23.328 | 1.00 | 46.88 | C |
| ATOM | 3887 | O | ILE | A | 502 | 109.152 | 54.354 | 24.509 | 1.00 | 51.64 | O |
| ATOM | 3888 | N | GLY | A | 503 | 109.755 | 54.174 | 22.330 | 1.00 | 49.47 | N |
| ATOM | 3889 | CA | GLY | A | 503 | 111.057 | 53.524 | 22.605 | 1.00 | 42.47 | C |
| ATOM | 3890 | C | GLY | A | 503 | 111.819 | 54.511 | 23.491 | 1.00 | 41.75 | C |
| ATOM | 3891 | O | GLY | A | 503 | 111.240 | 55.476 | 23.989 | 1.00 | 52.49 | O |
| ATOM | 3892 | N | GLY | A | 504 | 113.095 | 54.297 | 23.733 | 1.00 | 33.97 | N |
| ATOM | 3893 | CA | GLY | A | 504 | 113.853 | 55.252 | 24.542 | 1.00 | 36.81 | C |
| ATOM | 3894 | C | GLY | A | 504 | 115.188 | 55.666 | 23.959 | 1.00 | 35.32 | C |
| ATOM | 3895 | O | GLY | A | 504 | 115.831 | 56.598 | 24.454 | 1.00 | 44.34 | O |
| ATOM | 3896 | N | GLU | A | 505 | 115.603 | 54.992 | 22.898 | 1.00 | 43.81 | N |
| ATOM | 3897 | CA | GLU | A | 505 | 116.913 | 55.246 | 22.295 | 1.00 | 48.17 | C |
| ATOM | 3898 | CB | GLU | A | 505 | 117.241 | 54.250 | 21.160 | 1.00 | 54.81 | C |
| ATOM | 3899 | CG | GLU | A | 505 | 116.119 | 53.974 | 20.174 | 1.00 | 56.61 | C |
| ATOM | 3900 | CD | GLU | A | 505 | 115.051 | 53.045 | 20.748 | 1.00 | 79.51 | C |
| ATOM | 3901 | OE1 | GLU | A | 505 | 115.304 | 51.822 | 20.841 | 1.00 | 93.44 | O |
| ATOM | 3902 | OE2 | GLU | A | 505 | 113.963 | 53.544 | 21.129 | 1.00 | 59.14 | O |
| ATOM | 3903 | C | GLU | A | 505 | 117.085 | 56.684 | 21.798 | 1.00 | 42.11 | C |
| ATOM | 3904 | O | GLU | A | 505 | 116.139 | 57.351 | 21.354 | 1.00 | 39.97 | O |
| ATOM | 3905 | N | THR | A | 506 | 118.317 | 57.137 | 21.939 | 1.00 | 36.27 | N |
| ATOM | 3906 | CA | THR | A | 506 | 118.814 | 58.333 | 21.326 | 1.00 | 32.81 | C |
| ATOM | 3907 | CB | THR | A | 506 | 119.746 | 59.040 | 22.299 | 1.00 | 35.17 | C |
| ATOM | 3908 | OG1 | THR | A | 506 | 118.999 | 59.308 | 23.486 | 1.00 | 47.17 | O |
| ATOM | 3909 | CG2 | THR | A | 506 | 120.294 | 60.360 | 21.719 | 1.00 | 34.36 | C |
| ATOM | 3910 | C | THR | A | 506 | 119.550 | 57.949 | 20.052 | 1.00 | 25.53 | C |
| ATOM | 3911 | O | THR | A | 506 | 120.446 | 57.110 | 20.060 | 1.00 | 23.32 | O |
| ATOM | 3912 | N | LEU | A | 507 | 119.133 | 58.529 | 18.944 | 1.00 | 20.16 | N |
| ATOM | 3913 | CA | LEU | A | 507 | 119.884 | 58.446 | 17.709 | 1.00 | 17.44 | C |
| ATOM | 3914 | CB | LEU | A | 507 | 118.953 | 58.184 | 16.525 | 1.00 | 18.20 | C |
| ATOM | 3915 | CG | LEU | A | 507 | 118.249 | 56.821 | 16.361 | 1.00 | 18.08 | C |
| ATOM | 3916 | CD1 | LEU | A | 507 | 117.469 | 56.808 | 15.050 | 1.00 | 14.73 | C |
| ATOM | 3917 | CD2 | LEU | A | 507 | 119.205 | 55.620 | 16.388 | 1.00 | 21.65 | C |
| ATOM | 3918 | C | LEU | A | 507 | 120.613 | 59.788 | 17.561 | 1.00 | 16.84 | C |
| ATOM | 3919 | O | LEU | A | 507 | 119.981 | 60.845 | 17.537 | 1.00 | 15.02 | O |
| ATOM | 3920 | N | THR | A | 508 | 121.941 | 59.738 | 17.498 | 1.00 | 15.75 | N |
| ATOM | 3921 | CA | THR | A | 508 | 122.741 | 60.915 | 17.258 | 1.00 | 14.58 | C |
| ATOM | 3922 | CB | THR | A | 508 | 124.084 | 60.861 | 17.997 | 1.00 | 14.89 | C |
| ATOM | 3923 | OG1 | THR | A | 508 | 123.816 | 60.897 | 19.393 | 1.00 | 13.50 | O |
| ATOM | 3924 | CG2 | THR | A | 508 | 124.981 | 62.070 | 17.606 | 1.00 | 12.67 | C |
| ATOM | 3925 | C | THR | A | 508 | 122.991 | 61.000 | 15.776 | 1.00 | 14.73 | C |
| ATOM | 3926 | O | THR | A | 508 | 123.641 | 60.127 | 15.202 | 1.00 | 13.99 | O |
| ATOM | 3927 | N | ILE | A | 509 | 122.481 | 62.055 | 15.155 | 1.00 | 13.45 | N |
| ATOM | 3928 | CA | ILE | A | 509 | 122.656 | 62.225 | 13.732 | 1.00 | 13.06 | C |
| ATOM | 3929 | CB | ILE | A | 509 | 121.335 | 62.623 | 13.045 | 1.00 | 13.81 | C |
| ATOM | 3930 | CG1 | ILE | A | 509 | 120.222 | 61.628 | 13.419 | 1.00 | 13.63 | C |
| ATOM | 3931 | CD1 | ILE | A | 509 | 120.483 | 60.175 | 13.027 | 1.00 | 12.00 | C |
| ATOM | 3932 | CG2 | ILE | A | 509 | 121.561 | 62.728 | 11.532 | 1.00 | 14.78 | C |
| ATOM | 3933 | C | ILE | A | 509 | 123.699 | 63.303 | 13.482 | 1.00 | 11.43 | C |
| ATOM | 3934 | O | ILE | A | 509 | 123.563 | 64.418 | 13.971 | 1.00 | 11.35 | O |
| ATOM | 3935 | N | GLN | A | 510 | 124.739 | 62.930 | 12.744 | 1.00 | 10.14 | N |
| ATOM | 3936 | CA | GLN | A | 510 | 125.805 | 63.825 | 12.332 | 1.00 | 10.29 | C |
| ATOM | 3937 | CB | GLN | A | 510 | 127.129 | 63.049 | 12.223 | 1.00 | 11.16 | C |
| ATOM | 3938 | CG | GLN | A | 510 | 127.611 | 62.468 | 13.546 | 1.00 | 11.25 | C |
| ATOM | 3939 | CD | GLN | A | 510 | 129.004 | 61.863 | 13.472 | 1.00 | 12.20 | C |
| ATOM | 3940 | OE1 | GLN | A | 510 | 129.725 | 62.031 | 12.503 | 1.00 | 12.77 | O |
| ATOM | 3941 | NE2 | GLN | A | 510 | 129.393 | 61.184 | 14.530 | 1.00 | 11.54 | N |
| ATOM | 3942 | C | GLN | A | 510 | 125.487 | 64.436 | 10.981 | 1.00 | 9.89 | C |
| ATOM | 3943 | O | GLN | A | 510 | 124.807 | 63.805 | 10.149 | 1.00 | 10.54 | O |
| ATOM | 3944 | N | ALA | A | 511 | 126.006 | 65.641 | 10.752 | 1.00 | 9.74 | N |
| ATOM | 3945 | CA | ALA | A | 511 | 125.795 | 66.352 | 9.485 | 1.00 | 11.00 | C |
| ATOM | 3946 | CB | ALA | A | 511 | 126.543 | 67.704 | 9.468 | 1.00 | 9.61 | C |
| ATOM | 3947 | C | ALA | A | 511 | 126.209 | 65.478 | 8.294 | 1.00 | 10.27 | C |
| ATOM | 3948 | O | ALA | A | 511 | 127.285 | 64.861 | 8.297 | 1.00 | 9.35 | O |
| ATOM | 3949 | N | GLY | A | 512 | 125.359 | 65.439 | 7.274 | 1.00 | 9.80 | N |
| ATOM | 3950 | CA | GLY | A | 512 | 125.614 | 64.654 | 6.091 | 1.00 | 9.49 | C |
| ATOM | 3951 | C | GLY | A | 512 | 125.026 | 63.260 | 6.153 | 1.00 | 11.41 | C |
| ATOM | 3952 | O | GLY | A | 512 | 125.016 | 62.552 | 5.135 | 1.00 | 14.08 | O |
| ATOM | 3953 | N | GLU | A | 513 | 124.490 | 62.864 | 7.311 | 1.00 | 12.38 | N |
| ATOM | 3954 | CA | GLU | A | 513 | 123.914 | 61.529 | 7.484 | 1.00 | 14.25 | C |
| ATOM | 3955 | CB | GLU | A | 513 | 124.493 | 60.822 | 8.721 | 1.00 | 18.94 | C |
| ATOM | 3956 | CG | GLU | A | 513 | 126.007 | 60.915 | 8.911 | 1.00 | 26.20 | C |
| ATOM | 3957 | CD | GLU | A | 513 | 126.795 | 60.076 | 7.930 | 1.00 | 37.25 | C |
| ATOM | 3958 | OE1 | GLU | A | 513 | 126.248 | 59.070 | 7.430 | 1.00 | 40.40 | O |
| ATOM | 3959 | OE2 | GLU | A | 513 | 127.975 | 60.418 | 7.671 | 1.00 | 45.26 | O |
| ATOM | 3960 | C | GLU | A | 513 | 122.385 | 61.579 | 7.642 | 1.00 | 12.91 | C |
| ATOM | 3961 | O | GLU | A | 513 | 121.811 | 62.605 | 7.946 | 1.00 | 13.81 | O |
| ATOM | 3962 | N | ALA | A | 514 | 121.769 | 60.415 | 7.504 | 1.00 | 11.39 | N |
| ATOM | 3963 | CA | ALA | A | 514 | 120.333 | 60.222 | 7.574 | 1.00 | 11.17 | C |
| ATOM | 3964 | CB | ALA | A | 514 | 119.896 | 59.309 | 6.433 | 1.00 | 10.91 | C |
| ATOM | 3965 | C | ALA | A | 514 | 119.979 | 59.583 | 8.907 | 1.00 | 10.60 | C |
| ATOM | 3966 | O | ALA | A | 514 | 120.807 | 58.914 | 9.524 | 1.00 | 9.46 | O |
| ATOM | 3967 | N | LEU | A | 515 | 118.738 | 59.792 | 9.329 | 1.00 | 10.57 | N |
| ATOM | 3968 | CA | LEU | A | 515 | 118.141 | 59.089 | 10.460 | 1.00 | 10.43 | C |
| ATOM | 3969 | CB | LEU | A | 515 | 117.087 | 59.965 | 11.154 | 1.00 | 10.29 | C |
| ATOM | 3970 | CG | LEU | A | 515 | 116.529 | 59.410 | 12.461 | 1.00 | 11.73 | C |
| ATOM | 3971 | CD1 | LEU | A | 515 | 116.065 | 60.553 | 13.357 | 1.00 | 11.08 | C |
| ATOM | 3972 | CD2 | LEU | A | 515 | 115.395 | 58.389 | 12.259 | 1.00 | 11.49 | C |
| ATOM | 3973 | C | LEU | A | 515 | 117.498 | 57.831 | 9.909 | 1.00 | 10.90 | C |
| ATOM | 3974 | O | LEU | A | 515 | 116.594 | 57.915 | 9.073 | 1.00 | 10.60 | O |
| ATOM | 3975 | N | HIS | A | 516 | 117.970 | 56.678 | 10.370 | 1.00 | 10.55 | N |
| ATOM | 3976 | CA | HIS | A | 516 | 117.477 | 55.372 | 9.940 | 1.00 | 12.62 | C |
| ATOM | 3977 | CB | HIS | A | 516 | 118.649 | 54.398 | 9.694 | 1.00 | 13.48 | C |
| ATOM | 3978 | CG | HIS | A | 516 | 119.533 | 54.803 | 8.567 | 1.00 | 17.52 | C |
| ATOM | 3979 | ND1 | HIS | A | 516 | 119.294 | 54.427 | 7.296 | 1.00 | 20.78 | N |
| ATOM | 3980 | CE1 | HIS | A | 516 | 120.228 | 54.955 | 6.496 | 1.00 | 19.23 | C |
| ATOM | 3981 | NE2 | HIS | A | 516 | 121.070 | 55.667 | 7.251 | 1.00 | 18.35 | N |
| ATOM | 3982 | CD2 | HIS | A | 516 | 120.660 | 55.605 | 8.535 | 1.00 | 18.79 | C |
| ATOM | 3983 | C | HIS | A | 516 | 116.669 | 54.795 | 11.054 | 1.00 | 14.02 | C |
| ATOM | 3984 | O | HIS | A | 516 | 117.119 | 54.815 | 12.203 | 1.00 | 13.21 | O |
| ATOM | 3985 | N | ALA | A | 517 | 115.510 | 54.241 | 10.719 | 1.00 | 13.02 | N |
| ATOM | 3986 | CA | ALA | A | 517 | 114.621 | 53.630 | 11.713 | 1.00 | 14.34 | C |
| ATOM | 3987 | CB | ALA | A | 517 | 113.734 | 54.691 | 12.370 | 1.00 | 13.42 | C |
| ATOM | 3988 | C | ALA | A | 517 | 113.748 | 52.589 | 11.037 | 1.00 | 14.00 | C |
| ATOM | 3989 | O | ALA | A | 517 | 113.249 | 52.800 | 9.929 | 1.00 | 14.94 | O |
| ATOM | 3990 | N | GLU | A | 518 | 113.536 | 51.485 | 11.723 | 1.00 | 13.79 | N |
| ATOM | 3991 | CA | GLU | A | 518 | 112.557 | 50.505 | 11.336 | 1.00 | 15.60 | C |
| ATOM | 3992 | CB | GLU | A | 518 | 113.122 | 49.104 | 11.582 | 1.00 | 20.75 | C |
| ATOM | 3993 | CG | GLU | A | 518 | 112.350 | 47.992 | 10.896 | 1.00 | 26.36 | C |
| ATOM | 3994 | CD | GLU | A | 518 | 112.955 | 46.628 | 11.199 | 1.00 | 31.68 | C |
| ATOM | 3995 | OE1 | GLU | A | 518 | 114.174 | 46.471 | 10.994 | 1.00 | 36.57 | O |
| ATOM | 3996 | OE2 | GLU | A | 518 | 112.223 | 45.728 | 11.660 | 1.00 | 30.59 | O |
| ATOM | 3997 | C | GLU | A | 518 | 111.282 | 50.708 | 12.160 | 1.00 | 14.30 | C |
| ATOM | 3998 | O | GLU | A | 518 | 111.328 | 50.788 | 13.375 | 1.00 | 16.77 | O |
| ATOM | 3999 | N | VAL | A | 519 | 110.151 | 50.784 | 11.483 | 1.00 | 11.31 | N |
| ATOM | 4000 | CA | VAL | A | 519 | 108.854 | 50.902 | 12.126 | 1.00 | 10.69 | C |
| ATOM | 4001 | CB | VAL | A | 519 | 108.026 | 52.044 | 11.535 | 1.00 | 9.53 | C |
| ATOM | 4002 | CG1 | VAL | A | 519 | 106.686 | 52.199 | 12.295 | 1.00 | 7.75 | C |
| ATOM | 4003 | CG2 | VAL | A | 519 | 108.850 | 53.338 | 11.581 | 1.00 | 8.74 | C |
| ATOM | 4004 | C | VAL | A | 519 | 108.082 | 49.589 | 12.008 | 1.00 | 10.94 | C |
| ATOM | 4005 | O | VAL | A | 519 | 107.882 | 49.075 | 10.897 | 1.00 | 9.94 | O |
| ATOM | 4006 | N | PRO | A | 520 | 107.647 | 49.026 | 13.148 | 1.00 | 12.57 | N |
| ATOM | 4007 | CA | PRO | A | 520 | 106.867 | 47.763 | 13.066 | 1.00 | 14.58 | C |
| ATOM | 4008 | CB | PRO | A | 520 | 106.494 | 47.467 | 14.536 | 1.00 | 16.07 | C |
| ATOM | 4009 | CG | PRO | A | 520 | 107.485 | 48.235 | 15.356 | 1.00 | 17.24 | C |
| ATOM | 4010 | CD | PRO | A | 520 | 107.849 | 49.461 | 14.545 | 1.00 | 16.07 | C |
| ATOM | 4011 | C | PRO | A | 520 | 105.582 | 47.889 | 12.248 | 1.00 | 12.26 | C |
| ATOM | 4012 | O | PRO | A | 520 | 105.072 | 49.013 | 12.053 | 1.00 | 11.41 | O |
| ATOM | 4013 | N | ALA | A | 521 | 105.078 | 46.742 | 11.782 | 1.00 | 10.12 | N |
| ATOM | 4014 | CA | ALA | A | 521 | 103.731 | 46.642 | 11.197 | 1.00 | 10.96 | C |
| ATOM | 4015 | CB | ALA | A | 521 | 103.329 | 45.162 | 10.990 | 1.00 | 10.36 | C |
| ATOM | 4016 | C | ALA | A | 521 | 102.716 | 47.337 | 12.106 | 1.00 | 9.89 | C |
| ATOM | 4017 | O | ALA | A | 521 | 102.768 | 47.207 | 13.323 | 1.00 | 9.19 | O |
| ATOM | 4018 | N | HIS | A | 522 | 101.816 | 48.107 | 11.513 | 1.00 | 9.54 | N |
| ATOM | 4019 | CA | HIS | A | 522 | 100.814 | 48.843 | 12.269 | 1.00 | 9.64 | C |
| ATOM | 4020 | CB | HIS | A | 522 | 99.727 | 47.860 | 12.666 | 1.00 | 10.73 | C |
| ATOM | 4021 | CG | HIS | A | 522 | 99.191 | 47.110 | 11.469 | 1.00 | 10.88 | C |
| ATOM | 4022 | ND1 | HIS | A | 522 | 98.521 | 47.744 | 10.477 | 1.00 | 11.26 | N |
| ATOM | 4023 | CE1 | HIS | A | 522 | 98.216 | 46.859 | 9.510 | 1.00 | 12.49 | C |
| ATOM | 4024 | NE2 | HIS | A | 522 | 98.705 | 45.661 | 9.875 | 1.00 | 11.79 | N |
| ATOM | 4025 | CD2 | HIS | A | 522 | 99.310 | 45.787 | 11.087 | 1.00 | 9.87 | C |
| ATOM | 4026 | C | HIS | A | 522 | 101.407 | 49.618 | 13.431 | 1.00 | 11.25 | C |
| ATOM | 4027 | O | HIS | A | 522 | 100.931 | 49.558 | 14.575 | 1.00 | 11.49 | O |
| ATOM | 4028 | N | GLY | A | 523 | 102.466 | 50.367 | 13.114 | 1.00 | 11.52 | N |
| ATOM | 4029 | CA | GLY | A | 523 | 103.316 | 50.985 | 14.107 | 1.00 | 10.64 | C |
| ATOM | 4030 | C | GLY | A | 523 | 103.592 | 52.433 | 13.862 | 1.00 | 8.84 | C |
| ATOM | 4031 | O | GLY | A | 523 | 103.252 | 52.980 | 12.810 | 1.00 | 8.72 | O |
| ATOM | 4032 | N | VAL | A | 524 | 104.241 | 53.034 | 14.854 | 1.00 | 8.94 | N |
| ATOM | 4033 | CA | VAL | A | 524 | 104.610 | 54.432 | 14.845 | 1.00 | 8.56 | C |
| ATOM | 4034 | CB | VAL | A | 524 | 103.520 | 55.297 | 15.527 | 1.00 | 9.19 | C |
| ATOM | 4035 | CG1 | VAL | A | 524 | 103.171 | 54.762 | 16.928 | 1.00 | 8.21 | C |
| ATOM | 4036 | CG2 | VAL | A | 524 | 103.945 | 56.757 | 15.591 | 1.00 | 7.98 | C |
| ATOM | 4037 | C | VAL | A | 524 | 105.917 | 54.599 | 15.598 | 1.00 | 10.38 | C |
| ATOM | 4038 | O | VAL | A | 524 | 106.167 | 53.886 | 16.593 | 1.00 | 10.42 | O |
| ATOM | 4039 | N | ARG | A | 525 | 106.760 | 55.508 | 15.124 | 1.00 | 9.71 | N |
| ATOM | 4040 | CA | ARG | A | 525 | 107.859 | 56.028 | 15.940 | 1.00 | 10.60 | C |
| ATOM | 4041 | CB | ARG | A | 525 | 109.207 | 55.485 | 15.455 | 1.00 | 13.03 | C |
| ATOM | 4042 | CG | ARG | A | 525 | 109.297 | 53.949 | 15.562 | 1.00 | 18.03 | C |
| ATOM | 4043 | CD | ARG | A | 525 | 110.317 | 53.471 | 16.562 | 1.00 | 25.99 | C |
| ATOM | 4044 | NE | ARG | A | 525 | 111.397 | 52.876 | 15.804 | 1.00 | 34.69 | N |
| ATOM | 4045 | CZ | ARG | A | 525 | 112.674 | 52.854 | 16.145 | 1.00 | 28.95 | C |
| ATOM | 4046 | NH1 | ARG | A | 525 | 113.526 | 52.273 | 15.310 | 1.00 | 30.55 | N |
| ATOM | 4047 | NH2 | ARG | A | 525 | 113.114 | 53.398 | 17.284 | 1.00 | 32.10 | N |
| ATOM | 4048 | C | ARG | A | 525 | 107.827 | 57.551 | 15.888 | 1.00 | 10.27 | C |
| ATOM | 4049 | O | ARG | A | 525 | 107.502 | 58.135 | 14.849 | 1.00 | 10.33 | O |
| ATOM | 4050 | N | VAL | A | 526 | 108.118 | 58.194 | 17.008 | 1.00 | 9.16 | N |
| ATOM | 4051 | CA | VAL | A | 526 | 108.174 | 59.656 | 17.048 | 1.00 | 10.36 | C |
| ATOM | 4052 | CB | VAL | A | 526 | 107.051 | 60.261 | 17.917 | 1.00 | 10.49 | C |
| ATOM | 4053 | CG1 | VAL | A | 526 | 107.197 | 61.783 | 18.027 | 1.00 | 8.98 | C |
| ATOM | 4054 | CG2 | VAL | A | 526 | 105.665 | 59.895 | 17.362 | 1.00 | 10.91 | C |
| ATOM | 4055 | C | VAL | A | 526 | 109.524 | 59.987 | 17.629 | 1.00 | 11.51 | C |
| ATOM | 4056 | O | VAL | A | 526 | 109.798 | 59.616 | 18.764 | 1.00 | 14.38 | O |
| ATOM | 4057 | N | PHE | A | 527 | 110.383 | 60.617 | 16.839 | 1.00 | 9.91 | N |
| ATOM | 4058 | CA | PHE | A | 527 | 111.660 | 61.103 | 17.323 | 1.00 | 9.52 | C |
| ATOM | 4059 | CB | PHE | A | 527 | 112.749 | 60.797 | 16.305 | 1.00 | 9.92 | C |
| ATOM | 4060 | CG | PHE | A | 527 | 112.995 | 59.320 | 16.114 | 1.00 | 11.05 | C |
| ATOM | 4061 | CD1 | PHE | A | 527 | 113.911 | 58.643 | 16.915 | 1.00 | 13.35 | C |
| ATOM | 4062 | CE1 | PHE | A | 527 | 114.145 | 57.276 | 16.719 | 1.00 | 14.90 | C |
| ATOM | 4063 | CZ | PHE | A | 527 | 113.448 | 56.582 | 15.731 | 1.00 | 10.88 | C |
| ATOM | 4064 | CE2 | PHE | A | 527 | 112.567 | 57.255 | 14.922 | 1.00 | 10.96 | C |
| ATOM | 4065 | CD2 | PHE | A | 527 | 112.331 | 58.614 | 15.130 | 1.00 | 11.05 | C |
| ATOM | 4066 | C | PHE | A | 527 | 111.614 | 62.600 | 17.642 | 1.00 | 9.59 | C |
| ATOM | 4067 | O | PHE | A | 527 | 111.072 | 63.399 | 16.877 | 1.00 | 10.13 | O |
| ATOM | 4068 | N | LEU | A | 528 | 112.197 | 62.974 | 18.775 | 1.00 | 9.22 | N |
| ATOM | 4069 | CA | LEU | A | 528 | 112.126 | 64.342 | 19.272 | 1.00 | 9.68 | C |
| ATOM | 4070 | CB | LEU | A | 528 | 111.408 | 64.382 | 20.634 | 1.00 | 9.09 | C |
| ATOM | 4071 | CG | LEU | A | 528 | 110.002 | 63.775 | 20.684 | 1.00 | 9.41 | C |
| ATOM | 4072 | CD1 | LEU | A | 528 | 109.488 | 63.694 | 22.124 | 1.00 | 9.12 | C |
| ATOM | 4073 | CD2 | LEU | A | 528 | 109.030 | 64.599 | 19.823 | 1.00 | 8.64 | C |
| ATOM | 4074 | C | LEU | A | 528 | 113.515 | 64.909 | 19.449 | 1.00 | 9.76 | C |
| ATOM | 4075 | O | LEU | A | 528 | 114.362 | 64.267 | 20.049 | 1.00 | 10.21 | O |
| ATOM | 4076 | N | LEU | A | 529 | 113.719 | 66.115 | 18.932 | 1.00 | 10.18 | N |
| ATOM | 4077 | CA | LEU | A | 529 | 114.940 | 66.860 | 19.071 | 1.00 | 10.08 | C |
| ATOM | 4078 | CB | LEU | A | 529 | 115.575 | 67.129 | 17.695 | 1.00 | 9.32 | C |
| ATOM | 4079 | CG | LEU | A | 529 | 116.785 | 68.085 | 17.678 | 1.00 | 9.62 | C |
| ATOM | 4080 | CD1 | LEU | A | 529 | 118.000 | 67.439 | 18.343 | 1.00 | 11.82 | C |
| ATOM | 4081 | CD2 | LEU | A | 529 | 117.150 | 68.508 | 16.265 | 1.00 | 10.57 | C |
| ATOM | 4082 | C | LEU | A | 529 | 114.600 | 68.175 | 19.799 | 1.00 | 11.06 | C |
| ATOM | 4083 | O | LEU | A | 529 | 113.727 | 68.972 | 19.371 | 1.00 | 10.42 | O |
| ATOM | 4084 | N | ASP | A | 530 | 115.266 | 68.391 | 20.911 | 1.00 | 9.92 | N |
| ATOM | 4085 | CA | ASP | A | 530 | 115.041 | 69.596 | 21.704 | 1.00 | 11.43 | C |
| ATOM | 4086 | CB | ASP | A | 530 | 115.073 | 69.245 | 23.173 | 1.00 | 14.30 | C |
| ATOM | 4087 | CG | ASP | A | 530 | 114.798 | 70.443 | 24.083 | 1.00 | 16.65 | C |
| ATOM | 4088 | OD1 | ASP | A | 530 | 114.591 | 71.602 | 23.628 | 1.00 | 17.39 | O |
| ATOM | 4089 | OD2 | ASP | A | 530 | 114.822 | 70.196 | 25.287 | 1.00 | 18.37 | O |
| ATOM | 4090 | C | ASP | A | 530 | 116.132 | 70.578 | 21.331 | 1.00 | 10.62 | C |
| ATOM | 4091 | O | ASP | A | 530 | 117.187 | 70.577 | 21.922 | 1.00 | 10.38 | O |
| ATOM | 4092 | N | ALA | A | 531 | 115.894 | 71.359 | 20.292 | 1.00 | 10.14 | N |
| ATOM | 4093 | CA | ALA | A | 531 | 116.888 | 72.323 | 19.825 | 1.00 | 10.79 | C |
| ATOM | 4094 | CB | ALA | A | 531 | 117.950 | 71.649 | 18.941 | 1.00 | 9.75 | C |
| ATOM | 4095 | C | ALA | A | 531 | 116.201 | 73.426 | 19.042 | 1.00 | 10.66 | C |
| ATOM | 4096 | O | ALA | A | 531 | 115.168 | 73.230 | 18.375 | 1.00 | 10.28 | O |
| ATOM | 4097 | N | GLN | A | 532 | 116.791 | 74.592 | 19.134 | 1.00 | 10.54 | N |
| ATOM | 4098 | CA | GLN | A | 532 | 116.467 | 75.682 | 18.253 | 1.00 | 15.30 | C |
| ATOM | 4099 | CB | GLN | A | 532 | 117.405 | 76.857 | 18.575 | 1.00 | 16.44 | C |
| ATOM | 4100 | CG | GLN | A | 532 | 117.022 | 78.154 | 17.900 | 1.00 | 23.33 | C |
| ATOM | 4101 | CD | GLN | A | 532 | 117.997 | 79.280 | 18.191 | 1.00 | 25.77 | C |
| ATOM | 4102 | OE1 | GLN | A | 532 | 118.922 | 79.139 | 18.999 | 1.00 | 31.32 | O |
| ATOM | 4103 | NE2 | GLN | A | 532 | 117.787 | 80.405 | 17.544 | 1.00 | 26.18 | N |
| ATOM | 4104 | C | GLN | A | 532 | 116.657 | 75.241 | 16.781 | 1.00 | 13.76 | C |
| ATOM | 4105 | O | GLN | A | 532 | 117.622 | 74.559 | 16.467 | 1.00 | 11.93 | O |
| ATOM | 4106 | N | VAL | A | 533 | 115.762 | 75.666 | 15.897 | 1.00 | 12.61 | N |
| ATOM | 4107 | CA | VAL | A | 533 | 115.898 | 75.410 | 14.472 | 1.00 | 12.23 | C |
| ATOM | 4108 | CB | VAL | A | 533 | 114.625 | 75.848 | 13.697 | 1.00 | 12.90 | C |
| ATOM | 4109 | CG1 | VAL | A | 533 | 114.842 | 75.775 | 12.192 | 1.00 | 10.51 | C |
| ATOM | 4110 | CG2 | VAL | A | 533 | 113.429 | 74.979 | 14.107 | 1.00 | 10.72 | C |
| ATOM | 4111 | C | VAL | A | 533 | 117.122 | 76.148 | 13.920 | 1.00 | 13.56 | C |
| ATOM | 4112 | O | VAL | A | 533 | 117.261 | 77.351 | 14.113 | 1.00 | 12.02 | O |
| ATOM | 4113 | N | THR | A | 534 | 118.018 | 75.398 | 13.284 | 1.00 | 12.09 | N |
| ATOM | 4114 | CA | THR | A | 534 | 119.175 | 75.928 | 12.595 | 1.00 | 12.78 | C |
| ATOM | 4115 | CB | THR | A | 534 | 120.469 | 75.604 | 13.351 | 1.00 | 14.58 | C |
| ATOM | 4116 | OG1 | THR | A | 534 | 120.563 | 74.171 | 13.497 | 1.00 | 14.83 | O |
| ATOM | 4117 | CG2 | THR | A | 534 | 120.505 | 76.337 | 14.719 | 1.00 | 13.89 | C |
| ATOM | 4118 | C | THR | A | 534 | 119.381 | 75.376 | 11.189 | 1.00 | 13.87 | C |
| ATOM | 4119 | O | THR | A | 534 | 120.070 | 76.010 | 10.399 | 1.00 | 14.05 | O |
| ATOM | 4120 | N | GLU | A | 535 | 118.859 | 74.196 | 10.863 | 1.00 | 15.17 | N |
| ATOM | 4121 | CA | GLU | A | 535 | 119.104 | 73.617 | 9.521 | 1.00 | 16.86 | C |
| ATOM | 4122 | CB | GLU | A | 535 | 118.583 | 72.165 | 9.452 | 1.00 | 20.66 | C |
| ATOM | 4123 | CG | GLU | A | 535 | 118.387 | 71.568 | 8.065 | 1.00 | 28.44 | C |
| ATOM | 4124 | CD | GLU | A | 535 | 119.646 | 71.502 | 7.279 | 1.00 | 38.00 | C |
| ATOM | 4125 | OE1 | GLU | A | 535 | 120.694 | 71.256 | 7.907 | 1.00 | 43.92 | O |
| ATOM | 4126 | OE2 | GLU | A | 535 | 119.584 | 71.714 | 6.043 | 1.00 | 49.47 | O |
| ATOM | 4127 | C | GLU | A | 535 | 118.463 | 74.530 | 8.448 | 1.00 | 14.94 | C |
| ATOM | 4128 | O | GLU | A | 535 | 117.260 | 74.784 | 8.489 | 1.00 | 12.32 | O |
| ATOM | 4129 | N | PRO | A | 536 | 119.269 | 75.045 | 7.506 | 1.00 | 13.22 | N |
| ATOM | 4130 | CA | PRO | A | 536 | 118.787 | 76.103 | 6.607 | 1.00 | 14.11 | C |
| ATOM | 4131 | CB | PRO | A | 536 | 119.994 | 76.395 | 5.696 | 1.00 | 14.51 | C |
| ATOM | 4132 | CG | PRO | A | 536 | 121.184 | 75.893 | 6.427 | 1.00 | 16.92 | C |
| ATOM | 4133 | CD | PRO | A | 536 | 120.728 | 74.833 | 7.394 | 1.00 | 16.34 | C |
| ATOM | 4134 | C | PRO | A | 536 | 117.558 | 75.790 | 5.747 | 1.00 | 11.81 | C |
| ATOM | 4135 | O | PRO | A | 536 | 116.701 | 76.644 | 5.572 | 1.00 | 12.20 | O |
| ATOM | 4136 | N | THR | A | 537 | 117.485 | 74.589 | 5.207 | 1.00 | 10.03 | N |
| ATOM | 4137 | CA | THR | A | 537 | 116.364 | 74.177 | 4.361 | 1.00 | 11.44 | C |
| ATOM | 4138 | CB | THR | A | 537 | 116.758 | 72.938 | 3.522 | 1.00 | 13.05 | C |
| ATOM | 4139 | OG1 | THR | A | 537 | 116.976 | 71.808 | 4.374 | 1.00 | 16.34 | O |
| ATOM | 4140 | CG2 | THR | A | 537 | 118.049 | 73.203 | 2.750 | 1.00 | 11.25 | C |
| ATOM | 4141 | C | THR | A | 537 | 115.075 | 73.956 | 5.196 | 1.00 | 10.50 | C |
| ATOM | 4142 | O | THR | A | 537 | 113.969 | 74.280 | 4.774 | 1.00 | 9.54 | O |
| ATOM | 4143 | N | LEU | A | 538 | 115.247 | 73.473 | 6.411 | 1.00 | 9.49 | N |
| ATOM | 4144 | CA | LEU | A | 538 | 114.154 | 73.371 | 7.357 | 1.00 | 8.98 | C |
| ATOM | 4145 | CB | LEU | A | 538 | 114.604 | 72.593 | 8.595 | 1.00 | 8.29 | C |
| ATOM | 4146 | CG | LEU | A | 538 | 113.554 | 72.443 | 9.713 | 1.00 | 8.54 | C |
| ATOM | 4147 | CD1 | LEU | A | 538 | 112.371 | 71.621 | 9.205 | 1.00 | 8.35 | C |
| ATOM | 4148 | CD2 | LEU | A | 538 | 114.163 | 71.821 | 10.953 | 1.00 | 7.14 | C |
| ATOM | 4149 | C | LEU | A | 538 | 113.627 | 74.792 | 7.713 | 1.00 | 9.27 | C |
| ATOM | 4150 | O | LEU | A | 538 | 112.419 | 75.050 | 7.599 | 1.00 | 9.78 | O |
| ATOM | 4151 | N | ALA | A | 539 | 114.523 | 75.699 | 8.090 | 1.00 | 8.43 | N |
| ATOM | 4152 | CA | ALA | A | 539 | 114.134 | 77.080 | 8.399 | 1.00 | 10.03 | C |
| ATOM | 4153 | CB | ALA | A | 539 | 115.338 | 77.950 | 8.810 | 1.00 | 8.28 | C |
| ATOM | 4154 | C | ALA | A | 539 | 113.416 | 77.714 | 7.216 | 1.00 | 11.94 | C |
| ATOM | 4155 | O | ALA | A | 539 | 112.420 | 78.407 | 7.401 | 1.00 | 12.14 | O |
| ATOM | 4156 | N | ALA | A | 540 | 113.903 | 77.479 | 6.000 | 1.00 | 12.69 | N |
| ATOM | 4157 | CA | ALA | A | 540 | 113.261 | 78.090 | 4.829 | 1.00 | 13.57 | C |
| ATOM | 4158 | CB | ALA | A | 540 | 114.112 | 77.914 | 3.559 | 1.00 | 12.36 | C |
| ATOM | 4159 | C | ALA | A | 540 | 111.826 | 77.588 | 4.624 | 1.00 | 12.40 | C |
| ATOM | 4160 | O | ALA | A | 540 | 110.924 | 78.389 | 4.402 | 1.00 | 13.10 | O |
| ATOM | 4161 | N | ALA | A | 541 | 111.597 | 76.281 | 4.744 | 1.00 | 12.30 | N |
| ATOM | 4162 | CA | ALA | A | 541 | 110.260 | 75.730 | 4.607 | 1.00 | 12.06 | C |
| ATOM | 4163 | CB | ALA | A | 541 | 110.290 | 74.194 | 4.601 | 1.00 | 12.11 | C |
| ATOM | 4164 | C | ALA | A | 541 | 109.330 | 76.259 | 5.717 | 1.00 | 11.71 | C |
| ATOM | 4165 | O | ALA | A | 541 | 108.167 | 76.578 | 5.477 | 1.00 | 11.89 | O |
| ATOM | 4166 | N | LEU | A | 542 | 109.861 | 76.369 | 6.922 | 1.00 | 10.84 | N |
| ATOM | 4167 | CA | LEU | A | 542 | 109.081 | 76.820 | 8.061 | 1.00 | 10.57 | C |
| ATOM | 4168 | CB | LEU | A | 542 | 109.828 | 76.539 | 9.360 | 1.00 | 9.37 | C |
| ATOM | 4169 | CG | LEU | A | 542 | 110.008 | 75.053 | 9.792 | 1.00 | 9.27 | C |
| ATOM | 4170 | CD1 | LEU | A | 542 | 111.003 | 74.968 | 10.949 | 1.00 | 6.79 | C |
| ATOM | 4171 | CD2 | LEU | A | 542 | 108.679 | 74.412 | 10.154 | 1.00 | 7.05 | C |
| ATOM | 4172 | C | LEU | A | 542 | 108.746 | 78.325 | 7.946 | 1.00 | 12.02 | C |
| ATOM | 4173 | O | LEU | A | 542 | 107.635 | 78.736 | 8.295 | 1.00 | 9.27 | O |
| ATOM | 4174 | N | ASP | A | 543 | 109.720 | 79.121 | 7.481 | 1.00 | 10.97 | N |
| ATOM | 4175 | CA | ASP | A | 543 | 109.503 | 80.535 | 7.208 | 1.00 | 11.93 | C |
| ATOM | 4176 | CB | ASP | A | 543 | 110.778 | 81.211 | 6.677 | 1.00 | 11.96 | C |
| ATOM | 4177 | CG | ASP | A | 543 | 111.880 | 81.335 | 7.727 | 1.00 | 15.31 | C |
| ATOM | 4178 | OD1 | ASP | A | 543 | 111.587 | 81.321 | 8.945 | 1.00 | 14.43 | O |
| ATOM | 4179 | OD2 | ASP | A | 543 | 113.056 | 81.460 | 7.315 | 1.00 | 15.12 | O |
| ATOM | 4180 | C | ASP | A | 543 | 108.379 | 80.721 | 6.171 | 1.00 | 11.78 | C |
| ATOM | 4181 | O | ASP | A | 543 | 107.494 | 81.565 | 6.351 | 1.00 | 12.70 | O |
| ATOM | 4182 | N | ALA | A | 544 | 108.429 | 79.949 | 5.094 | 1.00 | 11.45 | N |
| ATOM | 4183 | CA | ALA | A | 544 | 107.393 | 80.014 | 4.057 | 1.00 | 13.07 | C |
| ATOM | 4184 | CB | ALA | A | 544 | 107.771 | 79.164 | 2.840 | 1.00 | 14.31 | C |
| ATOM | 4185 | C | ALA | A | 544 | 106.004 | 79.630 | 4.576 | 1.00 | 12.78 | C |
| ATOM | 4186 | O | ALA | A | 544 | 105.017 | 80.249 | 4.184 | 1.00 | 11.26 | O |
| ATOM | 4187 | N | ALA | A | 545 | 105.929 | 78.636 | 5.470 | 1.00 | 12.10 | N |
| ATOM | 4188 | CA | ALA | A | 545 | 104.653 | 78.258 | 6.101 | 1.00 | 11.76 | C |
| ATOM | 4189 | CB | ALA | A | 545 | 104.766 | 76.923 | 6.841 | 1.00 | 8.95 | C |
| ATOM | 4190 | C | ALA | A | 545 | 104.122 | 79.373 | 7.030 | 1.00 | 11.06 | C |
| ATOM | 4191 | O | ALA | A | 545 | 102.924 | 79.646 | 7.010 | 1.00 | 12.09 | O |
| ATOM | 4192 | N | MET | A | 546 | 104.994 | 80.051 | 7.789 | 1.00 | 10.46 | N |
| ATOM | 4193 | CA | MET | A | 546 | 104.555 | 81.173 | 8.640 | 1.00 | 11.25 | C |
| ATOM | 4194 | CB | MET | A | 546 | 105.658 | 81.652 | 9.618 | 1.00 | 11.09 | C |
| ATOM | 4195 | CG | MET | A | 546 | 106.097 | 80.590 | 10.628 | 1.00 | 11.56 | C |
| ATOM | 4196 | SD | MET | A | 546 | 104.767 | 80.106 | 11.774 | 1.00 | 12.53 | S |
| ATOM | 4197 | CE | MET | A | 546 | 104.589 | 81.658 | 12.694 | 1.00 | 10.43 | C |
| ATOM | 4198 | C | MET | A | 546 | 104.057 | 82.337 | 7.785 | 1.00 | 11.91 | C |
| ATOM | 4199 | O | MET | A | 546 | 103.026 | 82.922 | 8.103 | 1.00 | 13.59 | O |
| ATOM | 4200 | N | ALA | A | 547 | 104.776 | 82.674 | 6.719 | 1.00 | 12.15 | N |
| ATOM | 4201 | CA | ALA | A | 547 | 104.346 | 83.763 | 5.842 | 1.00 | 15.42 | C |
| ATOM | 4202 | CB | ALA | A | 547 | 105.393 | 84.097 | 4.786 | 1.00 | 14.98 | C |
| ATOM | 4203 | C | ALA | A | 547 | 102.997 | 83.411 | 5.188 | 1.00 | 16.22 | C |
| ATOM | 4204 | O | ALA | A | 547 | 102.121 | 84.251 | 5.111 | 1.00 | 17.52 | O |
| ATOM | 4205 | N | ASP | A | 548 | 102.815 | 82.159 | 4.777 | 1.00 | 15.85 | N |
| ATOM | 4206 | CA | ASP | A | 548 | 101.560 | 81.762 | 4.150 | 1.00 | 15.63 | C |
| ATOM | 4207 | CB | ASP | A | 548 | 101.582 | 80.321 | 3.666 | 1.00 | 16.71 | C |
| ATOM | 4208 | CG | ASP | A | 548 | 100.277 | 79.933 | 2.990 | 1.00 | 17.92 | C |
| ATOM | 4209 | OD1 | ASP | A | 548 | 100.047 | 80.384 | 1.859 | 1.00 | 23.54 | O |
| ATOM | 4210 | OD2 | ASP | A | 548 | 99.464 | 79.251 | 3.606 | 1.00 | 21.00 | O |
| ATOM | 4211 | C | ASP | A | 548 | 100.377 | 81.945 | 5.110 | 1.00 | 17.65 | C |
| ATOM | 4212 | O | ASP | A | 548 | 99.316 | 82.431 | 4.704 | 1.00 | 13.49 | O |
| ATOM | 4213 | N | ALA | A | 549 | 100.582 | 81.575 | 6.369 | 1.00 | 15.54 | N |
| ATOM | 4214 | CA | ALA | A | 549 | 99.535 | 81.640 | 7.373 | 1.00 | 19.94 | C |
| ATOM | 4215 | CB | ALA | A | 549 | 99.968 | 80.908 | 8.643 | 1.00 | 16.36 | C |
| ATOM | 4216 | C | ALA | A | 549 | 99.123 | 83.086 | 7.693 | 1.00 | 22.92 | C |
| ATOM | 4217 | O | ALA | A | 549 | 98.055 | 83.297 | 8.260 | 1.00 | 20.41 | O |
| ATOM | 4218 | N | ARG | A | 550 | 99.965 | 84.072 | 7.366 | 1.00 | 26.65 | N |
| ATOM | 4219 | CA | ARG | A | 550 | 99.590 | 85.480 | 7.565 | 1.00 | 30.18 | C |
| ATOM | 4220 | CB | ARG | A | 550 | 100.815 | 86.373 | 7.839 | 1.00 | 28.79 | C |
| ATOM | 4221 | CG | ARG | A | 550 | 101.406 | 86.152 | 9.227 | 1.00 | 29.17 | C |
| ATOM | 4222 | CD | ARG | A | 550 | 102.524 | 87.143 | 9.534 | 1.00 | 39.26 | C |
| ATOM | 4223 | NE | ARG | A | 550 | 103.742 | 86.795 | 8.804 | 1.00 | 47.80 | N |
| ATOM | 4224 | CZ | ARG | A | 550 | 104.685 | 85.943 | 9.230 | 1.00 | 51.10 | C |
| ATOM | 4225 | NH1 | ARG | A | 550 | 104.582 | 85.330 | 10.415 | 1.00 | 51.89 | N |
| ATOM | 4226 | NH2 | ARG | A | 550 | 105.744 | 85.696 | 8.457 | 1.00 | 33.57 | N |
| ATOM | 4227 | C | ARG | A | 550 | 98.725 | 86.069 | 6.442 | 1.00 | 33.84 | C |
| ATOM | 4228 | O | ARG | A | 550 | 98.128 | 87.120 | 6.637 | 1.00 | 31.87 | O |
| ATOM | 4229 | N | ARG | A | 551 | 98.619 | 85.392 | 5.301 | 1.00 | 36.87 | N |
| ATOM | 4230 | CA | ARG | A | 551 | 97.786 | 85.879 | 4.184 | 1.00 | 35.97 | C |
| ATOM | 4231 | CB | ARG | A | 551 | 98.063 | 85.076 | 2.913 | 1.00 | 34.44 | C |
| ATOM | 4232 | CG | ARG | A | 551 | 99.484 | 85.175 | 2.404 | 1.00 | 40.33 | C |
| ATOM | 4233 | CD | ARG | A | 551 | 99.671 | 84.462 | 1.064 | 1.00 | 50.67 | C |
| ATOM | 4234 | NE | ARG | A | 551 | 100.913 | 83.674 | 1.057 | 1.00 | 68.59 | N |
| ATOM | 4235 | CZ | ARG | A | 551 | 102.152 | 84.180 | 1.113 | 1.00 | 69.62 | C |
| ATOM | 4236 | NH1 | ARG | A | 551 | 102.359 | 85.496 | 1.159 | 1.00 | 70.63 | N |
| ATOM | 4237 | NH2 | ARG | A | 551 | 103.206 | 83.365 | 1.130 | 1.00 | 50.36 | N |
| ATOM | 4238 | C | ARG | A | 551 | 96.283 | 85.782 | 4.483 | 1.00 | 40.67 | C |
| ATOM | 4239 | O | ARG | A | 551 | 95.820 | 84.797 | 5.078 | 1.00 | 41.34 | O |
| ATOM | 4240 | N | SER | A | 552 | 95.528 | 86.788 | 4.038 | 1.00 | 45.37 | N |
| ATOM | 4241 | CA | SER | A | 552 | 94.055 | 86.754 | 4.083 | 1.00 | 48.64 | C |
| ATOM | 4242 | CB | SER | A | 552 | 93.466 | 88.064 | 3.546 | 1.00 | 45.77 | C |
| ATOM | 4243 | OG | SER | A | 552 | 93.565 | 89.086 | 4.511 | 1.00 | 56.51 | O |
| ATOM | 4244 | C | SER | A | 552 | 93.465 | 85.613 | 3.257 | 1.00 | 53.97 | C |
| ATOM | 4245 | O | SER | A | 552 | 93.988 | 85.285 | 2.195 | 1.00 | 44.21 | O |
| ATOM | 4246 | N | ARG | A | 553 | 92.400 | 84.997 | 3.776 | 1.00 | 58.75 | N |
| ATOM | 4247 | CA | ARG | A | 553 | 91.395 | 84.310 | 2.946 | 1.00 | 62.60 | C |
| ATOM | 4248 | CB | ARG | A | 553 | 91.490 | 82.774 | 2.990 | 1.00 | 53.31 | C |
| ATOM | 4249 | CG | ARG | A | 553 | 92.046 | 82.144 | 4.257 | 1.00 | 45.97 | C |
| ATOM | 4250 | CD | ARG | A | 553 | 93.550 | 81.923 | 4.128 | 1.00 | 48.06 | C |
| ATOM | 4251 | NE | ARG | A | 553 | 94.060 | 81.093 | 5.208 | 1.00 | 47.70 | N |
| ATOM | 4252 | CZ | ARG | A | 553 | 95.283 | 80.574 | 5.267 | 1.00 | 48.46 | C |
| ATOM | 4253 | NH1 | ARG | A | 553 | 96.176 | 80.798 | 4.300 | 1.00 | 43.23 | N |
| ATOM | 4254 | NH2 | ARG | A | 553 | 95.611 | 79.817 | 6.313 | 1.00 | 48.31 | N |
| ATOM | 4255 | C | ARG | A | 553 | 90.001 | 84.766 | 3.376 | 1.00 | 77.50 | C |
| ATOM | 4256 | O | ARG | A | 553 | 89.820 | 85.337 | 4.454 | 1.00 | 86.21 | O |
| ATOM | 4257 | OXT | ARG | A | 553 | 89.021 | 84.582 | 2.656 | 1.00 | 87.66 | O |
| ATOM | 4258 | CA | CA | A | 600 | 70.802 | 57.236 | 35.062 | 1.00 | 15.98 | CA |
| ATOM | 4259 | CA | CA | A | 601 | 63.208 | 65.968 | 29.650 | 1.00 | 13.29 | CA |
| ATOM | 4260 | CA | CA | A | 602 | 67.621 | 77.498 | 10.597 | 1.00 | 9.99 | CA |
| ATOM | 4680 | CL | CL | A | 603 | 72.381 | 53.223 | 16.908 | 1.00 | 13.61 | CL |
| ATOM | 4674 | O3 | GOL | B | 1 | 71.181 | 62.613 | 17.255 | 1.00 | 12.69 | O |
| ATOM | 4675 | C3 | GOL | B | 1 | 70.059 | 63.246 | 16.665 | 1.00 | 15.67 | C |
| ATOM | 4676 | C2 | GOL | B | 1 | 68.954 | 62.285 | 16.286 | 1.00 | 21.27 | C |
| ATOM | 4677 | O2 | GOL | B | 1 | 69.488 | 61.026 | 15.941 | 1.00 | 26.24 | O |
| ATOM | 4678 | C1 | GOL | B | 1 | 68.041 | 62.832 | 15.191 | 1.00 | 22.45 | C |
| ATOM | 4679 | O1 | GOL | B | 1 | 67.264 | 61.777 | 14.630 | 1.00 | 26.00 | O |
| ATOM | 4681 | O3 | GOL | B | 2 | 72.946 | 67.251 | 14.695 | 1.00 | 15.32 | O |
| ATOM | 4682 | C3 | GOL | B | 2 | 71.767 | 66.657 | 15.249 | 1.00 | 14.09 | C |
| ATOM | 4683 | C2 | GOL | B | 2 | 70.559 | 67.586 | 15.090 | 1.00 | 14.05 | C |
| ATOM | 4684 | O2 | GOL | B | 2 | 70.621 | 68.590 | 16.096 | 1.00 | 12.33 | O |
| ATOM | 4685 | C1 | GOL | B | 2 | 69.249 | 66.846 | 15.221 | 1.00 | 18.07 | C |
| ATOM | 4686 | O1 | GOL | B | 2 | 69.023 | 66.062 | 14.039 | 1.00 | 17.82 | O |
| ATOM | 4687 | O3 | GOL | B | 3 | 72.515 | 64.096 | 10.689 | 1.00 | 25.67 | O |
| ATOM | 4688 | C3 | GOL | C | 4 | 73.467 | 64.963 | 11.307 | 1.00 | 26.15 | C |
| ATOM | 4689 | C2 | GOL | C | 4 | 72.861 | 66.338 | 11.544 | 1.00 | 25.64 | C |
| ATOM | 4690 | O2 | GOL | C | 4 | 73.851 | 67.096 | 12.249 | 1.00 | 19.50 | O |
| ATOM | 4691 | C1 | GOL | C | 4 | 72.435 | 67.029 | 10.241 | 1.00 | 22.26 | C |
| ATOM | 4692 | O1 | GOL | C | 4 | 73.539 | 67.080 | 9.298 | 1.00 | 23.90 | O |
| ATOM | 4261 | O | HOH | W | 1 | 73.301 | 61.252 | 25.027 | 1.00 | 9.85 | O |
| ATOM | 4262 | O | HOH | W | 2 | 94.676 | 65.066 | 13.807 | 1.00 | 10.58 | O |
| ATOM | 4263 | O | HOH | W | 3 | 59.711 | 76.459 | 12.227 | 1.00 | 7.99 | O |
| ATOM | 4264 | O | HOH | W | 4 | 98.238 | 68.392 | 15.962 | 1.00 | 11.94 | O |
| ATOM | 4265 | O | HOH | W | 5 | 97.088 | 65.408 | 16.506 | 1.00 | 11.85 | O |
| ATOM | 4266 | O | HOH | W | 6 | 62.085 | 65.925 | 13.108 | 1.00 | 16.04 | O |
| ATOM | 4267 | O | HOH | W | 7 | 73.920 | 61.551 | 37.146 | 1.00 | 15.54 | O |
| ATOM | 4268 | O | HOH | W | 8 | 77.168 | 50.157 | 33.169 | 1.00 | 15.21 | O |
| ATOM | 4269 | O | HOH | W | 9 | 61.919 | 63.088 | 23.865 | 1.00 | 11.76 | O |
| ATOM | 4270 | O | HOH | W | 10 | 72.608 | 74.669 | 15.812 | 1.00 | 10.72 | O |
| ATOM | 4271 | O | HOH | W | 11 | 63.466 | 64.051 | 28.377 | 1.00 | 9.82 | O |
| ATOM | 4272 | O | HOH | W | 12 | 69.752 | 66.825 | 27.140 | 1.00 | 12.03 | O |
| ATOM | 4273 | O | HOH | W | 13 | 96.364 | 76.517 | 4.720 | 1.00 | 15.03 | O |
| ATOM | 4274 | O | HOH | W | 14 | 114.715 | 68.298 | 11.096 | 1.00 | 13.68 | O |
| ATOM | 4275 | O | HOH | W | 15 | 97.437 | 71.403 | 15.360 | 1.00 | 10.85 | O |
| ATOM | 4276 | O | HOH | W | 16 | 77.509 | 58.818 | 30.992 | 1.00 | 10.02 | O |
| ATOM | 4277 | O | HOH | W | 17 | 90.586 | 69.226 | 5.708 | 1.00 | 12.97 | O |
| ATOM | 4278 | O | HOH | W | 18 | 68.221 | 64.485 | 27.002 | 1.00 | 15.33 | O |
| ATOM | 4279 | O | HOH | W | 19 | 80.372 | 45.787 | 14.915 | 1.00 | 13.14 | O |
| ATOM | 4280 | O | HOH | W | 20 | 89.056 | 64.621 | 10.452 | 1.00 | 13.00 | O |
| ATOM | 4281 | O | HOH | W | 21 | 71.497 | 52.451 | 19.773 | 1.00 | 9.03 | O |
| ATOM | 4282 | O | HOH | W | 22 | 87.363 | 68.955 | 0.376 | 1.00 | 15.62 | O |
| ATOM | 4283 | O | HOH | W | 23 | 88.823 | 64.449 | 1.620 | 1.00 | 17.84 | O |
| ATOM | 4284 | O | HOH | W | 24 | 56.279 | 65.027 | 11.407 | 1.00 | 19.72 | O |
| ATOM | 4285 | O | HOH | W | 25 | 87.136 | 52.751 | 3.189 | 1.00 | 17.97 | O |
| ATOM | 4286 | O | HOH | W | 26 | 69.509 | 54.464 | 17.311 | 1.00 | 13.33 | O |
| ATOM | 4287 | O | HOH | W | 27 | 82.485 | 70.854 | 9.739 | 1.00 | 9.26 | O |
| ATOM | 4288 | O | HOH | W | 28 | 72.417 | 49.514 | 23.793 | 1.00 | 10.68 | O |
| ATOM | 4289 | O | HOH | W | 29 | 75.569 | 72.013 | 9.882 | 1.00 | 7.94 | O |
| ATOM | 4290 | O | HOH | W | 30 | 61.004 | 77.739 | 8.577 | 1.00 | 10.78 | O |
| ATOM | 4291 | O | HOH | W | 31 | 96.117 | 69.727 | 1.018 | 1.00 | 16.27 | O |
| ATOM | 4292 | O | HOH | W | 32 | 74.350 | 72.855 | 37.663 | 1.00 | 26.67 | O |
| ATOM | 4293 | O | HOH | W | 33 | 63.557 | 56.952 | 14.842 | 1.00 | 22.65 | O |
| ATOM | 4294 | O | HOH | W | 34 | 87.254 | 72.718 | −1.207 | 1.00 | 18.26 | O |
| ATOM | 4295 | O | HOH | W | 35 | 63.640 | 78.182 | 7.997 | 1.00 | 12.81 | O |
| ATOM | 4296 | O | HOH | W | 36 | 85.296 | 51.423 | 5.220 | 1.00 | 14.66 | O |
| ATOM | 4297 | O | HOH | W | 37 | 97.165 | 53.677 | 18.048 | 1.00 | 10.53 | O |
| ATOM | 4298 | O | HOH | W | 38 | 64.992 | 73.401 | 11.693 | 1.00 | 8.79 | O |
| ATOM | 4299 | O | HOH | W | 39 | 101.191 | 51.144 | 16.856 | 1.00 | 15.00 | O |
| ATOM | 4300 | O | HOH | W | 40 | 75.284 | 85.427 | 6.931 | 1.00 | 17.36 | O |
| ATOM | 4301 | O | HOH | W | 41 | 101.467 | 75.016 | 7.535 | 1.00 | 10.33 | O |
| ATOM | 4302 | O | HOH | W | 42 | 72.548 | 75.644 | −0.400 | 1.00 | 10.93 | O |
| ATOM | 4303 | O | HOH | W | 43 | 61.115 | 84.329 | 10.013 | 1.00 | 12.37 | O |
| ATOM | 4304 | O | HOH | W | 44 | 69.051 | 57.185 | 17.220 | 1.00 | 11.51 | O |
| ATOM | 4305 | O | HOH | W | 45 | 73.187 | 63.785 | 15.880 | 1.00 | 11.17 | O |
| ATOM | 4306 | O | HOH | W | 46 | 65.091 | 72.566 | 4.534 | 1.00 | 7.86 | O |
| ATOM | 4307 | O | HOH | W | 47 | 57.598 | 82.971 | 20.246 | 1.00 | 17.55 | O |
| ATOM | 4308 | O | HOH | W | 48 | 74.831 | 78.095 | 6.016 | 1.00 | 23.98 | O |
| ATOM | 4309 | O | HOH | W | 49 | 94.448 | 64.970 | 10.381 | 1.00 | 53.92 | O |
| ATOM | 4310 | O | HOH | W | 50 | 68.059 | 48.033 | 21.658 | 1.00 | 14.51 | O |
| ATOM | 4311 | O | HOH | W | 51 | 69.864 | 50.679 | 30.531 | 1.00 | 23.84 | O |
| ATOM | 4312 | O | HOH | W | 52 | 101.668 | 62.523 | 28.476 | 1.00 | 16.04 | O |
| ATOM | 4313 | O | HOH | W | 53 | 60.457 | 82.754 | 8.003 | 1.00 | 10.43 | O |
| ATOM | 4314 | O | HOH | W | 54 | 98.841 | 70.632 | 2.293 | 1.00 | 18.12 | O |
| ATOM | 4315 | O | HOH | W | 55 | 67.848 | 79.785 | 10.036 | 1.00 | 9.14 | O |
| ATOM | 4316 | O | HOH | W | 56 | 74.745 | 56.274 | 10.581 | 1.00 | 18.03 | O |
| ATOM | 4317 | O | HOH | W | 57 | 107.555 | 65.751 | 24.148 | 1.00 | 17.39 | O |
| ATOM | 4318 | O | HOH | W | 58 | 81.728 | 45.851 | 19.575 | 1.00 | 20.99 | O |
| ATOM | 4319 | O | HOH | W | 59 | 74.805 | 89.197 | 26.239 | 1.00 | 32.84 | O |
| ATOM | 4320 | O | HOH | W | 60 | 78.732 | 79.384 | 5.490 | 1.00 | 19.88 | O |
| ATOM | 4321 | O | HOH | W | 61 | 99.553 | 53.549 | −2.727 | 1.00 | 19.88 | O |
| ATOM | 4322 | O | HOH | W | 62 | 94.758 | 52.611 | 18.965 | 1.00 | 11.05 | O |
| ATOM | 4323 | O | HOH | W | 63 | 67.287 | 76.740 | 8.446 | 1.00 | 11.29 | O |
| ATOM | 4324 | O | HOH | W | 64 | 83.036 | 49.847 | 4.981 | 1.00 | 34.91 | O |
| ATOM | 4325 | O | HOH | W | 65 | 87.761 | 78.169 | 25.477 | 1.00 | 13.59 | O |
| ATOM | 4326 | O | HOH | W | 66 | 121.197 | 67.660 | 19.855 | 1.00 | 22.88 | O |
| ATOM | 4327 | O | HOH | W | 67 | 68.256 | 60.005 | 12.705 | 1.00 | 15.28 | O |
| ATOM | 4328 | O | HOH | W | 68 | 67.303 | 78.440 | 12.806 | 1.00 | 8.06 | O |
| ATOM | 4329 | O | HOH | W | 69 | 66.845 | 79.531 | −1.065 | 1.00 | 11.75 | O |
| ATOM | 4330 | O | HOH | W | 70 | 55.069 | 60.199 | 32.523 | 1.00 | 23.75 | O |
| ATOM | 4331 | O | HOH | W | 71 | 74.053 | 46.715 | 12.581 | 1.00 | 15.95 | O |
| ATOM | 4332 | O | HOH | W | 72 | 105.148 | 61.097 | −0.647 | 1.00 | 17.00 | O |
| ATOM | 4333 | O | HOH | W | 73 | 64.494 | 84.335 | 9.317 | 1.00 | 12.26 | O |
| ATOM | 4334 | O | HOH | W | 74 | 100.602 | 53.757 | 19.330 | 1.00 | 10.19 | O |
| ATOM | 4335 | O | HOH | W | 75 | 67.110 | 83.095 | 1.371 | 1.00 | 20.23 | O |
| ATOM | 4336 | O | HOH | W | 76 | 103.582 | 73.620 | 6.383 | 1.00 | 13.21 | O |
| ATOM | 4337 | O | HOH | W | 77 | 63.044 | 58.060 | 30.204 | 1.00 | 10.84 | O |
| ATOM | 4338 | O | HOH | W | 78 | 83.815 | 86.086 | 17.713 | 1.00 | 20.16 | O |
| ATOM | 4339 | O | HOH | W | 79 | 123.110 | 58.293 | 10.793 | 1.00 | 22.58 | O |
| ATOM | 4340 | O | HOH | W | 80 | 90.442 | 77.202 | 11.004 | 1.00 | 12.85 | O |
| ATOM | 4341 | O | HOH | W | 81 | 57.217 | 86.597 | 21.382 | 1.00 | 23.04 | O |
| ATOM | 4342 | O | HOH | W | 82 | 53.312 | 61.917 | 29.205 | 1.00 | 18.81 | O |
| ATOM | 4343 | O | HOH | W | 83 | 111.915 | 70.094 | 23.094 | 1.00 | 25.06 | O |
| ATOM | 4344 | O | HOH | W | 84 | 89.625 | 78.270 | 13.483 | 1.00 | 18.96 | O |
| ATOM | 4345 | O | HOH | W | 85 | 96.516 | 63.335 | 23.952 | 1.00 | 14.76 | O |
| ATOM | 4346 | O | HOH | W | 86 | 58.334 | 56.896 | 7.380 | 1.00 | 27.79 | O |
| ATOM | 4347 | O | HOH | W | 87 | 99.460 | 74.622 | 25.157 | 1.00 | 22.95 | O |
| ATOM | 4348 | O | HOH | W | 88 | 104.112 | 48.169 | 5.268 | 1.00 | 18.97 | O |
| ATOM | 4349 | O | HOH | W | 89 | 99.458 | 68.003 | 1.873 | 1.00 | 15.08 | O |
| ATOM | 4350 | O | HOH | W | 90 | 88.400 | 80.289 | 2.682 | 1.00 | 17.29 | O |
| ATOM | 4351 | O | HOH | W | 91 | 75.965 | 70.423 | 4.608 | 1.00 | 14.51 | O |
| ATOM | 4352 | O | HOH | W | 92 | 53.399 | 62.729 | 10.490 | 1.00 | 24.51 | O |
| ATOM | 4353 | O | HOH | W | 93 | 68.022 | 85.521 | 7.670 | 1.00 | 17.66 | O |
| ATOM | 4354 | O | HOH | W | 94 | 105.909 | 78.173 | 24.075 | 1.00 | 26.48 | O |
| ATOM | 4355 | O | HOH | W | 95 | 101.655 | 73.227 | 23.203 | 1.00 | 23.79 | O |
| ATOM | 4356 | O | HOH | W | 96 | 103.701 | 50.954 | 0.929 | 1.00 | 23.11 | O |
| ATOM | 4357 | O | HOH | W | 97 | 59.352 | 53.719 | 26.555 | 1.00 | 23.04 | O |
| ATOM | 4358 | O | HOH | W | 98 | 101.037 | 80.238 | 12.096 | 1.00 | 14.80 | O |
| ATOM | 4359 | O | HOH | W | 99 | 104.659 | 69.936 | 3.574 | 1.00 | 14.04 | O |
| ATOM | 4360 | O | HOH | W | 100 | 103.458 | 57.078 | 25.658 | 1.00 | 18.60 | O |
| ATOM | 4361 | O | HOH | W | 101 | 53.805 | 58.863 | 12.913 | 1.00 | 19.64 | O |
| ATOM | 4362 | O | HOH | W | 102 | 99.745 | 78.300 | 10.665 | 1.00 | 14.90 | O |
| ATOM | 4363 | O | HOH | W | 103 | 77.791 | 89.583 | 25.879 | 1.00 | 23.54 | O |
| ATOM | 4364 | O | HOH | W | 104 | 67.035 | 91.326 | 12.980 | 1.00 | 13.78 | O |
| ATOM | 4365 | O | HOH | W | 105 | 98.933 | 81.009 | 13.428 | 1.00 | 19.24 | O |
| ATOM | 4366 | O | HOH | W | 106 | 75.580 | 70.650 | 7.511 | 1.00 | 11.53 | O |
| ATOM | 4367 | O | HOH | W | 107 | 59.161 | 75.386 | 2.056 | 1.00 | 24.13 | O |
| ATOM | 4368 | O | HOH | W | 108 | 66.095 | 86.539 | 5.769 | 1.00 | 49.22 | O |
| ATOM | 4369 | O | HOH | W | 109 | 70.790 | 48.831 | 21.889 | 1.00 | 17.12 | O |
| ATOM | 4370 | O | HOH | W | 110 | 97.251 | 66.013 | 23.525 | 1.00 | 13.20 | O |
| ATOM | 4371 | O | HOH | W | 111 | 90.643 | 65.822 | 0.466 | 1.00 | 17.40 | O |
| ATOM | 4372 | O | HOH | W | 112 | 77.830 | 47.523 | 31.224 | 1.00 | 18.87 | O |
| ATOM | 4373 | O | HOH | W | 113 | 75.241 | 65.944 | 15.456 | 1.00 | 9.63 | O |
| ATOM | 4374 | O | HOH | W | 114 | 60.785 | 50.522 | 27.718 | 1.00 | 31.80 | O |
| ATOM | 4375 | O | HOH | W | 115 | 64.668 | 86.557 | 24.982 | 1.00 | 23.71 | O |
| ATOM | 4376 | O | HOH | W | 116 | 101.098 | 77.558 | 8.202 | 1.00 | 22.22 | O |
| ATOM | 4377 | O | HOH | W | 117 | 106.242 | 44.285 | 12.903 | 1.00 | 21.32 | O |
| ATOM | 4378 | O | HOH | W | 118 | 123.211 | 57.118 | 17.184 | 1.00 | 30.89 | O |
| ATOM | 4379 | O | HOH | W | 119 | 49.666 | 66.238 | 12.695 | 1.00 | 21.19 | O |
| ATOM | 4380 | O | HOH | W | 120 | 71.195 | 75.320 | 36.843 | 1.00 | 25.49 | O |
| ATOM | 4381 | O | HOH | W | 121 | 81.772 | 73.968 | 38.012 | 1.00 | 18.19 | O |
| ATOM | 4382 | O | HOH | W | 122 | 106.975 | 54.241 | 19.306 | 1.00 | 35.36 | O |
| ATOM | 4383 | O | HOH | W | 123 | 95.547 | 54.279 | −2.956 | 1.00 | 27.88 | O |
| ATOM | 4384 | O | HOH | W | 124 | 87.005 | 79.057 | 18.173 | 1.00 | 14.23 | O |
| ATOM | 4385 | O | HOH | W | 125 | 79.393 | 54.321 | 37.906 | 1.00 | 37.60 | O |
| ATOM | 4386 | O | HOH | W | 126 | 94.507 | 47.383 | 26.763 | 1.00 | 23.32 | O |
| ATOM | 4387 | O | HOH | W | 127 | 84.879 | 52.791 | 7.568 | 1.00 | 17.75 | O |
| ATOM | 4388 | O | HOH | W | 128 | 55.781 | 62.401 | 29.883 | 1.00 | 18.64 | O |
| ATOM | 4389 | O | HOH | W | 129 | 110.793 | 64.343 | 1.131 | 1.00 | 31.33 | O |
| ATOM | 4390 | O | HOH | W | 130 | 74.853 | 47.091 | 10.115 | 1.00 | 25.36 | O |
| ATOM | 4391 | O | HOH | W | 131 | 68.626 | 48.003 | 15.032 | 1.00 | 19.30 | O |
| ATOM | 4392 | O | HOH | W | 132 | 52.375 | 86.184 | 10.402 | 1.00 | 20.12 | O |
| ATOM | 4393 | O | HOH | W | 133 | 61.832 | 62.184 | 10.783 | 1.00 | 14.63 | O |
| ATOM | 4394 | O | HOH | W | 134 | 59.866 | 87.352 | 20.211 | 1.00 | 18.08 | O |
| ATOM | 4395 | O | HOH | W | 135 | 53.092 | 75.022 | 28.267 | 1.00 | 29.34 | O |
| ATOM | 4396 | O | HOH | W | 136 | 71.633 | 51.059 | 32.204 | 1.00 | 27.11 | O |
| ATOM | 4397 | O | HOH | W | 137 | 65.108 | 44.264 | 19.073 | 1.00 | 15.72 | O |
| ATOM | 4398 | O | HOH | W | 138 | 94.400 | 49.865 | 27.884 | 1.00 | 30.99 | O |
| ATOM | 4399 | O | HOH | W | 139 | 50.035 | 63.741 | 26.686 | 1.00 | 25.35 | O |
| ATOM | 4400 | O | HOH | W | 140 | 46.992 | 72.601 | 19.528 | 1.00 | 23.56 | O |
| ATOM | 4401 | O | HOH | W | 141 | 58.833 | 62.898 | 33.585 | 1.00 | 24.60 | O |
| ATOM | 4402 | O | HOH | W | 142 | 44.590 | 64.528 | 17.631 | 1.00 | 22.37 | O |
| ATOM | 4403 | O | HOH | W | 143 | 111.934 | 67.262 | 22.863 | 1.00 | 21.36 | O |
| ATOM | 4404 | O | HOH | W | 144 | 120.388 | 70.373 | 16.578 | 1.00 | 14.72 | O |
| ATOM | 4405 | O | HOH | W | 145 | 79.376 | 74.934 | 5.640 | 1.00 | 26.11 | O |
| ATOM | 4406 | O | HOH | W | 146 | 90.390 | 44.528 | 20.624 | 1.00 | 19.00 | O |
| ATOM | 4407 | O | HOH | W | 147 | 76.952 | 72.281 | 2.898 | 1.00 | 17.25 | O |
| ATOM | 4408 | O | HOH | W | 148 | 62.874 | 55.052 | 36.871 | 1.00 | 21.58 | O |
| ATOM | 4409 | O | HOH | W | 149 | 109.178 | 61.001 | 0.007 | 0.50 | 12.71 | O |
| ATOM | 4410 | O | HOH | W | 150 | 56.846 | 65.884 | 26.764 | 1.00 | 16.72 | O |
| ATOM | 4411 | O | HOH | W | 151 | 71.021 | 82.380 | 33.675 | 1.00 | 35.41 | O |
| ATOM | 4412 | O | HOH | W | 152 | 87.683 | 55.474 | 36.044 | 1.00 | 25.03 | O |
| ATOM | 4413 | O | HOH | W | 153 | 123.483 | 60.711 | 3.281 | 1.00 | 21.36 | O |
| ATOM | 4414 | O | HOH | W | 154 | 120.007 | 74.411 | 17.561 | 1.00 | 23.51 | O |
| ATOM | 4415 | O | HOH | W | 155 | 104.565 | 50.945 | 16.980 | 1.00 | 26.60 | O |
| ATOM | 4416 | O | HOH | W | 156 | 50.788 | 68.696 | 12.297 | 1.00 | 13.87 | O |
| ATOM | 4417 | O | HOH | W | 157 | 64.965 | 81.214 | 5.914 | 1.00 | 19.59 | O |
| ATOM | 4418 | O | HOH | W | 158 | 77.322 | 74.978 | 4.042 | 1.00 | 30.33 | O |
| ATOM | 4419 | O | HOH | W | 159 | 90.308 | 65.171 | −2.317 | 1.00 | 25.76 | O |
| ATOM | 4420 | O | HOH | W | 160 | 60.365 | 48.834 | 20.546 | 0.50 | 16.06 | O |
| ATOM | 4421 | O | HOH | W | 161 | 106.038 | 63.576 | 23.717 | 1.00 | 17.05 | O |
| ATOM | 4422 | O | HOH | W | 162 | 72.774 | 83.675 | 31.704 | 1.00 | 24.11 | O |
| ATOM | 4423 | O | HOH | W | 163 | 115.099 | 80.456 | 11.855 | 1.00 | 21.57 | O |
| ATOM | 4424 | O | HOH | W | 164 | 83.838 | 59.796 | 3.811 | 1.00 | 32.77 | O |
| ATOM | 4425 | O | HOH | W | 165 | 96.519 | 81.214 | 9.486 | 1.00 | 24.49 | O |
| ATOM | 4426 | O | HOH | W | 166 | 110.093 | 46.455 | 13.328 | 1.00 | 25.75 | O |
| ATOM | 4427 | O | HOH | W | 167 | 88.789 | 80.875 | 13.101 | 1.00 | 22.74 | O |
| ATOM | 4428 | O | HOH | W | 168 | 93.993 | 42.594 | 18.292 | 1.00 | 35.15 | O |
| ATOM | 4429 | O | HOH | W | 169 | 114.470 | 62.504 | 3.385 | 1.00 | 17.97 | O |
| ATOM | 4430 | O | HOH | W | 170 | 78.601 | 75.108 | −3.396 | 1.00 | 18.66 | O |
| ATOM | 4431 | O | HOH | W | 171 | 93.427 | 74.507 | 26.479 | 1.00 | 18.67 | O |
| ATOM | 4432 | O | HOH | W | 172 | 97.843 | 81.288 | 16.078 | 1.00 | 23.16 | O |
| ATOM | 4433 | O | HOH | W | 173 | 54.469 | 71.301 | 27.298 | 1.00 | 15.33 | O |
| ATOM | 4434 | O | HOH | W | 174 | 93.660 | 76.333 | 5.563 | 1.00 | 17.81 | O |
| ATOM | 4435 | O | HOH | W | 175 | 84.853 | 68.817 | −2.631 | 1.00 | 22.36 | O |
| ATOM | 4436 | O | HOH | W | 176 | 121.180 | 70.239 | 19.170 | 1.00 | 29.32 | O |
| ATOM | 4437 | O | HOH | W | 177 | 108.298 | 83.418 | 12.138 | 1.00 | 25.06 | O |
| ATOM | 4438 | O | HOH | W | 178 | 83.712 | 78.028 | 32.000 | 1.00 | 20.46 | O |
| ATOM | 4439 | O | HOH | W | 179 | 67.141 | 81.712 | 7.552 | 1.00 | 21.89 | O |
| ATOM | 4440 | O | HOH | W | 180 | 62.541 | 68.408 | 33.461 | 1.00 | 29.28 | O |
| ATOM | 4441 | O | HOH | W | 181 | 65.408 | 70.134 | 3.389 | 1.00 | 27.44 | O |
| ATOM | 4442 | O | HOH | W | 182 | 113.444 | 63.788 | 0.119 | 1.00 | 33.32 | O |
| ATOM | 4443 | O | HOH | W | 183 | 99.202 | 79.019 | 23.664 | 1.00 | 33.88 | O |
| ATOM | 4444 | O | HOH | W | 184 | 81.811 | 56.321 | 8.309 | 1.00 | 18.01 | O |
| ATOM | 4445 | O | HOH | W | 185 | 104.695 | 48.686 | 1.991 | 1.00 | 29.99 | O |
| ATOM | 4446 | O | HOH | W | 186 | 91.166 | 47.945 | 8.668 | 1.00 | 22.78 | O |
| ATOM | 4447 | O | HOH | W | 187 | 76.861 | 76.447 | −1.977 | 1.00 | 29.90 | O |
| ATOM | 4448 | O | HOH | W | 188 | 96.399 | 56.342 | −4.528 | 1.00 | 36.18 | O |
| ATOM | 4449 | O | HOH | W | 189 | 121.474 | 73.020 | 15.923 | 1.00 | 25.26 | O |
| ATOM | 4450 | O | HOH | W | 190 | 75.992 | 79.362 | 8.074 | 1.00 | 15.32 | O |
| ATOM | 4451 | O | HOH | W | 191 | 54.002 | 55.741 | 33.109 | 1.00 | 27.06 | O |
| ATOM | 4452 | O | HOH | W | 192 | 57.016 | 67.916 | 7.091 | 1.00 | 27.85 | O |
| ATOM | 4453 | O | HOH | W | 193 | 65.809 | 67.983 | 6.395 | 1.00 | 26.22 | O |
| ATOM | 4454 | O | HOH | W | 194 | 66.750 | 47.235 | 25.205 | 1.00 | 21.34 | O |
| ATOM | 4455 | O | HOH | W | 195 | 95.184 | 73.954 | 0.422 | 1.00 | 22.08 | O |
| ATOM | 4456 | O | HOH | W | 196 | 75.333 | 75.333 | −0.000 | 0.50 | 13.56 | O |
| ATOM | 4457 | O | HOH | W | 197 | 81.217 | 56.678 | 3.652 | 1.00 | 37.14 | O |
| ATOM | 4458 | O | HOH | W | 198 | 125.030 | 60.203 | 11.952 | 1.00 | 17.94 | O |
| ATOM | 4459 | O | HOH | W | 199 | 76.598 | 89.434 | 12.516 | 1.00 | 22.60 | O |
| ATOM | 4460 | O | HOH | W | 200 | 112.635 | 73.309 | 24.326 | 1.00 | 20.41 | O |
| ATOM | 4461 | O | HOH | W | 201 | 84.628 | 74.145 | 37.911 | 1.00 | 29.11 | O |
| ATOM | 4462 | O | HOH | W | 202 | 72.195 | 61.400 | 12.843 | 1.00 | 21.93 | O |
| ATOM | 4463 | O | HOH | W | 203 | 102.947 | 60.372 | −2.389 | 1.00 | 19.32 | O |
| ATOM | 4464 | O | HOH | W | 204 | 82.537 | 67.759 | 41.630 | 1.00 | 21.13 | O |
| ATOM | 4465 | O | HOH | W | 205 | 69.650 | 90.264 | 20.249 | 1.00 | 25.92 | O |
| ATOM | 4466 | O | HOH | W | 206 | 75.713 | 82.524 | 5.830 | 1.00 | 19.46 | O |
| ATOM | 4467 | O | HOH | W | 207 | 47.260 | 68.787 | 16.787 | 1.00 | 23.79 | O |
| ATOM | 4468 | O | HOH | W | 208 | 57.044 | 54.098 | 29.348 | 1.00 | 27.99 | O |
| ATOM | 4469 | O | HOH | W | 209 | 101.703 | 64.874 | 29.441 | 1.00 | 35.49 | O |
| ATOM | 4470 | O | HOH | W | 210 | 87.050 | 58.665 | 37.767 | 1.00 | 28.29 | O |
| ATOM | 4471 | O | HOH | W | 211 | 84.684 | 88.336 | 19.151 | 1.00 | 28.61 | O |
| ATOM | 4472 | O | HOH | W | 212 | 73.249 | 79.393 | 37.007 | 1.00 | 36.47 | O |
| ATOM | 4473 | O | HOH | W | 213 | 59.038 | 83.563 | 5.692 | 1.00 | 35.37 | O |
| ATOM | 4474 | O | HOH | W | 214 | 67.372 | 56.095 | 9.127 | 1.00 | 31.02 | O |
| ATOM | 4475 | O | HOH | W | 215 | 83.573 | 74.823 | −2.801 | 1.00 | 19.22 | O |
| ATOM | 4476 | O | HOH | W | 216 | 68.447 | 47.207 | 26.924 | 1.00 | 42.51 | O |
| ATOM | 4477 | O | HOH | W | 217 | 81.010 | 91.223 | 14.982 | 1.00 | 30.59 | O |
| ATOM | 4478 | O | HOH | W | 218 | 87.303 | 68.371 | −2.354 | 1.00 | 27.16 | O |
| ATOM | 4479 | O | HOH | W | 219 | 110.154 | 50.325 | 17.049 | 1.00 | 47.88 | O |
| ATOM | 4480 | O | HOH | W | 220 | 60.787 | 73.335 | −1.445 | 1.00 | 24.21 | O |
| ATOM | 4481 | O | HOH | W | 221 | 123.179 | 57.853 | 6.627 | 1.00 | 26.28 | O |
| ATOM | 4482 | O | HOH | W | 222 | 50.620 | 61.766 | 13.066 | 1.00 | 28.43 | O |
| ATOM | 4483 | O | HOH | W | 223 | 94.515 | 48.871 | −1.271 | 1.00 | 39.99 | O |
| ATOM | 4484 | O | HOH | W | 224 | 65.325 | 42.750 | 11.974 | 1.00 | 26.08 | O |
| ATOM | 4485 | O | HOH | W | 225 | 90.620 | 42.752 | 14.084 | 1.00 | 28.44 | O |
| ATOM | 4486 | O | HOH | W | 226 | 97.230 | 50.969 | 1.379 | 1.00 | 26.27 | O |
| ATOM | 4487 | O | HOH | W | 227 | 87.263 | 60.639 | −1.472 | 1.00 | 22.95 | O |
| ATOM | 4488 | O | HOH | W | 228 | 71.204 | 48.313 | 26.160 | 1.00 | 37.66 | O |
| ATOM | 4489 | O | HOH | W | 229 | 47.164 | 73.476 | 16.870 | 1.00 | 27.02 | O |
| ATOM | 4490 | O | HOH | W | 230 | 57.026 | 82.251 | 5.185 | 1.00 | 35.48 | O |
| ATOM | 4491 | O | HOH | W | 231 | 121.634 | 72.538 | 11.669 | 1.00 | 22.09 | O |
| ATOM | 4492 | O | HOH | W | 232 | 63.830 | 58.417 | 37.824 | 1.00 | 22.78 | O |
| ATOM | 4493 | O | HOH | W | 233 | 109.779 | 64.696 | 25.610 | 1.00 | 27.71 | O |
| ATOM | 4494 | O | HOH | W | 234 | 127.112 | 62.797 | 3.298 | 1.00 | 21.77 | O |
| ATOM | 4495 | O | HOH | W | 235 | 92.909 | 70.007 | 29.855 | 1.00 | 19.69 | O |
| ATOM | 4496 | O | HOH | W | 236 | 53.860 | 79.833 | 30.827 | 1.00 | 25.84 | O |
| ATOM | 4497 | O | HOH | W | 237 | 76.482 | 90.500 | 18.681 | 1.00 | 25.12 | O |
| ATOM | 4498 | O | HOH | W | 238 | 90.052 | 68.736 | 1.081 | 1.00 | 19.11 | O |
| ATOM | 4499 | O | HOH | W | 239 | 86.447 | 86.342 | 17.699 | 1.00 | 49.63 | O |
| ATOM | 4500 | O | HOH | W | 240 | 96.085 | 44.177 | 17.449 | 1.00 | 42.22 | O |
| ATOM | 4501 | O | HOH | W | 241 | 66.922 | 49.221 | 7.857 | 1.00 | 47.05 | O |
| ATOM | 4502 | O | HOH | W | 242 | 106.498 | 61.383 | 25.442 | 1.00 | 28.21 | O |
| ATOM | 4503 | O | HOH | W | 243 | 100.486 | 45.422 | 4.123 | 1.00 | 27.75 | O |
| ATOM | 4504 | O | HOH | W | 244 | 58.307 | 51.452 | 28.048 | 1.00 | 35.59 | O |
| ATOM | 4505 | O | HOH | W | 245 | 85.984 | 79.272 | 1.854 | 1.00 | 26.64 | O |
| ATOM | 4506 | O | HOH | W | 246 | 84.234 | 69.367 | −5.526 | 1.00 | 29.58 | O |
| ATOM | 4507 | O | HOH | W | 247 | 87.115 | 52.747 | 35.439 | 1.00 | 34.73 | O |
| ATOM | 4508 | O | HOH | W | 248 | 70.792 | 87.469 | 26.838 | 1.00 | 26.97 | O |
| ATOM | 4509 | O | HOH | W | 249 | 96.459 | 49.952 | 5.974 | 1.00 | 21.53 | O |
| ATOM | 4510 | O | HOH | W | 250 | 47.767 | 70.550 | 13.770 | 1.00 | 29.95 | O |
| ATOM | 4511 | O | HOH | W | 251 | 86.560 | 73.668 | 39.293 | 1.00 | 47.51 | O |
| ATOM | 4512 | O | HOH | W | 252 | 45.832 | 61.785 | 15.244 | 1.00 | 21.66 | O |
| ATOM | 4513 | O | HOH | W | 253 | 108.580 | 44.328 | 14.260 | 1.00 | 35.28 | O |
| ATOM | 4514 | O | HOH | W | 254 | 95.902 | 46.926 | 2.969 | 1.00 | 34.16 | O |
| ATOM | 4515 | O | HOH | W | 255 | 56.550 | 63.161 | 32.245 | 1.00 | 37.13 | O |
| ATOM | 4516 | O | HOH | W | 256 | 60.187 | 76.165 | −0.446 | 1.00 | 35.06 | O |
| ATOM | 4517 | O | HOH | W | 257 | 72.684 | 70.326 | 36.840 | 1.00 | 25.08 | O |
| ATOM | 4518 | O | HOH | W | 258 | 54.750 | 60.357 | 10.750 | 1.00 | 18.22 | O |
| ATOM | 4519 | O | HOH | W | 259 | 52.763 | 72.610 | 5.100 | 1.00 | 50.83 | O |
| ATOM | 4520 | O | HOH | W | 260 | 117.089 | 79.182 | 5.585 | 1.00 | 22.93 | O |
| ATOM | 4521 | O | HOH | W | 261 | 103.172 | 74.524 | 24.878 | 1.00 | 41.61 | O |
| ATOM | 4522 | O | HOH | W | 262 | 72.037 | 43.960 | 19.954 | 1.00 | 26.38 | O |
| ATOM | 4523 | O | HOH | W | 263 | 79.804 | 50.175 | 7.642 | 1.00 | 31.24 | O |
| ATOM | 4524 | O | HOH | W | 264 | 84.826 | 46.412 | 11.054 | 1.00 | 32.09 | O |
| ATOM | 4525 | O | HOH | W | 265 | 98.104 | 77.912 | 6.547 | 1.00 | 30.32 | O |
| ATOM | 4526 | O | HOH | W | 266 | 62.993 | 82.801 | 7.209 | 1.00 | 17.64 | O |
| ATOM | 4527 | O | HOH | W | 267 | 67.160 | 45.671 | 23.036 | 1.00 | 17.48 | O |
| ATOM | 4528 | O | HOH | W | 268 | 88.333 | 80.250 | 27.199 | 1.00 | 21.70 | O |
| ATOM | 4529 | O | HOH | W | 269 | 65.119 | 65.664 | 5.972 | 1.00 | 42.90 | O |
| ATOM | 4530 | O | HOH | W | 270 | 97.353 | 78.743 | 9.238 | 1.00 | 21.99 | O |
| ATOM | 4531 | O | HOH | W | 271 | 73.949 | 91.306 | 18.938 | 1.00 | 36.21 | O |
| ATOM | 4532 | O | HOH | W | 272 | 91.649 | 76.339 | 26.681 | 1.00 | 22.47 | O |
| ATOM | 4533 | O | HOH | W | 273 | 69.562 | 90.168 | 17.110 | 1.00 | 32.94 | O |
| ATOM | 4534 | O | HOH | W | 274 | 125.965 | 68.961 | 22.198 | 1.00 | 53.30 | O |
| ATOM | 4535 | O | HOH | W | 275 | 89.524 | 60.937 | −4.011 | 1.00 | 36.66 | O |
| ATOM | 4536 | O | HOH | W | 276 | 73.712 | 71.887 | 25.613 | 1.00 | 11.78 | O |
| ATOM | 4537 | O | HOH | W | 277 | 71.697 | 72.645 | 23.573 | 1.00 | 11.89 | O |
| ATOM | 4538 | O | HOH | W | 278 | 69.689 | 71.131 | 22.527 | 1.00 | 9.20 | O |
| ATOM | 4539 | O | HOH | W | 279 | 67.801 | 71.824 | 20.327 | 1.00 | 11.05 | O |
| ATOM | 4540 | O | HOH | W | 280 | 83.110 | 63.463 | 16.520 | 1.00 | 14.63 | O |
| ATOM | 4541 | O | HOH | W | 281 | 84.730 | 61.296 | 16.825 | 1.00 | 13.02 | O |
| ATOM | 4542 | O | HOH | W | 282 | 82.330 | 64.752 | 14.305 | 1.00 | 11.14 | O |
| ATOM | 4543 | O | HOH | W | 283 | 79.849 | 65.958 | 14.376 | 1.00 | 12.95 | O |
| ATOM | 4544 | O | HOH | W | 284 | 77.982 | 64.918 | 12.545 | 1.00 | 9.14 | O |
| ATOM | 4545 | O | HOH | W | 285 | 87.321 | 60.005 | 15.505 | 1.00 | 10.28 | O |
| ATOM | 4546 | O | HOH | W | 286 | 86.315 | 57.787 | 16.752 | 1.00 | 7.53 | O |
| ATOM | 4547 | O | HOH | W | 287 | 84.249 | 54.156 | 16.640 | 1.00 | 12.46 | O |
| ATOM | 4548 | O | HOH | W | 288 | 84.416 | 51.453 | 16.932 | 1.00 | 13.71 | O |
| ATOM | 4549 | O | HOH | W | 289 | 54.396 | 71.736 | 18.322 | 1.00 | 9.78 | O |
| ATOM | 4550 | O | HOH | W | 290 | 53.429 | 69.712 | 19.975 | 1.00 | 14.49 | O |
| ATOM | 4551 | O | HOH | W | 291 | 60.518 | 74.256 | 19.629 | 1.00 | 12.46 | O |
| ATOM | 4552 | O | HOH | W | 292 | 61.215 | 72.779 | 21.734 | 1.00 | 10.28 | O |
| ATOM | 4553 | O | HOH | W | 293 | 73.011 | 64.617 | 26.681 | 1.00 | 11.58 | O |
| ATOM | 4554 | O | HOH | W | 294 | 70.799 | 63.140 | 27.201 | 1.00 | 11.06 | O |
| ATOM | 4555 | O | HOH | W | 295 | 75.251 | 65.494 | 28.347 | 1.00 | 10.71 | O |
| ATOM | 4556 | O | HOH | W | 296 | 70.115 | 59.305 | 30.735 | 1.00 | 16.03 | O |
| ATOM | 4557 | O | HOH | W | 297 | 69.647 | 58.706 | 33.452 | 1.00 | 10.27 | O |
| ATOM | 4558 | O | HOH | W | 298 | 99.000 | 55.080 | 10.817 | 1.00 | 11.69 | O |
| ATOM | 4559 | O | HOH | W | 299 | 100.425 | 53.056 | 12.524 | 1.00 | 12.45 | O |
| ATOM | 4560 | O | HOH | W | 300 | 66.242 | 65.428 | 14.776 | 1.00 | 16.43 | O |
| ATOM | 4561 | O | HOH | W | 301 | 81.405 | 80.063 | 12.880 | 1.00 | 12.57 | O |
| ATOM | 4562 | O | HOH | W | 302 | 79.412 | 80.396 | 11.081 | 1.00 | 10.27 | O |
| ATOM | 4563 | O | HOH | W | 303 | 68.824 | 60.209 | 41.465 | 1.00 | 31.92 | O |
| ATOM | 4564 | O | HOH | W | 304 | 59.552 | 55.604 | 29.512 | 1.00 | 17.67 | O |
| ATOM | 4565 | O | HOH | W | 305 | 60.083 | 55.562 | 36.754 | 1.00 | 33.95 | O |
| ATOM | 4566 | O | HOH | W | 306 | 69.237 | 44.346 | 23.441 | 1.00 | 24.40 | O |
| ATOM | 4567 | O | HOH | W | 307 | 69.770 | 40.875 | 12.372 | 1.00 | 26.72 | O |
| ATOM | 4568 | O | HOH | W | 308 | 95.206 | 62.760 | 30.095 | 1.00 | 25.02 | O |
| ATOM | 4569 | O | HOH | W | 309 | 95.950 | 59.484 | 34.012 | 1.00 | 27.04 | O |
| ATOM | 4570 | O | HOH | W | 310 | 56.711 | 66.265 | 31.461 | 1.00 | 30.50 | O |
| ATOM | 4571 | O | HOH | W | 311 | 55.298 | 65.451 | 28.963 | 1.00 | 25.06 | O |
| ATOM | 4572 | O | HOH | W | 312 | 90.647 | 71.911 | 33.467 | 1.00 | 23.47 | O |
| ATOM | 4573 | O | HOH | W | 313 | 50.077 | 73.625 | 22.368 | 1.00 | 19.98 | O |
| ATOM | 4574 | O | HOH | W | 314 | 49.389 | 72.201 | 24.596 | 1.00 | 35.15 | O |
| ATOM | 4575 | O | HOH | W | 315 | 58.934 | 84.452 | 11.723 | 1.00 | 22.56 | O |
| ATOM | 4576 | O | HOH | W | 316 | 69.713 | 73.707 | −3.172 | 1.00 | 14.53 | O |
| ATOM | 4577 | O | HOH | W | 317 | 71.077 | 73.328 | −0.554 | 1.00 | 16.15 | O |
| ATOM | 4578 | O | HOH | W | 318 | 64.017 | 79.240 | 0.019 | 1.00 | 26.81 | O |
| ATOM | 4579 | O | HOH | W | 319 | 79.020 | 83.776 | 31.548 | 1.00 | 29.75 | O |
| ATOM | 4580 | O | HOH | W | 320 | 85.533 | 76.663 | 33.378 | 1.00 | 30.19 | O |
| ATOM | 4581 | O | HOH | W | 321 | 73.452 | 69.182 | 7.589 | 1.00 | 17.09 | O |
| ATOM | 4582 | O | HOH | W | 322 | 71.448 | 86.128 | 10.831 | 1.00 | 19.34 | O |
| ATOM | 4583 | O | HOH | W | 323 | 83.503 | 89.728 | 21.490 | 1.00 | 22.84 | O |
| ATOM | 4584 | O | HOH | W | 324 | 87.715 | 86.894 | 21.851 | 1.00 | 23.54 | O |
| ATOM | 4585 | O | HOH | W | 325 | 79.121 | 60.500 | 5.937 | 1.00 | 27.41 | O |
| ATOM | 4586 | O | HOH | W | 326 | 86.368 | 65.385 | 0.562 | 1.00 | 27.35 | O |
| ATOM | 4587 | O | HOH | W | 327 | 95.444 | 50.430 | 8.518 | 1.00 | 15.26 | O |
| ATOM | 4588 | O | HOH | W | 328 | 94.301 | 57.944 | −3.286 | 1.00 | 29.04 | O |
| ATOM | 4589 | O | HOH | W | 329 | 116.537 | 52.721 | 3.638 | 1.00 | 27.13 | O |
| ATOM | 4590 | O | HOH | W | 330 | 76.275 | 59.378 | 7.337 | 1.00 | 40.19 | O |
| ATOM | 4591 | O | HOH | W | 331 | 70.747 | 63.822 | 13.042 | 1.00 | 28.63 | O |
| ATOM | 4592 | O | HOH | W | 332 | 84.440 | 47.986 | 34.116 | 1.00 | 18.43 | O |
| ATOM | 4593 | O | HOH | W | 333 | 83.154 | 43.293 | 29.434 | 1.00 | 25.96 | O |
| ATOM | 4594 | O | HOH | W | 334 | 98.697 | 47.505 | 21.570 | 1.00 | 27.02 | O |
| ATOM | 4595 | O | HOH | W | 335 | 102.772 | 52.282 | 20.064 | 1.00 | 32.70 | O |
| ATOM | 4596 | O | HOH | W | 336 | 110.632 | 58.836 | 4.205 | 1.00 | 26.47 | O |
| ATOM | 4597 | O | HOH | W | 337 | 111.849 | 61.745 | 3.152 | 1.00 | 44.94 | O |
| ATOM | 4598 | O | HOH | W | 338 | 101.125 | 49.787 | −0.988 | 1.00 | 33.96 | O |
| ATOM | 4599 | O | HOH | W | 339 | 117.159 | 66.545 | 21.836 | 1.00 | 21.84 | O |
| ATOM | 4600 | O | HOH | W | 340 | 120.244 | 56.609 | 12.191 | 1.00 | 23.32 | O |
| ATOM | 4601 | O | HOH | W | 341 | 115.170 | 51.278 | 14.257 | 1.00 | 30.44 | O |
| ATOM | 4602 | O | HOH | W | 342 | 111.491 | 80.833 | 3.165 | 1.00 | 29.82 | O |
| ATOM | 4603 | O | HOH | W | 343 | 101.953 | 82.610 | 10.773 | 1.00 | 25.43 | O |
| ATOM | 4604 | O | HOH | W | 344 | 46.011 | 67.990 | 20.238 | 1.00 | 19.80 | O |
| ATOM | 4605 | O | HOH | W | 345 | 115.554 | 79.243 | 14.726 | 1.00 | 22.39 | O |
| ATOM | 4606 | O | HOH | W | 346 | 87.673 | 44.927 | 20.726 | 1.00 | 20.86 | O |
| ATOM | 4607 | O | HOH | W | 347 | 57.443 | 78.585 | 2.560 | 1.00 | 23.83 | O |
| ATOM | 4608 | O | HOH | W | 348 | 60.383 | 74.826 | 31.955 | 1.00 | 23.36 | O |
| ATOM | 4609 | O | HOH | W | 349 | 108.102 | 83.941 | 7.708 | 1.00 | 25.74 | O |
| ATOM | 4610 | O | HOH | W | 350 | 86.983 | 47.544 | 34.355 | 1.00 | 26.63 | O |
| ATOM | 4611 | O | HOH | W | 351 | 82.239 | 52.554 | 8.350 | 1.00 | 23.73 | O |
| ATOM | 4612 | O | HOH | W | 352 | 79.115 | 47.275 | 26.274 | 1.00 | 27.40 | O |
| ATOM | 4613 | O | HOH | W | 353 | 50.652 | 70.037 | 24.709 | 1.00 | 25.78 | O |
| ATOM | 4614 | O | HOH | W | 354 | 114.054 | 65.476 | 22.808 | 1.00 | 25.31 | O |
| ATOM | 4615 | O | HOH | W | 355 | 111.136 | 48.957 | 4.228 | 1.00 | 26.53 | O |
| ATOM | 4616 | O | HOH | W | 356 | 104.951 | 81.706 | 2.014 | 1.00 | 23.31 | O |
| ATOM | 4617 | O | HOH | W | 357 | 117.608 | 69.017 | 3.208 | 1.00 | 26.80 | O |
| ATOM | 4618 | O | HOH | W | 358 | 60.255 | 54.205 | 15.233 | 1.00 | 21.92 | O |
| ATOM | 4619 | O | HOH | W | 359 | 125.742 | 61.809 | 20.938 | 1.00 | 23.68 | O |
| ATOM | 4620 | O | HOH | W | 360 | 71.318 | 45.857 | 21.866 | 1.00 | 28.16 | O |
| ATOM | 4621 | O | HOH | W | 361 | 64.336 | 48.484 | 29.783 | 1.00 | 25.84 | O |
| ATOM | 4622 | O | HOH | W | 362 | 64.410 | 70.021 | 33.218 | 1.00 | 28.32 | O |
| ATOM | 4623 | O | HOH | W | 363 | 99.662 | 51.728 | 26.853 | 1.00 | 25.37 | O |
| ATOM | 4624 | O | HOH | W | 364 | 57.619 | 85.004 | 23.472 | 1.00 | 26.78 | O |
| ATOM | 4625 | O | HOH | W | 365 | 109.513 | 85.026 | 14.107 | 1.00 | 31.48 | O |
| ATOM | 4626 | O | HOH | W | 366 | 71.356 | 78.874 | 7.358 | 1.00 | 27.32 | O |
| ATOM | 4627 | O | HOH | W | 367 | 56.265 | 89.128 | 21.290 | 1.00 | 31.78 | O |
| ATOM | 4628 | O | HOH | W | 368 | 115.927 | 67.059 | −0.659 | 1.00 | 28.23 | O |
| ATOM | 4629 | O | HOH | W | 369 | 86.347 | 87.208 | 6.215 | 1.00 | 28.36 | O |
| ATOM | 4630 | O | HOH | W | 370 | 100.399 | 47.343 | 16.164 | 1.00 | 28.80 | O |
| ATOM | 4631 | O | HOH | W | 371 | 125.082 | 66.017 | 15.408 | 1.00 | 29.06 | O |
| ATOM | 4632 | O | HOH | W | 372 | 106.891 | 75.678 | 3.454 | 1.00 | 25.59 | O |
| ATOM | 4633 | O | HOH | W | 373 | 88.447 | 43.131 | 24.920 | 1.00 | 29.14 | O |
| ATOM | 4634 | O | HOH | W | 374 | 48.419 | 79.853 | 13.172 | 1.00 | 26.52 | O |
| ATOM | 4635 | O | HOH | W | 375 | 47.642 | 75.779 | 22.363 | 1.00 | 34.45 | O |
| ATOM | 4636 | O | HOH | W | 376 | 114.861 | 82.439 | 9.087 | 1.00 | 35.35 | O |
| ATOM | 4637 | O | HOH | W | 377 | 74.958 | 81.956 | 35.600 | 1.00 | 28.35 | O |
| ATOM | 4638 | O | HOH | W | 378 | 111.202 | 79.501 | 1.078 | 1.00 | 32.07 | O |
| ATOM | 4639 | O | HOH | W | 379 | 97.594 | 63.105 | 29.216 | 1.00 | 30.45 | O |
| ATOM | 4640 | O | HOH | W | 380 | 89.237 | 84.051 | 11.598 | 1.00 | 28.28 | O |
| ATOM | 4641 | O | HOH | W | 381 | 97.163 | 60.858 | 31.713 | 1.00 | 37.20 | O |
| ATOM | 4642 | O | HOH | W | 382 | 111.105 | 76.736 | 1.164 | 1.00 | 37.60 | O |
| ATOM | 4643 | O | HOH | W | 383 | 89.108 | 80.551 | 20.138 | 1.00 | 31.32 | O |
| ATOM | 4644 | O | HOH | W | 384 | 109.208 | 54.650 | 0.012 | 0.50 | 20.78 | O |
| ATOM | 4645 | O | HOH | W | 385 | 56.533 | 53.431 | 9.966 | 1.00 | 29.03 | O |
| ATOM | 4646 | O | HOH | W | 386 | 70.988 | 87.241 | 12.976 | 1.00 | 30.02 | O |
| ATOM | 4647 | O | HOH | W | 387 | 59.625 | 58.605 | 35.175 | 1.00 | 33.71 | O |
| ATOM | 4648 | O | HOH | W | 388 | 86.544 | 43.124 | 22.806 | 1.00 | 33.84 | O |
| ATOM | 4649 | O | HOH | W | 389 | 87.274 | 64.588 | 39.751 | 1.00 | 33.06 | O |
| ATOM | 4650 | O | HOH | W | 390 | 72.877 | 87.061 | 6.624 | 1.00 | 32.33 | O |
| ATOM | 4651 | O | HOH | W | 391 | 71.897 | 80.278 | 5.628 | 1.00 | 26.70 | O |
| ATOM | 4652 | O | HOH | W | 392 | 69.516 | 42.838 | 9.000 | 1.00 | 32.63 | O |
| ATOM | 4653 | O | HOH | W | 393 | 109.629 | 83.070 | 9.904 | 1.00 | 30.86 | O |
| ATOM | 4654 | O | HOH | W | 394 | 78.125 | 92.023 | 24.704 | 1.00 | 30.52 | O |
| ATOM | 4655 | O | HOH | W | 395 | 78.953 | 91.893 | 22.367 | 1.00 | 27.36 | O |
| ATOM | 4656 | O | HOH | W | 396 | 77.655 | 92.424 | 20.194 | 1.00 | 27.78 | O |
| ATOM | 4657 | O | HOH | W | 397 | 95.558 | 64.394 | 32.011 | 1.00 | 41.48 | O |
| ATOM | 4658 | O | HOH | W | 398 | 68.942 | 73.097 | 35.691 | 1.00 | 31.00 | O |
| ATOM | 4659 | O | HOH | W | 399 | 123.235 | 58.210 | 19.901 | 1.00 | 37.19 | O |
| ATOM | 4660 | O | HOH | W | 400 | 81.026 | 75.783 | 39.765 | 1.00 | 33.48 | O |
| ATOM | 4661 | O | HOH | W | 401 | 89.218 | 44.167 | 31.825 | 1.00 | 29.29 | O |
| ATOM | 4662 | O | HOH | W | 402 | 75.739 | 46.600 | 22.208 | 1.00 | 32.70 | O |
| ATOM | 4663 | O | HOH | W | 403 | 93.604 | 58.674 | 35.287 | 1.00 | 33.41 | O |
| ATOM | 4664 | O | HOH | W | 404 | 114.143 | 53.316 | 2.795 | 1.00 | 36.89 | O |
| ATOM | 4665 | O | HOH | W | 405 | 62.314 | 77.901 | −1.690 | 1.00 | 31.79 | O |
| ATOM | 4666 | O | HOH | W | 406 | 119.132 | 74.672 | 20.486 | 1.00 | 38.01 | O |
| ATOM | 4667 | O | HOH | W | 407 | 89.827 | 72.251 | 40.058 | 1.00 | 37.03 | O |
| ATOM | 4668 | O | HOH | W | 408 | 62.078 | 85.872 | 25.358 | 1.00 | 38.89 | O |
| ATOM | 4669 | O | HOH | W | 409 | 66.149 | 86.250 | 27.163 | 1.00 | 39.99 | O |
| ATOM | 4670 | O | HOH | W | 410 | 111.238 | 48.481 | 15.078 | 1.00 | 38.99 | O |
| ATOM | 4671 | O | HOH | W | 411 | 122.333 | 65.281 | 8.119 | 1.00 | 26.63 | O |
| ATOM | 4672 | O | HOH | W | 412 | 104.767 | 59.363 | 26.560 | 1.00 | 33.01 | O |
| ATOM | 4673 | O | HOH | W | 413 | 91.741 | 81.149 | 13.569 | 1.00 | 36.22 | O |

1. An isolated nucleic acid comprising the nucleotide sequence of SEQ ID NO: 1 or of a degenerate variant of SEQ ID NO: 1.

2. An isolated nucleic acid comprising a sequence that encodes a polypeptide consisting of the amino acid sequence of SEQ ID NO: 3.

3. An isolated nucleic acid comprising a sequence that hybridizes under stringent conditions to a hybridization probe the nucleotide sequence of which consists of SEQ ID NO: 1, or the complement of SEQ ID NO: 1.

4. An isolated nucleic acid comprising a sequence at least 66%, 67%, 68%, 69%, 70%, 75%, 80%, 85%, 90%, 95%, 98%, 99%, or 99.5% identical to SEQ ID NO: 1.

5. The isolated nucleic acid of claim 4 wherein the nucleic acid encodes a polypeptide that has starch hydrolysis activity.

6. An isolated nucleic acid comprising a sequence that encodes a polypeptide at least 66%, 67%, 68%, 69%, 70%, 75%, 80%, 85%, 90%, 95%, 98%, 99%, or 99.5% identical to SEQ ID NO: 3, wherein the polypeptide has starch hydrolysis activity.

7. An isolated nucleic acid comprising a sequence that encodes a polypeptide comprising the sequence of SEQ ID NO: 3, or SEQ ID NO: 3 with up to 50 conservative amino acid substitutions, wherein the polypeptide has starch hydrolysis activity.

8. A purified polypeptide, the amino acid sequence of which comprises a sequence at least 66%, 67%, 68%, 69%, 70%, 75%, 80%, 85%, 90%, 95%, 98%, 99%, or 99.5% identical to SEQ ID NO: 3.

9. A purified polypeptide comprising the amino acid sequence of SEQ ID NO: 3, but with 0 to 20 conservative amino acid substitutions.

10. An expression vector comprising the nucleic acid sequence of claim 1 operably linked to an expression control sequence.

11. A cultured cell comprising the vector of claim 10.

12. A cultured cell comprising the nucleic acid of claim 1 operably linked to an expression control sequence.

13. A cultured cell transfected with the vector of claim 10, or a progeny of said cell, wherein the cell expresses the nucleic acid to form a polypeptide.

14. A method of producing a protein, the method comprising culturing the cell of claim 11 under conditions permitting expression of the polypeptide.

15. A method of using the polypeptide of claim 8, the method comprising including the polypeptide in any of: starch liquefaction, starch saccharification, textile desizing, starch modification in the paper and pulp industry, brewing, baking, production of syrups for the food industry, production of feedstocks for fermentation processes, animal feed, and, removal of starchy soils and/or stains during dishwashing and/or laundry washing.

16. A composition comprising the polypeptide of claim 8, and at least one accessory enzyme selected from the group consisting of phytase, protease, pullulanase, β-amylase, isoamylase, a different amylase, alpha-glucosidase, cellulase, xylanase, hemicellulase, beta-glucosidase, transferase, pectinase, lipase, cutinase, esterase, choline oxidases, peroxidases/oxidases, pectate lyases, mannanases, cutinases, laccases, phospholipases, lysophospholipases, acyltransferases, perhydrolases, arylesterases, and redox enzymes.


SEQUENCE LISTING (6 sequences)

SEQ ID NO: 1 - DNA, 1659 bp, Artificial Sequence
  Synthetic full-length nucleotide sequence for AmyMG
cacaccccgacgacccggcaggccgattactacggcacgctggagccgtttgcgcgtgaagcggtgtacttcgtgatgaccgatcgcttcgtcaacggcgaccccggcaacgaccaccgcgaccaaggcggcgccctgggcacgttcgacatcccgctgccgccatgcaatggcgtgtccggcaacatcggctacctgggtggcgacttcaagggcctggccgatcatctggattacatccgcgaaatgggcttcaccgcggtgtggatcacgccgatcgtggacaatccggaccagcgcttcactggcggcagcgcaccaacctgcggcggcattctggctgaccagggcaaagccggctatcacggctactggggcgtgaatttctaccaagtggacgagcacctgcccagcccaggcatggacttccgcgacctggcggcggcgatgcatcgcaagggcatgaagctggtgctggacatcgtggggaaccacggctcgccggcctggggcatggccttcgaccagcccaagttcggcaagatctacgacaaggacggcacgctgattgccgatcaccagaacctgccgccgcagcagctggatcccgagcacaacccgctgcaccgcttctacaacacggtcggcccggtggacggggccaagggatcgatcttcgacggcaatctggcccagctgtcggatctcaatgaacgcaacccggacgtgctggactatctggtcggggcctatctgcaatggatcgaccagggtgccgatgcgtttcgcatcgacaccatcgcctggatgccggacagcttctggcaggccttcaccacccgcatccgggcaaagcaccccggctttttcatgttcggcgaggccttcgactacgacgccgccaggattgccacccacaccctgcccggccacggcgaaaccagcgtgctggacttcccgatgaaacaggcgatggaagaggtcttcgggcgcaagcaggccggttttgaacggatgataccggcgctgcatctgactggcggcccgtatgccaacccctacgagctggccaccttctacgacaatcacgacatgccgcggctggatgccagcgatgaaggcttcattgatgcacacaactggctgttcaccgcgcgtggcattccggtggtctattacggctcggaaatgggcttcatgcgcggccgacccgagcacggcggcaaccgcaactacttcggcaccgaaggcattgccgccgccaaggcaagcccgatccgggcagcgctgacccgcattgcgcaggtgcgtgccgcttcaccagcgctgcagcgcgggctgcaactcaatctggagctgcaaggcaaccgcgccgcgttctatcgggtgtaccagcacgacggtgtgcaccagatcgcgctggtcctgctcaacaagggcgacgccccggaacactttgccgtccagacgatgctgcaacccggccgctggcatgacgcgattggcggtgagacgctgaccatacaggctggcgaggcgctgcacgccgaggttccggcgcatggcgtgcgggtctttctgctcgacgcccaggtgactgagccgacgctggcggctgcgctggatgccgccatggccgatgcacgccggtcacggtaa

SEQ ID NO: 2 - DNA, 75 bp, Artificial Sequence
  Synthetic nucleotide sequence for the native signal sequence for AmyMG
atgatcaccatgcccctgcgttctgcccgcctcggcctgagtttgctttgcgcgcttgcctcgacggcctgtgca

SEQ ID NO: 3 - protein, 552 aa, Artificial Sequence
  Synthetic full-length amino acid sequence for AmyMG
HTPTTRQADYYGTLEPFAREAVYFVMTDRFVNGDPGNDHRDQGGALGTFDIPLPPCNGVSGNIGYLGGDFKGLADHLDYIREMGFTAVWITPIVDNPDQRFTGGSAPTCGGILADQGKAGYHGYWGVNFYQVDEHLPSPGMDFRDLAAAMHRKGMKLVLDIVGNHGSPAWGMAFDQPKFGKIYDKDGTLIADHQNLPPQQLDPEHNPLHRFYNTVGPVDGAKGSIFDGNLAQLSDLNERNPDVLDYLVGAYLQWIDQGADAFRIDTIAWMPDSFWQAFTTRIRAKHPGFFMFGEAFDYDAARIATHTLPGHGETSVLDFPMKQAMEEVFGRKQAGFERMIPALHLTGGPYANPYELATFYDNHDMPRLDASDEGFIDAHNWLFTARGIPVVYYGSEMGFMRGRPEHGGNRNYFGTEGIAAAKASPIRAALTRIAQVRAASPALQRGLQLNLELQGNRAAFYRVYQHDGVHQIALVLLNKGDAPEHFAVQTMLQPGRWHDAIGGETLTIQAGEALHAEVPAHGVRVFLLDAQVTEPTLAAALDAAMADARRSR

SEQ ID NO: 4 - protein, 25 aa, Artificial Sequence
  Synthetic amino acid sequence for the native signal peptide
MITMPLRSARLGLSLLCALASTACA

SEQ ID NO: 5 - DNA, 96 bp, Artificial Sequence
  Synthetic construct aprE signal nucleic acid sequence plus AGK nucleic acid sequence
gtgagaagcaaaaaattgtggatcagcttgttgtttgcgttaacgttaatctttacgatggcgttcagcaacatgagcgcgcaggcagctggtaaa

SEQ ID NO: 6 - protein, 33 aa, Artificial Sequence
  Synthetic construct aprE signal amino acid sequence plus AGK amino acid sequence
MRSKKLWISLLFALALIFTMAFGSTSSAQAAGK